SEQUENCE LISTING (19 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Simian virus 40
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2022/0226464 A1
(43) Pub. Date: Jul. 21, 2022

(54) METHODS AND COMPOSITIONS FOR MODULATING IMMUNE RESPONSES

(71) Applicant: Massachusetts Institute of Technology, Cambridge, MA (US)

(72) Inventors: Samuel Kazer, Cambridge, MA (US); Alexander K. Shalek, Cambridge, MA (US)

(21) Appl. No.: 17/614,602

(22) PCT Filed: May 28, 2020
(86) PCT No.: PCT/US2020/035008

§ 371 Date: Nov. 29, 2021

Related U.S. Application Data
(60) Provisional application No. 62/853,644, filed on May 28, 2019.

(51) Int. Cl. A61K 39/21; A61K 38/46; A61K 31/7088; A61K 39/39; A61P 31/18; A61P 37/04

(52) U.S. Cl. CPC A61K 39/21; A61K 38/465; A61P 37/04; A61K 39/39; A61P 31/18; A61K 31/7088

(57) ABSTRACT

A method of modulating immune response to an infection in a subject, the method comprising contacting CD4+ T cells, monocytes, cytotoxic lymphocytes (CTLs), natural killer (NK) cells, and/or proliferating T cells in the subject with one or more modulating agents, wherein the one or more modulating agents modulate genes in one or more of pathways and cell populations.

### CROSS-REFERENCE TO RELATED APPLICATION

This application claims the benefit of U.S. Provisional Application No. 62/853,644, filed May 28, 2019. The entire contents of the above-identified application are hereby fully incorporated herein by reference.

### STATEMENT REGARDING FEDERALLY SPONSORED RESEARCH

This invention was made with government support under Grant Nos. AI089992, HL095791, CA217377, AI039671, AI118672, HG006193, CA202820, AI138546, AI067073, HL126554, DA046277, CA233195, and AI118538 awarded by the National Institutes of Health. The government has certain rights in the invention.

### REFERENCE TO AN ELECTRONIC SEQUENCE LISTING

The contents of the electronic sequence listing (“BROD-4250WP_ST25.txt”; 7804 bytes; created on May 5, 2020) is herein incorporated by reference in its entirety.

### TECHNICAL FIELD

The subject matter disclosed herein is generally directed to methods and compositions for modulating immune responses.

### BACKGROUND

Understanding the dynamics of host-pathogen interactions during acute viral infection in humans has been hindered by limited sample availability and technical complications associated with comprehensively profiling heterogeneous cellular ensembles. A better understanding of the interplay between innate and adaptive immune responses at the early stages of a viral infection, and its impact on long-term disease, could reveal principles to accelerate prevention efforts. Human Immunodeficiency Virus (HIV) has been the subject of thorough study and thus is a well-considered model system for examining host responses to a pathogen. Moreover, although the development of antiretroviral therapy (ART), as well as implementation of pre-exposure prophylaxis (PrEP) and combination prevention efforts, has improved the lives of persons living with HIV, increased life expectancies, and reduced the number of new infections, there were still two million new cases of HIV-1 infection in 2017. This highlights a pressing need for effective HIV vaccines informed by an understanding of natural host-pathogen dynamics.

### SUMMARY

In one aspect, the present disclosure provides a method of treating or preventing a viral infection, the method comprising administrating an effective amount of an modulating agent that induces proliferation of γδ T cells and/or Natural killer (NK) cells to a subject in need thereof. In some embodiments, the viral infection is a chronic viral infection. In some embodiments, the viral infection is a human immunodeficiency virus (HIV) infection.

In another aspect, the present disclosure provides a method of treating or preventing a viral infection, the method comprising administrating an effective amount of a vaccine composition to a subject in need thereof, the vaccine composition comprising one or more modulating agents that induces proliferation of γδ T cells and/or NK cells. In some embodiments, the one or more modulating agent modulates one or more biomarkers GAPDH, STMN1, KIAA0101, MKI67, MALAT1, TXNIP, IL7R, and KLRB1. In some embodiments, the one or more modulating agents increases KLRB1 expression in the γδ T cells and/or NK cells.

In another aspect, the present disclosure provides method of modulating an immune response to reduce baseline inflammation comprising administrating an effective amount of one or more modulating agents that increases expression or activity of APOBEC3A, IFITM1, IFITM3, or a combination thereof in one or more immune cells.

In some embodiments, the one or more immune cells comprise monocytes, CD4+ T cells, cytotoxic T lymphocytes (CTLs), proliferating T cells, NK cells, B cells, plasmablasts, and myeloid dendritic cells. In some embodiments, the immune response is to a viral infection. In some embodiments, the viral infection is an HIV infection.

In another aspect, the present disclosure provides a method of modulating an immune response comprising administering an effective amount of one or more modulating agents that increases activity or expression of PRF1 and/or GZMB in proliferating CTLs and/or one or more modulating agents that increases activity or expression of CCL3 and/or CCL4 in NK cells. In some embodiments, the immune response is to a viral infection. In some embodiments, the viral infection is an HIV infection.

In another aspect, the present disclosure provides a method of modulating an immune response comprising administering one or more modulating agents that induces formation of polyfunctional monocytes. In some embodiments, the polyfunctional monocytes express one or more anti-viral and inflammatory expression modules. In some embodiments, the one or more anti-viral and inflammatory expression modules comprise RIG-1, STAT1, HLA-G, APOBEC3B, ISG20, MX1, ISG15, IFI27, or a combination thereof. In some embodiments, the one or more anti-viral and inflammatory expression modules comprise RIG-1, APOBEC3B, MX1, or a combination thereof. In some embodiments, the one or more anti-viral and inflammatory expression modules comprise SLAMF7, DUSP6, WARS, USP18, or a combination thereof.

In another aspect, the present disclosure provides a method of treating or preventing a viral infection, the method comprising administrating an effective amount of an modulating agent that modulate expression and/or activity of IL-6, IL-8, IL-17, or a combination thereof to a subject in need thereof.

In another aspect, the present disclosure provides a method of treating or preventing a viral infection, the method comprising administrating an effective amount of: one or more modulating agents that modulate IFN-α, IFN-γ, or a combination thereof in proliferating T cells, CD4+ T cells, CTLs, monocytes, and NK cells; one or more modulating agents that modulate IL-15, IL-12, IL-21, or a combination thereof in CTLs, NK cells, and proliferating T cells; one or more modulating agents that modulate IL-1β, TNF, or a combination thereof in CD4+ T cells; or any combination thereof.

In another aspect, the present disclosure provides a method of detecting a stage of viral infection comprising: detecting an expression level of IFI44IL, IFI6, IFIT3, ISG15, XAF1, APOBEC3A, IF27, STAT1 or a combination thereof, wherein the expression level relative to a suitable control indicates a hyper-acute or acute stage of viral infection.

In some embodiments, the method further comprises detection of CXCL10, DEFB1, IFI27L1, or a combination thereof. In some embodiments, the method further comprises detection of PARP9, STAT1, or a combination thereof. In some embodiments, the method further comprises detection of CD52, TIGIT, TRAC, or a combination thereof. In some embodiments, the method further comprises detection of CX3CR1, ICAM2, or a combination thereof. In some embodiments, the method further comprises detection of SLAMF7, DUSP6, WARS, USP18, or a combination thereof. In some embodiments, the method further comprises administering one or more modulating agents to modulate expression and/or activity of the detected one or more biomarkers.

In another aspect, the present disclosure provides a method for treating a subject with an infection, the method comprising: a. detecting expression or activity of one or more biomarkers in one or more types of immune cells; b. administering one or more modulating agents to modulate expression and/or activity of the detected one or more biomarkers.

In another aspect, the present disclosure provides a method of screening for one or more agents capable of modulating an immune response, the method comprising: a. contacting one or more immune cells with one or more candidate modulating agents; b. detecting expression and/or activity of one or more biomarkers in response to the one or more candidate modulating agents; and c. selecting modulating agents that cause change in expression and/or activity of one or more biomarkers compared to expression and/or activity of the one or more biomarkers before (a). In some embodiments, the immune response is to an viral infection. In some embodiments, the one or more immune cells comprise CD4+ T cells, cytotoxic T lymphocytes (CTLs), proliferating T cells, NK cells, B cells, plasmablasts, myeloid dendritic cells, or a combination thereof.

In another aspect, the present disclosure provides a method of modulating immune response to an infection in a subject, the method comprising contacting CD4+ T cells, monocytes, cytotoxic lymphocytes (CTLs), natural killer (NK) cells, and/or proliferating T cells in the subject with one or more modulating agents, wherein the one or more modulating agents modulate biomarkers in one or more of the following pathways and cell populations: a. adhesion of T cells, Cdc42 signaling, cytokine signaling, regulation by calpain, endocytic virus entry, or a combination thereof, in CD+4 T cells; b. allograft rejection signaling, Cdc42 signaling, antigen presentation, IL-4 signaling, OX40 signaling, or a combination thereof, in monocytes; c. CTL killing or target cells, graft-vs-host disease signaling, Granzyme B signaling, interferon signaling, hypercytokinemia in flu, or a combination thereof, in CTLs; d. chemokinesis of leukocytes, CTL killing of target cells, innate-adaptive crosstalk, OX40 signaling, dendric cell (DC)-NK crosstalk, or a combination thereof, in NK cells; or e. innate-adaptive crosstalk, CTL killing of target cells, degranulation of cells, granzyme B signaling, and interferon signaling, or a combination thereof, in proliferating T cells.

In some embodiments, the one or more modulating agents further modulate biomarkers in one or more pathways in Table 6A. In some embodiments, the one or more modulating agents modulate a. biomarkers in cluster 1 of Table 2 in CD4+ T cells; b. biomarkers in cluster 2 of Table 2 in resting monocytes; c. biomarkers in cluster 3 of Table 2 in cytotoxic lymphocytes; d. biomarkers in cluster 4 of Table 2 in inflammatory monocytes; e. biomarkers in cluster 5 of Table 2 in B cells; f. biomarkers in cluster 6 of Table 2 in non-classical monocytes; g. biomarkers in cluster 7 of Table 2 in proliferating T cells; h. biomarkers in cluster 8 of Table 2 in anti-viral monocytes; i. biomarkers in cluster 9 of Table 2 in plasmablasts; j. biomarkers in cluster 10 of Table 2 in CD1C+ dendric cells (DCs); k. biomarkers in cluster 11 of Table 2 in both anti-viral monocytes and inflammatory monocytes; 1. biomarkers in cluster 12 of Table 2 in CD1C+ plasmacytoid dendric cells (pDCs); or m. any combination thereof.

In some embodiments, the one or more modulating agents modulate: a. biomarkers in one or more of the following modules in Tables 3A-3D: P1.B.M1, P1.B.M2, P1.B.M3, P2.B.M1, P2.B.M2, P2.B.M3, P2.B.M4, P3.B.M1, P3.B.M2, P3.B.M3, P3.B.M4, P3.B.M5, P4.B.M1, P4.B.M2, in B cells; b. biomarkers one or more of the following modules in Tables 3A-3D: P1.CD4.M1, P1.CD4.M2, P1.CD4.M3, P1.CD4.M4, P1.CD4.M5, P1.CD4.M6, P1.CD4.M7, P2.CD4.M1, P2.CD4.M2, P3.CD4.M1, P3.CD4.M2, P3.CD4.M3, P3.CD4.M4, P4.CD4.M1, P4.CD4.M2, P4.CD4.M3, in CD4+ T cells; c. biomarkers one or more of the following modules in Tables 3A-3D: P1. P1.ProlifT.M1, P1.Prolif.T.M2, P1.ProlifT.M3, P2.Prolif.T.M1, P2.Prolif.T.M2, P3.Prolif T.M1, P3.Prolif.T.M2, P3.Prolif.T.M3, P4.Prolif T.M1, P4.Prolif T.M2, P4.Prolif.T.M3, in proliferating T cells; d. biomarkers one or more of the following modules in Tables 3A-3D: P1.DC.M1, P1.DC.M2, P2.DC.M1, P2.DC.M2, P2.DC.M3, P3.DC.M1, P3.DC.M2, P4.DC.M1, P4.DC.M2, in dendric cells; e. biomarkers one or more of the following modules in Tables 3A-3D: P1Mono.M1, P1.Mono.M2, P1.Mono.M3, P1.Mono.M4, P1.Mono.M5, P1.Mono.M6, P1.Mono.M7, P1.Mono.M8, P2.Mono.M1, P2.Mono.M2, P2.Mono.M3, P2.Mono.M4, P2.Mono.M5, P3.Mono.M1, P3.Mono.M2, P3.Mono.M3, P3.Mono.M4, P3.Mono.M5, P3.Mono.M6, P3.Mono.M7, P3.Mono.M8, P4.Mono.M1, P4.Mono.M2, P4.Mono.M3, P4.Mono.M4, P4.Mono.M5, P4.Mono.M6, in monocytes; f. biomarkers one or more of the following modules in Tables 3A-3D: P1.NK.M1, P1.NK.M2, P1.NK.M3, P1.NK.M4, P2.NK.M1, P2.NK.M2, P2.NK.M3, P2.NK.M4, P3.NK.M1, P3.NK.M2, P3.NK.M3, P3.NK.M4, P3.NK.M5, P3.NK.M6, P4.NK.M1, P4.NK.M2, P4.NK.M3, P4.NK.M4, in NK cells; or g. biomarkers one or more of the following modules in Tables 3A-3D: P2.PB.M1, P2.PB.M2, P3.PB.M1, P4.PB.M1, P4.PB.M2, in plasmablasts.

In some embodiments, the one or more modulating agents modulate IFI27, IFI44L, IFI6, IFIT3, ISG15, XAF1, or a combination thereof. In some embodiments, the one or more modulating agents modulate CXCL10, DEFB1, IFI27L1, or a combination thereof, in monocytes. In some embodiments, the one or more modulating agents modulate PARP9, STAT1, or a combination thereof, in dendric cells. In some embodiments, the one or more modulating agents modulate CD52, TIGIT, TRAC, or a combination thereof, in CD4+ T cells. In some embodiments, the one or more modulating agents modulate CX3CR1, ICAM2, or a combination thereof, in NK cells. In some embodiments, the one or more modulating agents modulate CXCL10, LGALS3BP, or a combination thereof in monocytes and/or DC cells. In some embodiments, the one or more modulating agents modulate B2M, S100A4, KLF6, ANXA1, ITGB1, SYNE2, EZR, S100A6, AHNAK, CD52, IL32, or a combination thereof, in CD4+ T cells. In some embodiments, the one or more modulating agents modulate HLA-DQB1, HLA-DPB1, HLA-DPA1, CD74, HLA-DRA, HLA-DQA1, HLA-DRB1, CD52, or a combination thereof, in monocytes. In some embodiments, the one or more modulating agents modulate GZMB, GZMH, GNLY, FGFBP2, NKG7, PRF1, KLRD1, CCL5, or a combination thereof, in CTLs. In some embodiments, the one or more modulating agents modulate GNPTAB, PRSS23, GZMB, GNLY, B2M, FGFBP2, NKG7, PRF1, LGALS1, TMSB4X, TMSB10, CST7, or a combination thereof in NK cells. In some embodiments, the one or more modulating agents modulate GPR56, CST7, GZMA, KLRD1, FGFBP2, GZMH, NKG7, CCL5, CCL4, CTSW, HOPX, PRF1, GZMB, GNLY, PLEK, ID2, CD8A, UBB, SPON2, FCGR3A, or a combination thereof, in proliferating T cells. In some embodiments, the one or more modulating agents modulate PRF1, GZMB, GNYL, NKG7, FGFBP2, or a combination thereof, in CTLs, NK cells, and proliferating T cells.

In some embodiments, the one or more modulating agents modulate CD52 in CD4+ T cells and monocytes. In some embodiments, the one or more modulating agents modulate B2M in CD4+ T cells and NK cells. In some embodiments, the one or more modulating agents modulate GZMH, CCL5, KLRD1, or a combination thereof, in CTLs and proliferating T cells. In some embodiments, the one or more modulating agents modulate CST7 in NK cells and proliferating T cells. In some embodiments, the one or more modulating agents modulate PRF1, GZMB, GNYL, NKG7, FGFBP2, or a combination thereof, in NK cells, proliferating T cells, and CTL.

In some embodiments, the method comprises contacting monocytes with the one or more modulating agents that modulate genes in the following pathways: IFNα response, IFNγ response, complement, inflammatory response, TNF signaling via NF-κB, LPS stimulation, anti-TREM1 stimulation, PI3K inhibition, NFκB inhibition of HCMV inflammatory monocytes, or a combination thereof. In some embodiments, the method comprises contacting monocytes with the one or more modulating agents that modulate SERPINB2, CXCL3, CCL4, CCL3, IL1B, RPL5, STAT2, ICAM2, MIF, HLA-A, APOBEC3G, CD302, RPS16, SLAMF7, DUSP6, WARS, USP18, FCGR1B, CXCL1, CD300E, CCR1, IL6, CCL2, RIG-1, STAT1, HLA-G, APOBEC3B, ISG20, MX1, ISG15, IF127, or a combination thereof. In some embodiments, the method comprises modulating: a. biomarkers in clusters 0 in Table 7C in CD8+ T cells, b. biomarkers in clusters 1 in Table 7C in hyper-proliferative CD8+ T cells, c. biomarkers in clusters 2 in Table 7C in naïve CD4+ T cells, d. biomarkers in clusters 30 in Table 7C in CD8−/TRDC+/FCGR3A+ T cells, or e. a combination thereof. In some embodiments, the one or more modulating agents modulate CD8A, TNFAIP3, RGS1, HIST1H4C, PCNA, TOP2A, CCR7, ISG20, CD27, GZMK, TRDC, KLRF1, GZMB, XCL2, FCGR3A, or a combination thereof. In some embodiments, the one or more modulating agents that modulate one or more biomarkers in Table 7C.

In some embodiments, the one or more modulating agents modulate IL7R, LTB, TRBC2, LYZ, MNDA, CD14, NKG7, CCL5, GZMB, IL8, IL1B, CXCL2, MS4A1, CD79A, CD74, CD16, LST1, RHOC, STMN1, MKI67, CD8A, TNFSF10, ISG15, APOBEC3A, IGJ, IGHG1, MZB1, CD1C, HLA-DRA, CCL2, CCL4, UGCG, SERPINF1, or a combination thereof. In some embodiments, the method further comprises contacting resting monocytes, inflammatory monocytes, CD16+ monocytes, anti-viral monocytes, anti-viral/inflammatory monocytes, CD1C+ dendric cells, plasmacytoid dendric cells, B cells, plasmablasts, or a combination thereof. In some embodiments, the method comprises contacting plasmacytoid dendric cells with the one or more modulating agents that modulate IFITM1, IFI44L, ISG15, LY6E, IFI6, SAMD9L, IFI44, MX1, OAS3, EPSTI1, EEF1A1, SFT2D2, FOSB, FOS, ANKRD36BP1, UCP2, RPLP0, RHOA, RPL9, PSAP, or a combination thereof.

In some embodiments, the method comprises contacting B cells with the one or more modulating agents that modulate biomarkers in one or more of following pathways: B cell development, BCR signaling, psoriatic arthritis, proliferation of immune cells, or atherosclerosis signaling. In some embodiments, the method comprises contacting inflammatory monocytes with the one or more modulating agents that modulate BCL2A1, C5AR1, CCL3, CO83, CTSS, CXCL2, CXCL3, DUSP2, EREG, FTH1, G0S2, GADD45B, GPR183, IER3, IL1 B, IL8, NAM PT, NFKBIA, NFKBIZ, NLRP3, PDE4B, PLAUR, PPP1R15A, PTGS2, SAMSN1, SERPINB2, SOD2, SRGN, THBS1, TIPARP, TNFAIP3, TNFAIP6, ZFP36, or a combination thereof. In some embodiments, the method comprises contacting anti-viral monocytes with the one or more modulating agents that modulate APOBEC3A, APOBEC3B, B2M, CXCL10, EPSTl1, GBP1, GBP4, IFl27, IFl27L 1, IFl44L, IFl6, IFIT1, IFIT2, IFIT3, IFITM1, IFITM3, IGJ, ISG15, ISG20, L Y6E, MARCKS, MX1, NT5C3A, OAS1, PLAC8, RSAD2, SAT1, TNFSF10, TXNIP, XAF1, or a combination thereof.

In some embodiments, the method comprises contacting CTLs with the one or more modulating agents that modulate one or more biomarkers in Table 7A. In some embodiments, the method comprises contacting CTLs and/or proliferating T cells with the one or more modulating agents that modulate one or more biomarkers in Table 7B. In some embodiments, the method comprises contacting proliferating T cells with the one or more modulating agents that modulate one or more TRBV28, TRAV4, TRBV20-1, or a combination thereof.

In some embodiments, the one or more modulating agents further modulate CIITA, EBI3, G-CSF, HRAS, IL6, IFNA, IL10, Ig, IL12, IL4, IL2, TBX21, IFNG, IL21, IL27, STAT1, IL15, PDCD1, IL18, or a combination thereof. In some embodiments, the one or more modulating agents further modulate IFNG, TGFB1, STAT1, IFNA, PRDM1, SMARCA4, TP53, CIITA, G-CSF, EBI3, IL27, or a combination thereof. In some embodiments, the one or more modulating agents further modulate IL2, IFNA, IFNG, TNF, KRAS, CD3, IL15, IL4, IL1B, TGFB1, OSM, or a combination thereof. In some embodiments, the modulating agents further modulate IL4, G-CSF, IL2, IL27, IFNA, IFNG, IL6, STAT3, IL12, Ig, IL15, IL21, TBX21, or a combination thereof. In some embodiments, the modulating agents further modulate G-CSF, IL12, IFNA, IL18, CD40LG, IL4, Ig, IL15, IL2, IFNG, STAT1, IL27, PDCD1, IL21, IL6, TBX21, STAT3, TGFB1, or a combination thereof. In some embodiments, the method comprises contacting CD4+ T cells with the one or more modulating agents that modulate IFNA, OSM, IFNG, TNF, CD3, IL15, IL1B, TGFB1, KRAS, IL2, IL4, IL6, or a combination thereof. In some embodiments, the method comprises contacting monocytes with the one or more modulating agents that modulate CIITA, G-CSF, EBI3, IL27, IFNG, IFNA, STAT1, TGFB1, PRDM1, SMARCA4, TP53, or a combination thereof. In some embodiments, the method comprises contacting NK cells with the one or more modulating agents that modulate CIITA, IFNA, IFNG, STAT1, IL27, HRAS, IL15, EBI3, G-CSF, IL18, IL10, IL4, IL2, TBX21, PDCD1, IL21, IL6, Ig, IL12, or a combination thereof. In some embodiments, the method comprises contacting CTLs with the one or more modulating agents that modulate G-CSF, IL4, IFNG, IFNA, IL15, IL6, STAT3, IL27, IL21, Ig, IL2, TBX21, IL18, IL12, TGFB1, PDCD1, or a combination thereof. In some embodiments, the method comprises contacting proliferating T cells with the one or more modulating agents that modulate G-CSF, IL12, IFNA, IL18, IL15, TBX21, PDCD1, STAT3, IFNG, STAT1, IL27, IL21, IL6, Ig, IL2, IL4, TGFB1, CD40LG or a combination thereof.

In some embodiments, the one or more modulating agents modulate one or more biomarkers in Table 6B. In some embodiments, the one or more modulating agents are administered 1 week, 2 weeks, 3 weeks, 4 weeks, 6 months, or 1 year after the infection. In some embodiments, the subject does not have the infection. In some embodiments, the infection is a virus infection. In some embodiments, the infection is HIV infection. In some embodiments, the infection is an acute infection or hyper-acute infection. In some embodiments, the infection is a chronic infection. In some embodiments, the subject has viremia.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1E: Longitudinal profiling of peripheral immune cells in hyper-acute and acute HIV-infection by single-cell RNA-sequencing. (FIG. 1A) Representation of the typical trajectory of HIV viral load in the plasma during hyper-acute and acute HIV infection, and the timepoints sampled in this study. Since participants were tested twice weekly, there was an uncertainty of up to 3 days in where on the viral load curve the first detectable viremia occurs. The exact days sampled were available in Table 1. (FIG. 1B) Viral load and CD4 T cell count for the four individuals assayed in this study. Dotted lines indicate a missing data point for the metric. (FIG. 1C) tSNE analysis of PBMCs from all individuals and timepoints sampled (n=65,842). Cells are annotated based on differential expression analysis on orthogonally discovered clusters. (FIG. 1D) tSNE in C annotated by timepoint (left) and individual (right). (FIG. 1E) Scatter plot depicting the correlation between cell frequencies of CD4+ and CD8+ T cells measured by Seq-Well and FACS. R-squared values reflect variance described by a linear model. * p<0.05; ** p<0.01; *** p<0.001.

FIGS. 2A-2E: Gene module discovery revealed ubiquitous response to interferon with cell type specific features. (FIG. 2A) Schema depicting temporal gene module discovery (see Methods). This procedure was repeated for each major cell type (monocytes, CD4+ T cells, CTLs, proliferating T cells, NK cells, B cells, plasmablasts, and mDCs) on an individual-by-individual basis. (FIG. 2B) In P1, six gene modules across multiple cell types exhibited similar temporal profiles with peak module scores at the same timepoint as peak viremia was measured. (FIG. 2C) Number of occurrences of genes across the modules in FIG. 2B. (FIG. 2D) Module scores for interferon response modules in each individual. The timepoint where peak viral load occurs is indicated by a dotted line. (FIG. 2E) Luminex measurements of IP10 (left) and MIG (right) in matching plasma samples. Points are averages of duplicate measurements.

FIGS. 3A-3H: Modules with sustained expression conserved among individuals suggest shared and cell type specific drivers of immune response. Module Scores (left), gene overlaps between modules (middle), and enriched pathways for each module (right) in (FIG. 3A) CD4+ T cells, (FIG. 3B) monocytes, (FIG. 3C) CTLs, (FIG. 3D) NK cells, and (FIG. 3E) proliferating T cells. (FIG. 3F) Network of upstream drivers of modules in FIGS. 3A-3E. Edge width and grey scale reflect the number of shared genes (width) in the gene sets of the upstream drivers for a given cell-type. (FIG. 3G) Median gene set scores for significantly temporally variant (p<0.05) upstream drivers in P1. Scores are grouped by k-means clustering; k=5. (FIG. 3H) Summary table of immune responses to related and distinct stimuli with similar temporal dynamics.

FIGS. 4A-4E. One individual who goes on to control infection presents a poly-functional subset of monocytes at HIV detection. (FIG. 4A) Inflammatory and anti-viral scores of monocytes in P3 (left) and P1 (right) derived from gene lists created from merging modules among individuals. Ellipses drawn at 95% confidence interval for cells from each timepoint. (FIG. 4B) Principal component analysis (PCA) of all monocytes from P3 (left) and P1 (right). Density of cells in PC1 vs PC2 space annotated by timepoint are depicted, and the top loading genes for PC1 and PC2 are also annotated. (FIG. 4C) Heatmap of differentially expressed genes between monocytes at the peak response timepoint (0 weeks/1 week) vs pre-infection. Arrows indicate genes specific to P3 and P1. (FIG. 4D) Enriched pathways for the differentially expressed genes in FIG. 4C, using the MSigDB Hallmark Gene Sets. (FIG. 4E) Viral load by RT-PCR of the plasma of the four individuals assayed out to 2.75 years. Controllers of HIV maintain levels of plasma viremia less than 1,000 viral copies (vc)/mL. P1 initiated ART before the 2.3 year timepoint.

FIGS. 5A-5E. Controllers exhibited higher frequencies of proliferating CTLs and a precocious subset of NK cells 1 week after detection of HIV viremia. (FIG. 5A) Proportion of proliferating T cells of total CTLs as a function of time and individual measured by Seq-Well. (FIG. 5B) PCA of proliferating T cells from all four individuals. Cells assayed from the 1-week timepoint strongly separate along PC1 and PC2; Mann Whitney-U Test, *** p<0.001. (FIG. 5C) SNN clustering over the top 6 PCs reveals four sub-clusters (left) with distinct gene programs (right). (FIG. 5D) Percentage of cells in each sub-cluster by timepoint. (FIG. 5E) Number of cells from each individual within the cells sampled at 0 weeks and 1 week in the NK cell cluster (4-lilac; black box in FIG. 5D).

FIGS. 6A-6D: Patient and time point breakdown by cluster and cluster annotation. (FIG. 6A) Time point and (FIG. 6B) patient cell frequency by annotated cell type (left) and shared-nearest neighbors (SNN) clusters (right). (FIG. 6C) Heatmap of the top 10 genes differentiating each SNN cluster (Wilcoxon rank sum test). (FIG. 6D) tSNE embedding of dataset labeled by SNN cluster and annotated based on genes in FIG. 6C and Table 2.

FIGS. 7A-7B: Cell frequency by individual and cell type. (FIG. 7A) Representative gating scheme for CD4+ and CD8+ T cells. (FIG. 7B) Cell type frequency calculated from total cells measured within an array. Lines represent average between duplicate arrays. Columns are separated by individual.

FIGS. 8A-8D: Gene modules that aligned near peak viremia were enriched for response to interferon and match responses observed in acute SIV infection in rhesus macaques. (FIG. 8A) Enrichment of modules from P1 in FIG. 2B against the REACTOME: Response to Type I Interferon gene set; FDR corrected hypergeometric test. (FIG. 8B) Differential expression results for IRF7 in each cell type (except plasmablasts and mDCs which did not have enough cells to test, n<4) between cells from 2 weeks and pre-infection+1 year; implemented using the “bimod” likelihood ratio test in Seurat. (FIG. 8C) Median expression of genes upregulated in SIV infection of rhesus macaques compared to day 0 (fold change>2) in Bosinger et al. (47). (FIG. 8D) Same as A for the modules in FIG. 2D for P2, P3, and P4.

FIGS. 9A-9D: Plasmacytoid Dendritic Cells (pDCs) demonstrated similar interferon responses at the same time as other cell types. (FIG. 9A) Representative gating scheme for single-cell pDC sorts. (FIG. 9B) Heatmap of genes differentially expressed between pDCs captured at the same time points as peak interferon responses and 1-year post HIV infection; implemented using a Wilcoxon rank sum test. (FIG. 9C) Scoring of pDCs in each individual using a core interferon signature specific to that individual. (FIG. 9D) Heatmap of gene frequency across interferon response modules in each individual.

FIG. 10: All significant temporally variant modules in all individuals grouped by fuzzy c-means clustering. Modules grouped by fuzzy c-means clustering (see Methods in Example 1 for choice of c) reside in the same gray box. Each group of modules, or meta module (MM), were then aligned across patients based on overall temporal trend (left column). Some individuals had multiple MM with similar temporal dynamics and were grouped within the same MM. Since fuzzy c-means clustering assigns membership values to each member of a cluster, Applicants report any modules that demonstrated low cluster membership with t.

FIGS. 11A-11D: Sustained B cell modules and shared genes and upstream drivers between individuals. (FIG. 11A) B cell modules in MM1 with high cluster membership. (FIG. 11B) Euler diagram of conserved overlapping genes between cell types from FIGS. 3A-3E, see Tables 5A-5B. (FIG. 11C) FIG. 3F displayed with only the edges from a given cell type. (FIG. 11D) Luminex measurements of IP10 (left), MIG (center), and IL-12 (right) in matching plasma samples. Points are averages of duplicate measurements.

FIG. 12: Upstream driver scores highlight variable response dynamics across cell types. Median gene set scores for significantly temporally variant (p<0.05) upstream drivers in all individuals. Gray boxes indicate that the upstream driver was not significantly variant in that cell type and individual.

FIGS. 13A-13E: Two cases of similar temporal modules: variable correlation and variable co-expression. (FIG. 13A) Module scores in NK cells for NK M3 and NK M4 in P3. Ellipses drawn at 95% confidence interval for cells from each time point. (FIG. 13B) Correlation (spearman's rho) between the scores for NK M3 and NK M4 at each time point. FDR corrected q-value: N.S=not significant; *q<0.05; ** q<0.01, * ** q<0.001. (FIG. 13C & FIG. 13D) Same as in FIG. 13A & FIG. 13B but for monocyte modules Mono M1 and Mono M3 in P3. (FIG. 13E) Gene set enrichment analysis of the genes in Mono M1 and Mono M3 against the following MSigDB collections: Hallmark, C2, C3, C5, and C7. FDR corrected hypergeometric test.

FIGS. 14A-14D: Core anti-viral, inflammatory, and non-classical programs in monocytes. (FIG. 14A) The genes shared between individuals (present in at least two modules) that make up the inflammatory and anti-viral scores used in FIG. 4A, as well as in FIG. 14B and FIG. 14C of this figure. (FIG. 14B & FIG. 14C) Inflammatory and anti-viral scores of monocytes by time point in P2 (FIG. 14B) and P4 (FIG. 14C). Ellipses drawn at 95% confidence interval for cells from each time point. (FIG. 14D) Percent of Non-Classical (CD16+) monocytes of total monocytes as a function of time in each individual. Percentage calculated from cluster assignment (see FIG. 6D).

FIGS. 15A-15G: Non-proliferating and proliferating cytotoxic T cells. (FIG. 15A) Principal component analysis of non-proliferating CTLs with patient density annotated along PC1 and PC2. (FIG. 15B) Volcano plot of differentially expressed genes between the individuals who control (P3/P4) and those who do not (P1/P2); implemented using a Wilcoxon rank sum test. (FIG. 15C) Expression of GZMB and PRF1 in all CTLs and proliferating T cells. (FIG. 15D) Volcano plot of differentially expressed genes between non-proliferating CTLs and proliferating T cells; implemented using a Wilcoxon rank sum test. (FIG. 15E) Heatmap of detected TCR-α and TCR-βvariable chain genes in proliferating T cell clusters 0 & 1. (FIG. 15F) Same as in A but over proliferating T cells. (FIG. 15G) CD8 T cell (top), γδT cell (middle), and NK cell (bottom) scores for each proliferating T cell cluster (see FIG. 5C), 500 randomly sampled CTLs, and 500 randomly sampled NK cells. Signatures were established from differential expression over the single-cell dataset published by Gutierrez-Arcelus et al. (21). See Tables 7A-7C for all differentially expressed genes and signature score gene lists.

FIGS. 16A-16E: Longitudinal profiling of peripheral immune cells in hyperacute and acute HIV infection by scRNA-seq. (FIG. 16A) Depiction of the typical trajectory of HIV viral load in the plasma during hyperacute and acute HIV infection and the timepoints sampled in this study. Since participants were tested twice weekly, there was an uncertainty of up to 3 d in where on the viral load curve the first detectable viremia occurred (error bar is representative). (FIG. 16B) Viral load and CD4+ T cell count for four participants assayed in this study. Dotted lines indicate a missing data point for the metric. (FIG. 16C) t-distributed stochastic neighbor embedding (tSNE) analysis of PBMCs from all participants and timepoints sampled (n=59,162). Cells are annotated based on differential expression analysis on orthogonally discovered clusters. (FIG. 16D) tSNE in c annotated by timepoint (top) and participant (bottom). (FIG. 16E) Scatter-plot depicting the correlation between cell frequencies of CD4+ and CD8+ T cells measured by Seq-Well (n=2 array replicates) and FACS (n=1 flow replicate). R2 values reflect variance described by an F-test for linear regression.

FIGS. 17A-17F: GM discovery reveals ubiquitous response to IFN with cell-type-specific features. (FIG. 17A) Schema depicting temporal GM discovery. This procedure was repeated for each major cell type (monocytes, CD4+ T cells, CTLs, proliferating T cells, NK cells, B cells, plasmablasts and mDCs) on a participant-by-participant basis, generating 0-8 GMs per cell type. Modules were arbitrarily numbered within a given cell type and participant. mDC, myeloid dendritic cell; PCA, principal-component analysis; PC, principal component; TOM, topological overlap matrix. (FIG. 17B) In P1, six GMs across multiple cell types exhibited similar temporal expression profiles; each GM's score peaked at the same timepoint as for peak viremia (line-and-dot plot). (FIG. 17C) Number of occurrences of gene membership for all genes present across the six GMs in FIG. 17B. (FIG. 17D) GM expression scores for IFN response modules in each participant. Normalized GM score is depicted by heat, whereas raw module score is depicted by box size. The timepoint closest to peak viral load is indicated by a dotted line. (FIG. 17E) Mean expression of ISG15 separated by timepoint and individual. Shaded area denotes 95% CI of the mean. (FIG. 17F) Luminex measurements of IP-10, MIG and IFN-γ and ELISA of soluble CD14 (sCD14) in matching plasma samples. Points are averages of duplicate measurements. PI, pre-infection; PV, peak viremia; 9 M, 9 months post-detection.

FIGS. 18A-18K—Distinct modules across different cell types share temporal expression patterns in acute HIV infection and suggest shared and cell-type-specific drivers of immune response. (FIG. 18A) Normalized module expression scores for the six GMs clustered into meta modules: gradual positive (MMgp) in P2. † indicates GMs with MM membership score <0.25. (FIG. 18B) Mean gene expression of representative genes from modules in FIG. 18A. (FIG. 18C) Select enriched pathways for the genes in each module from FIG. 18A; gene-set enrichment performed in ingenuity pathway analysis (IPA). (FIG. 18D) Putative cell-cell signaling network. Nodes represent gene products with either measured gene upregulation in the modules in a or predicted drivers from IPA. Edges were drawn from connections nominated by IPA and curated from the literature. (FIGS. 18E-18I) Module scores (left), gene overlaps between modules (middle) and enriched pathways (right; IPA) for modules grouped in MMgp and shared across participants in CD4+ T cells. (FIG. 18E) monocytes. (FIG. 18F) CTLs. (FIG. 18G) NK cells (FIG. 18H) and proliferating T cells. (FIG. 18I) Enriched pathways were determined using a right-tailed Fisher's exact test. (FIG. 18J) Putative cell-cell signaling network derived from genes shared across ≥2 participants from modules in FIGS. 18E-18I. Nodes and edges are drawn as in FIG. 18D. Here, Applicants highlight those molecules interacting with or measured in CD4+ T cells; the full network is presented in FIG. 26B. To reduce complexity, Applicants omitted nodes depicting expression of GZMB, PRF1 and GNLY by both NK cells and proliferating T cells and CCL5 by proliferating T cells. (FIG. 18K) Summary table of immune responses to related and distinct stimuli with similar temporal dynamics, defined by transient increased module expression for several weeks after peak viremia.

FIGS. 19A-19F: Future controllers exhibit higher frequencies of proliferating CTLs and a precocious subset of NK cells 1 week after detection of HIV viremia. (FIG. 19A) Viral load by PCR with reverse transcription of the plasma of four participants assayed out to 2.75 years. Controllers of HIV maintain levels of plasma viremia <1,000 viral copies ml-1. P1 initiated ART before the 2.3-year timepoint. (FIG. 19B) Proportion of proliferating T cells of total CTLs as a function of time and individual measured by Seq-Well. (FIG. 19C) PCA of proliferating T cells from all four individuals. Cells assayed from the 1-week timepoint strongly separated along PC1 and PC2; two-sample Mann-Whitney U-test; 174 cells (1 week) versus 2,465 cells (all other timepoints); ***P<2.2×10-16. (FIG. 19D) Shared-nearest neighbor (SNN) clustering over the top six PCs reveals four subclusters (left) with distinct gene programs (right). Two-sample Wilcoxon rank-sum test was used for analysis; numbers of cells per cluster: 1—1,081; 2—929; 3—359; 4—270. (FIG. 19E) Percentage of cells in each subcluster by timepoint. (FIG. 19F) Number of cells from each individual within the cells sampled at 0 weeks and 1 week in the NK cell cluster (4, lilac; black box in FIG. 19E).

FIGS. 20A-20G Participant and time point breakdown by cluster and cluster annotation. (FIG. 20A) Plasma viral load for the 12 participants studied in Ndhlovu et al.37 with the four individuals characterized here annotated. (FIG. 20B) Average ratio of number of reads per number of UMIs measured per single cell. R-squared values reflect variance described by a linear model. Number of cells per participant: P1—15,259; P2—13,128; P3—15,927; P4—15,425. (FIG. 20C) Time point and (FIG. 20D) participant cell frequency by annotated cell type (left) and shared-nearest neighbors (SNN) clusters (right). (FIG. 20E) Relative expression levels of exemplary marker genes used for cell type identification projected on the FItSNE. (FIG. 20F) Heatmap of the top 10 genes differentiating each SNN cluster. Up to 500 random cells are depicted for each cluster. Two-sample Wilcoxon rank sum test. (FIG. 20G) tSNE embedding of dataset labeled by SNN cluster and annotated based on genes in FIGS. 20E and 20F.

FIGS. 21A-21B: Cell frequency by participant and cell type. (FIG. 21A) Representative gating scheme for CD4+ and CD8+ T cells. n=30 samples. (FIG. 21B) Cell type frequency calculated from total cells measured within an array. Lines represent average between duplicate arrays. Columns are separated by participant. (FIG. 21C) Monocyte count from whole blood for 12 participants studied in Ndhlovu et al. Two-sided paired t test, n=12.

FIGS. 22A-22I: Gene modules that align near peak viremia and are differentially expressed in pDCs are enriched for response to interferon. (FIG. 22A) Enrichment of modules from P1 in FIG. 2b against the IPA Interferon Signaling canonical pathway; FDR corrected Right-Tailed Fisher's Exact Test. (FIG. 22B) Heatmap of median expression of genes upregulated in PBMCs from SIV infection of rhesus macaques (n=8) compared to day 0 (fold change >2) in Bosinger et al. depicted independently for each cell type in P1. Only genes differentially expressed across all time points by ANOVA in a given cell type are shown. (FIG. 22C) Differential expression results for IRF7 in each cell type (except plasmablasts and mDCs which do not have enough cells to test, n<4) between cells from 2 weeks and pre-infection+1 year; implemented using the “bimod” likelihood ratio test in Seurat. (FIG. 22D) Representative gating scheme for single-cell pDC sorts. n=8 samples. (FIG. 22E) Heatmap of genes differentially expressed between pDCs captured at the same timepoints as peak interferon responses and 1-year post HIV infection. Two-sided Wilcoxon rank sum test; number of cells per timepoint: Peak—159; 1-Year-184. (FIG. 22F) Same as a for the modules in FIG. 17D for P2, P3, and P4. (FIG. 22G) Mean gene expression (log scaled) of MX1 and CXCL10 over time in each individual separated by cell type. Shaded area denotes 95% confidence interval of the mean. (FIG. 22H) Scoring of pDCs in each participant using a core interferon signature specific to that participant. Number of cells per participant and time point: P1 Peak—31; P1 1—Year—79; P2 Peak—48; P2 1—Year—20; P3 Peak—42; P3 1—Year—28; P4 Peak—16; P4 1—Year—57. (FIG. 22I) Heatmap of gene frequency across interferon response GMs in each participant.

FIG. 23: All significant temporally variant modules in all participants grouped by fuzzy c-means clustering. Modules grouped by fuzzy c-means clustering (see Methods for choice of c) reside in the same gray box. Each group of modules, or meta module (MM), were then aligned across participants based on overall temporal trend (left column). Some participants had multiple MM with similar temporal dynamics and were grouped within the same MM. Since fuzzy c-means clustering assigns membership values to each member of a cluster, Applicants report any modules that demonstrated low cluster membership with t.

FIGS. 24A-24D: Cross-participant module discovery recapitulates aspects determined by calculating participant-specific modules. (FIG. 24A) Timepoints chosen for testing significant changes in module expression over time across all participants. Timepoints were chosen based on peak expression of modules in MM1 and MM3. Applicants used an ANOVA model to account for participant-specific features. (FIG. 24B) Significant cross-participant modules that map to participant-specific modules (share at least 5 genes), separated by cell type. Median module expression is plotted for each module split by participant. Error bars depict the upper and lower quartiles for all cells across all four individuals at each time point. Boxed modules demonstrate consistent directional trends in expression between each pair of timepoints in at least 3/4 participants. P-I=pre-infection; P=peak; P-P=post-peak; 1-Y=1 year. (FIG. 24C), Significant cross-participant modules that do not correspond to participant-specific modules. (FIG. 24D) Sankey diagram demonstrating the gene overlap between participant specific modules (left) and cross-participant modules (right). Node size correlates with the number of genes within the module and edge width correlates with the number of shared genes between modules. Only overlaps consisting of ≥5 genes have edges depicted.

FIGS. 25A-25F: Sustained B cell modules and shared genes and upstream drivers between participants. (FIG. 25A) B cell modules in MM1 with high cluster membership. (FIG. 25B) Euler diagram of conserved overlapping genes between cell types from FIGS. 18E-18I. (FIG. 25C) Mean expression of ANXA1, HLA-DRA, CCL5, PRF1, and CD8A in CD4+ T cells, monocytes, CTLs, NK cells, and proliferating T cells, respectively. Shaded area denotes 95% confidence interval of the mean. Participants who did not have modules with shared temporal expression pattern as outlined in FIGS. 18E-18I are shown as dashed lines. (FIG. 25D) Network of predicted upstream drivers of modules in FIGS. 18E-18I. Edge width and gray scale reflect the number of shared genes (width) in the gene sets of the upstream drivers for a given cell-type. (FIG. 25E) displayed with only the edges from a given cell type. (FIG. 25F) Luminex measurements of IP-10, MIG, IFN-γ, IL-12 in matching plasma samples. Points are averages of duplicate measurements. PI=pre-infection; 4W=4 weeks post-detection; 9M=9 months post-detection.

FIGS. 26A-26B: Putative upstream drivers highlight variable response dynamics and cell-cell signaling. (FIG. 26A) Median gene set scores for significantly temporally variant (p<0.05) upstream drivers in all participants. Gray boxes indicate that the upstream driver was not significantly variant in that cell type and participant. Right-Tailed Fisher Exact Test. (FIG. 26B) Putative cell-cell network described in FIG. 18J, but all nodes and connections depicted. Nodes represent genes with either measured upregulation in the modules in FIGS. 18E-18I or predicted drivers from IPA. Edges were drawn from connections nominated by IPA and curated from the literature.

FIGS. 27A-27F: Two cases of similar temporal modules in NK cells and monocytes: variable correlation and variable co-expression. (FIG. 27A) Module scores in NK cells for NK GM3 and NK GM4 in P3. Ellipses drawn at 95% confidence interval for cells from each timepoint. (FIG. 27B) Correlation (spearman's rho) between the scores for NK GM3 and NK GM4 at each timepoint. Two-sided asymptotic t approximation; FDR corrected q-value: N.S=not significant; *q<0.05; ** q<0.01, *** q<0.001. (FIG. 27C) Gene expression heatmap of all NK cells in P3 for those genes in NK GM3 and NK GM4. Cells are separated based on k-means (k=3) over the depicted genes. (FIGS. 27D-27E) Same as in a & b but for monocyte modules Mono GM1 and Mono GM3 in P3. (FIG. 27F) Gene set enrichment analysis of the genes in Mono GM1 and Mono GM3 against the following MSigDB collections: Hallmark, C2, C3, C5, and C7. FDR corrected hypergeometric test; number of genes: GM1—33, GM3—52.

FIGS. 28A-28G: Participants demonstrate diverse monocyte responses prior to and immediately after HIV detection, wherein one participant who goes on to control infection presents a poly-functional subset of monocytes. (FIG. 28A) Inflammatory and anti-viral genes shared between participants (present in at least two modules. (FIG. 28B) Inflammatory and anti-viral scores of monocytes in each participant using gene lists in FIG. 28A. Ellipses drawn at 95% confidence interval for cells from each timepoint. (FIG. 28C) Principal component analysis (PCA) of all monocytes from each participant. Density of cells in PC1 vs PC2 space annotated by timepoint are depicted, and 3/5 of the top loading genes for PC1 and PC2 are also annotated. (FIG. 28D) Percent of Non-Classical (CD16+/FCGR3A) monocytes of total monocytes as a function of time in each participant. Percentage calculated from cluster assignment (see FIG. 20D). (FIG. 28E) Heatmap of differentially expressed genes (FDR corrected q<0.05 in at least one participant) between monocytes at the peak response timepoint (0 weeks/1 week) vs. pre-infection. Arrows indicate genes specific to P3 and P1. Two-sided Wilcoxon rank sum test. (FIG. 28F) Enriched pathways for the differentially expressed genes in e, using the MSigDB Hallmark Gene Sets. Hypergeometric test; FDR corrected q values; number of differentially expressed genes: P1—1,350; P2—436; P3—857; P4—514. (FIG. 28G) Violin plot of the Inflammatory Module Score (see a for genes in the module) for monocytes at pre-infection (Pre) and peak transcriptional response time points (Peak) in each participant.

FIGS. 29A-29H: Non-proliferating and proliferating cytotoxic T cells. (FIG. 29A) Principal component analysis of non-proliferating CTLs with participant density annotated along PC1 and PC2. Number of cells: P1—1828; P2—968; P3—1503; P4—670. (FIG. 29B) Volcano plot of differentially expressed genes between the participants who control (P3/P4) and those who do not (P1/P2); implemented using a two-sided Wilcoxon rank sum test. (FIG. 29C) Expression of GZMB and PRF1 in all CTLs and proliferating T cells. Number of cells: CTL—4,969; Proliferating T cells—2,639. (FIG. 29D) Volcano plot of differentially expressed genes between non-proliferating CTLs and proliferating T cells; implemented using a two-sided Wilcoxon rank sum test; see FIG. 29C for cell numbers. (FIG. 29E) Heatmap of detected TCR-β CDR3s in proliferating T cell clusters 0 & 1 at 2 weeks, 3 weeks, and 4 weeks post-HIV detection. (FIG. 29F) Distribution of ranked TCR-β CDR3 clones (by total cell number) and singletons measured from all T cells (CD4+ T cells, CTLs, and proliferating T cells) detected at 2 weeks, 3 weeks, and 4 weeks post-HIV detection in at least two single cells in each participant. Here, except for the singletons, each sliver represents the percentage of CDR3s ascribed to the top n-n+1 clones for that timepoint and participant. (FIG. 29G) Same as in a but over proliferating T cells. Number of cells: P1—483; P2—273; P3—1193; P4—690. (FIG. 29H) CD8 T cell (top), γδT cell (middle), and NK cell (bottom) scores for each proliferating T cell cluster (see FIG. 29G for cell numbers) and 500 randomly sampled CTLs, and 500 randomly sampled NK cells. Signatures were established from differential expression over the single-cell dataset published by Gutierrez-Arcelus et al. Box plots features depict: minimum=25th percentile—1.5*inter-quartile range (IQR; smallest value within); lower=25th percentile; center=50th percentile; upper=75th percentile; maximum=75th percentile+1.5*IQR (largest value within).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M.J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for methods and compositions for modulating the immune response to an infection in a subject. In general, the methods comprise modulating a gene or a combination of genes. The genes may be involved (in a subject's response to an infection in one or more specific types of cells and/or at one or more specific time points. In some embodiments, the expressions of the gene or gene combination change in response to the infection. In some examples, the expressions of the gene or gene combination change in certain type(s) of cells after the infection. Alternatively or additionally, the expressions of the gene or gene combination change at certain time point(s) after the infection. Modulating the expressions of the gene or gene combination in specific types of cells and/or at specific time points after infection may allow for more effective and safer treatment for diseases related to the infection.

### Method of Treatment

In an aspect, the present disclosure provides methods for modulating immune responses (e.g., triggered by an infection such as HIV infection) in cells, tissues, organs, or a subject. In some embodiments, the method comprises contacting one or more types of cells with one or more modulating agents. The modulating agents may modulate the expression of a gene or a combination of genes in one or more signaling pathways.

As used herein, the terms “treat”, “treating” and “treatment” refer to the alleviation or measurable lessening of one or more symptoms or measurable markers of an injury, disease or disorder. Measurable lessening includes any statistically significant decline in a measurable marker or symptom. In some embodiments, treatment is prophylactic treatment.

The treatment method may include administering a therapeutically effective amount of agent. The term “therapeutically effective amount” refers to an amount effective, at dosages and for periods of time necessary, to achieve the desired therapeutic result, e.g., a diminishment or prevention of effects associated with various disease states or conditions. The term “therapeutically effective amount” refers to an amount of a target gene or gene product modulator effective to treat or prevent a disease or disorder in a mammal. A therapeutically effective amount of a target gene or gene product modulator can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an “effective amount”, which as used herein, refers to the amount of therapeutic agent of pharmaceutical composition to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of an immune or autoimmune disease which are appropriate measures by those skilled in the art. It should be noted that a target gene or gene product modulator as disclosed herein can be administered as a pharmaceutically acceptable salt and can be administered alone or as an active ingredient in combination with pharmaceutically acceptable carriers, diluents, adjuvants and vehicles.

The treatment method may include administering a prophylactically effective amount of agent. The term “prophylactically effective amount” refers to an amount of a target gene or gene product modulator which is effective, at dosages and for periods of time necessary, to achieve the desired prophylactic result, e.g., the amount of a target gene or gene product modulator. Typically, since a prophylactic dose of a target gene or gene product modulator is administered to a subject prior to or at an earlier stage of a disease, and in some embodiments, a prophylactically effective amount is less than the therapeutically effective amount. A prophylactically effective amount of a target gene or gene product modulator is also one in which any toxic or detrimental effects of the compound are outweighed by the beneficial effects.

As used herein, the terms “prevent”, “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “administering” and “introducing” are used interchangeably herein and refer to the placement of the agents of metabolic regulators of the present invention into a subject by a method or route which results in at least partial localization of a target gene or gene product modulator at a desired site. The compounds of the present invention can be administered by any appropriate route which results in an effective treatment in the subject. In some embodiments, administering is not systemic administration.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration”, “administered systemically”, “peripheral administration” and “administered peripherally” as used herein mean the administration of a modulator such that it enters the animal's system and, thus, is subject to metabolism and other like processes, for example, subcutaneous administration.

### Target Genes and Pathways for Modulating Immune Responses

### Signaling Pathways

The methods may comprise modulating (e.g., using modulating agents) one or more signaling pathways. Modulating of a signaling pathway may include modulating one or more genes in the signaling pathway. The term “signaling pathway” refers to a series of cellular components involved in the intracellular or intercellular communication or transfer of information, including cell surface receptors, nuclear receptors, signal regulatory proteins, and intracellular signaling components. As used herein, a particular “signaling pathway” may be named according to the ligand or the cell surface receptor that triggers the cascade of intracellular signaling (e.g., TNFα pathway), or according to any of the components involved in the intracellular signaling (e.g., PI3K pathway). In some cases, a pathway is named according to a function of the pathway, e.g., antigen presentation pathway. In certain cases, a pathway include genes related to a disease or disorder, and may be named according to that disease or disorder, e.g., adhesion of T cells.

Examples of signaling pathways include those in Table 6A.

In some embodiments, the methods comprise modulating genes in one or more signaling pathways in a specific type of cells. For examples, the method may comprise modulating one or more genes in adhesion of T cells, Cdc42 signaling, cytokine signaling, regulation by calpain, endocytic virus entry, or a combination thereof, in CD+4 T cells. The method may comprise modulating one or more genes in allograft rejection signaling, Cdc42 signaling, antigen presentation, IL-4 signaling, OX40 signaling, or a combination thereof, in monocytes. The method may comprise modulating one or more genes in CTL killing or target cells, graft-vs-host disease signaling, Granzyme B signaling, interferon signaling, hypercytokinemia in flu, or a combination thereof, in CTLs. The method may comprise modulating one or more genes in chemokinesis of leukocytes, CTL killing of target cells, innate-adaptive crosstalk, OX40 signaling, dendric cell (DC)-NK crosstalk, or a combination thereof, in NK cells. The method may comprise modulating one or more genes innate-adaptive crosstalk, CTL killing of target cells, degranulation of cells, granzyme B signaling, and interferon signaling, or a combination thereof, in proliferating T cells. The method may comprise modulating one or more genes in any combination of the pathways herein. The method may comprise modulating one or more genes in any combination of the types of cells herein. The method may comprise modulating one or more genes in any combination of the pathways in the specific types of cells herein.

In some embodiments, the method comprises modulating IFNα response, IFNγ response, complement, inflammatory response, TNF signaling via NF-κB, LPS stimulation, anti-TREM1 stimulation, PI3K inhibition, NFκB inhibition of HCMV inflammatory monocytes, or a combination of the pathways in monocytes. The method may comprise contacting monocytes with one or more modulating agents that modulate gene(s) in the pathway(s).

In some embodiments, the method comprises modulating B cell development, BCR signaling, psoriatic arthritis, proliferation of immune cells, or atherosclerosis signaling, or a combination of the pathways in B cells. The method may comprise contacting B cells with one or more modulating agents that modulate gene(s) in the pathway(s).

### Genes

The expression and/or activity of one or more genes (e.g., one or more genes encoding components of signaling pathways) may be modulated. Modulating a gene may include modulating the expression, concentration, and/or activity of the gene or encoded product thereof. As used herein, the term “gene” refers to a nucleic acid comprising an open reading frame encoding a polypeptide, including both exon and (optionally) intron sequences. A gene may include to coding sequence of a gene product, as well as non-coding regions of the gene product, including 5′UTR and 3′UTR regions, introns and the promoter of the gene product. The coding region of a gene can be a nucleotide sequence coding for an amino acid sequence or an RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene may also be the segment of DNA involved in producing a polypeptide chain, it includes regions preceding and following the coding region as well as intervening sequences (introns and non-translated sequences, e.g., 5′- and 3′-untranslated sequences and regulatory sequences) between individual coding segments (exons). A gene may also be an amplified nucleic acid molecule produced in vitro comprising all or a part of the coding region and/or 5′- or 3′-untranslated sequences linked thereto.

The one or more genes may be biomarkers. Biomarkers in the context of the present disclosure encompass, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein. Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject. The biomarkers of the present disclosure are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments. In some cases, biomarkers are used interchangeably with genes.

All gene name symbols refer to the gene as commonly known in the art. Gene symbols may be those referred to by the HUGO Gene Nomenclature Committee (HGNC). Any reference to the gene symbol is a reference made to the entire gene or variants of the gene. The HUGO Gene Nomenclature Committee is responsible for providing human gene naming guidelines and approving new, unique human gene names and symbols. All human gene names and symbols can be searched at www.genenames.org, the HGNC website, and the guidelines for their formation are available there (www.genenames.org/guidelines).

In some embodiments, the methods comprise modulating one or more genes in specific types of cells. In some examples, the methods comprise modulating one or more genes in cluster 1 of Table 2 in CD4+ T cells. In some examples, the methods comprise modulating one or more genes in cluster 2 of Table 2 in resting monocytes. In some examples, the methods comprise modulating one or more genes in cluster 3 of Table 2 in cytotoxic lymphocytes. In some examples, the methods comprise modulating one or more genes in cluster 4 of Table 2 in inflammatory monocytes. In some examples, the methods comprise modulating one or more genes in cluster 5 of Table 2 in B cells. In some examples, the methods comprise modulating one or more genes in cluster 6 of Table 2 in non-classical monocytes. In some examples, the methods comprise modulating one or more genes in cluster 7 of Table 2 in proliferating T cells. In some examples, the methods comprise modulating one or more genes in cluster 8 of Table 2 in anti-viral monocytes. In some examples, the methods comprise modulating one or more genes in cluster 9 of Table 2 in plasmablasts. In some examples, the methods comprise modulating one or more genes in cluster 10 of Table 2 in CD1C+ dendric cells (DCs). In some examples, the methods comprise modulating one or more genes in cluster 11 of Table 2 in both anti-viral monocytes and inflammatory monocytes. In some examples, the methods comprise modulating one or more genes in cluster 12 of Table 2 in CD1C+ plasmacytoid dendric cells (pDCs).

In some embodiments, the methods comprise modulating one or more genes in a module. The term “gene module” or “module” refers to a group, set, or collection of genes. Genes in the same module may be co-regulated. For example, the expression of the genes in a module may change in response to a stimulus or event, e.g., an infection. In some examples, genes in a module belong to the same metabolic pathway. In some examples, genes in a module are co-expressed, e.g., the same set of transcription factors binds to the genes of the module to modulate expression of the genes of the module. In some examples, the genes of a module are provided together on a nucleic acid (e.g. genomic DNA).

In some examples, the methods comprise modulating one or more genes in one or more of the following modules in Tables 3A-3D: P1.B.M1, P1.B.M2, P1.B.M3, P2.B.M1, P2.B.M2, P2.B.M3, P2.B.M4, P3.B.M1, P3.B.M2, P3.B.M3, P3.B.M4, P3.B.M5, P4.B.M1, P4.B.M2, or any combination thereof. In certain cases, the methods comprise modulating one or more genes in one or more of the modules in B cells.

In some examples, the methods comprise modulating one or more genes in one or more of the following modules in Tables 3A-3D: P1.CD4.M1, P1.CD4.M2, P1.CD4.M3, P1.CD4.M4, P1.CD4.M5, P1.CD4.M6, P1.CD4.M7, P2.CD4.M1, P2.CD4.M2, P3.CD4.M1, P3.CD4.M2, P3.CD4.M3, P3.CD4.M4, P4.CD4.M1, P4.CD4.M2, P4.CD4.M3, or any combination thereof. In certain cases, the methods comprise modulating one or more genes in one or more of the modules in CD4+ T cells.

In some examples, the methods comprise modulating one or more genes in one or more of the following modules in Tables 3A-3D: P1.CTL.M1, P1.CTL.M2, P1.CTL.M3, P1.CTL.M4, P1.CTL.M5, P2.CTL.M1, P2.CTL.M2, P2.CTL.M3, P2.CTL.M4, P3.CTL.M1, P3.CTL.M2, P3.CTL.M3, P3.CTL.M4, P4.CTL.M1, P4.CTL.M2, P4.CTL.M3, or any combination thereof. In certain cases, the methods comprise modulating one or more genes in one or more of the modules in cytotoxic T cells (CTLs).

In some examples, the methods comprise modulating one or more genes in one or more of the following modules in Tables 3A-3D: P1.ProlifT.M1, P1.ProlifT.M2, P1.ProlifT.M3, P2.Prolif.T.M1, P2.Prolif T.M2, P3.Prolif.T.M1, P3.Prolif.T.M2, P3.Prolif T.M3, P4.Prolif T.M1, P4.Prolif.T.M2, P4.Prolif.T.M3, or any combination thereof. In certain cases, the methods comprise modulating one or more genes in one or more of the modules in proliferating T cells.

In some examples, the methods comprise modulating one or more genes in one or more of the following modules in Tables 3A-3D: P1.DC.M1, P1.DC.M2, P2.DC.M1, P2.DC.M2, P2.DC.M3, P3.DC.M1, P3.DC.M2, P4.DC.M1, P4.DC.M2, or any combination thereof. In certain cases, the methods comprise modulating one or more genes in one or more of the modules in dendric cells, e.g., myeloid dendric cells.

In some examples, the methods comprise modulating one or more genes in one or more of the following modules in Tables 3A-3D: P1Mono.M1, P1.Mono.M2, P1.Mono.M3, P1.Mono.M4, P1.Mono.M5, P1.Mono.M6, P1.Mono.M7, P1.Mono.M8, P2.Mono.M1, P2.Mono.M2, P2.Mono.M3, P2.Mono.M4, P2.Mono.M5, P3.Mono.M1, P3.Mono.M2, P3.Mono.M3, P3.Mono.M4, P3.Mono.M5, P3.Mono.M6, P3.Mono.M7, P3.Mono.M8, P4.Mono.M1, P4.Mono.M2, P4.Mono.M3, P4.Mono.M4, P4.Mono.M5, P4.Mono.M6, or any combination thereof. In certain cases, the methods comprise modulating one or more genes in one or more of the modules in monocytes.

In some examples, the methods comprise modulating one or more genes in one or more of the following modules in Tables 3A-3D: P1.NK.M1, P1.NK.M2, P1.NK.M3, P1.NK.M4, P2.NK.M1, P2.NK.M2, P2.NK.M3, P2.NK.M4, P3.NK.M1, P3.NK.M2, P3.NK.M3, P3.NK.M4, P3.NK.M5, P3.NK.M6, P4.NK.M1, P4.NK.M2, P4.NK.M3, P4.NK.M4, or any combination thereof. In certain cases, the methods comprise modulating one or more genes in one or more of the modules in natural killer cells.

In some examples, the methods comprise modulating one or more genes in one or more of the following modules in Tables 3A-3D: P2.PB.M1, P2.PB.M2, P3.PB.M1, P4.PB.M1, P4.PB.M2, or any combination thereof. In certain cases, the methods comprise modulating one or more genes in one or more of the modules in plasmablasts.

In some embodiments, some genes are shared in multiple modules. The expression and/or activity of the shared genes may be co-regulated or change with multiple groups of other genes. In certain cases, the expression and/or activity of some genes is co-regulated or change in multiple types of cells in response to a stimulus (e.g., an infection). In some examples, the methods comprise modulating IFI27, IFI44L, IFI6, IFIT3, ISG15, XAF1, or a combination thereof. These genes may be modulated in all of the following types of cells: monocytes, CD4+ T cells, CTLs, proliferating T cells, NK cells, B cells, plasmablasts, and dendric cells (e.g., myeloid dendric cells). In some examples, the methods comprise modulating CXCL10, DEFB1, IFI27L1, or a combination thereof. These genes may be modulated in monocytes. In some examples, the methods comprise modulating PARP9, STAT1, or a combination thereof. In some cases, these genes may be modulated in dendric cells. In some examples, the methods comprise modulating CD52, TIGIT, TRAC, or a combination thereof. In some cases, these genes may be modulated in CD4+ T cells. In some examples, the methods comprise modulating CX3CR1, ICAM2, or a combination thereof. In some cases, these genes may be modulated in NK cells. In some examples, the methods comprise modulating B2M, S100A4, KLF6, ANXA1, ITGB1, SYNE2, EZR, S100A6, AHNAK, CD52, IL32, or a combination thereof. In some cases, these genes may be modulated in CD4+ T cells. In some examples, the methods comprise modulating HLA-DQB1, HLA-DPB1, HLA-DPA1, CD74, HLA-DRA, HLA-DQA1, HLA-DRB1, CD52, or a combination thereof. In some cases, these genes may be modulated in in monocytes. In some examples, the methods comprise modulating GZMB, GZMH, GNLY, FGFBP2, NKG7, PRF1, KLRD1, CCL5, or a combination thereof. In some cases, these genes may be modulated in CTLs. In some examples, the methods comprise modulating GNPTAB, PRSS23, GZMB, GNLY, B2M, FGFBP2, NKG7, PRF1, LGALS1, TMSB4X, TMSB10, CST7, or a combination thereof. In some cases, these genes may be modulated in NK cells. In some examples, the methods comprise modulating GPR56, CST7, GZMA, KLRD1, FGFBP2, GZMH, NKG7, CCL5, CCL4, CTSW, HOPX, PRF1, GZMB GNLY, PLEK, ID2, CD8A, UBB, SPON2, FCGR3A, or a combination thereof. In some cases, these genes may be modulated in proliferating T cells. In some examples, the methods comprise modulating PRF1, GZMB, GNYL, NKG7, FGFBP2, or a combination thereof. In some cases, these genes may be modulated in CTLs, NK cells, and proliferating T cells. In some examples, the methods comprise modulating CD52. In some cases, these genes may be modulated in CD4+ T cells and monocytes. In some examples, the methods comprise modulating B2M. In some cases, these genes may be modulated in CD4+ T cells and NK cells. In some examples, the methods comprise modulating GZMH, CCL5, KLRD1, or a combination thereof. In some cases, these genes may be modulated in CTLs and proliferating T cells. In some examples, the methods comprise modulating CST7. In some cases, the gene may be modulated in NK cells and proliferating T cells. In some examples, the methods comprise modulating PRF1, GZMB, GNYL, NKG7, FGFBP2, or a combination thereof. In some cases, these genes may be modulated in NK cells, proliferating T cells, and CTL.

In some examples, the methods comprise modulating SERPINB2, CXCL3, CCL4, CCL3, IL1B, RPL5, STAT2, ICAM2, MIF, HLA-A, APOBEC3G, CD302, RPS16, SLAMF7, DUSP6, WARS, USP18, FCGR1B, CXCL1, CD300E, CCR1, IL6, CCL2, RIG-1, STAT1, HLA-G, APOBEC3B, ISG20, MX1, ISG15, IF127, or a combination thereof. In some cases, these genes may be modulated in monocytes.

In some examples, the methods comprise modulating CD8A, TNFAIP3, RGS1, HIST1H4C, PCNA, TOP2A, CCR7, ISG20, CD27, GZMK, TRDC, KLRF1, GZMB, XCL2, FCGR3A, or a combination thereof.

In some examples, the methods comprise modulating IL7R, LTB, TRBC2, LYZ, MNDA, CD14, NKG7, CCL5, GZMB, IL8, IL1B, CXCL2, MS4A1, CD79A, CD74, CD16, LST1, RHOC, STMN1, MKI67, CD8A, TNFSF10, ISG15, APOBEC3A, IGJ, IGHG1, MZB1, CD1C, HLA-DRA, CCL2, CCL4, UGCG, SERPINF1, or a combination thereof.

In some examples, the methods comprise modulating IFITM1, IFI44L, ISG15, LY6E, IFI6, SAMD9L, IFI44, MX1, OAS3, EPSTI1, EEF1A1, SFT2D2, FOSB, FOS, ANKRD36BP1, UCP2, RPLP0, RHOA, RPL9, PSAP, or a combination thereof. In some cases, these genes may be modulated in plasmacytoid dendric cells.

In some examples, the methods comprise modulating BCL2A1, C5AR1, CCL3, CO83, CTSS, CXCL2, CXCL3, DUSP2, EREG, FTH1, G0S2, GADD45B, GPR183, IER3, IL 1 B, IL8, NAM PT, NFKBIA, NFKBIZ, NLRP3, PDE4B, PLAUR, PPP1R15A, PTGS2, SAMSN1, SERPINB2, SOD2, SRGN, THBS1, TIPARP, TNFAIP3, TNFAIP6, ZFP36, or a combination thereof. In some cases, these genes may be modulated in inflammatory monocytes.

In some examples, the methods comprise modulating APOBEC3A, APOBEC3B, B2M, CXCL10, EPSTl1, GBP1, GBP4, IFl27, IFl27L 1, IFl44L, IFl6, IFIT1, IFIT2, IFIT3, IFITM1, IFITM3, IGJ, ISG15, ISG20, L Y6E, MARCKS, MX1, NT5C3A, OAS1, PLAC8, RSAD2, SAT1, TNFSF10, TXNIP, XAF1, or a combination thereof. In some cases, these genes may be modulated in anti-viral monocytes. In some examples, the methods comprise modulating RIG-I, APOBEC3B, and/or MX1 in monocytes.

In some examples, the methods comprise modulating TRBV28, TRAV4, TRBV20-1, or a combination thereof. In some cases, these genes may be modulated in proliferating T cells.

In some embodiments, the expression and/or activity of one or more genes may change in response to a treatment or medical intervention. One of more of the modulating agents may be administered to a subject to modulate such genes. For examples, the modulating agents may be used to achieve a similar or improved effect on the expression and/or activity of the genes compared to a treatment or medical intervention. In some cases, the methods comprise modulating one or more genes in Table 7A. For example, these genes may be modulated in CTLs.

In some embodiments, the methods comprise modulating one or more genes in Table 7B. For example, these genes may be modulated in CTLs. In certain examples, these genes may be modulated in proliferating T cells. In certain examples, these genes may be modulated in CTLs and proliferating T cells.

In some embodiments, the methods comprise modulating one or more genes of cluster 0 in Table 7C. For example, these genes may be modulated in regular (“traditional) CD8+ T cells. In some embodiments, the methods comprise modulating one or more genes of cluster 1 in Table 7C. For example, these genes may be modulated in hyper-proliferative CD8+ T cells. In some embodiments, the methods comprise modulating one or more genes of cluster 2 in Table 7C. For example, these genes may be modulated in naïve CD4+ T cells. In some embodiments, the methods comprise modulating one or more genes of cluster 3 in Table 7C. For example, these genes may be modulated in CD8−/TRDC+/FCGR3A+ T cells.

In some examples, the method comprises modulating PRF1 and/or GZMB in proliferating CTLs, CCL3 and/or CCL4 in NK cells, or a combination thereof. In some examples, the method comprises modulating IL-6, IL-8, IL-17, or a combination thereof in a cell. In some examples, the method comprise modulating IFN-α, IFN-γ, or a combination thereof in proliferating T cells, CD4+ T cells, CTLs, monocytes, and NK cells; one or more modulating agents that modulate IL-15, IL-12, IL-21, or a combination thereof in CTLs, NK cells, and proliferating T cells; one or more modulating agents that modulate IL-1β, TNF, or a combination thereof in CD4+ T cells; or a combination thereof.

### Upstream Drivers

In some embodiments, the methods comprise modulating one or more genes that are upstream drivers. In certain cases, the methods comprise modulating the one or more upstream driver genes in addition to modulation of the genes (e.g., in the modules) herein. Upstream drivers may refer to genes that genes that induce the alteration of expression and/or activity of the genes in a module.

In some examples, the methods comprise modulating IFN-α and/or IFN-γ. In some examples, the methods comprise modulating IL-15 and/or IL-2 in lymphocytes. In some examples, the methods comprise modulating IL-4, IL-12, and/or IL-21.

In some examples, the methods comprise modulating one or more of the following upstream driver genes: CIITA, EBI3, G-CSF, HRAS, IL6, IFNA, IL10, Ig, IL12, IL4, IL2, TBX21, IFNG, IL21, IL27, STAT1, IL15, PDCD1, or IL18. In some examples, the methods comprise modulating one or more of the following upstream driver genes: IFNG, TGFB1, STAT1, IFNA, PRDM1, SMARCA4, TP53, CIITA, G-CSF, EBI3, or IL27. In some examples, the methods comprise modulating one or more of the following upstream driver genes: IL2, IFNA, IFNG, TNF, KRAS, CD3, IL15, IL4, IL1B, TGFB1, or OSM. In some examples, the methods comprise modulating one or more of the following upstream driver genes: IL4, G-CSF, IL2, IL27, IFNA, IFNG, IL6, STAT3, IL12, Ig, IL15, IL21, or TBX21. In some examples, the methods comprise modulating one or more of the following upstream driver genes: G-CSF, IL12, IFNA, IL18, CD40LG, IL4, Ig, IL15, IL2, IFNG, STAT1, IL27, PDCD1, IL21, IL6, TBX21, STAT3, or TGFB1.

In some embodiments, the methods comprise modulating one or more upstream driver genes in specific types of cells. In some examples, the methods comprise modulating one or more of the following upstream driver genes: IFNA, OSM, IFNG, TNF, CD3, IL15, IL1B, TGFB1, KRAS, IL2, IL4, or IL6. In some cases, these upstream driver genes are modulated in CD4+ T cells. In some examples, the methods comprise modulating TNF, IL-1B, and/or OSM in CD4+ T cells.

In some examples, the methods comprise modulating one or more of the following upstream driver genes: CIITA, G-CSF, EBI3, IL27, IFNG, IFNA, STAT1, TGFB1, PRDM1, SMARCA4, or TP53. In some cases, these upstream driver genes are modulated in monocytes.

In some examples, the methods comprise modulating one or more of the following upstream driver genes in: CIITA, IFNA, IFNG, STAT1, IL27, HRAS, IL15, EBI3, G-CSF, IL18, IL10, IL4, IL2, TBX21, PDCD1, IL21, IL6, Ig, or IL12. In some cases, these upstream driver genes are modulated in NK cells. In some examples, the methods comprise modulating CIITA and/or EBI3 in NK cells.

In some examples, the methods comprise modulating one or more of the following upstream driver genes: G-CSF, IL4, IFNG, IFNA, IL15, IL6, STAT3, IL27, IL21, Ig, IL2, TBX21, IL18, IL12, TGFB1, or PDCD1. In some cases, these upstream driver genes are modulated in CTLs.

In some examples, the methods comprise modulating one or more of the following upstream driver genes: G-CSF, IL12, IFNA, IL18, IL15, TBX21, PDCD1, STAT3, IFNG, STAT1, IL27, IL21, IL6, Ig, IL2, IL4, TGFB1, or CD40LG. In some cases, these upstream driver genes are modulated in proliferating T cells.

In some examples, the methods comprise modulating one or more of the upstream driver genes in Table 6B.

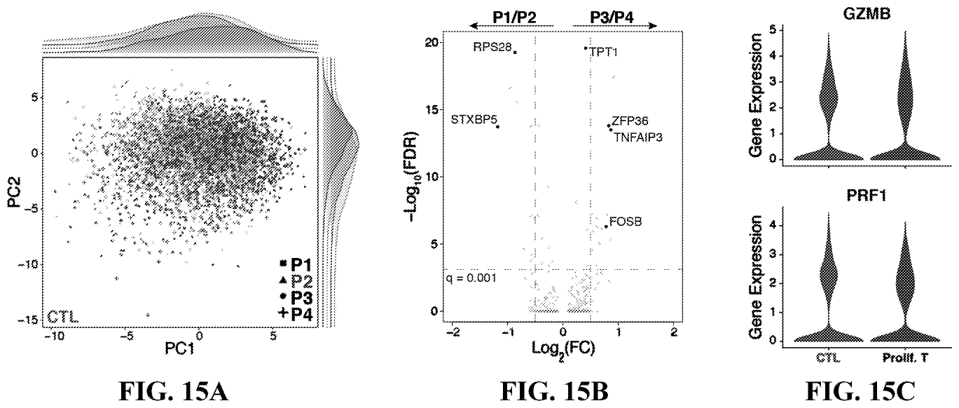

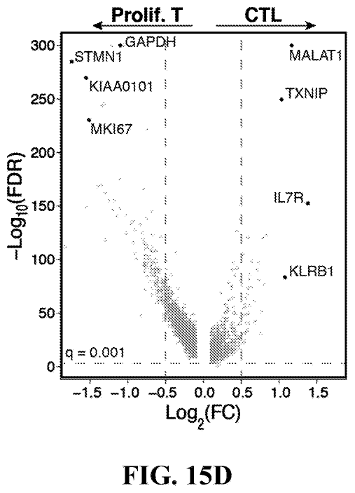

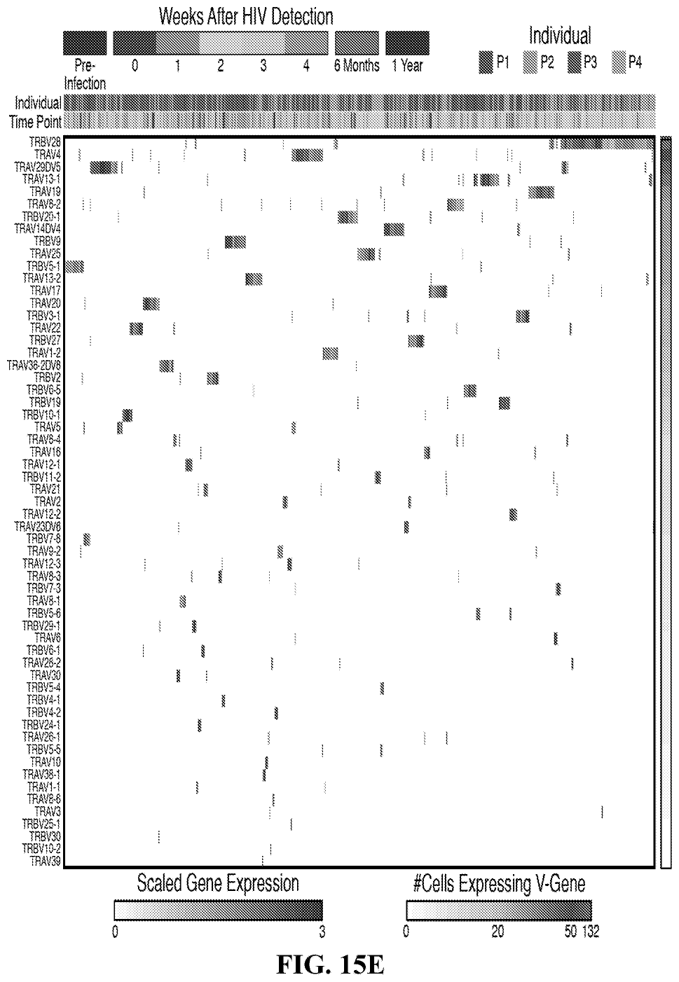

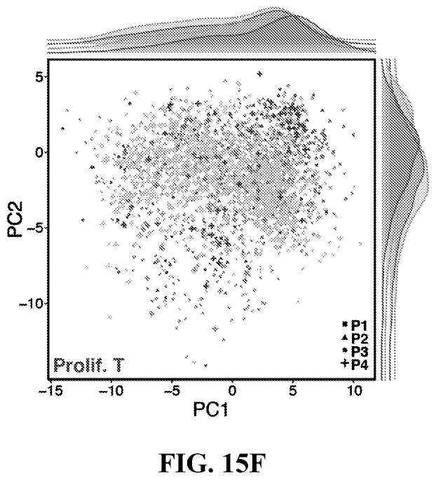

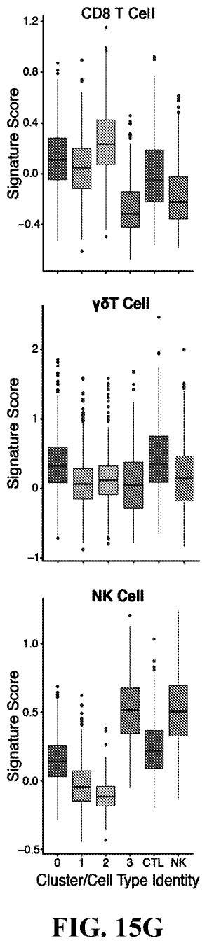

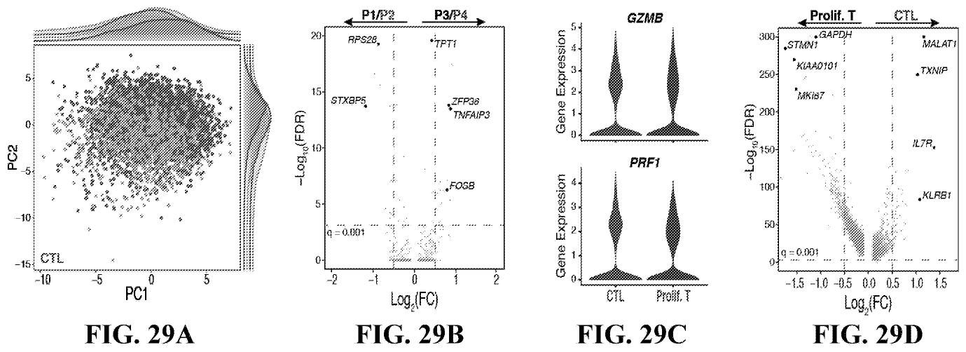

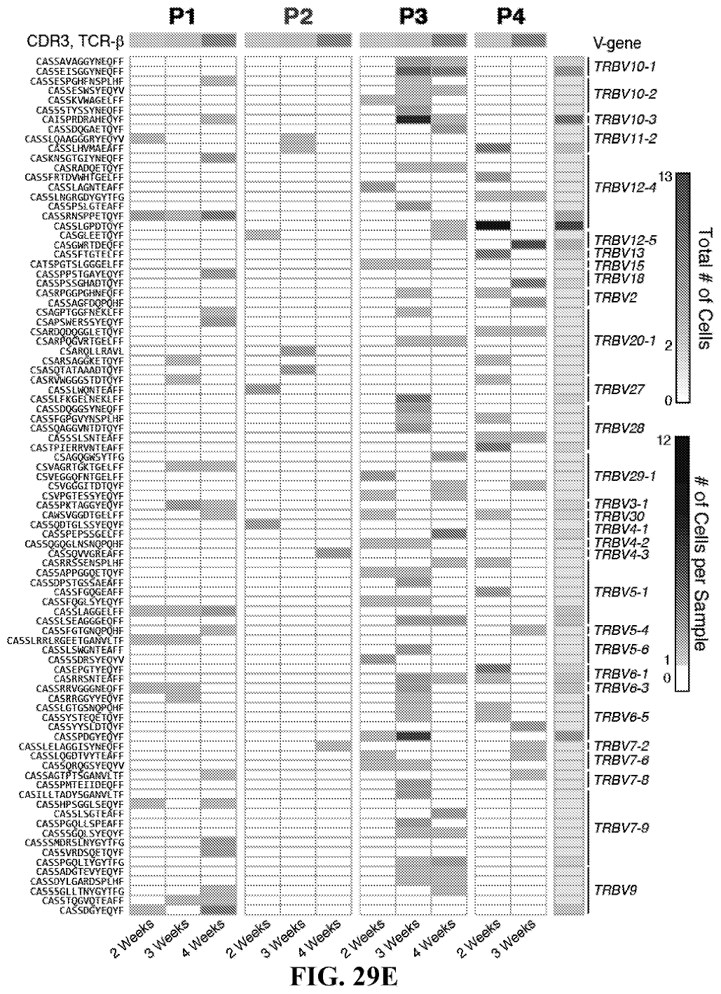

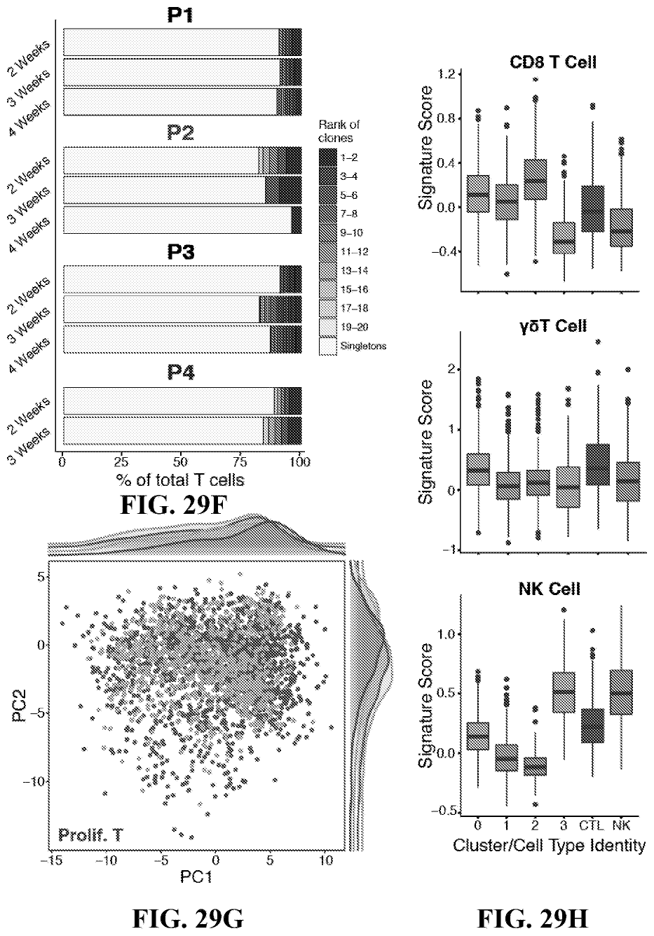

In some embodiments, the present disclosure provides methods of treating or preventing a viral infection (e.g., a chronical viral infection), the method comprising administrating an effective amount of an modulating agent that induces proliferation of γδ T cells and/or Natural killer (NK) cells to a subject in need thereof. The methods of treating or preventing a viral infection may comprise administrating an effective amount of a vaccine composition to a subject in need thereof, the vaccine composition comprising one or more modulating agents that induces proliferation of γδ T cells and/or NK cells. The one or more modulating agent modulates one or more biomarkers in FIGS. 15E and 29E. The one or more modulating agents increases KLRB1 expression in the γδ T cells and/or NK cells. In some embodiments, a method of modulating an immune response to reduce baseline inflammation comprises administrating an effective amount of one or more modulating agents that increases expression or activity of APOBEC3A, IFITM1, IFITM3, or a combination thereof in one or more immune cells. The one or more immune cells comprise monocytes, CD4+ T cells, cytotoxic T lymphocytes (CTLs), proliferating T cells, NK cells, B cells, plasmablasts, and myeloid dendritic cells. In some embodiments, a method of modulating an immune response comprises administering an effective amount of one or more modulating agents that increases activity or expression of PRF1 and/or GZMB in proliferating CTLs. In some embodiments, a method of modulating an immune response comprises administering one or more modulating agents that induces formation of polyfunctional monocytes. In some cases, the polyfunctional monocytes express one or more anti-viral and inflammatory genes. In some cases, the one or more anti-viral and inflammatory genes comprise RIG-1, STAT1, HLA-G, APOBEC3B, ISG20, MX1, ISG15, IFI27, or a combination thereof. In some cases, the one or more anti-viral and inflammatory genes comprise RIG-1, APOBEC3B, MX1, or a combination thereof. In some cases, the one or more anti-viral and inflammatory genes comprise SLAMF7, DUSP6, WARS, USP18, or a combination thereof.

### Cell Types

The methods comprise modulating the gene(s) and/or pathway(s) in one or more types of cells. The cells may include cells related to immune responses. In some cases, the cells are immune cells. As used herein, the term “immune cell” is intended to include a cell which plays a role in specific immunity (e.g., is involved in an immune response) or plays a role in natural immunity. Examples of immune cells include all distinct classes of lymphocytes (T lymphocytes, such as helper T cells and cytotoxic T cells, B lymphocytes, and natural killer cells), monocytes, macrophages, other antigen presenting cells, dendritic cells, and leukocytes (e.g., neutrophils, eosinophils, and basophils). In a preferred embodiment, the antigen is one which interacts with a T lymphocyte in the recipient (e.g., the antigen normally binds to a receptor on the surface of a T lymphocyte). Examples of the types of cells herein include CD+4 T cells, monocytes, cytotoxic lymphocytes, national killer (NK) cells, proliferating T cells, resting monocytes, inflammatory monocytes, CD16+ monocytes, anti-viral monocytes, anti-viral/inflammatory monocytes, CD1C+ dendric cells, plasmacytoid dendric cells, B cells, plasmablasts, or any combination thereof.

### Modulating Agents

Modulating one or more genes in the cells herein may be performed by administering one or more modulating agents to the cells. For example, the methods may comprise contacting the cells with the modulating agent(s). In some embodiments, the methods herein include administering one or more agents that modulate the expression and/or activity of gene(s).

For example, the methods may include administering at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100 modulating agents.

Modulating a gene may include modulating the expression of the gene. Modulating a gene may also include modulating the expression, the level, and/or the activity of a product encoded by the gene, e.g., a RNA or a protein. As will be clear to the skilled person, “modulating” can also involve affecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

“Altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Modulation herein may include increasing, decreasing, abolishing, expression and/or activity of the one or more genes. The terms “increased” or “increase” or “upregulated” or “upregulate” as used herein generally mean an increase by a statically significant amount compared to a reference. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e., absent level as compared to a reference sample), or any decrease between 10-100% as compared to a reference level, as that term is defined herein. The term “abolish” or “abolished” may in particular refer to a decrease by 100%, i.e., absent level as compared to a reference sample.

The term “agent” as used herein generally refers to any substance or composition, such as a chemical entity or biological product, or combination of chemical entities or biological products, capable of achieving a desired effect in a system, more particularly in a biological system, e.g., in a cell, tissue, organ, or an organism. In the present context, an agent may be exposed to, contacted with or introduced into an immune cell to modify at least one characteristic of the immune cell, such as to (inducibly) alter the expression or activity of the one or more genes or gene products as taught herein by the immune cell. Further in the present context, an agent may be administered to a subject to treat or prevent or control a disease or condition, for example by (inducibly) altering the expression or activity of the one or more genes or gene products as taught herein by immune cells of the subject.

In alternative embodiments, agents useful in the methods as disclosed herein are proteins and/or peptides or fragment thereof, which inhibit the gene expression of a target gene or gene product, or the function of a target protein. Such agents include, for example, but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to, mutated proteins; therapeutic proteins and truncated proteins, wherein the protein is normally absent or expressed at lower levels in the cell. Proteins can also be selected from a group comprising mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

In some embodiments, the one or more agents may be small molecules. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

In certain embodiments, the modulating agent can refer to a protein-binding agent that permits modulation or activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof.

As used herein, a “blocking” antibody or an antibody “antagonist” is one which inhibits or reduces biological activity of the antigen(s) it binds. For example, an antagonist antibody may bind a surface receptor or ligand and inhibit the ability of the receptor and ligand to induce an ILC class 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., International Patent Publication No. WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Methods for administering antibodies for therapeutic use is well known to one skilled in the art. In certain embodiments, small particle aerosols of antibodies or fragments thereof may be administered, preferably for treating a respiratory inflammatory disease (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as inhibitors or antagonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In some embodiments, the agents may be nucleic acid molecule. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In certain embodiments, an agent may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumor necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Gene Editing Systems

In certain embodiments, the one or more modulating agents may be one or more components of a gene editing system. Examples of gene editing systems include a CRISPR-Cas system, a zinc finger nuclease system, a TALEN, and a meganuclease system.

### CRISPR-Cas System

In some embodiments, the one or more modulating agents may be one or more components of a CRISPR-Cas system. In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

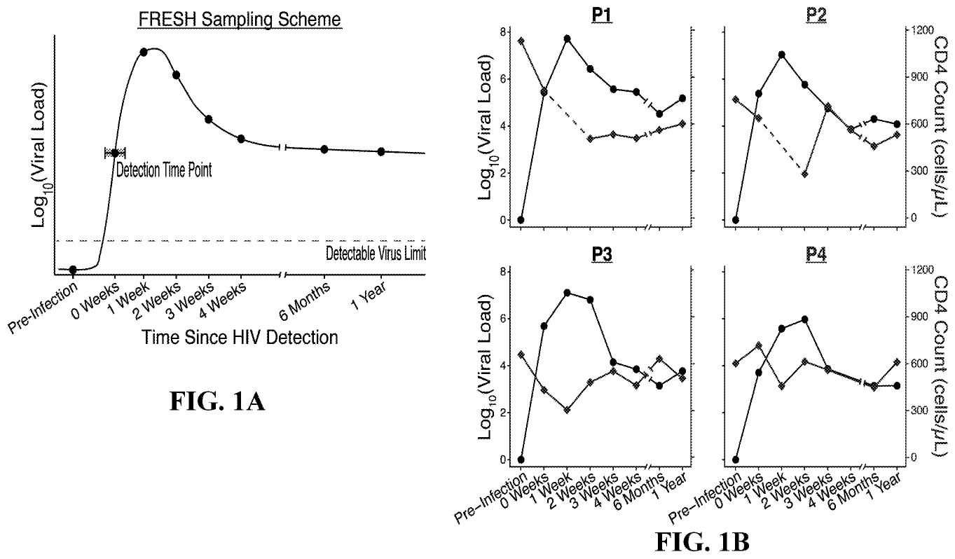

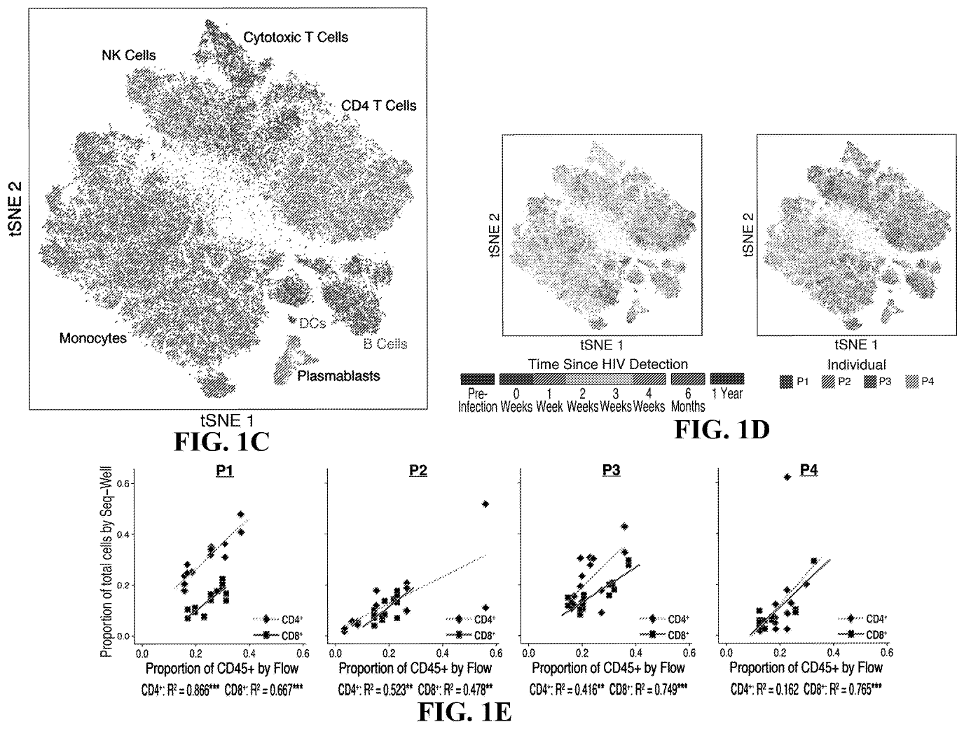

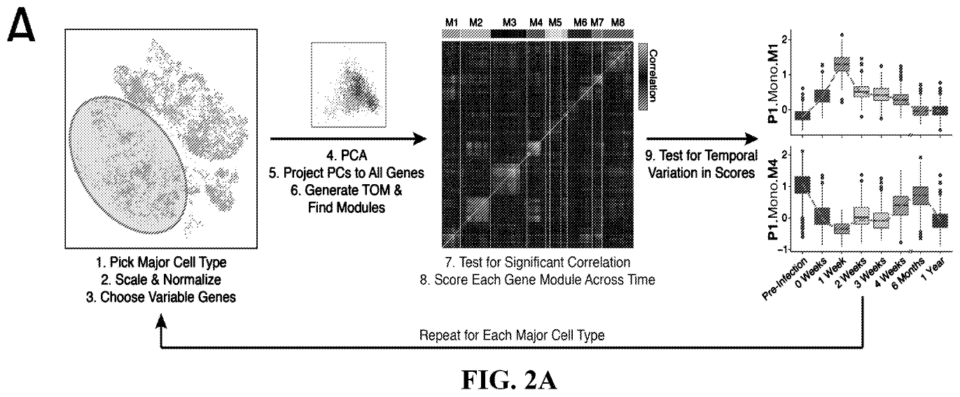

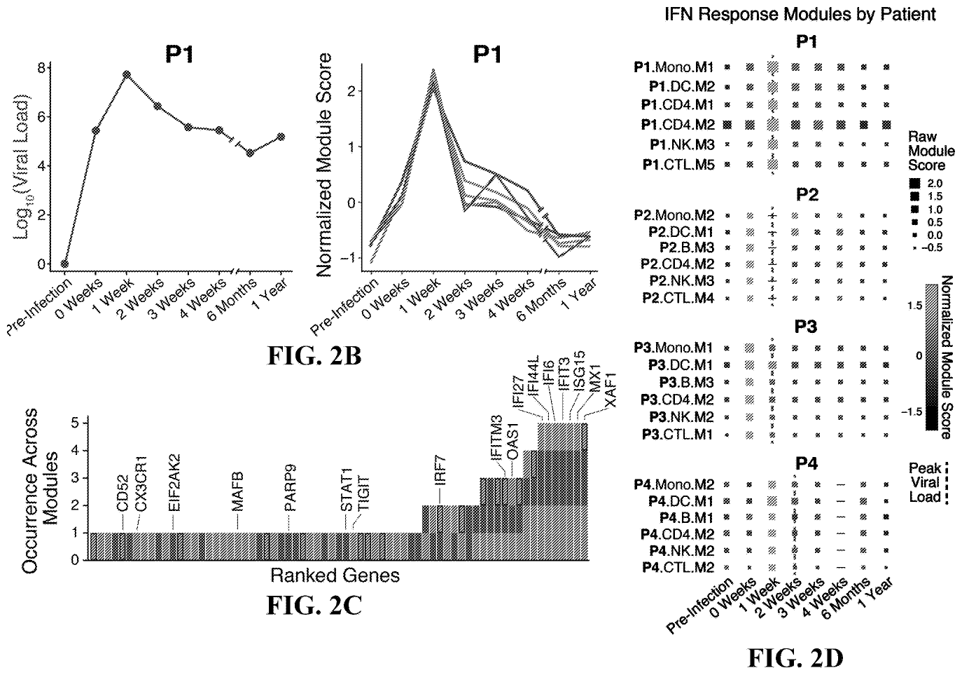

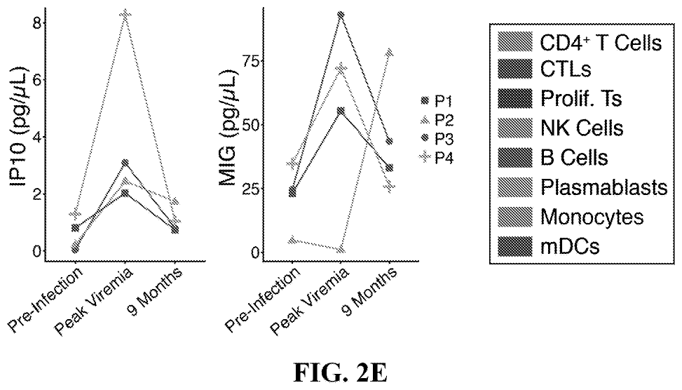

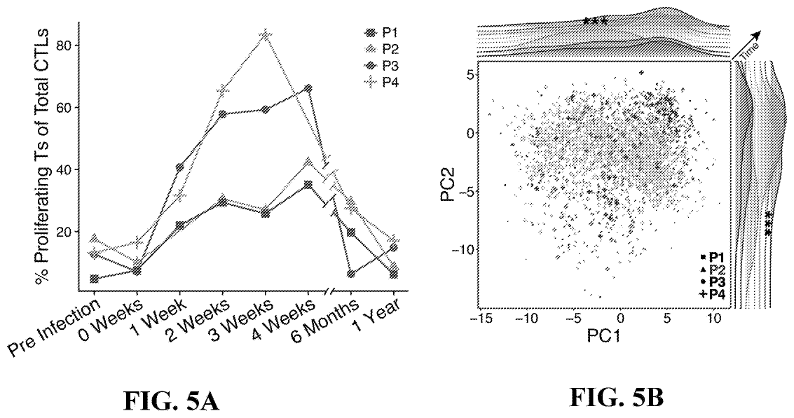

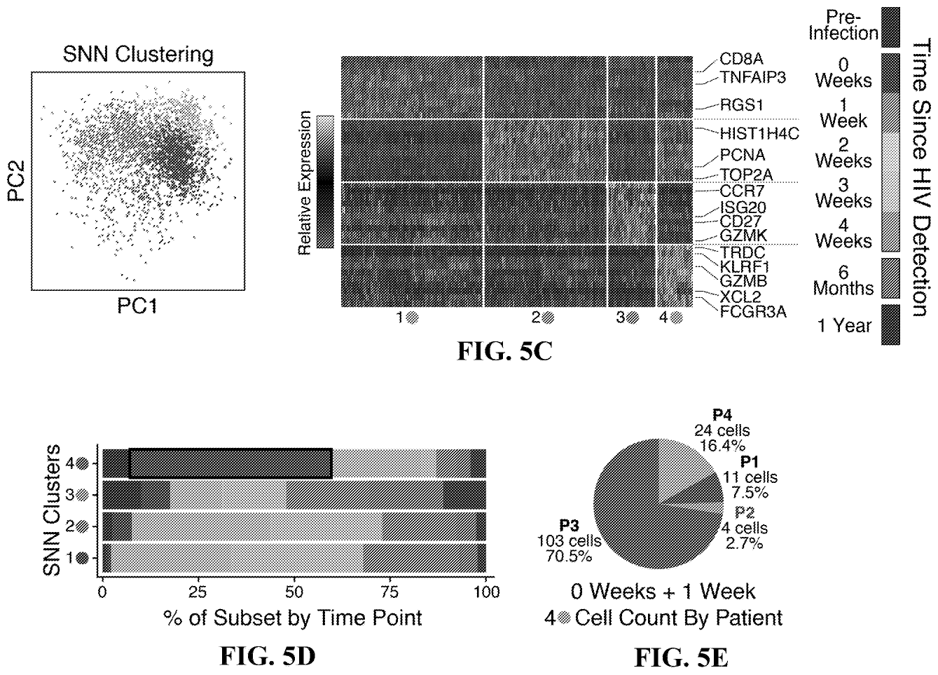

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade, in particular Class1 proteins, can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprising one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpfl), Cas12b (C2c1), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

In some embodiments, the gene editing system (e.g., a class 2, Type VI systems herein) may modify a target RNA. Such systems may knock down target RNA molecules (e.g., transcripts of target genes herein) without permanent modification of the DNA sequences of the genes. This approach may provide temporal control in modulating the expression of target genes.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication No. WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, the Cas split domains (e.g., RuvC and HNH domains in the case of Cas9) can be simultaneously or sequentially introduced into the cell such that the split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

Examples of base editing systems include those described in International Patent Publication NOs. WO 2019/071048 (e.g. paragraphs [0933]-0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO2019126762 (e.g., paragraphs [0309]-[0438]), WO 2019/126774 (e.g., paragraphs [0511]-[0670]), Cox D B T, et al., RNA editing with CRISPR-Cas13, Science. 2017 Nov. 24; 358(6366):1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of A⋅T to G⋅C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties.

### Prime Editors

In some embodiments, a polynucleotide of the present invention described elsewhere herein can be modified using a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. In some examples, a pegRNA is a sgRNA comprising a primer binding sequence (PBS) and a template containing a desired RNA sequence (e.g., added at the 3′ end). Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

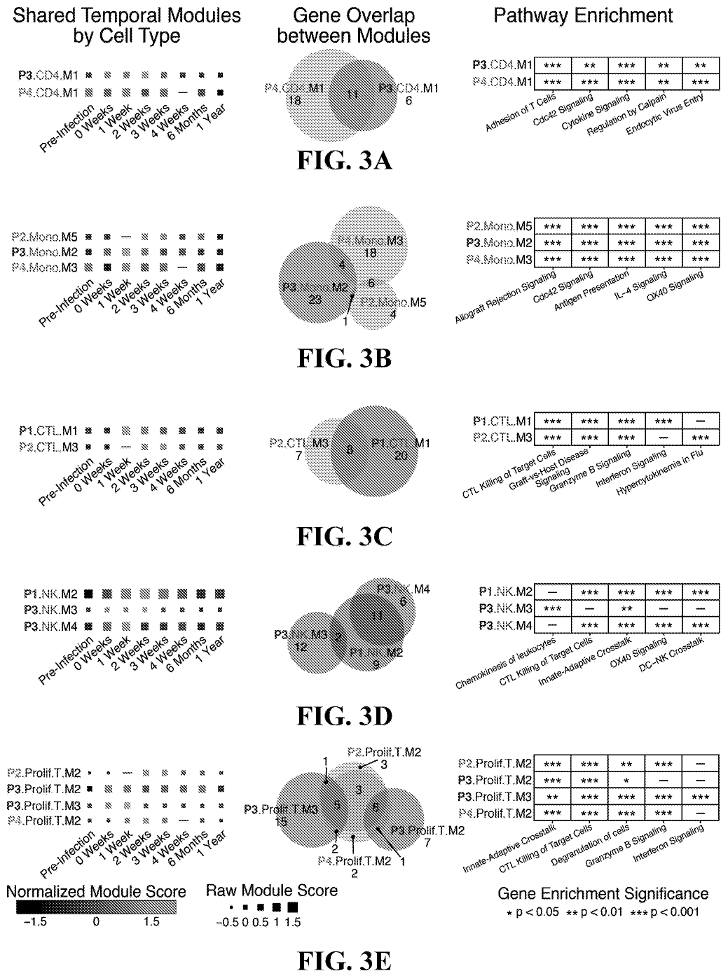

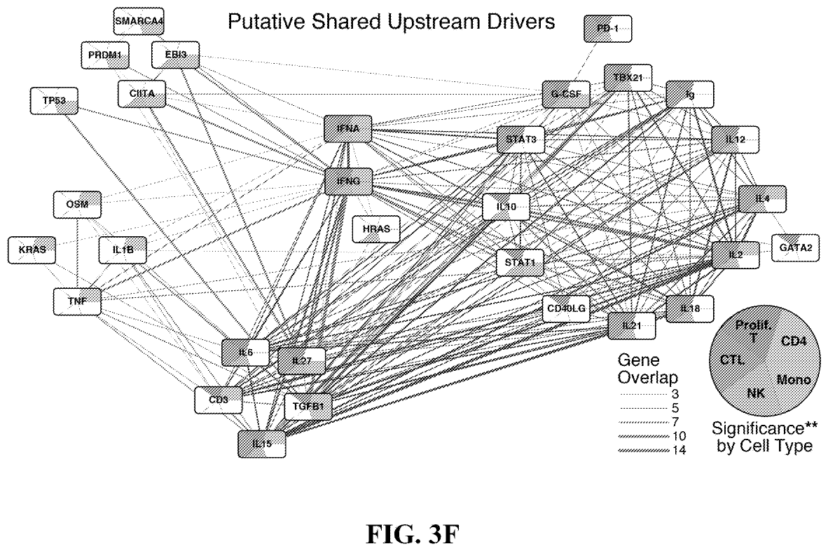

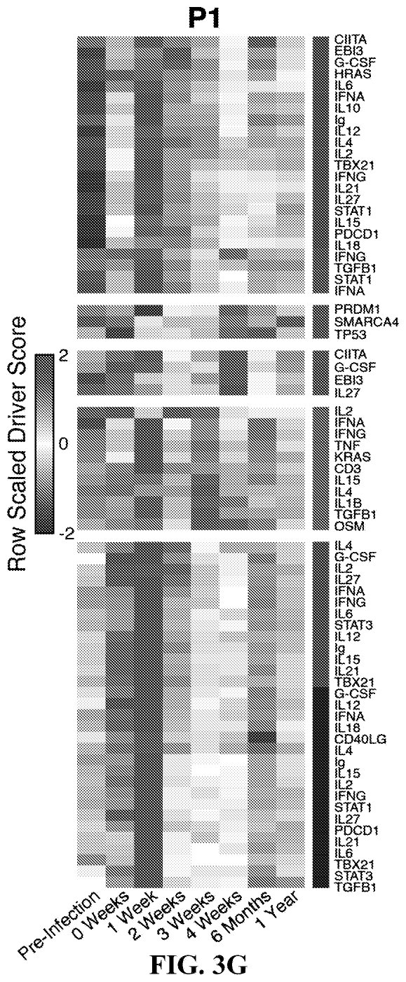

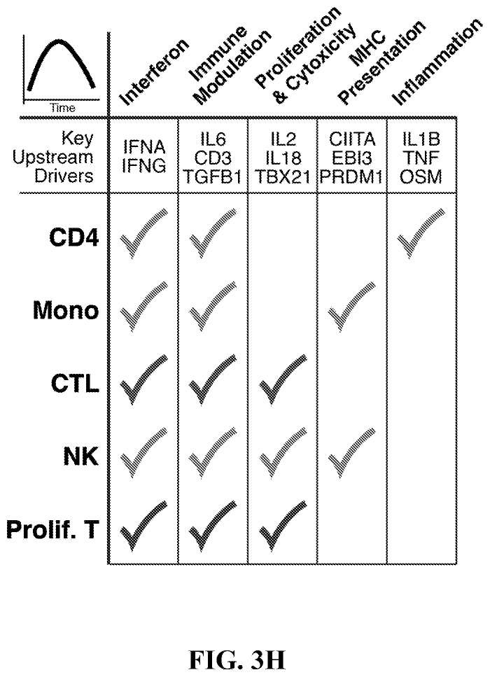

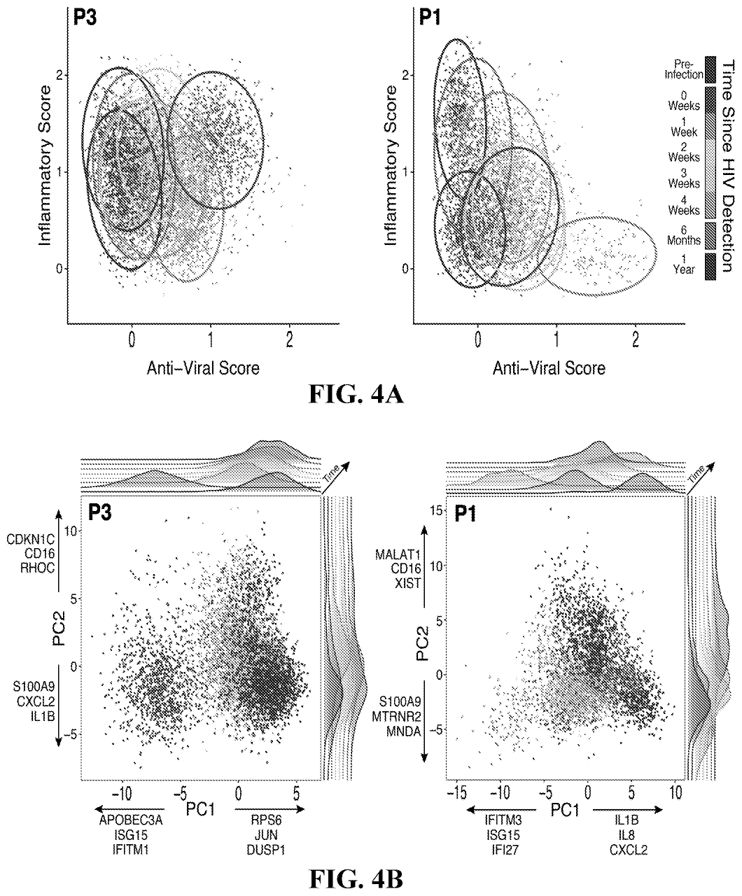

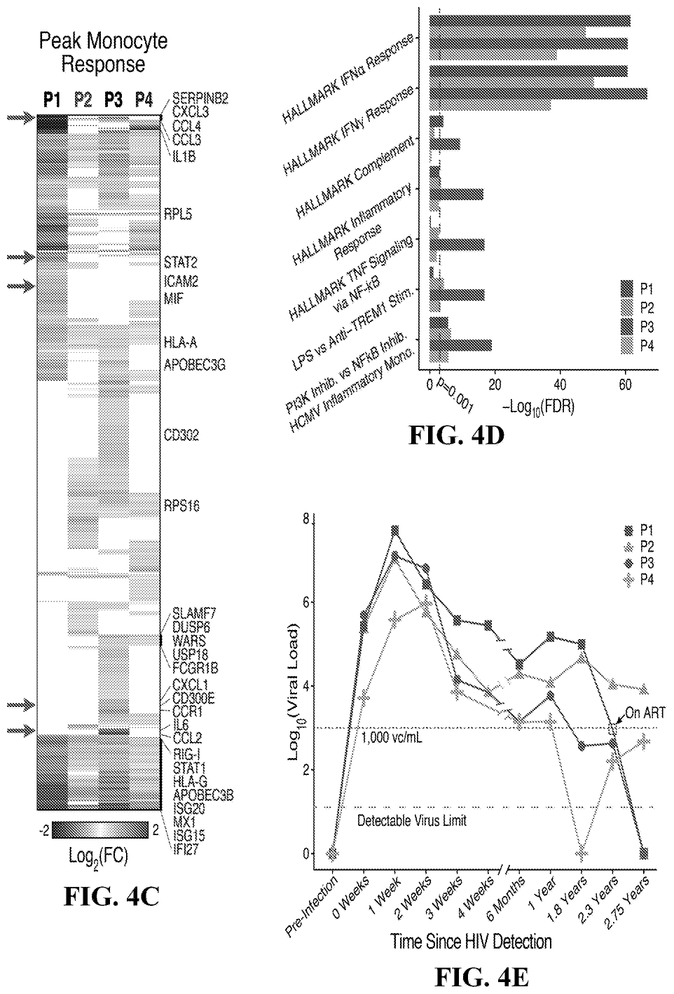

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 (from Gleditzsch et al. 2019) below shows several Cas polypeptides and the PAM sequence they recognize.

Example PAM Sequences

| | |
|---|---|
| Cas | PAM |
| Protein | Sequence |
| SpCas9 | NGG/NRG |
| SaCas9 | NGRRT or |
| | NGRRN |
| NmeCas9 | NNNNGATT |
| CjCas9 | NNNNRYAC |
| StCas9 | NNAGAAW |
| Cas12a | TTTV |
| (Cpf1) | |
| (including | |
| LbCpf1 | |
| and | |
| AsCpf1) | |
| Cas12b | TTT, TTA, |
| (C2c1) | and TTC |
| Cas12c | TA |
| (C2c3) | |
| Cas12d | TA |
| (CasY) | |
| Cas12e | 5′-TTCN-3′ |
| (CasX) | |

In a preferred embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpfl Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAsl3a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include a sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Systems

The composition may comprise one or more components of a TALE system. The composition may also comprise nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

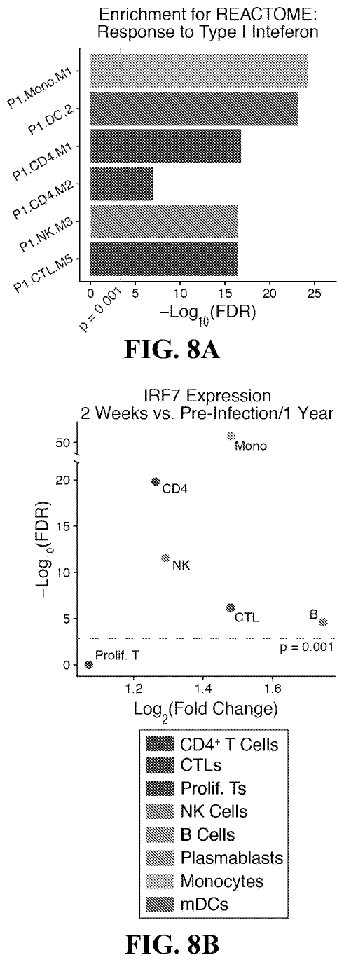

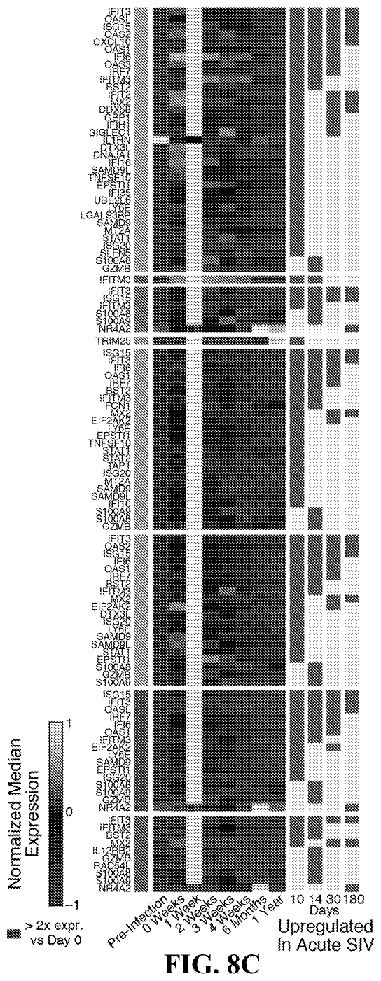

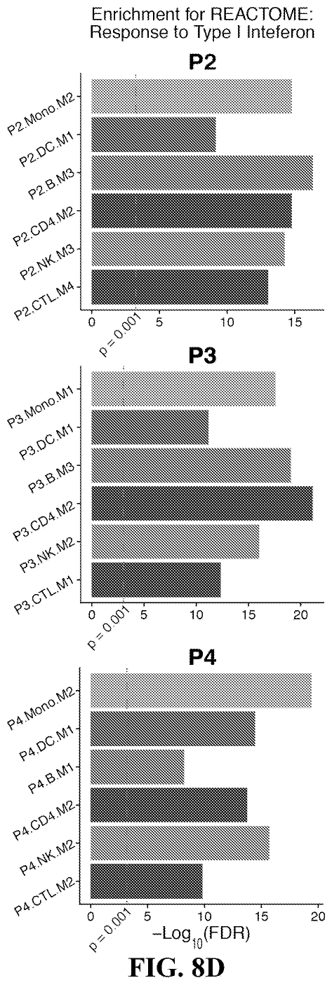

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
MDPIRSRTPSPARELLSGPQPDGVQPTADRGVSP
PAGGPLDGLPARRTMSRTRLPSPPAPSPAFSADS
FSDLLRQFDPSLFNTSLFDSLPPFGAHHTEAATG
EWDEVQSGLRAADAPPPTMRVAVTAARPPRAKPA
PRRRAAQPSDASPAAQVDLRTLGYSQQQQEKIKP
KVRSTVAQHHEALVGHGFTHAHIVALSQHPAALG
TVAVKYQDMIAALPEATHEAIVGVGKQWSGARAL
EALLTVAGELRGPPLQLDTGQLLKIAKRGGVTAV
EAVHAWRNALTGAPLN.

An exemplary amino acid sequence of a C-terminal capping region is:

(SEQ ID NO: 19)
RPALESIVAQLSRPDPALAALTNDHLVALACLG
GRPALDAVKKGLPHAPALIKRTNRRIPERTSHR
VADHAQVVRVLGFFQCHSHPAQAFDDAMTQFGM
SRHGLLQLFRRVGVTELEARSGTLPPASQRWDR
ILQASGMKRAKPSPTSTQTPDQASLHAFADSLE
RDLDAPSPMHEGDQTRAS.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

The composition may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated herein by reference.

### Meganucleases

The composition may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### Interfering RNA

In some embodiments, the modulating agents may be interfering RNAs. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Other Examples of Modulating Agents

The one or more other types of modulating agents may also be used. Agents useful in the methods as disclosed herein are proteins and/or peptides or fragment thereof, which inhibit the gene expression of a target gene or gene product, or the function of a target protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

### Exogenous Genes

In some embodiments, the modulating agents are exogenous genes or the coded products, e.g., RNA or proteins. Such exogenous genes may be any genes described herein. In certain cases, the exogenous genes may be delivered on a vector (e.g., plasmid). The expression level of the exogenous genes may be different (e.g., higher) than an endogenous gene. The exogenous gene may comprise one or more mutations or truncations compared to an endogenous counterpart gene. In certain cases, the exogenous genes may be a fusion product of multiple genes or functional fragments thereof.

### Regulatory Sequences

When the modulating agents comprise nucleic acids, the nucleic acid may be operably linked to one or more regulatory sequences. In some cases, the regulatory sequences may direct the expression of the nucleic acids in specific types. The term “operably linked” as used herein refers to linkage of a regulatory sequence to from a DNA sequence such that the regulatory sequence regulates the mediates transcription of the DNA sequence. Regulatory sequences include transcription control sequences, e.g., sequences which control the initiation, elongation and termination of transcription. In some cases, regulatory sequences include those control transcriptions. Examples of such regulatory sequences include promoters, enhancers, operators, repressor, transcription terminator sequences.

### Promoters

In some examples, the regulatory sequences are promoters. A promoter refers to a nucleic acid sequence that directs the transcription of a operably linked sequence into mRNA. The promoter or promoter region may provide a recognition site for RNA polymerase and the other factors necessary for proper initiation of transcription. When a sequence operably linked to a promoter is controlled or driven by the promoter. A promoter may include at least the Core promoter, e.g., a sequence for initiating transcription. The promoter may further at least the Proximal promoter, e.g., a proximal sequence upstream of the gene that tends to contain primary regulatory elements. The promoter may also include the Distal promoter, e.g., the distal sequence upstream of the gene that may contain additional regulatory elements.

The promoters may be from about 50 to about 2000 base pairs (bp), from about 100 to about 1000, from about 50 to about 150, from about 100 to about 200, from about 150 to about 250, from about 200 to about 300, from about 250 to about 350, from about 300 to about 400, from about 350 to about 450, from about 400 to about 500, from about 450 to about 550, from about 500 to about 600, from about 550 to about 650, from about 600 to about 700, from about 650 to about 750, from about 700 to about 800, from about 750 to about 850, from about 800 to about 900, from about 850 to about 950, from about 900 to about 1000, from about 950 to about 1050, from about 1000 to about 1100 in length.

The promoters may include sequences that bind to regulatory proteins. In some examples, the regulatory sequences may be sequences that bind to transcription activators. In certain examples, the regulatory sequences may be sequences that bind to transcription repressors.

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, β-actin promoter, phosphoglycerol kinase (PGK) promoter, ubiquitin C, U5 snRNA, U7 snRNA, tRNA promoters or EF1α promoter. In certain cases, the promoter may be a tissue-specific promoter may direct expression primarily in a desired tissue of interest, such as muscle, neuron, bone, skin, blood, specific organs (e.g. liver, pancreas), or particular cell types (e.g. lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific. In certain cases, the promoter may be an inducible promoter, e.g., can be activated by a chemical such as doxycycline.

In some cases, a promoter is specific to one or more genes. For examples, the promoter may only regulate (e.g., activates) transcription of the one or more genes, not other genes.

In some cases, the promoters may be cell-specific, tissue-specific, or organ-specific promoters. In some examples, the promoters may be CD+4 T cell specific promoters, monocyte specific promoters, cytotoxic lymphocyte specific promoters, natural killer (NK) cell specific promoters, proliferating T cell specific promoters, resting monocyte specific promoters, inflammatory monocyte specific promoters, CD16+ monocyte specific promoters, anti-viral monocyte specific promoters, anti-viral/inflammatory monocyte specific promoters, CD1C+ dendric cell specific promoters, plasmacytoid dendric cell specific promoters, B cell specific promoters, plasmablast specific promoters, dendric cell specific promoters, or any combination thereof. Examples of the cell-specific promoters include B29 promoters (for B cells), CD14 prooters (for monocytes), CD43 promoters (leukocytes and platelets), CD68 promoters (for macrophages). Other examples of tissue-specific promoters for lymphocytes include the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′transcriptional enhancer, and the human NK and T cell specific activation (NKG5) promoter.

Example of cell-specific, tissue-specific, or organ-specific promoters include promoter for creatine kinase, (for expression in muscle and cardiac tissue), immunoglobulin heavy or light chain promoters (for expression in B cells), smooth muscle alpha-actin promoter. Exemplary tissue-specific promoters for the liver include HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter. Exemplary tissue-specific promoters for the prostate include the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific promoters for gastric tissue include H+/K+-ATPase alpha subunit promoter. Exemplary tissue-specific expression elements for the pancreas include pancreatitis associated protein promoter (PAP), elastase 1 transcriptional enhancer, pancreas specific amylase and elastase enhancer promoter, and pancreatic cholesterol esterase gene promoter. Exemplary tissue-specific promoters for the endometrium include, the uteroglobin promoter. Exemplary tissue-specific promoters for adrenal cells include cholesterol side-chain cleavage (SCC) promoter. Exemplary tissue-specific promoters for the general nervous system include gamma-gamma enolase (neuron-specific enolase, NSE) promoter. Exemplary tissue-specific promoters for the brain include the neurofilament heavy chain (NF-H) promoter. Exemplary tissue-specific promoters for the colon include pp60c-src tyrosine kinase promoter, organ-specific neoantigens (OSNs) promoter, and colon specific antigen-P promoter. Exemplary tissue-specific promoters for breast cells include the human alpha-lactalbumin promoter. Exemplary tissue-specific promoters for the lung include the cystic fibrosis transmembrane conductance regulator (CFTR) gene promoter.

Examples of cell-specific, tissue-specific, or organ-specific promoters may also include those used for expressing the barcode or other transcripts within a particular plant tissue (See e.g., International Patent Publication No. WO 2001/098480A2, “Promoters for regulation of plant gene expression”). Examples of such promoters include the lectin (Vodkin, Prog. Clinc. Biol. Res., 138:87-98 (1983); and Lindstrom et al., Dev. Genet., 11:160-167 (1990)), corn alcohol dehydrogenase 1 (Dennis et al., Nucleic Acids Res., 12:3983-4000 (1984)), corn light harvesting complex (Becker, Plan tMol Biol., 20(1): 49-60 (1992); and Bansal et al., Proc. Natl. Acad. Sci. U.S.A., 89:3654-3658 (1992)), corn heat shock protein (Odell et al., Nature (1985) 313:810-812; and Marrs et al., Dev. Genet., 14(1):27-41 (1993)), small subunit RuBP carboxylase (Waksman et al., Nucleic Acids Res., 15(17):7181 (1987); and Berry-Lowe et al., J. Mol. Appl. Genet., 1(6):483-498 (1982)), Ti plasmid mannopine synthase (Ni et al., Plan tMol. Biol., 30(1):77-96 (1996)), Ti plasmid nopaline synthase (Bevan, Nucleic Acids Res., 11(2):369-385 (1983)), petunia chalcone isomerase (Van Tunen et al., EMBO J., 7:1257-1263 (1988)), bean glycine rich protein 1 (Keller et al., Genes Dev., 3:1639-1646 (1989)), truncated CaMV 35s (Odell et al., Nature (1985) 313:810-812), potato patatin (Wenzler et al., Plant Mol. Biol., 13:347-354 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90-98 1987; Wandelt and Feix, Nucleic Acids Res., 17:2354 (1989); Langridge and Feix, Cell, 34:1015-1022 (1983); and Reina et al., Nucleic Acids Res., 18:7449 (1990)), globulin-1 (Belanger et al., Genetics, 129:863-872 (1991)), α-tubulin, cab (Sullivan et al., Mol. Gen. Genet., 215:431-440 (1989)), PEPCase (Cushman et al., Plant Cell, 1(7):715-25 (1989)), R gene complex-associated promoters (Chandler et al., Plant Cell, 1: 1175-1183 (1989)), and chalcone synthase promoters (Franken et al., EMBO J., 10:2605-2612, 1991)). Examples of tissue-specific promoters also include those described in the following references: Yamamoto et al., Plant J (1997) 12(2):255-265; Kawamata et al., Plant Cell Physiol. (1997) 38(7):792-803; Hansen et al., Mol. Gen Genet. (1997) 254(3):337); Russell et al., Transgenic Res. (1997) 6(2):157-168; Rinehart et al., Plant Physiol. (1996) 112(3):1331; Van Camp et al., Plant Physiol. (1996) 112(2):525-535; Canevascini et al., Plant Physiol. (1996) 112(2):513-524; Yamamoto et al., Plant Cell Pkysiol. (1994) 35(5):773-778; Lam, Results Probl. Cell Differ. (1994) 20:181-196; Orozco et al., Plant Mol. Biol. (1993) 23(6):1129-1138; Matsuoka et al., Proc Natl. Acad. Sci. USA (1993) 90(20):9586-9590; and Guevara-Garcia et al., Plant J. (1993) 4(3):495-505; maize phosphoenol carboxylase (PEPC) has been described by Hudspeth & Grula (Plant Molec Biol 12: 579-589 (1989)); leaf-specific promoters such as those described in Yamamoto et al., Plant J. (1997) 12(2):255-265; Kwon et al., Plant Physiol. (1994) 105:357-367; Yamamoto et al., Plant Cell Physiol. (1994) 35(5):773-778; Gotor et al., Plant J. (1993) 3:509-518; Orozco et al., Plant Mol. Biol. (1993) 23(6):1129-1138; and Matsuoka et al., Proc. Natl. Acad. Sci. USA (1993) 90(20):9586-9590.

### Adoptive Cell Therapy

The compositions, systems, and methods described herein can be used to modify cells for an adoptive cell therapy. In some cases, the modified cells may be used for treating viral infection. For example, the cells (e.g., T cells and/or NK cells) may be modified ex vivo and used to treat or prevent viral infections.

In an aspect of the invention, methods and compositions which involve editing a target nucleic acid sequence, or modulating expression of a target nucleic acid sequence, and applications thereof in connection with treating infectious diseases are comprehended by adapting the composition, system, of the present invention. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as antigens derived from viruses. Examples of such approaches include those described in, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In certain embodiments, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and 3, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIRI, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PDi.

In certain embodiments, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In certain embodiments, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In certain embodiments, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MIIC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MIIC molecule of interest and any antigen of interest as described herein. Specific epitopes to be used in this context can be identified using numerous assays known in the art. For example, the ability of a polypeptide to bind to MIIC class I may be evaluated indirectly by monitoring the ability to promote incorporation of 125I labeled β2-microglobulin (β2m) into MIIC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m²/day.

Other examples of modulating agents include those for:
APOBEC3A (e.g., as described in Olson M E, Harris R S, Harki D A. APOBEC Enzymes as Targets for Virus and Cancer Therapy. Cell Chem Biol. 2018; 25(1):36-49. doi: 10.1016/j.chembiol.2017.10.007);
IFITM1/IFTIM3 (e.g., as described in Bailey C C, Zhong G, Huang I C, Farzan M. IFITM-Family Proteins: The Cell's First Line of Antiviral Defense. Annu Rev Virol. 2014; 1:261-283. doi:10.1146/annurev-virology-031413-085537);
GZMB (e.g., as described in Bailey C C, Zhong G, Huang I C, Farzan M. IFITM-Family Proteins: The Cell's First Line of Antiviral Defense. Annu Rev Virol. 2014; 1:261-283. doi: 10.1146/annurev-virology-031413-085537);
RIG-1 (e.g., as described in Kasumba et al. “Therapeutic Targeting of RIG-1 and MDA5 Might Not lead to the same Rome” Trends in Pharmacological Sicneces, 2019, 40(2):116-127);
STAT1 (e.g., as described in Miklossy G, Hilliard T S, Turkson J. Therapeutic modulators of STAT signalling for human diseases. Nat Rev Drug Discov. 2013; 12(8):611-629. doi:10.1038/nrd4088);
ISG15 (e.g., as described in Fernandez et al. “Strategies to Target ISG16 and USP18 Toward Therapeutic Applications,” Font Chem. 2020);
SLAMF7 (e.g., as described in Friend R, Bhutani M, Voorhees P M, Usmani S Z. Clinical potential of SLAMF7 antibodies—focus on elotuzumab in multiple myeloma. Drug Des Devel Ther. 2017; 11:893-900. Published 2017 Mar. 20. doi: 10.2147/DDDT.S98053);
CXCL10 (e.g., as described in Wilson N O, Solomon W, Anderson L, et al. Pharmacologic inhibition of CXCL10 in combination with anti-malarial therapy eliminates mortality associated with murine model of cerebral malaria. PLoS One. 2013; 8(4):e60898. Published 2013 Apr. 5. doi: 10.1371/journal.pone.0060898);
CD52 (e.g., as described in Blatt K, Herrmann H, Hoermann G, et al. Identification of campath-1 (CD52) as novel drug target in neoplastic stem cells in 5q-patients with MDS and AML. Clin Cancer Res. 2014; 20(13):3589-3602. doi: 10.1158/1078-0432);
CCR (e.g., 13-2811);
TIGIT (e.g., MK-7864);
TRAC (e.g., as described in Solomon B L, Garrido-Laguna I. TIGIT: a novel immunotherapy target moving from bench to bedside. Cancer Immunol Immunother. 2018; 67(11):1659-1667. doi:10.1007/s00262-018-2246-5);
CX3CR1 (e.g., as described in Ridderstad Wollberg A, Ericsson-Dahlstrand A, Juréus A, et al. Pharmacological inhibition of the chemokine receptor CX3CR1 attenuates disease in a chronic-relapsing rat model for multiple sclerosis. Proc Natl Acad Sci USA. 2014; 111(14):5409-5414. doi:10.1073/pnas.1316510111);
USP18 (see IGS15) ⋅B2M (e.g., as described in Wang D, Quan Y, Yan Q, Morales J E, Wetsel R A. Targeted Disruption of the β2-Microglobulin Gene Minimizes the Immunogenicity of Human Embryonic Stem Cells. Stem Cells Transl Med. 2015; 4(10):1234-1245. doi: 10.5966/sctm.2015-0049);
EZR (Exemplary modulating agents of EZR gene and/or gene product(s) such as antibodies (see e.g. abeam Cat. No. ab231907 www.abcam.com/ezrin-antibody-ab231907.html; abeam Cat. No. ab4069, www.abcam.com/ezrin-antibody-3c12-ab4069.html) and small molecule agents (see e.g., Celik et al. Mol Cancer Ther. 2015 November; 14(11): 2497-2507, doi: 10.1158/1535-7163.MCT-15-0511; and Bulut et al., Oncogene. 2012 Jan. 19; 31(3): 269-281, doi: 10.1038/onc.2011.245);
S100A6 (Gene product—S100 Calcium-binding protein A6 (aka calcyclin) involved in wide range of cellular process including cell cycle progression and differentiation. Research antibodies known. Described as marker in some cancer types. Agents that stimulate oxidative stress stimulated S100A6 gene expression. Hormones siRNA described as therapy for endometriosis. Exemplary modulating agents of S100A6 gene and/or gene product(s) include antibodies (see e.g. lifespan BioSciences Cat. No. LS-B5528) and small molecule agents (see e.g., Bresnick et al. Biophys Rev. 2018 December; 10(6): 1617-1629; Lesniak et al., Biochimica et Biophysica Acta (BBA)—Molecular Cell Research Volume 1744, Issue 1, 15 May 2005, Pages 29-37, https://doi.org/10.1016/j.bbamcr.2004.11.003; and Yang et al., Life Sc. 2006 Jan. 11; 78(7):753-60. doi: 10.1016/j.lfs.2005.05.100));
AHNAK (Gene product neuroblast differentiation-associated protein (aka desmoyokin; Exemplary modulating agents of AHNAK gene and/or gene product(s) include antibodies (see e.g. ThermoFisher Scientific Cat. No. MA1-10050));
IL31 (e.g., Therapeutic antibody known named Lokivetmab is available for trx of canine atopic dermatitis. IL-31 Receptor therapeutic antibodies are known. Exemplary modulating agents of IL31 gene and/or gene product(s) include antibodies (see e.g.
Lokivetmab (Hilde Moyaert et al.: A blinded, randomized clinical trial evaluating the efficacy and safety of lokivetmab compared to ciclosporin in client owned dogs with atopic dermatitis. In: Vet. Dermatology, September 2017 doi:10.1111/vde.12478; Nakashima et al., Dermatol. 2018 April; 27(4):327-331. doi: 10.1111/exd.13533; nemolizumab (Ruzicka et al., N Engl J Med 2017; 376:826-835 DOI: 10.1056/NEJMoa1606490));
CD74 (Exemplary modulating agents of CD74 gene and/or gene product(s) include antibodies (see e.g. Frolich et al., Arthritis Res Ther. 2012; 14(2): R54, doi: 10.1186/ar3767) and antibody-dug conjugates (see e.g. Abrahams et al., Oncotarget. 2018 Dec. 28; 9(102): 37700-37714., doi: 10.18632/oncotarget.26491);
GZMH (antibodies. (see e.g., Themo-Fisher Cat. No. PA5-42227, RND Systems MAB1377));
SERPINB1 (e.g., as described in Wang et al., J Immunol. 2013 Feb. 1; 190(3):1319-30. doi: 10.4049/jimmunol.1202542. Epub 2012 Dec. 26. PMID: 23269243);
GNLY (e.g., as described in—WO2014106235A1 describes anti-granulysin antibodies and uses thereof);
PRF1 (e.g., T-cell gene therapy for PRF1 gene described (Ghosh et al., J. Allergy and Clinical Immunology. Volume 142, Issue 3, September 2018, Pages 904-913.e3, doi.org/10.1016/j.jaci.2017.11.050); Small molecule inhibitors: Miller et al., Bioorg Med Chem Lett. 2016 Jan. 15; 26(2): 355-360; Spicer et al., Bioorg Med Chem Lett. 2017 Feb. 15; 27(4): 1050-1054; Spicer et al., J. Med. Chem. 2020, 63, 5, 2229-2239, doi.org/10.1021/acs.jmedchem.9b00881);
KLRD1 (e.g., CD94 therapeutic antibody (Monalizumab) McWilliams et al., Oncoimmunology. 2016; 5(10): e1226720, doi: 10.1080/2162402X.2016.1226720
CCL5 (e.g., Therapeutic antibody known in development with NovImmune CreativeBiolabs: NI-0701 (Cat. No. TAB-098CL), US20190002571A1; Scalley-Kim et al., 2012. PLOS One https://doi.org/10.1371/journal.pone.0043332)
LGALS1 (e.g., Galectin 1 inhibitors: Blanchard et al., Expert Opin Ther Pat. 2016 May; 26(5):537-54, DOI: 10.1517/13543776.2016.1163338; St-Pierre et al., Antimicrobial Agents and Chemotherapy. 2012. 56(1): 154-162; Mukherjee et al., International Journal of Obesity volume 39, pages 1349-1358(2015); Ito et al., CANCER AND METASTASIS REVIEW 31(3-4):763-78; Astorgues-Xerri et al., E. J. Cancer. Volume 50, Issue 14, September 2014, Pages 2463-2477; Dahlqvist et al., ACS Omega 2019, 4, 4, 7047-7053; Goud et al.,: Mini-Reviews in Medicinal Chemistry. 2019. DOI: 10.2174/1389557519666190304120821; Dahlqvist et al., Beilstein J. Org. Chem. 2019, 15, 1046-1060. doi: 10.3762/bjoc.15.102; Wu et al., 2020. RCS Advances. 10: 19636-19642; Collins et al., Chem Biol Drug Des. 2012. 79:339-346);
TMSB4X (e.g., as described in Suh et al., J Obstet Gynecol. 1985 Feb. 15; 151(4):544-9. doi: 10.1016/0002-9378(85)90286-8; U.S. Pat. Nos. 8,632,827; 9,114,089);
GPR56 (e.g., Therapeutic antibody e.g., as described in Tokoro et al., Exp Hematol. 2018 March; 59:51-59.el. doi: 10.1016/j.exphem.2017.12.001. Epub 2017 Dec. 7; Modulators: Stoveken et al., 2016. Mol. Pharmacol. 90:214-224; Chiang et al., Journal of Cell Science 2016 129: 2156-2169; doi: 10.1242/jcs.174458);
CTSW (e.g., Inhibitors e.g., as described in Snood et al., Pharmaceuticals (Basel). 2019 June; 12(2): 87);
HOPX (e.g., Modulators e.g., as described in Waraya et al: BMC Cancer volume 12, Article number: 397 (2012));
ID2 (e.g., Modulators e.g., as described in Zhu et al., Cell Mol Life Sci. 2003 January; 60(1):212-8. doi: 10.1007/s000180300015);
CD8A (e.g., as described in Clement et al., J Immunol. 2011 Jul. 15; 187(2): 654-663.; Wooldridge et al., J Immunol Dec. 15, 2003, 171 (12) 6650-6660; DOI:
doi.org/10.4049/jimmunol.171.12.6650);
FCGR3A (e.g., Therapeutic Antibodies e.g., as described in Reusch et al., mAbs. 2014. 6:727-738 https://doi.org/10.4161/mabs.28591; Wu et al., Journal of Hematology & Oncology volume 8, Article number: 96 (2015); Li et al., Exp. Molec. Pathol. Volume 101, Issue 2, October 2016, Pages 281-289);
IL1B (e.g., Modulators e.g., as described in Dinarello et al., Nat Rev Drug Discov. 2012 August; 11(8): 633-652);
MIF (e.g., Therapeutic Antibody e.g., as described in Kerschbaumer et al., J. Biol Chem. 2012 Mar. 2; 287(10):7446-55. doi: 10.1074/jbc.M111.329664; Bloom et al., Expert Opin Ther Targets. 2016 December; 20(12):1463-1475, Clinical Trail No. NCT01765790; Modulators: Kok et al., Drug Discov Today. 2018 November; 23(11): 1910-1918; Tilstam et al., J. Biol. Chem. doi: 10.1074/jbc.RA119.009860; Cheng et al., Scientific Reports volume 10, Article number: 6741 (2020));
CD302 (e.g., Therapeutic Antibodies e.g., as described in Lo et al., PLoS One. 2019; 14(5): e0216368).
FCGR1B (e.g., Antibody: Akinrinmade et al., Biomedicines. 2017 September; 5(3): 56; Balaian et al., Leukemia Res. Volume 28, Issue 8, August 2004, Pages 821-829; Modulators: Lu et al., www.jbc.org/cgi/doi/10.1074/jbc.M109.035683);
CXCL1 (e.g., Therapeutic antibodies: Miyake et al., Theranostics 2019; 9(3):853-867. doi:10.7150/thno.29553; Parkunan et al., J Leukoc Biol. 2016 November; 100(5): 1125-1134.; Modulators: Wang et al., J Exp Med (2006) 203 (4): 941-951);
CCR1 (e.g., Modulators: Hesselgesser et al., J. Biol. Chem. 273:15687-15692; BX-471 (ZK811752) https://doi.org/10.1016/B978-1-4160-6068-0.00020-6IL6; Kath et al., Bioorganic Medicinal Chem. Lett. Volume 14, Issue 9, 3 May 2004, Pages 2169-2173; Liang et al., E. J. Pharmacol. Volume 389, Issue 1, 11 Feb. 2000, Pages 41-49; Sabroe et al., J. Biol. Chem. 275:25985-25992);
TNFAIP3 (e.g., as described in Momtazi et al., Am J Physiol Lung Cell Mol Physiol. 2019 Mar. 1; 316(3):L456-L469; doi: 10.1152/ajplung.00335.2018);
PCNA (see, e.g. molecular targeting using cell penetrating peptide, Smith et al., Mol.
Therapy Oncolytics, v. 17, 26 Jun. 2020, 250-256; doi: 10.1016/j.omto.2020.03.025; small molecule Dillehay et al., Mol. Cancer Ther. 2014 13(12); doi:10.1158/1535-7163.MCT-14-0522);
TOP2A (e.g., as described in Jain et al, Endocr Relat Cancer. 2013 May 21; 20(3):361-70. doi: 10.1530/ERC-12-0403; Belluti, et al. Cell Death Dis 4, e756 (2013); doi: 10.1038/cddis.2013.287);
CCR7 (e.g., as described in Sorfi et al., Transplantation. 2006 Sep. 27; 82(6):826-34. doi: 10.1097/01.tp.0000235433.03554.4f; Ferreira et al., Nature Genetics 49, 1752-1757 (2017) at Table 2 showing CCR7 antagonists (asthma));
XCL2 (e.g., as described in Fox et al., Cytokine. 2015 February; 71(2): 302-311; doi: 10.1016/j.cyto.2014.11.010);
LTB (e.g., as described in Hicks et al., Expert Opin Investig Drugs. 2007 December; 16(12):1909-20. doi: 10.1517/13543784.16.12.1909 (antagonists for inflammatory disease));
TRBC2 (e.g., as described in Maciocia, P., Wawrzyniecka, P., Philip, B. et al. Targeting the T cell receptor β-chain constant region for immunotherapy of T cell malignancies. Nat Med 23, 1416-1423 (2017); doi: 10.1038/nm.4444));
CD14 (e.g., as described in Tunheim et al., J Leukoc Biol. 2005 March; 77(3):303-10. doi: 10.1189/jlb.0804480 (monoclonal antibodies));
CCL5 (e.g., as described in Aldinucci, et al., Int J Mol Sci. 2018 May 16; 19(5):1477. doi: 10.3390/ijms19051477 (cancer));
IL1B (e.g., as described in Dinarello, et al. Nat Rev Drug Discov. 2012 August; 11(8): 633-652);
CXCL2 (e.g., as described in Guo et al., Clinical and Experimental Hypertension 42 (5), 2020; doi: 10.1080/10641963.2019.1693585);
CD79A (e.g., as described in Polson et al., Blood. 2007 Jul. 15; 110(2):616-23. doi: 10.1182/blood-2007-01-066704 (antibody-drug conjugates));
CD74 (e.g., as described in Abrahams, et al., Oncotarget. 2018 Dec. 28; 9(102): 37700-37714. doi: 10.18632/oncotarget.26491 (antibody-drug conjugates));
CD16 (e.g., as described in Romee, et al., Blood. 2013 May 2; 121(18):3599-608. doi: 10.1182/blood-2012-04-425397 (regulation by a Disintegrin and metalloprotease-17 (ADAM17));
RHOC (e.g., as described in Xu et al., Onco Targets Ther. 2017; 10: 1827-1834. doi: 10.2147/OTT.S93164 (siRNAs for cancer));
STMN1 (e.g., as described in Wang R, Wang Z, Yang J, Liu X, Wang L, Guo X, Zeng F, Wu M, Li G. LRRC4 inhibits the proliferation of human glioma cells by modulating the expression of STMN1 and microtubule polymerization. J Cell Biochem. 2011; 1122):3621-9; Li J, Hu G H, Kong F J, Wu K M, He B, Song K, Sun W J. Reduced STMN1 expression induced by RNA interference inhibits the bioactivity of pancreatic cancer cell line Panc-1. Neoplasma. 2014; 61(2):144-52.);
TNFSF10 (e.g., as described in Cantarella, et al. Brain, Volume 138, Issue 1, January 2015, Pages 203-216, doi: 10.1093/brain/awu318);
IGJ (e.g., as described in Cole, S., Walsh, A, Yin, X. et a. Integrative analysis reveals CD38 as a therapeutic target for plasma cell-rich pre-disease and established rheumatoid arthritis and systemic lupus erythematosus. Arthritis Res Ther 20, 85 (2018). doi: 10.1186/s13075-018-1578-z) ⋅IGHG1 (e.g., as described in Pan, et al, Mol Biol Rep. 2013 January; 40(1):27-33. doi: 10.1007/s11033-012-1944-x. (siRNAs in cancer));
CD1C (e.g., as described in Allen, et al., J Immunol May 1, 2011, 186 (9) 5261-5272; doi: 10.4049/jimmunol.1003615);
UGCG (e.g., as described in Schomel, N., Hancock, S. E., Gruber, L. et al. UGCG influences glutamine metabolism of breast cancer cells. Sci Rep 9, 15665 (2019). doi:10.1038/s41598-019-52169-7);
IFI44L (e.g., as described in Luo et al., Auto-Immunity. 136(8), Aug. 1, 2016; doi: 10.1016/j.jid.2016.05.007);
LY6E (e.g., as described in Asundi, et al., Clin. Canc. Res. July 2015 doi: 10.1158/1078-0432.CCR-15-0156);
SAMD9L (e.g., as described in Zhang, et al., J. Vir. June 2019 93:12, doi: 10.1128/JVI.00225-19);
OAS3 (e.g., as described in Gonzalez, et al., Comput Biol Chem. 2020 April; 85:107211. doi: 10.1016/j.compbiolchem.2020.107211);
EEF1A1 (e.g., as described in Kobayashi, Y., Yonehara, S. Novel cell death by downregulation of eEF1A1 expression in tetraploids. Cell Death Differ 16, 139-150 (2009). doi: 10.1038/cdd.2008.136);
FOSB (e.g., as described in Cates, et al., eNeuro 20 Mar. 2019, 6 (2) ENEURO.0325-18.2019; DOI: 10.1523/ENEURO.0325-18.2019);
UCP2 (e.g., as described in Donadelli, M., Dando, I., Fiorini, C. et al. UCP2, a mitochondrial protein regulated at multiple levels. Cell. Mol. Life Sci. 71, 1171-1190 (2014). Doi: 10.1007/s00018-013-1407-0);
EREG (e.g., as described in Gene silencing of EREG mediated by DNA methylation and histone modification in human gastric cancers Jiyeon Yun, Sang-Hyun Song, Jinah Park,
Hwang-Phill Kim, Young-Kwang Yoon, Kyung-Hun Lee, Sae-Won Han, Do-Youn Oh, Seock-Ah Im, Yung-Jue Bang & Tae-You Kim Laboratory Investigation volume 92, pages 1033-1044(2012));
GADD45B (e.g., as described in Cretu A, Sha X, Tront J, Hoffman B, Liebermann D A. Stress sensor Gadd45 genes as therapeutic targets in cancer. Cancer Ther. 2009; 7(A):268-276);
IL1B (e.g., as described in Lorena Arranz 1, Maria Del Mar Arriero 2, Alicia Villatoro Blood Rev 2017 September; 31(5):306-317. doi: 10.1016/j.blre.2017.05.001. Epub 2017 May 3. Interleukin-1β as Emerging Therapeutic Target in Hematological Malignancies and Potentially in Their Complications);
NAMPT (e.g., as described in Mari Nowell 1, Laura Evans, Anwen Williams Future Med Chem. 2012 April; 4(6):751-69. doi: 10.4155/fmc.12.34. PBEF/NAMPT/visfatin: A Promising Drug Target for Treating Rheumatoid Arthritis?);
NFKBIA (e.g., as described in Heavey S, Godwin P, Baird A M, et al. Strategic targeting of the PI3K-NF [symbol] B axis in cisplatin-resistant NSCLC. Cancer Biol Ther. 2014; 15(10):1367-1377. doi:10.4161/cbt.29841);
NFKBIZ (e.g., as described in Willems M, Dubois N, Musumeci L, Bours V, Robe P A. I [symbol] Bζ: an emerging player in cancer. Oncotarget. 2016; 7(40):66310-66322. doi: 10.18632/oncotarget.11624);
NLRP3 (e.g., as described in Published: 20 Jul. 2018 Mangan, M., Olhava, E., Roush, W. et al. Targeting the NLRP3 inflammasome in inflammatory diseases. Nat Rev Drug Discov 17, 588-606 (2018). doi.org/10.1038/nrd.2018.97);
PDE4B (e.g., as described in Richter W, Menniti F S, Zhang H T, Conti M. PDE4 as a target for cognition enhancement. Expert Opin Ther Targets. 2013; 17(9):1011-1027. doi:10.1517/14728222.2013.818656);
SRGN (e.g., as described in Li X J, Qian C N. Serglycin in human cancers. Chin J Cancer. 2011; 30(9):585-589. doi: 10.5732/cjc.011.10314);
TIPARP (e.g., as described in Cheng L, Li Z, Huang Y Z, et al. TCDD-Inducible Poly-ADP-Ribose Polymerase (TiIPARP), A Novel Therapeutic Target Of Breast Cancer. Cancer Manag Res. 2019; 11:8991-9004. Published 2019 Oct. 18. doi:10.2147/CMAR. S219289);
TNFAIP3 (e.g., as described in Momtazi G, Lambrecht B N, Naranjo J R, Schock B C. Regulators of A20 (TNFAIP3): new drug-able targets in inflammation. Am J Physiol Lung Cell Mol Physiol. 2019; 316(3):LA56-L469. doi: 10.1152/ajplung.00335.2018);
ZFp36 (e.g., as described in Patial S, Blackshear P J. Tristetraprolin as a Therapeutic Target in Inflammatory Disease. Trends Pharmacol Sci. 2016; 37(10):811-821. doi: 10.1016/j.tips.2016.07.002);
IFIT2 (e.g., as described in Feng, X., Wang, Y., Ma, Z. et al. MicroRNA-645, up-regulated in human adencarcinoma of gastric esophageal junction, inhibits apoptosis by targeuing tumor suppressor IFIT2. BMC Cancer 14, 633 (2014). https://doi.org/10.1186/1471-2407-14-633);
MARCKS (e.g., as described in Yang et al. “targeting phosphor-MARCKS overcomes drug-resistance and induces antitumor activity in preclinicalmodels of multiple myeloma” Leukemia (2015) 29, 715-726);
TXNIP (e.g., as described in Alhawiti N M, Al Mahri S, Aziz M A, Malik S S, Mohammad S. TXNIP in Metabolic Regulation: Physiological Role and Therapeutic Outlook. Curr Drug Targets. 2017; 18(9):1095-1103. doi:10.2174/1389450118666170130145514);
XAF1 (e.g., as described in Huang et al. “XAF1 as a prognostic biomarker and therapeutic target in pancreatic cancer” Cancer Science 2010, 101(2):559-567).

### Pharmaceutical Compositions

The present disclosure also provides for pharmaceutical compositions comprising the one or more modulating agents. In certain cases, the methods of treatment comprise administering the pharmaceutical composition(s) to a subject in need thereof. A “pharmaceutical composition” refers to a composition that usually contains an excipient, such as a pharmaceutically acceptable carrier that is conventional in the art and that is suitable for administration to cells or to a subject.

In certain embodiments, the methods of the disclosure include administering to a subject in need thereof an effective amount (e.g., therapeutically effective amount or prophylactically effective amount) of the treatments provided herein. Such treatment may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Delivery of Modulating Agents and Pharmaceutical Compositions

Various delivery systems are known and can be used to administer the agents and pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

Delivery of Modulating Agents that are Polynucleotides

In cases the modulating agents are polynucleotides, they may be delivered to cell using suitable methods. In some embodiments, the polynucleotides may be packaged in viruses or particles, or conjugated to a vehicle for delivering into cells.

In some embodiments, the methods include packaging the polynucleotides in viruses and transducing cell with the viruses. Transduction or transducing herein refers to the delivery of a polynucleotide molecule to a recipient cell either in vivo or in vitro, by infecting the cells with a virus carrying that polynucleotide molecule. The virus may be a replication-defective viral vector. In some examples, the viruses may be virus (e.g. retroviruses, replication defective retroviruses, adenoviruses, replication defective adenoviruses, and adeno-associated viruses (AAVs)).

In some examples, the viruses are lentiviruses. Lentiviruses are complex retroviruses that have the ability to infect and express their genes in both mitotic and post-mitotic cells. Examples of lentiviruses include human immunodeficiency virus (HIV) (e.g., strain 1 and strain 2), simian immunodeficiency virus (SIV), feline immunodeficiency virus (FIV), BLV, EIAV, CEV, and visna virus. Lentiviruses may be used for nondividing or terminally differentiated cells such as neurons, macrophages, hematopoietic stem cells, retinal photoreceptors, and muscle and liver cells, cell types for which previous gene therapy methods could not be used. A vector containing such a lentivirus core (e.g. gag gene) can transduce both dividing and non-dividing cells.

In certain embodiments, the viruses are adeno-associated viruses (AAVs). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. In some cases, an AAV vector may include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, and International Patent Publication Nos. WO 91/17424 and WO 91/16024. Delivery can be to cells (e.g. in vitro or ex vivo administration) or target tissues (e.g. in vivo administration). Physical methods of introducing polynucleotides may also be used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides.

Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (International Patent Publication No. WO 2016/106236A1), which is incorporated by reference herein in its entirety.

In some cases, the methods include delivering the barcode construct and/or another element (e.g., a perturbation element) to cells. In such cases, the barcode construct and/or another element (e.g., a perturbation element) may be RNA molecules.

### Examples of Diseases and Conditions

The methods and compositions may be used for treating diseases and conditions related to the genes and/or pathways described herein. In some examples, the diseases and conditions may include inflammatory and immune diseases, allergic diseases, infections, particularly HIV infection, and diseases associated with the infection, psychoneurotic diseases, cerebral diseases, cardiovascular diseases, metabolic diseases, and cancerous diseases.

In some examples, the diseases and conditions may be infections, e.g., HIV infections. In some cases, the infection is an hyper-acute infection. A hyper-acute infection may be infection time points prior to the peak viral load. In some cases, the infection is an acute infection. An acute infection may be an infection time points after the peak viral load but before 6 months from the initial infection. In certain cases, the infection is a chronic infection. Examples of infections include, for example, HIV infection, various infections caused by streptococcus (Group A β-hemolytic streptococcus, Streptococcus pneumoniae, etc.), Staphylococcus aureus (MSSA, MRSA), Staphylococcus epidermidis, enterococcus, Listeria, meningococcus, gonococcus, E. coli bacteria (O157:H7, etc.), klebsiella (Klebsiella pneumoniae), Proteus, tussis convulsiva, Pseudomonas aeruginosa, Serratia marcescens, Shiorobactar, Ashinetobactar, Enterobactar, mycoplasma, chlamydia, and Crostorigeum, cholera, diphtheria, dysentery, scarlet fever, anthrax, trachoma, syphilis, tetanus, Hansen's disease, legionella, Reptospira, Lyme disease, tularaemia, Q fever, meningitis, encephalitis, rhinitis, sinusitis, pharyngitis, laryngitis, orbital cellulitis, thyroiditis, Lemierre syndrome, pneumonia, bronchitis, tuberculosis, infectious endocarditis, pericarditis, myocarditis, infectious aortitis, septicemia, cholecystitis, cholangitis, hepatitis, liver abscess, acute pancreatitis, splenic abscess, enteritis, iliopsoas abscess, pyelonephritis, cystitis, prostatitis, colpitis, Pelvic inflammatory disease, cellulitis, panniculitis, gas gangrene, furuncle, carbuncle, contagious impetigo, staphylococcal scalded skin syndrome, herpes zoster, varicella, measles, rubella, impetigo, scabies, infectious arthritis, osteomyelitis, fasciitis, myositis, and lymphadenitis.

The diseases and conditions may be those associated with infections, particularly HIV infection, include acquired immunodeficiency syndrome (AIDS), candidiasis, Pneumocystis carinii pneumonia, Cytomegalovirus retinitis, Kaposi's sarcoma, malignant lymphoma, AIDS encephalopathy, bacterial sepsis and the like.

The diseases and conditions include infections caused by other types of viruses, or diseases and conditions caused by such infections. Examples of the viruses include Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2†225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

Other examples of diseases and conditions include inflammatory and immune diseases such as rheumatoid arthritis, arthritis, retinopathy, systemic erythematosus, gout, rejection of transplanted organ, graft-versus-host disease (GVHD), nephritis, psoriasis, rhinitis, conjunctivitis, multiple sclerosis, ulcerative colitis, Crohn's disease, shock associated with bacterial infection, pulmonary fibrosis, systemic inflammatory response syndrome (SIRS), acute lung injury, diabetes and the like. Examples of the allergic disease include asthma, atopic dermatitis, rhinitis, conjunctivitis and the like.

In some embodiments, the methods and compositions may be used for regeneration therapy for the purpose of in vitro or in vivo amplification of stem cells for gene therapy as well as peripheral blood stem cells mobilization and tissue repair. For example, the diseases and conditions also include immune rejection related to transplantations, such as including bone marrow transplantation, peripheral blood stem cell transplantation and tissue repair among in the regeneration therapy.

### Methods of Diagnosis

In another aspect, the present disclosure provides methods of diagnosis of an infection. The methods may also comprise detecting and/or monitoring immune responses to the infection. For example, the methods may comprise detecting the status of an immune response, e.g., whether it is a hyper-acute or acute response. Base on the status, diagnosis and/or treatment plans may be made.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The term “monitoring” generally refers to the follow-up of a disease or a condition in a subject for any changes which may occur over time. The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In some embodiments, the methods of detecting and/or monitoring an immune response comprise detecting in one or more types of immune cells one or more biomarkers. The biomarkers include the genes that can be modulated in methods of treatment described herein. In some embodiments, the one or more biomarkers comprise IFI27, IFI44L, IFI6, IFIT3, ISG15, XAF1, or a combination thereof. In some embodiments, the one or more types of immune cells are monocytes, and the one or more biomarkers comprise CXCL10, DEFB1, IFI27L1, or a combination thereof. In some embodiments, the one or more types of immune cells are dendritic cells, and the one or more biomarkers comprise PARP9, STAT1, or a combination thereof. In some embodiments, the one or more types of immune cells are CD4+ T cells, and the one or more biomarkers comprise CD52, TIGIT, TRAC, or a combination thereof. In some embodiments, the one or more types of immune cells are NK cells, and the one or more biomarkers comprise CX3CR1, ICAM2, or a combination thereof. In some embodiments, the one or more types of immune cells are NK cells, and the one or more biomarkers comprise CX3CR1, ICAM2, or a combination thereof. In some embodiments, the one or more types of immune cells are monocytes and/or DCs, and the one or more biomarkers comprise CXCL10 and LGALS3BP, or a combination thereof. In some embodiments, the method detects whether the immune response is a hyper-acute or acute immune response. In some embodiments, the one or more types of cells are monocytes, and the one or more biomarkers comprise SLAMF7, DUSP6, WARS, USP18, or a combination thereof.

In some embodiments, treatments can be administered based on the diagnosis. For example the methods may further comprise administering one or more modulating agents to modulate expression and/or activity of the detected one or more biomarkers. In some cases, a method for treating a subject with an infection, the method comprising detecting expression or activity of one or more biomarkers in one or more types of immune cells; administering one or more modulating agents to modulate expression and/or activity of the detected one or more biomarkers. In some cases, a method for treating a subject with an infection, the method comprising: detecting expression or activity of one or more biomarkers in one or more types of immune cells; administering one or more modulating agents to modulate expression and/or activity of the detected one or more biomarkers.

### Methods of Screening Modulating Agents

In another aspect, the present disclosure includes methods of screening modulating agents. The modulating agents may be capable of modulating an immune response. The methods may comprise (a) contacting one or more immune cells with one or more candidate modulating agents; (b) detecting expression and/or activity of one or more biomarkers in response to the one or more candidate modulating agents; and (c) selecting modulating agents that cause change in expression and/or activity of one or more biomarkers compared to expression and/or activity of the one or more biomarkers before (a).

### EXAMPLES

### Example 1—Integrated Single-Cell Analysis of Multicellular Immune Dynamics During Hyper-Acute HIV-1 Infection

Cellular immunity is critical for controlling intracellular pathogens, but the dynamics and cooperativity of the evolving host response to infection are not well defined. Here, Applicants applied single-cell RNA-sequencing to longitudinally profile pre- and immediately post-HIV infection peripheral immune responses of multiple cell types in four untreated individuals. Onset of viremia induces a strong transcriptional interferon response integrated across most cell types, with subsequent pro-inflammatory T cell differentiation, monocyte MHC-II upregulation, and cytolytic killing. With longitudinal sampling, Applicants nominated key intra- and extracellular drivers that induce these programs, and assigned their multi-cellular targets, temporal ordering, and duration in acute infection. Two individuals studied developed spontaneous viral control, associated with initial elevated frequencies of proliferating cytotoxic cells, inclusive of a previously unappreciated proliferating natural killer (NK) cell subset. The study presents a unified framework for characterizing immune evolution during a persistent human viral infection at single-cell resolution, and highlights programs that may drive response coordination and influence clinical trajectory.

Single-cell RNA-sequencing revealed cell types, states, coordinated transcriptional programs, and molecular drivers induced by hyper-acute viral infection, as well as candidate cellular features that may associate with HIV-1 control in chronic viremia.

Understanding the dynamics of host-pathogen interactions during acute viral infection in humans has been hindered by limited sample availability and technical complications associated with comprehensively profiling heterogeneous cellular ensembles. To date, microarray and bulk transcriptomic studies of yellow fever vaccination (1) and influenza infection (2) have highlighted complex cellular responses that vary as a function of time, largely characterizing a common systemic interferon stimulated gene (ISG) program. In each instance, additional insights might be gleaned through more sensitive, discretized systems-approaches that can elucidate the contributions of individual cellular components and nominate features that drive productive responses essential to improve vaccines.

Recently, high-throughput single-cell RNA-sequencing (scRNA-Seq) has emerged as a powerful tool to characterize, transcriptome-wide, complex human systems in health and disease at single-cell resolution (3-9). When applied to a collection of samples across a disease setting, this approach provides a platform for investigating cell types, states, interactions, and drivers associated with that disease; this information can be used to develop testable hypotheses on therapeutic modulations that may ameliorate disease state (7, 8). Meanwhile, within an individual, longitudinal sampling provides an opportunity to decipher, at unprecedented resolution and absent potentially confounding inter-individual variability (7), shifts in these same variables, and to associate observed changes with internal or external perturbations (10-12). Such sampling of a host's exposure to a pathogen could provide foundational insights into essential cellular response features and their coordination, empowering the rational design of improved prophylactic interventions.

A better understanding of the interplay between innate and adaptive immune responses at the very earliest stages of a viral infection, and its impact on long-term disease, may reveal principles to accelerate prevention efforts. Human Immunodeficiency Virus (HIV) has been the subject of thorough study, and thus is a well-considered model system for examining host responses to a pathogen. Moreover, although the development of antiretroviral therapy (ART) (13), as well as implementation of pre-exposure prophylaxis (PrEP) (14) and combination prevention efforts, has improved the lives of persons living with HIV, increased life expectancies, and reduced the number of new infections, there were still 2 million new cases of HIV-1 infection in 2017 (15). This highlights a pressing need for effective HIV vaccines informed by an understanding of natural host-pathogen dynamics.

Here, Applicants apply scRNA-Seq to perform an integrated longitudinal analysis of implicated cell programs and drivers during the critical earliest stages of HIV infection. By examining individuals in the Females Rising through Education, Support and Health (FRESH) study (16, 17)—a unique prospective cohort of uninfected young women at high risk of contracting HIV who are monitored for acute viremia by twice weekly plasma sampling—and focusing on those who were enrolled at a time when standard of care did not include treatment during acute disease, Applicants comprehensively examined untreated cellular immune dynamics during the evolution of hyper-acute infection into chronic viremia. Among over 65,000 cells obtained from repeated sampling of peripheral blood, Applicants identified cell types, states, gene modules, and molecular drivers associated with coordinated immune responses to a viral pathogen. Further, these data suggested candidate cellular features that may influence the magnitude of chronic viremia, known to identify long-term infection outcome. Overall, the longitudinal, granular approach captures multiple dynamic and coordinated immune responses—both shared and distinct between cell types and individuals—and provided a framework for their elucidation in health and disease.

### Results

### Longitudinal Single-Cell Transcriptomic Profiling Captures Major and Granular Immune Subsets in Hyper-Acute Infection

In order to globally and longitudinally examine host immune responses during a hyper-acute infection, Applicants performed scRNA-Seq on peripheral blood mononuclear cells (PBMCs) from four individuals enrolled in FRESH who became infected with HIV, assessing multiple timepoints from pre-infection through one year following initial detection of viremia (FIG. 1A, table 1). In the study, hyper-acute infection referred to timepoints at and prior to peak-viral load, whereas acute infection refers to timepoints after peak viral load but before 6 months. Samples were processed in duplicate using Seq-Well (18)—a portable, low-input massively-parallel scRNA-Seq platform designed for clinical specimens—allowing for robust single-cell transcriptional analysis of PBMC subsets. All individuals studied demonstrated the expected rapid rose in plasma viremia and drop in CD4+ T cell counts that typify hyper-acute and acute HIV infection (FIG. 1B). Among all individuals, Applicants captured 65,842 cells after eliminating low quality cells and multiplets (see Methods), with an average of 2,195 cells per individual per timepoint. Alignment to a combined human and HIV genome at peak infection timepoints yielded few reads mapping to HIV; therefore, alignment for all samples was conducted using a human-only reference.

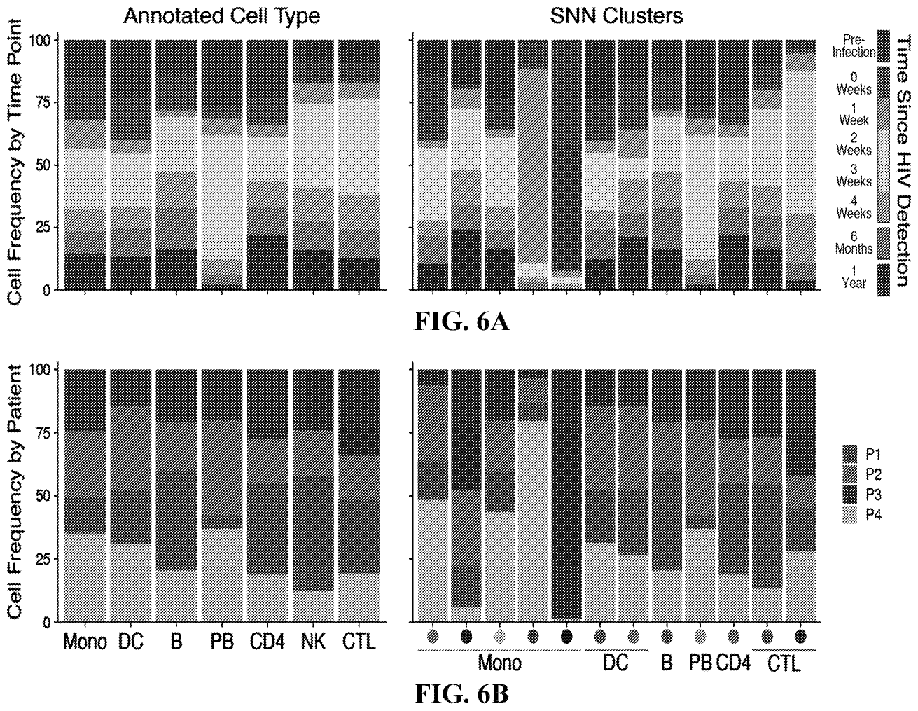

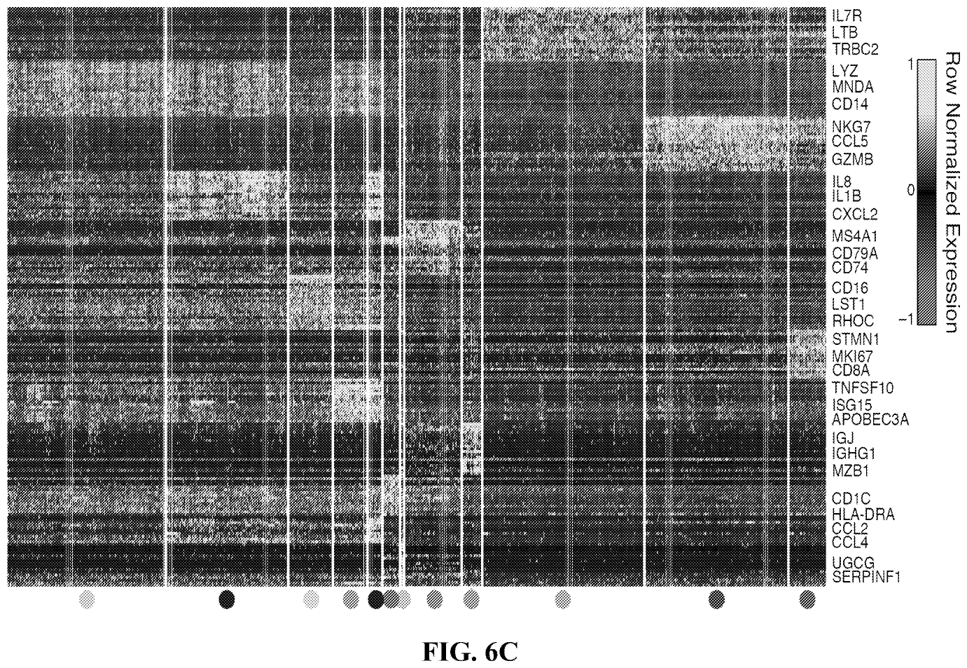

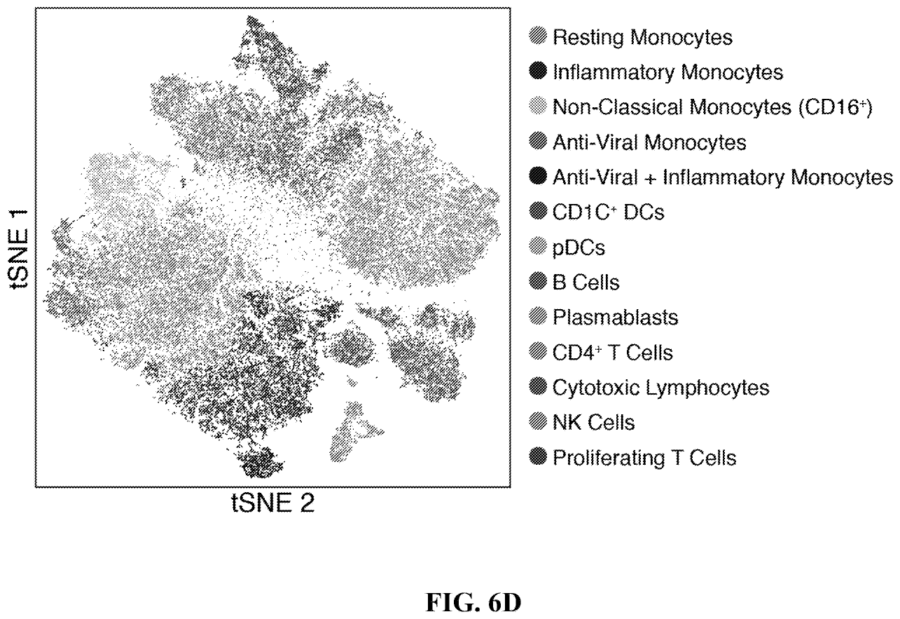

To assign cellular identity, Applicants performed variable gene selection, dimensionality reduction, clustering, and embedding en masse across data collected from all individuals and timepoints (see Methods). Samples were combined for cell type/phenotype identification to find common transcriptional features of ubiquitous cell subsets and to improve statistical power on classifying small/rare cell types. Importantly, combined analyses yielded few individual-specific features in the resulting clustering and embedding, suggesting that disease biology, rather than technical batch, was the main driver of variation and subsequent clustering (FIG. 1D, 6A, 6B). Applicants annotated identified clusters by comparing differentially expressed (DE) genes that defined each to known lineage markers and previously published scRNA-Seq datasets (19-21) (FIG. 6C, see Table 2 for list of DE markers). These clusters recapitulated several well-annotated PBMC subsets (FIG. 1C), in addition to revealing phenotypic groupings of monocytes (anti-viral, inflammatory, non-classical) and cytotoxic T cells (CD8+ CTL, proliferating; see FIG. 6D). Thus, Applicants readily and reproducibly mapped the cellular players and phenotypes present along the course of disease progression.

Cell Frequency Over Time is Readily Obtained from Transcript-Assigned Cellular Identity

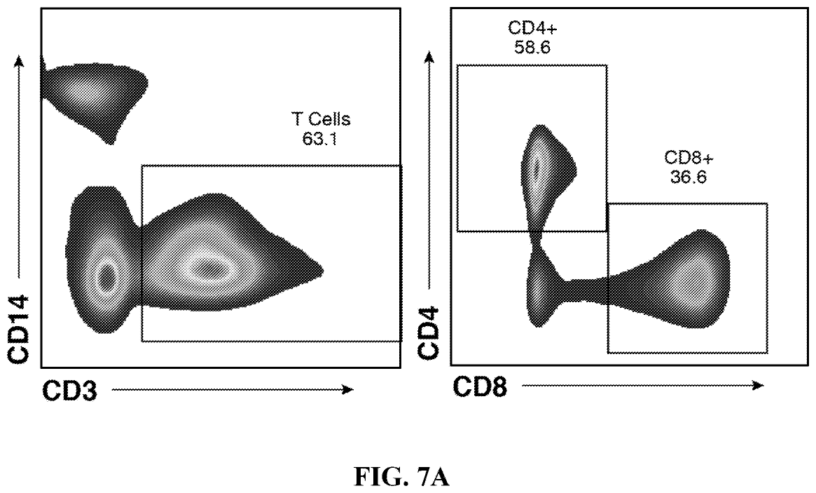

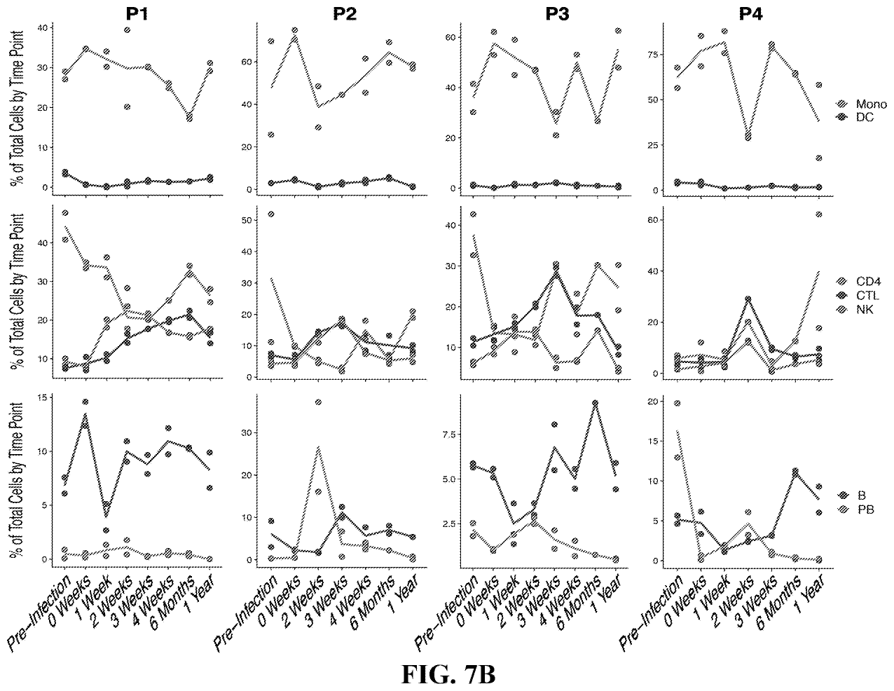

Applicants next examined cellular dynamics over the course of infection, beginning with a pre-infection time point. Onset of HIV infection was typically accompanied by an initial depletion of CD4⁺ T cells in the blood and a subsequent small rebound before continued depletion in chronic infection (22). To ensure that the estimated frequencies recapitulated conventional measurements of the samples, in parallel, Applicants employed flow cytometry to independently establish the frequencies of T cell subsets (FIG. 7A). Linear regression of the measured CD3⁺CD4⁺ and CD3⁺CD8⁺ flow populations (% of total CD45⁺ cells) with their respective single-cell transcriptome clusters (% of total single cells) across time yielded strong correlations (linear regression, F-test): average CD4⁺−R²=0.491, p=0.0416; average CD8⁺- R²=0.665, p=0.00158 (FIG. 1E). Subsequently, Applicants calculated frequencies for the other cell types in the scRNA-Seq data as a function of time (FIG. 7B). In each individual, Applicants measured an expansion in monocytes at HIV detection and in NK cells that peaked at 3- or 4-weeks post-detection, in-line with studies of influenza and murine cytomegalovirus (MCMV) demonstrating expansion and recruitment of monocytes and NK cells to sites of infection, though on shorter time-scales (23-25). Altogether, the data elucidate dynamic temporal shifts in the abundance of different cellular subsets during hyper-acute and acute HIV infection aligned with flow cytometry; more importantly, with whole transcriptome information, they enable further global characterization of subcellular activity within and between these subsets.

### Discovering Structured Variation in Cell Phenotypes Over Time in Response to Infection

To understand how the identified cell types—monocytes, dendritic cells (DCs), plasmablasts, B cells, natural killer (NK) cells, CD4⁺ T cells, CD8⁺ T cells, and proliferating T cells (a sub-cluster of CTLs, see FIG. 7D)—varied in phenotype over the course of infection, Applicants assessed coordinated changes in gene expression within each cell type that significantly varied in time. Since the immune responses and time courses of infection were heterogeneous among participants due to the sampling scheme and natural human variability, Applicants performed analyses on an individual-by-individual and cell-type-by-cell-type basis. In this way, the results were sensitive to both intra- and inter-individual changes in gene expression.

To identify tightly co-regulated modules (M) of genes for each type for each individual, Applicants applied weighted gene correlation network analysis (WGCNA) (26, 27) on all cells classified as a particular cell type across all timepoints (FIG. 2A; see Methods for details). Strongly correlated gene modules (permutation test for within-module similarity, FDR corrected q<0.05) were then tested for significant variation over time by scoring cells at each timepoint against the genes within a module, followed by tests for shifts in score distribution between pairs of timepoints (Wilcoxon rank sum test, FDR corrected q<0.05). This generated 0-8 temporal modules per cell type (for a list of all significant modules see Tables 3A-3D for gene membership and Tables 4A-4D for median module scores over time).

Across cell types within an individual, these gene program trajectories demonstrated common transient patterns along the course of infection, indicating the utility of this approach in identifying groups of genes acting in concert. While a similar approach is possible using bulk RNA-seq data, here, Applicants are powered to identify temporally similar modules active in distinct subsets of cells both within and across time. Compared to a directed approach, this discovery-based identification of temporally-variant modules enabled unbiased selection of coordinated genes and pathways, and immediately revealed differences in response dynamics among cell types, states, and individuals.

### Temporal Module Analysis Reveals Shared and Unique Responses to Interferon Across Cell Subsets Near Peak Viremia

With distinct, temporally-variant modules across all cell types and individuals in hand, Applicants next sought to understand these response modules and their association with plasma viral load, the main clinical parameter linked with disease progression rate and clinical outcome (28, 29). Beginning with one individual (P1), Applicants identified a set of 6 significant gene modules spanning multiple cell types that all shared their highest relative module score at the peak viremia timepoint (FIG. 2B). Upon inspection of the genes within each, Applicants uncovered a core set of genes shared among the modules from all cell types: IFI27, IFI44L, IFI6, IFIT3, ISG15, and XAF. These genes, in addition to many others belonging to one or multiple of these peak viral load modules, are all induced by type I interferon (IFN-I) stimulation in cell lines and ex-vivo primary cells (30-32) (FIG. 2C, FIG. 8A). Since these modules were generated de-novo, the results also report cell type specific genes and functions that correlate with the core measured IFN response signature: anti-viral activity (CXCL10, DEFB1, IFI27L1) in monocytes (33, 34), DC activation (PARP9, STAT1) likely through sensing of HIV by pattern recognition receptors and interferon by interferon receptors (35-37), differentiation of naïve CD4+ T cells (CD52, TIGIT, TRAC) potentially into HIV-specific T helper cells (38-41), and NK cell trafficking (CX3CR1, ICAM2) shown to occur in other viral infections (42-44).

As transcriptional work in humans has been limited to late-acute stage and treated infection (45), Applicants sought to contextualize the data against the massive IFN response measured in acute SIV infection (46-49), specifically in rhesus macaques (RM, see FIG. 8B) (47). In SIV models, natural hosts of the infecting virus (e.g., sooty mangabeys) resolve IFN immune activation more quickly than susceptible hosts, positing that time to resolution may reflect future control in chronic infection (>180 days). By comparison, Applicants found that many IFN stimulated genes induced in RM for 2+ weeks arose and resolved within one week (i.e., upregulate at one timepoint). Here, Applicants were powered to assign the cells expressing these various response genes. For example, upregulation of RIG-I (DDX58) was limited to myeloid cells—though RIG-I signaling has been shown to be subverted by HIV (50)—whereas only CD4+ T cells exhibit higher levels of STAT2, suggesting a polarization towards a T_{H}1 phenotype (51).

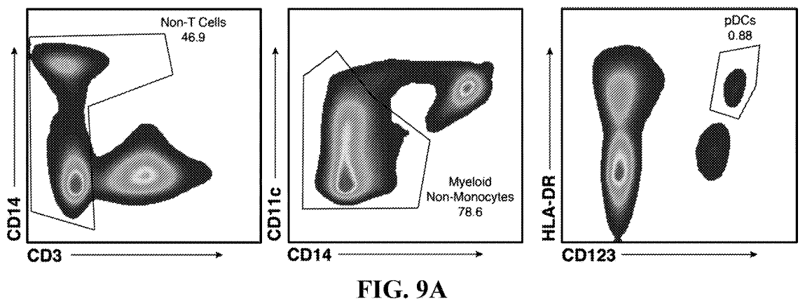

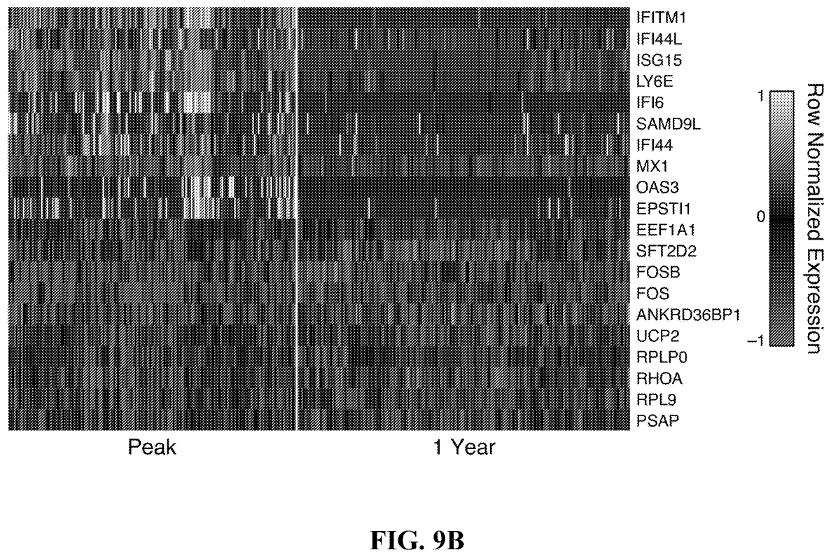

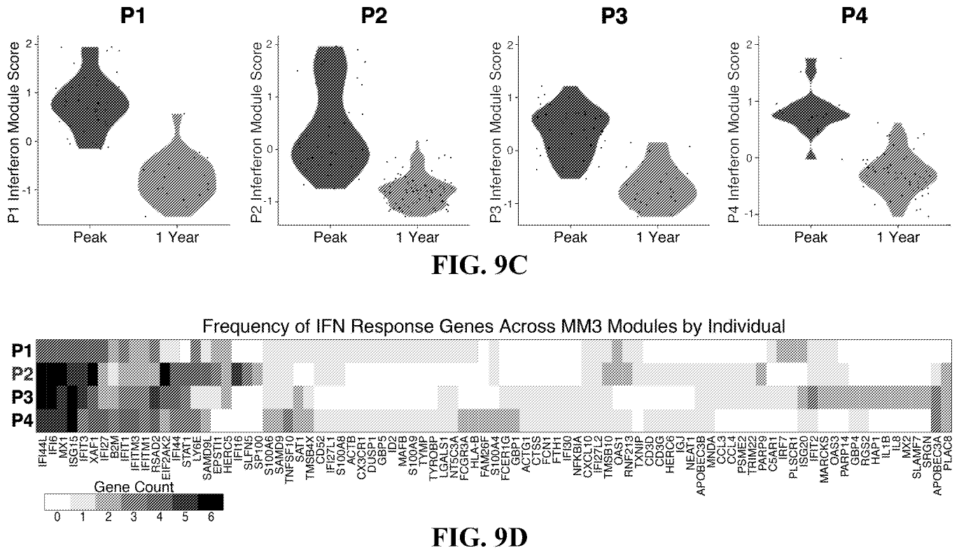

Subsequently, Applicants examined the expression of IRF7, one of the interferon regulatory factors that was responsible for anti-viral mediated IFN-I production in SIV/HIV (52, 53) and other viral infections, to determine which cells might be generating this pervasive wave of IFN. In individual P1, almost all cell types demonstrated higher expression of IRF7 compared to pre-infection and 1-year timepoints (FIG. 8C), highlighting the pervasiveness of IFN-I in response to high levels of viremia and potentially indicative of the positive feedback loop it induces (54-56). Since plasmacytoid DCs (pDCs) are known to produce IFN-α and IFN-β in response to HIV (57), Applicants also assayed single pDCs at peak viremia and 1-year post-infection using a plate-based scRNA-Seq method compatible with enrichment by FACS (Smart-Seq2 (58)) (FIG. 9A). At both times, type I IFNs were undetectable. Comparing pDCs between them, Applicants observe modestly increased expression of IRF7 at peak viremia (Wilcoxon rank sum test, FDR corrected q<1, log(Fold Change)=0.42). However, these cells also upregulated several ISGs that were present in the modules of other cell types (FIG. 9B).

Applicants next sought to identify whether similar gene expression programs typified responses in the other three individuals assayed. Applicants discovered a similar set of modules around the time of peak viremia in each individual (FIG. 2D and FIG. 8D), as well as shared responses among pDCs (FIG. 9C). Comparing modules across the cohort, Applicants noted common response genes (present in 3 or more cell-types) either shared (ISG15, IFIT3, XAF1) or specific (APOBEC3A, IFI27, STAT1) to subsets of individuals, suggesting potential core programming and the possibility for the same immune drivers to induce distinct gene responses (FIG. 9D). Finally, to confirm the presence of downstream cytokines from IFN stimulation, Applicants measured MIG (CXCL9) and IP10 (CXCL10) levels in plasma at pre-infection, peak viremia, and 9-months post infection (FIG. 2E; Methods). All four individuals demonstrated higher levels of IP10 at peak viremia, and three demonstrated elevated levels of MIG. Together, these data highlight the ability of the approach to ascertain a short, pervasive wave of IFN responses in most peripheral immune cells that coincides with, or precedes, peak viremia in hyper-acute HIV infection. Moreover, Applicants uncovered nuanced differences among individuals and cellular subsets in this response, as might be expected for an infection associated with diverse clinical courses (e.g., differences in plasma viremia; FIG. 1B).

### Individuals Demonstrate Diverse, Yet Coordinated, Immune Responses During the First Month of Infection

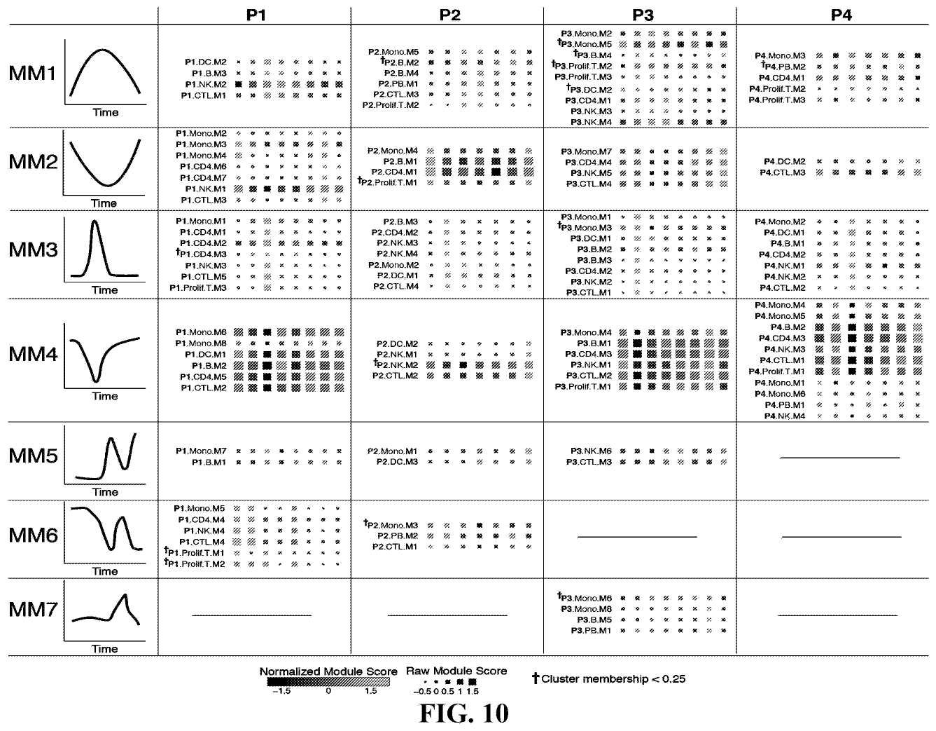

To investigate other groups of temporally similar modules, Applicants next applied fuzzy c-means clustering (59, 60) to the median module scores at each timepoint across all cell types on an individual-by-individual basis to generate clusters of modules, hereafter referred to as meta-modules (MMs). Applicants subsequently grouped these MMs by temporal shape (FIG. 10 and see Methods for choice of c). MMs represented gene programming in distinct cell types that demonstrate coordinated temporal patterns—here, various cell-types responding simultaneously to infection—enabling us to link discrete transcriptional responses to their propagators. In addition to the aforementioned MM that contained the majority of the IFN response modules (labeled MM3), the only other MM that spanned the majority of cell types was one enriched for ribosomal protein coding genes (labeled MM5, see Tables 3A-3D)—known to indicate cellular quiescence (61). MM5 demonstrated temporal profiles defined by minimum module scores (i.e., significantly downregulated) around peak viremia, anti-concordant with the immune activation (i.e., significant upregulation) seen in MM3.

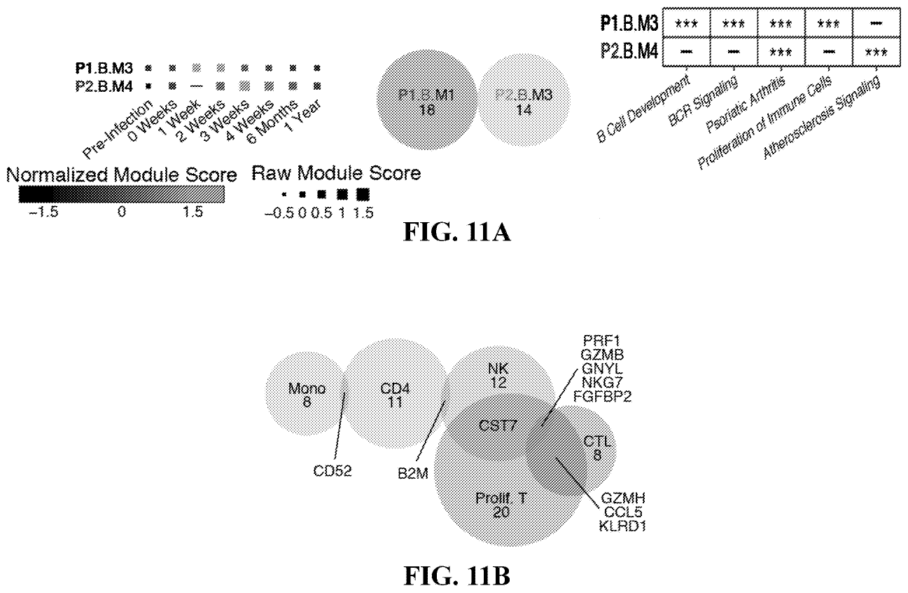

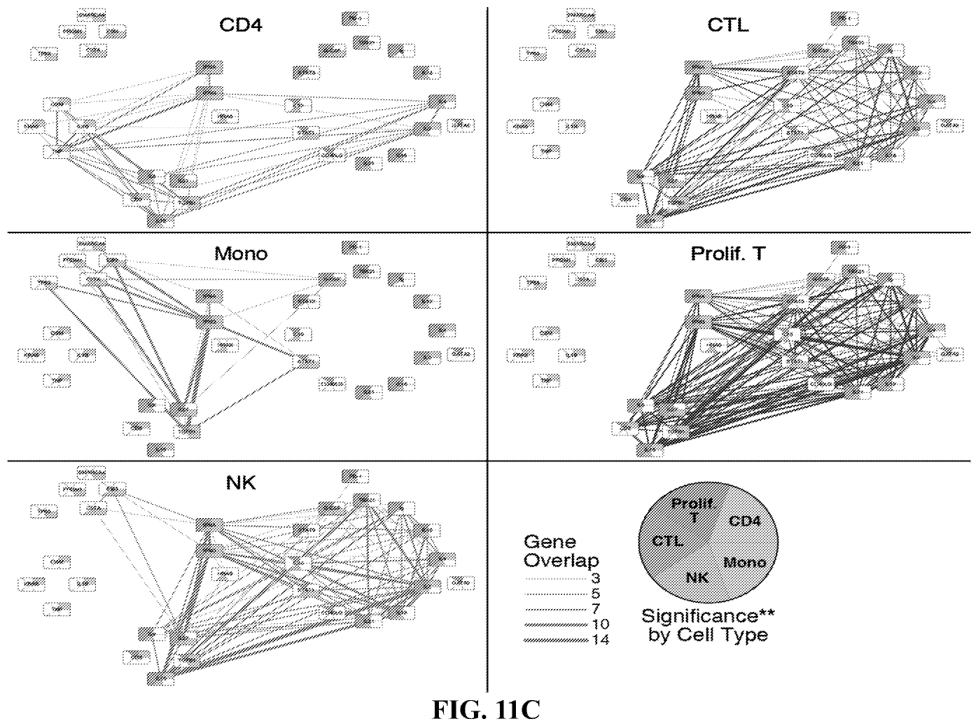

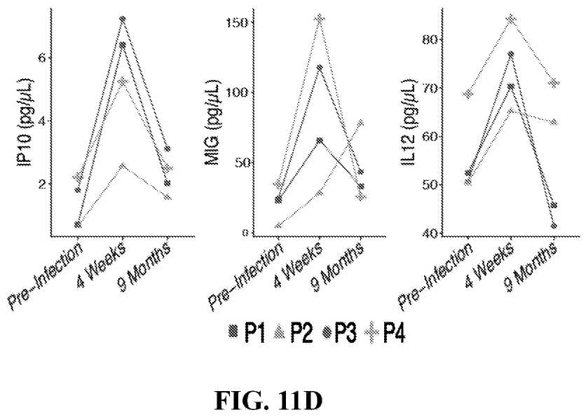

Another MM that shared similar temporal immune responses across individuals was MI, comprised of responses sustained throughout one-month post-detection. In at least 2 of the 4 individuals studied, Applicants identified sustained response modules with shared genes in CD4⁺ T cells, monocytes, NK cells, CTLs, and proliferating T cells (FIGS. 3A-3E, see Tables 5A-5B for overlapping genes). While DCs and B cells also expressed multiple modules within this MM, some modules had low MM membership scores and were excluded (membership <0.25, labeled with † in FIG. 10) or did not share any genes across individuals (FIG. 11A).

As each module within MM1 is distinct, Applicants performed gene set enrichment analyses (see Methods) to discern if, in addition to sharing genes, modules from the same cell type shared functional annotations across individuals (FIGS. 3A-3E). In every cell type, modules across individuals were significantly enriched for many of the same underlying pathways (see Tables 6A-6B for full list), despite slightly variable temporal dynamics and unique gene membership. CD4⁺ T cells expressed genes associated with non-classical viral entry by endocytosis (62) as well as adhesion, potentially suggesting migration and viral dissemination throughout the body. Monocytes expressed genes associated with antigen presentation and IL-4 signaling (mainly HLA-DR subunits), which may reflect generalized interferon responses, or the potential to promote active T helper and CTL responses. NK cells, CTLs, and proliferating T cells all upregulated genes associated with killing of target cells by perforin and granzyme release, highlighting the joint role of innate and adaptive cells in combating viremia (see Tables 5A-5B and FIG. 11B for all shared responses across cell types) (63, 64). Thus, the results indicate common functional enrichments supported by gene sets that vary across cell types and individuals in response to infection.

### Distinct Cell Types Respond to Common and Unique Upstream Drivers Induced in Infection.

To identify common and cell-type specific inducers of these measured transient responses extending past peak viremia, Applicants generated a list of upstream drivers of each module (see Tables 6A-6B). Selecting highly significant hits in at least two modules, Applicants drew a network of identified upstream drivers (nodes) colored by significance in each cell type with edges connecting nodes with shared enriched genes (FIGS. 3F, 11C, and see Methods). Strikingly, IFN-α and IFN-γ were identified upstream drivers of these sustained responses for all five cell types even though these modules do not contain the typical ISGs; in chronic HIV infection, prolonged IFN-I stimulation has been shown to maintain viral suppression but also blunt other immune functions in a humanized mouse model (65, 66). Matching Luminex data confirmed elevated levels of IP-10 and MIG at one-month post HIV detection (FIG. 11D). IL-15 and IL-2, known to induce T and NK cell proliferation but to lead to defects in chronic infection (67-69), were enriched as drivers for all lymphocytes explored. However, they also shared enriched genes with several other interleukins, including IL-4, IL-12 (also elevated in plasma, see FIG. 11D), and IL-21. Interestingly, only CD4⁺ T cell modules were enriched for TNF, IL-1B, and OSM, suggesting the directed induction of pro-inflammatory T helper cells. Meanwhile, monocytes and NK cells were enriched for CIITA and EBI3 (a subunit of IL-27), which regulate MHC-II and MHC-I genes, respectively (71, 72).

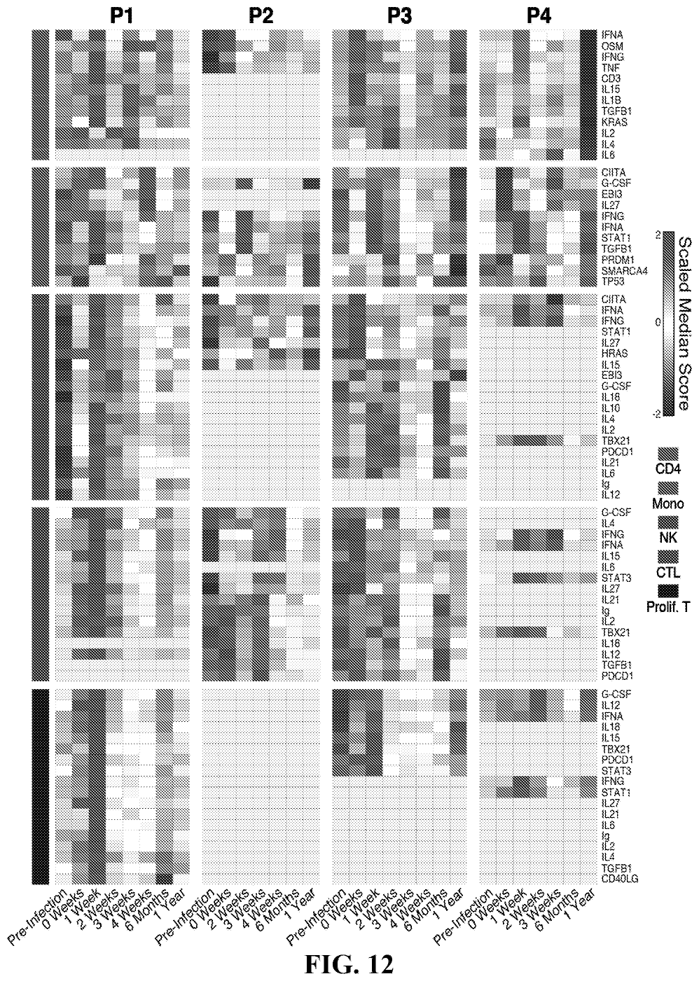

Applicants also contextualized observed responses to these upstream drivers temporally by re-scoring against enriched genes for each driver. This analysis revealed variable kinetics in the onset, intensity, and length of immune responses across different cell types (FIGS. 3G, 12). Applicants noted the following gene-programming upregulation trends in most individuals: CD4⁺ T cells are activated from before peak viremia throughout 3-4 weeks post infection, and CTL and proliferating T cell programs are induced for 2-3 weeks around peak viremia, whereas NK cell and monocyte activity extends throughout the first month of infection.

Based on shared cell-type enrichments, genes, and functions, Applicants summarized the multitude of common immune responses displaying sustained gene expression over the course of first month of HIV infection, and their potential drivers, across individuals (FIG. 3H). While the IFN stimulated gene programs did not extend past hyper-acute infection, the data suggest that persistent IFN activation could manifest in different ways in each cell type, leading long-term to previously shown dysfunction partially mediated by IFN in chronic infection (73). This analysis also supported more complex cytokine interactions—some previously described as synergistic (e.g. IL-2 & IL-18 (74)) or antagonistic (e.g. IL-6 & IL-27 (75))—occurring in acute infection, and delineated how they may affect various cell types. Though dozens of cytokines were known to elevate in plasma during acute HIV infection (76), here Applicants presented a schematic of which cell types they modulate alongside other extracellular proteins and transcription factors active during this time frame. Furthermore, the analysis established a blueprint of multi-cellular responses to several stimuli along the course of hyper-acute and acute infection to be edified by application to other pathogens.

Two Instances of Temporally Similar Modules within a Cell Type Discerned by scRNA-Seq

After discovering temporally variant modules in the dataset, Applicants observed a few sets that demonstrated similar temporal response patterns in a given cell type, but were not combined into a single module by the framework. Applicants thus sought to understand how these modules might be linked by looking across single cells for module co-expression. Here, single-cell expression data were important to distinguish response circuitry among cells.

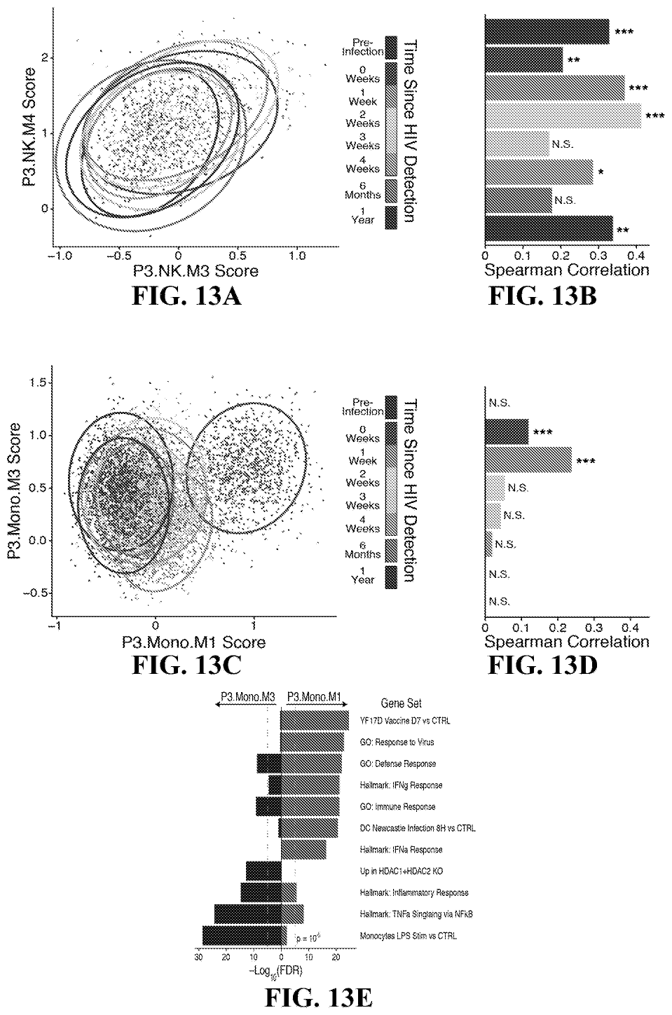

A good example of multiple modules being co-expressed with the same temporal pattern in the same cell type from the analysis was the NK activated M3 module (CCL3, CCL4, CD38) and the cytotoxic M4 module (PRF1, GZMB, HLA-A) in P3 (FIG. 3D), both part of MM1. Enrichment analysis demonstrated little overlap between the significant pathways associated with these modules, implying orthogonal biological function. Applicants therefore investigated whether the gene programs for these modules were highly co-expressed in the same single cells and thus co-varied among single cells across time (FIG. 13A). While Applicants did not observe differential simultaneous upregulation of these modules between time points, they found variation in the correlation of cell-scores for the pair as a function of time across single cells, with the strongest correlation one to two weeks after HIV detection (FIG. 13B). Variation in the correlation of M3 and M4 may reflect a synergizing of these gene programs (77) within NK cells to combat HIV as viremia declines post peak.

In examining MM3 (FIG. 10)—containing the majority of the IFN response modules—Applicants observed that P3 also exhibited a set of temporally similar modules in monocytes (M1 & M3); however, these modules did not variably correlate in expression score as a function of time. Instead, these gene programs were highly co-expressed but only at HIV-detection (FIGS. 13C-13D). Gene set analysis readily demonstrated that monocyte M1 included IFN response genes, while M3 was enriched for genes associated with inflammation (FIG. 13E). IFN has been shown to stunt the production of pro-inflammatory cytokines in monocytes similar to the phenotype observed in these cells in viremic persons (78, 79), but the co-expression of anti-viral and pro-inflammatory signals in the same single cells has not yet been described to Applicants' knowledge. As these module scores were generated independently for each single cell, individual monocytes in this person at the time of HIV detection are simultaneously expressing both anti-viral and inflammatory gene programs. The longitudinal granular, single-cell approach facilitated the study of variation in gene module correlation and co-upregulation, suggesting key cellular circuitry, and its coordination, during response to infection.

### One Individual Who Naturally Controls Infection Displays a Polyfunctional Subset of Monocytes at HIV Detection

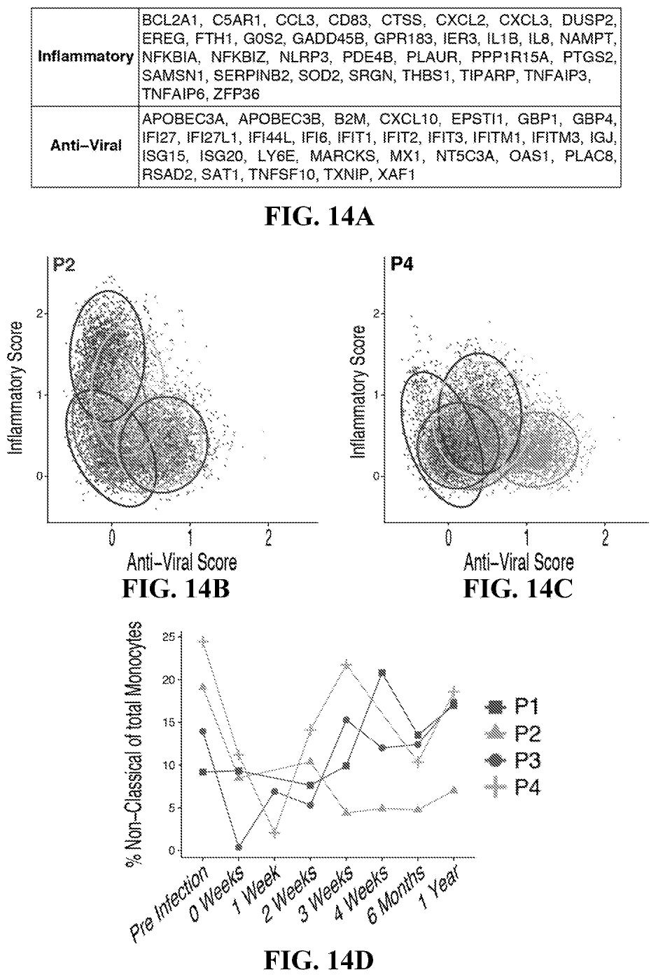

Intrigued by the appearance of these polyfunctional monocytes in one individual, Applicants next explored whether the other individuals assayed developed similar cells after infection. Scoring monocytes from each individual on inflammatory and anti-viral gene lists derived from discovered modules (FIG. 14A), Applicants were unable to identify these polyfunctional monocytes in the other three individuals (FIGS. 4A-4B, 14B-14C). In fact, looking at structured gene variation in monocytes over time in principal component analysis (PCA) space revealed that the major axis of variation (PC1) in P1 and P2 not only reflected sample timepoint, but also separated monocytes based on their expression of anti-viral and inflammatory genes. In P3 and P4, however, these gene programs contributed to different principal components, suggesting their independence in defining monocyte phenotype.

In all four individuals, Applicants saw dramatic structuring of the monocytes in PC space by time. Specifically, monocytes sampled at HIV detection (0 weeks) or 1-week post-detection were strongly separated along either PC1 or PC2, indicating a pervasive hyper-acute response to infection. Interestingly, non-classical monocytes (see FIG. 6D and Table 2), which may be more susceptible to infection (80), displayed disparate temporal dynamics across individuals, even though they drove significant variation in PCA space (FIG. 14D). Comparing DE genes at these peak response timepoints (vs. pre-infection) highlighted not only the specificity of the co-inflammatory/anti-viral monocytes to P3, but also other person specific differences in monocyte phenotype (FIG. 4C). Gene set analysis on upregulated genes in each individual confirmed that monocytes in all individuals produced strong anti-viral factors (e.g., RIG-I, APOBEC3B, MX1) with significant enrichment (MHC hypergeometric test, q<0.001) for response to IFN-α and IFN-γ (FIG. 4D). Moreover, corroborating the scoring on inflammatory genes, only P2 and P3 were significantly enriched for inflammatory responses, and only P3 for TNF signaling via NF-kB (MHC hypergeometric test, q<0.001). In fact, P1 and P2 demonstrated downregulation of genes associated with inflammation compared to pre-infection.

Subsequently, Applicants investigated known clinical parameters in the cohort for features of infection that might be related to the appearance of these polyfunctional cells. As the level of viral load in chronic infection correlates with disease outcome (28), Applicants compared the viral load setpoints of these individuals at 1.8, 2.3, and 2.75 years after HIV detection. Two of the four individuals (P3 & P4) maintained low levels of viremia (<1,000 viral copies (vc)/mL) out to 2.75 years in the absence of ART (FIG. 4E). HIV infected persons who naturally maintained low levels of viremia in chronic infection (controllers) have been shown to have enhanced immune responses in chronic infection (7, 81, 82). However, whether early events in acute HIV infection reflect or contribute to long-term control is unknown. In the hyper-acute monocyte responses (FIG. 4C), Applicants found a small set of upregulated genes shared only by P3 and P4, including SLAMF7, whose activation was recently described to downregulate CCR5 on monocytes and reduced their infection capacity by HIV (83), suggesting a potential difference in monocyte susceptibility and phenotype in these individuals during hyper-acute infection. Moreover, referring back to the initial cell type clustering of the data (FIG. 6), Applicants noted that the peak response monocytes in P3 (0 weeks) clustered separately from other monocytes, and that P4 made up >75% of the anti-viral monocytes detected at 1-week post-infection. Identifying a potential correlate of future viral control otherwise obscured by bulk transcriptomics and sparse longitudinal sampling, Applicants next searched for other unique immune responses enriched in either or both of the two controllers.

### Future Controllers Exhibit Higher Frequencies of Proliferating CTLs and a Precocious Subset of NK Cells Before Traditional HIV-Specific CD8+ T Cells

As CD8+ T cells are known to play a part in controlling chronic HIV infection (82, 84, 85), Applicants turned to the CTLs in the study to look for differences between the individuals who controlled infection long-term and those who did not. Through the module discovery approach, Applicants found that CTLs produced increasing levels of PRF1 and GZMB along the course of hyper-acute infection (FIG. 3C). Further unsupervised and directed approaches did not elucidate meaningful or significant differences in CTL responses across individuals by outcome of viral control (FIGS. 15A-15B and Tables 7A-7D).

Then Applicants turned to the proliferating T cells in the study to look for differences in response based on long-term viral control. En masse, the proliferating T cells expressed similar levels of cytotoxic genes as non-proliferating CTLs (FIG. 15C). DE analysis highlighted genes associated with cell-cycle (e.g. STMN1, HISTIHIB, MKI67) and memory (e.g. IL7R, KLRB1) (see FIG. 15D and Tables 7A-7D) for proliferating and non-proliferating CTLs, respectively. While sparsely detected due to the method of library construction in Seq-Well, Applicants did measure a limited number of TCR variable genes in the proliferating CTLs (FIG. 15E). In fact, Applicants noted enrichment of TRBV and TRAV genes known to construct prevalent CDR3 sequences that bind common HIV epitopes (87, 88): TRBV28 (QW9/FL8/KF11/KK10/NV9, χ² test p=2.4*10⁻²⁶), TRAV4 (KK10, χ² test p=3.5*10⁻⁶), and TRBV20-1 (KK10/KF11/GY9/NV9, χ² test p=0.059). The single-cell data here expand the recently published bulk RNA-Seq data on HIV-specific CTLs in this cohort (89), but also enabled Applicants to elucidate heterogeneity in this proliferating cytotoxic response as a function of time.

Grouping proliferating T cells with the other CTLs, Applicants sought to understand if these two controllers demonstrated differences in the frequency of proliferating T cells among the total CTL pool overtime. Strikingly, both controllers (P3 & P4) displayed much higher frequencies of proliferating T cells within the first month of infection (FIG. 5A). While all four individuals developed proliferating T cells at 1-week post HIV detection, P3 and P4 exhibited a higher fraction of these cells 1 week after HIV detection (30-40%).

Applicants next utilized unsupervised analyses to explore differences in proliferating T cell responses over time among individuals (FIG. 5B, FIG. 15F). Proliferating T cells captured at 1-week post-infection strongly separated in PCA across both PC1 and PC2 (p<0.001). Clustering over all proliferating T cells (see Methods), Applicants identified four clusters of cells with distinct gene programs (see FIG. 5C and Table 12): traditional CD8+ T cells (1-red), hyper-proliferative CD8+ T cells (2-green), naïve CD4+ T cells (3-cyan), and a subset of cells that is CD8− but TRDC+ and FCGR3A+(CD16) (4-lilac). To determine whether these TRDC⁺CD16⁺ cells were γδT or NK cells, Applicants scored them, as well as non-proliferating CTLs and NK cells, against gene signatures described in that study (FIG. 15G). Based on score similarity to NK cells, and the relative down-regulation of CD3 compared to the other proliferating T cell subsets (Wilcoxon rank sum test; CD3D: log(FC)=−0.895, q=2.7×10⁻⁴²; CD3G: log(FC)=−0.923, q=8.9×10⁻³⁷), Applicants determined cluster 4 (lilac) to be proliferating NK cells. Looking at the distribution of timepoints within each of these clusters, this NK cluster (4-lilac) contained the highest proportion of cells assayed at HIV detection and 1 week thereafter (FIGS. 5D, 5E). Within these earliest proliferating NK cells, the majority were detected from P3 and P4. Together, these data suggested that individuals who go on control HIV infection without ART exhibit a subset of proliferative, cytotoxic NK cells before the majority of HIV-specific CD8+ T cells arise. Thus, investigating the classically induced cytotoxic cells in viral infection on a single-cell level revealed unappreciated heterogeneity in the anti-viral response, implicating innate immune responses in controlling infection.

### Discussion

Here, Applicants applied both unsupervised and directed approaches to a unique longitudinal human infection data set to characterize conserved immune response dynamics, as well as early cellular events associated with the individuals studied here who go on to control infection without treatment. Sampling prior to and immediately upon HIV infection, Applicants assayed longitudinal PBMC samples in four individuals from a prospective cohort, the FRESH Study (16, 17) using Seq-Well (18). This systems-level approach revealed parameters shared across all cell types examined (e.g., response to IFN), as well as subtle variations among cellular types and individuals missed in previous bulk studies of infection. Further, it defined cell-type specific responses (e.g., inflammatory induction of CD4+ T cells), and their interaction dynamics following infection. Moreover, leveraging the resolution and high-throughput capability of scRNA-Seq methods, Applicants were able to uncover previously unappreciated cellular features in the PBMCs of two individuals who went on to control infection naturally, including subsets of poly-functional monocytes and proliferating NK cells limited to hyper-acute infection, that may correspond to better infection outcome.

To systematically identify immune cells responding with similar temporal dynamics, Applicants adapted WGCNA (26, 27) (FIG. 2A and see Methods) to discover modules of genes that significantly changed in expression within a given cell type over time. Cellular responses to infection can happen on the order of hours to days; therefore, even with the biweekly HIV testing in the FRESH Study, Applicants anticipated these individuals would not align immune responses in absolute time. After applying the module analysis, the strongest and most pervasive module across cell types and all individuals assayed was the interferon induced anti-viral response (FIG. 2D). While known to be a key factor in controlling HIV replication (30, 65) and the major response in NHP SIV infection models (52, 90), the timing of response and extent to which it pervades all peripheral cell subsets in humans has not yet been described. Of note, both controllers (P3 & P4) exhibited interferon response modules the week before peak viremia, consistent with the faster resolution of interferon response in natural SIV hosts compared to non-natural hosts (46-49). Moreover, multiple modules from P3 & P4 uniquely contained APOBEC3A, shown to restrict HIV infection in myeloid cells (91), and IFITM1 and IFITM3 which can inhibit HIV translation in transfected cells in-vitro (92).

Due to the ability to determine enriched modules within individual cells, Applicants were able to unveil a second layer of regulation, which might otherwise be drowned out by the overwhelming IFN signature (FIGS. 3F-3H). This highlighted upstream drivers that were unique to CD4+ T cells, monocytes, NK cells, or shared amongst many cell types. Downstream genes (many shared) were significantly enriched for many known drivers of lymphocyte proliferation, emphasizing the presence of mounting large cytotoxic responses in more than just HIV-specific CD8+ T cells during acute infection. Some of these molecules were also upstream of CD4+ T cells, potentially increasing their susceptibility to infection (IL-15) (69) and inducing maturation (IL-2) (67) and differentiation (IL-4) (93). Cell-type specific drivers, like IL-1B & TNF upstream of CD4+ T cells, also suggested T helper subset differentiation during this time frame (70). The integrated multi-cellular analysis laid the foundation for future characterization of the complex, dynamic immune responses to an infection. A potential method to pinpoint the effects of the various cytokines produced in acute infection might utilize in-vitro assays that couple PBMCs from healthy individuals with and without autologously HIV infected CD4+ T cells.

Empowered by the single-cell resolution and cognizant of the role HIV-specific T cells play in long-term control (82, 84, 94), Applicants were intrigued to find not only higher frequencies of proliferating CTLs in P3/P4, but also the presence of a subset of a previously unappreciated proliferating NK cells preceding the well-described HIV-specific responses (FIGS. 5C-5E), given the multi-faceted role of NK cells in viral control (64).

Collectively, the single-cell transcriptional study of hyper-acute and acute HIV infection in FRESH provided several key insights into the dynamics of host-immune responses to infection on a systems-level. It also afforded a key reference data set for studying the earliest moments of viral infection after detection. Applicants were able to nominate potential early responses that may inform long-term viral control and thus guide HIV vaccine efforts.

Non-Overlapping B Cell Modules in MM1

While B cell modules were present in two individuals (P1 & P2) in MMI, they actually expressed divergent gene expression patterns (FIG. 11B): B cells from P1 upregulated IGHM, CXCR4, and IL4R, genes associated with naïve B cells (110, 111); B cells from P2, meanwhile, upregulated mitochondrial genes, a potential sign of cellular stress (M4). Peaking at 6 months post-HIV detection, P2 also upregulated IGHG1-4, CD52, and HLA-DRA (M2), genes indicative of mature, class-switched cells; P1 demonstrated a similar module in time and gene membership (M1) of these B cells, but this module clustered into MM5 in this individual.

### REFERENCES

1. T. D. Querec et al., Systems biology approach predicts immunogenicity of the yellow fever vaccine in humans. Nature Immunology. 10, 116-125 (2009).
2. C. Li et al., Host Regulatory Network Response to Infection with Highly Pathogenic H5N1 Avian Influenza Virus. Journal of Virology. 85, 10955-10967 (2011).
3. J. T. Gaublomme et al., Single-Cell Genomics Unveils Critical Regulators of Thl7 Cell Pathogenicity. Cell. 163, 1400-1412 (2015).
4. L. Jerby-Arnon et al., A Cancer Cell Program Promotes T Cell Exclusion and Resistance to Checkpoint Blockade. Cell. 175, 984-997.e24 (2018).
5. I. Tirosh et al., Dissecting the multicellular ecosystem of metastatic melanoma by single-cell RNA-seq. Science. 352, 189-196 (2016).
6. G. Ledergor et al., Single cell dissection of plasma cell heterogeneity in symptomatic and asymptomatic myeloma. Nat. Med 24, 1867-1876 (2018).
7. E. Martin-Gayo et al., A Reproducibility-Based Computational Framework Identifies an Inducible, Enhanced Antiviral State in Dendritic Cells from HIV-1 Elite Controllers. Genome Biology. 19, 10 (2018).
8. J. Ordovas-Montanes et al., Allergic inflammatory memory in human respiratory epithelial progenitor cells. Nature. 560, 649 (2018).
9. Y. Steuerman et al., Dissection of Influenza Infection In Vivo by Single-Cell RNA Sequencing. Cell Syst. 6, 679-691.e4 (2018).
10. M. Sade-Feldman et al., Defining T Cell States Associated with Response to Checkpoint Immunotherapy in Melanoma. Cell. 175, 998-1013.e20 (2018).
11. K. Davie et al., A Single-Cell Transcriptome Atlas of the Aging Drosophila Brain. Cell. 174, 982-998.e20 (2018).
12. J. Cao et al., The single-cell transcriptional landscape of mammalian organogenesis. Nature. 566, 496 (2019).
13. E. J. Arts, D. J. Hazuda, HIV-1 Antiretroviral Drug Therapy. Cold Spring Harb Perspect Med. 2, a007161 (2012).
14. W. D. F. Venter, F. Cowan, V. Black, K. Rebe, L.-G. Bekker, Pre-exposure prophylaxis in Southern Africa: feasible or not? Journal of the International AIDS Society. 18, 19979 (2015).
15. Joint United Nations Programme on HIV/AIDS (UNAIDS), “UNAIDS Data 2018” (Joint United Nations Programme on HIV/AIDS (UNAIDS), 2018).
16. K. L. Dong et al., Detection and treatment of Fiebig stage I HIV-1 infection in young at-risk women in South Africa: a prospective cohort study. The Lancet HIV. 5, e35-e44 (2018).
17. T. Ndung'u, K. L. Dong, D. S. Kwon, B. D. Walker, A FRESH approach: Combining basic science and social good. Science Immunology. 3, eaau2798 (2018).
18. T. M. Gierahn et al., Seq-Well: portable, low-cost RNA sequencing of single cells at high throughput. Nature Methods. 14, 395-398 (2017).
19. A.-C. Villani et al., Single-cell RNA-seq reveals new types of human blood dendritic cells, monocytes, and progenitors. Science. 356 (2017), doi:10.1126/science.aah4573.
20. H. M. Kang et al., Multiplexed droplet single-cell RNA-sequencing using natural genetic variation. Nature Biotechnology. 36, 89-94 (2018).
21. M. Gutierrez-Arcelus et al., Lymphocyte innateness defined by transcriptional states reflects a balance between proliferation and effector functions. Nat Commun. 10, 687 (2019).
22. D. C. Douek, L. J. Picker, R. A. Koup, T cell dynamics in HIV-1 infection. Annu. Rev. Immunol. 21, 265-304 (2003).
23. H. Sprenger et al., Selective induction of monocyte and not neutrophil-attracting chemokines after influenza A virus infection. J Exp. Med 184, 1191-1196 (1996).
24. L. E. Carlin, E. A. Hemann, Z. R. Zacharias, J. W. Heusel, K. L. Legge, Natural Killer Cell Recruitment to the Lung During Influenza A Virus Infection Is Dependent on CXCR3, CCR5, and Virus Exposure Dose. Front Immunol. 9 (2018), doi:10.3389/fimmu.2018.00781.
25. T. P. Salazar-Mather, J. S. Orange, C. A. Biron, Early murine cytomegalovirus (MCMV) infection induces liver natural killer (NK) cell inflammation and protection through macrophage inflammatory protein lalpha (MIP-lalpha)-dependent pathways. J. Exp. Med. 187, 1-14 (1998).
26. B. Zhang, S. Horvath, A general framework for weighted gene co-expression network analysis. Sta tAppl Genet Mol Biol. 4, Article 17 (2005).
27. P. Langfelder, S. Horvath, WGCNA: an R package for weighted correlation network analysis. BMC Bioinformatics. 9, 559 (2008).
28. J. W. Mellors et al., Quantitation of HIV-1 RNA in plasma predicts outcome after seroconversion. Ann. Intern. Med. 122, 573-579 (1995).
29. L. Lavreys et al., Higher Set Point Plasma Viral Load and More-Severe Acute HIV Type 1 (HIV-1) Illness Predict Mortality among High-Risk HIV-1-Infected African Women. Clin Infect Dis. 42, 1333-1339 (2006).
30. N. Grandvaux, B. R. tenOever, M. J. Servant, J. Hiscott, The interferon antiviral response: from viral invasion to evasion. Curr. Opin. Infect. Dis. 15, 259-267 (2002).
31. S. A. Samarajiwa, S. Forster, K. Auchettl, P. J. Hertzog, INTERFEROME: the database of interferon regulated genes. Nucleic Acids Res. 37, D852-D857 (2009).
32. J. W. Schoggins, C. M. Rice, Interferon-stimulated genes and their antiviral effector functions. Curr Opin Virol. 1, 519-525 (2011).
33. B. Corleis et al., Early type I Interferon response induces upregulation of human (3-defensin 1 during acute HIV-1 infection. PLoS ONE. 12, e0173161 (2017).
34. D. A. Vargas-Inchaustegui et al., CXCL10 production by human monocytes in response to Leishmania braziliensis infection. Infect. Immun. 78, 301-308 (2010).
35. L. Wu, V. N. KewalRamani, Dendritic-cell interactions with HIV: infection and viral dissemination. Nat. Rev. Immunol. 6, 859-868 (2006).
36. J. Luban, Innate immune sensing of HIV-1 by dendritic cells. Cell Host Microbe. 12, 408-418 (2012).
37. D. Ng, J. L. Gommerman, The Regulation of Immune Responses by DC Derived Type I IFN. Front Immunol. 4 (2013), doi:10.3389/fimmu.2013.00094.
38. C. Riou et al., Distinct kinetics of Gag-specific CD4+ and CD8+ T cell responses during acute HIV-1 infection. J. Immunol. 188, 2198-2206 (2012).
39. A. G. Schrum, L. A. Turka, The Proliferative Capacity of Individual Naive CD4+ T Cells Is Amplified by Prolonged T Cell Antigen Receptor Triggering. J Exp Med. 196, 793-803 (2002).
40. S. Kurtulus et al., TIGIT predominantly regulates the immune response via regulatory T cells. J Clin Invest. 125, 4053-4062 (2015).
41. B. Samten, CD52 as both a marker and an effector molecule of T cells with regulatory action: Identification of novel regulatory T cells. Cell Mol Immunol. 10, 456-458 (2013).
42. E. Lugli, E. Marcenaro, D. Mavilio, N K Cell Subset Redistribution during the Course of Viral Infections. Front Immunol. 5 (2014), doi:10.3389/fimmu.2014.00390.
43. R. K. Reeves, T. I. Evans, J. Gillis, R. P. Johnson, Simian Immunodeficiency Virus Infection Induces Expansion of 4 7+ and Cytotoxic CD56+NK Cells. Journal of Virology. 84, 8959-8963 (2010).
44. L. E. Carlin-Brown, C. A. Fullenkamp, J. McGill, K. L. Legge, J. W. Heusel, The Journal of Immunology, in press.
45. R. Mehla, V. Ayyavoo, Gene Array Studies in HIV-1 Infection. Curr HIV/AIDS Rep. 9, 34-43 (2012).
46. S. Lederer et al., Transcriptional profiling in pathogenic and non-pathogenic SIV infections reveals significant distinctions in kinetics and tissue compartmentalization. PLoS Pathog. 5, e1000296 (2009).
47. S. E. Bosinger et al., Global genomic analysis reveals rapid control of a robust innate response in SIV-infected sooty mangabeys. J. Clin. Invest. 119, 3556-3572 (2009).
48. Z.-W. Yang et al., Coexpression Network Analysis of Benign and Malignant Phenotypes of SIV-Infected Sooty Mangabey and Rhesus Macaque. PLoS ONE. 11, e0156170 (2016).
49. B. Jacquelin et al., Nonpathogenic SIV infection of African green monkeys induces a strong but rapidly controlled type I IFN response. J. Clin. Invest. 119, 3544-3555 (2009).
50. M. Solis et al., RIG-I-Mediated Antiviral Signaling Is Inhibited in HIV-1 Infection by a Protease-Mediated Sequestration of RIG-I. Journal of Virology. 85, 1224-1236 (2011).
51. J. D. Farrar et al., Selective loss of type I interferon-induced STAT4 activation caused by a minisatellite insertion in mouse Stat2. Nature Immunology. 1, 65 (2000).
52. S. E. Bosinger et al., Intact Type I Interferon Production and IRF7 Function in Sooty Mangabeys. PLOS Pathogens. 9, e1003597 (2013).
53. K. Honda, A. Takaoka, T. Taniguchi, Type I Inteferon Gene Induction by the Interferon Regulatory Factor Family of Transcription Factors. Immunity. 25, 349-360 (2006).
54. M. Sato et al., Positive feedback regulation of type I IFN genes by the IFN-inducible transcription factor IRF-7. FEBS Lett. 441, 106-110 (1998).
55. F. Ma et al., Positive Feedback Regulation of Type I IFN Production by the IFN-Inducible DNA Sensor cGAS. The Journal of Immunology. 194, 1545-1554 (2015).
56. A. Michalska, K. Blaszczyk, J. Wesoly, H. A. R. Bluyssen, A Positive Feedback Amplifier Circuit That Regulates Interferon (IFN)-Stimulated Gene Expression and Controls Type I and Type II IFN Responses. Front. Immunol. 9 (2018), doi:10.3389/fimmu.2018.01135.
57. M. O'Brien, O. Manches, N. Bhardwaj, Plasmacytoid Dendritic Cells in HIV Infection. Adv Exp Med Biol. 762, 71-107 (2013).
58. J. J. Trombetta et al., Preparation of Single-Cell RNA-Seq Libraries for Next Generation Sequencing. Curr Protoc Mol Biol. 107 (2014), doi:10.1002/0471142727.mb0422s107.
59. M. E. Futschik, B. Carlisle, Noise-robust soft clustering of gene expression time-course data. J Bioinform Comput Biol. 3, 965-988 (2005).
60. L. Kumar, M. E. Futschik, Mfuzz: A software package for soft clustering of microarray data. Bioinformation. 2, 5-7 (2007).
61. E. I. Athanasiadis et al., Single-cell RNA-sequencing uncovers transcriptional states and fate decisions in haematopoiesis. Nature Communications. 8, 2045 (2017).
62. R. D. Sloan et al., Productive Entry of HIV-1 during Cell-to-Cell Transmission via Dynamin-Dependent Endocytosis. Journal of Virology. 87, 8110-8123 (2013).
63. N. Gulzar, K. F. T. Copeland, CD8+ T-cells: function and response to HIV infection. Curr. HIV Res. 2, 23-37 (2004).
64. E. Scully, G. Alter, N K Cells in HIV Disease. Curr HIV AIDS Rep. 13, 85-94 (2016).
65. L. Cheng et al., Type I interferons suppress viral replication but contribute to T cell depletion and dysfunction during chronic HIV-1 infection. JCI Insight. 2, doi:10.1.172/jci.insight.94366.
66. A. Zhen et al., Targeting type I interferon-mediated activation restores immune function in chronic HIV infection. J. Clin. Invest. 127, 260-268 (2017).
67. I. Sereti et al., IL-2-induced CD4+ T-cell expansion in HIV-infected patients is associated with long-term decreases in T-cell proliferation. Blood. 104, 775-780 (2004).
68. O. M. Anton, S. Vielkind, M. E. Peterson, Y. Tagaya, E. O. Long, NK Cell Proliferation Induced by IL-15 Transpresentation Is Negatively Regulated by Inhibitory Receptors. The Journal of Immunology. 195, 4810-4821 (2015).
69. L. Manganaro et al., IL-15 regulates susceptibility of CD4+ T cells to HIV infection. PNAS. 115, E9659-E9667 (2018).
70. K. Hebel et al., IL-1 and TGF-Act Antagonistically in Induction and Differentially in Propagation of Human Proinflammatory Precursor CD4+ T Cells. The Journal of Immunology. 187, 5627-5635 (2011).
71. J. A. Harton, J. P.-Y. Ting, Class II Transactivator: Mastering the Art of Major Histocompatibility Complex Expression. Mol Cell Biol. 20, 6185-6194 (2000).
72. G. Carbotti et al., IL-27 mediates HLA class I up-regulation, which can be inhibited by the IL-6 pathway, in HLA-deficient Small Cell Lung Cancer cells. JExp Clin Cancer Res. 36, 140 (2017).
73. M. Zeng et al., Lymphoid Tissue Damage in HIV-1 Infection Depletes Naïve T Cells and Limits T Cell Reconstitution after Antiretroviral Therapy. PLOS Pathogens. 8, e1002437 (2012).
74. Y. I. Son et al., Interleukin-18 (IL-18) synergizes with IL-2 to enhance cytotoxicity, interferon-gamma production, and expansion of natural killer cells. Cancer Res. 61, 884-888 (2001).
75. C. Petes, M. K. Mariani, Y. Yang, N. Grandvaux, K. Gee, Interleukin (IL)-6 Inhibits IL-27- and IL-30-Mediated Inflammatory Responses in Human Monocytes. Front Immunol. 9 (2018), doi:10.3389/fimmu.2018.00256.
76. A. R. Stacey et al., Induction of a striking systemic cytokine cascade prior to peak viremia in acute human immunodeficiency virus type 1 infection, in contrast to more modest and delayed responses in acute hepatitis B and C virus infections. J. Virol. 83, 3719-3733 (2009).
77. M. J. Robertson, Role of chemokines in the biology of natural killer cells. J. Leukoc. Biol. 71, 173-183 (2002).
78. J. C. Tilton et al., Diminished Production of Monocyte Proinflammatory Cytokines during Human Immunodeficiency Virus Viremia Is Mediated by Type I Interferons. Journal of Virology. 80, 11486-11497 (2006).
79. F. Porcheray, B. Samah, C. Leone, N. Dereuddre-Bosquet, G. Gras, Macrophage activation and human immunodeficiency virus infection: HIV replication directs macrophages towards a pro-inflammatory phenotype while previous activation modulates macrophage susceptibility to infection and viral production. Virology. 349, 112-120 (2006).
80. P. J. Ellery et al., The CD16+ Monocyte Subset Is More Permissive to Infection and Preferentially Harbors HIV-1 In Vivo. The Journal of Immunology. 178, 6581-6589 (2007).
81. S. G. Deeks, B. D. Walker, Human immunodeficiency virus controllers: mechanisms of durable virus control in the absence of antiretroviral therapy. Immunity. 27, 406-416 (2007).
82. A. R. Hersperger et al., Increased HIV-specific CD8+ T-cell cytotoxic potential in HIV elite controllers is associated with T-bet expression. Blood. 117, 3799-3808 (2011).
83. P. O'Connell et al., SLAMF7 Is a Critical Negative Regulator of IFN-α-Mediated CXCL10 Production in Chronic HIV Infection. The Journal of Immunology. 202, 228-238 (2019).
84. A. R. Hersperger et al., Perforin expression directly ex vivo by HIV-specific CD8 T-cells is a correlate of HIV elite control. PLoS Pathog. 6, e1000917 (2010).
85. R. A. Koup et al., Temporal association of cellular immune responses with the initial control of viremia in primary human immunodeficiency virus type 1 syndrome. Journal of Virology. 68, 4650-4655 (1994).
86. Z. M. Ndhlovu et al., Magnitude and Kinetics of CD8+ T Cell Activation during Hyperacute HIV Infection Impact Viral Set Point. Immunity. 43, 591-604 (2015).
87. J. A. Conrad et al., Dominant clonotypes within HIV-specific T cell responses are PD-1hi and CD127low and display reduced variant cross-reactivity. J Immunol. 186, 6871-6885 (2011).
88. D. J. van Bockel et al., Persistent Survival of Prevalent Clonotypes within an Immunodominant HIV Gag-Specific CD8+ T Cell Response. The Journal of Immunology. 186, 359-371 (2011).
89. Z. M. Ndhlovu et al., Augmentation of HIV-specific T cell function by immediate treatment of hyperacute HIV-1 infection. Science Translational Medicine, in press.
90. N. G. Sandler et al., Type I interferon responses in rhesus macaques prevent SIV infection and slow disease progression. Nature. 511, 601-605 (2014).
91. G. Berger et al., APOBEC3A Is a Specific Inhibitor of the Early Phases of HIV-1
Infection in Myeloid Cells. PLOS Pathogens. 7, e1002221 (2011).
92. W.-Y. J. Lee, R. M. Fu, C. Liang, R. D. Sloan, IFITM proteins inhibitHIV-1 protein synthesis. Scientific Reports. 8, 14551 (2018).
93. J. L. Silva-Filho, C. Caruso-Neves, A. A. S. Pinheiro, IL-4: an important cytokine in determining the fate of T cells. Biophys Rev. 6, 111-118 (2014).
94. G. Gaiha et al., Structural Topology Defines Protective CD8+ T cell Epitopes in the HIV Proteome. Science, in press.
95. K. A. O'Connell, Y. Han, T. M. Williams, R. F. Siliciano, J. N. Blankson, Role of Natural Killer Cells in a Cohort of Elite Suppressors: Low Frequency of the Protective KIR3DS1 Allele and Limited Inhibition of Human Immunodeficiency Virus Type 1 Replication In Vitro. Journal of Virology. 83, 5028-5034 (2009).
96. C. W. Pohlmeyer et al., Journal of Virology, in press, doi:10.1128/JVI.01790-18.
97. A. Kurioka et al., CD161 Defines a Functionally Distinct Subset of Pro-Inflammatory Natural Killer Cells. Front Immunol. 9 (2018), doi:10.3389/fimmu.2018.00486.
98. B. Foley et al., Natural Killer (NK) Cells Respond to CMV Reactivation After Allogeneic Transplantation with An Increase in NKG2C+CD57+ Self-KIR+ NK Cells with Potent IFNγ Production. Blood. 118, 356-356 (2011).
99. B. Foley et al., Human Cytomegalovirus (CMV)-Induced Memory-like NKG2C+ NK Cells Are Transplantable and Expand In Vivo in Response to Recipient CMV Antigen. The Journal ofImmunology. 189, 5082-5088 (2012).
100. N. K. Bjorkstrom et al., Rapid expansion and long-term persistence of elevated NK cell numbers in humans infected with hantavirus. JExp Med. 208, 13-21 (2011).
101. R. K. Reeves et al., Antigen-specific NK cell memory in rhesus macaques. Nat. Immunol. 16, 927-932 (2015).
102. A. Butler, P. Hoffman, P. Smibert, E. Papalexi, R. Satija, Integrating single-cell transcriptomic data across different conditions, technologies, and species. Nature Biotechnology. 36, 411-420 (2018).
103. G. C. Linderman, M. Rachh, J. G. Hoskins, S. Steinerberger, Y. Kluger, Fast interpolation-based t-SNE for improved visualization of single-cell RNA-seq data. Nature Methods. 16, 243 (2019).
104. A. Liberzon et al., The Molecular Signatures Database Hallmark Gene Set Collection. Cell Systems. 1, 417-425 (2015).
105. J. Godec et al., Compendium of Immune Signatures Identifies Conserved and Species-Specific Biology in Response to Inflammation. Immunity. 44, 194-206 (2016).
106. B. Li, C. N. Dewey, RSEM: accurate transcript quantification from RNA-Seq data with or without a reference genome. BMC Bioinformatics. 12, 323 (2011).
107. C. Trapnell, L. Pachter, S. L. Salzberg, TopHat: discovering splice junctions with RNA-Seq. Bioinformatics. 25, 1105-1111 (2009).
108. A.-S. Beignon et al., Endocytosis of HIV-1 activates plasmacytoid dendritic cells via Toll-like receptor-viral RNA interactions. J. Clin. Invest. 115, 3265-3275 (2005).
109. B. Malleret et al., Primary infection with simian immunodeficiency virus: plasmacytoid dendritic cell homing to lymph nodes, type I interferon, and immune suppression. Blood. 112, 4598-4608 (2008).
110. M. Becker, E. Hobeika, H. Jumaa, M. Reth, P. C. Maity, CXCR4 signaling and function require the expression of the IgD-class B-cell antigen receptor. Proc Natl Acad Sci USA. 114, 5231-5236 (2017).
111. E. F. Wagner et al., Novel Diversity in IL-4-Mediated Responses in Resting Human Naive B Cells Versus Germinal Center/Memory B Cells. The Journal of Immunology. 165, 5573-5579 (2000).
112. S. Moir, A. S. Fauci, B cells in HIV infection and disease. Nat Rev Immunol. 9, 235-245 (2009).
113. J. M. Mabuka et al., Plasma CXCL13 but Not B Cell Frequencies in Acute HIV Infection Predicts Emergence of Cross-Neutralizing Antibodies. Front Immunol. 8, 1104 (2017).
114. T. Bradley et al., Pentavalent HIV-1 vaccine protects against simian-human immunodeficiency virus challenge. Nat Commun. 8 (2017), doi:10.1038/ncomms15711.
115. M. G. Pauthner et al., Vaccine-Induced Protection from Homologous Tier 2 SHIV Challenge in Nonhuman Primates Depends on Serum-Neutralizing Antibody Titers. Immunity. 50, 241-252.e6 (2019).

### Materials and Methods

### Study Subjects

All individuals in this study were participants in the FRESH Cohort (16, 17). This study enrolled HIV negative women, ages 18-24, and tests for HIV-1 RNA in the plasma twice a week for one year. Each time the women came to the study center, they participate in peer-support groups and receive a stipend. In addition to semi-weekly virus testing by RT-PCR, whole blood was collected 4 times (including during enrollment) throughout the year from participants. If a plasma test came back positive, the participant was asked to come back to the clinic that day to collect a blood sample. Samples were then collected weekly through the first 6 weeks of infection, and regularly afterward as long as the individual continues to return to the study center. In the arm of the study described herein, subjects were initiated on anti-retroviral therapy (ART) when their CD4 count fell below 350 cells/μL, per standard treatment guidelines at the time of enrollment. A second arm of the study was initiated in 2014; in it, individuals who tested positive for viral RNA were initiated on ART when they were called back into the study center for their first post-infection sample collection. To the best of Applicants' knowledge, all individuals in this study had not yet started ART for the time points processed here. FRESH was performed in accordance with protocols approved by the institutional review board at Partners (Massachusetts General Hospital, Boston, USA), MIT (Cambridge, USA) and the biomedical research ethics committee of the University of KwaZulu-Natal (Durban, South Africa).

### Cell Preparation, Flow Cytometry, and Cell Sorting

Frozen peripheral blood mononuclear cells (PBMCs) were thawed and washed twice with warm RPMI supplemented with 10% fetal bovine serum. Next, the cells were resuspended in FACS buffer (PBS supplemented with 1% FBS) and stained with antibodies on ice for 30 minutes in FACS buffer. Antibodies used include Alexa Fluor 700—CD45 (Biolegend, clone 2D1), BUV737—CD3 (BD Biosciences, clone UCHT1), BV711—CD4 (Biolegend, clone OKT4), BUV395—CD8 (BD Biosciences, clone RPA-T8), BV605—CD14 (Biolegend, clone M5E2), BV510—HLA-DR (BD Biosciences, clone G46-6), and BV650—CD123 (Biolegend, clone 6H6); subsets of these markers were used to identify immune cells (CD45⁺), CD4⁺ (CD45⁺CD14⁻CD3⁺CD4⁺) and CD8⁺ (CD45⁺CD14⁻CD3⁺CD8⁺) T cells, and pDCs (CD45⁺CD14⁻CD3⁻CD11c⁻HLA-DR⁺CD123⁺⁺). Afterward, the cells were washed and stained with the viability stain Calcein Blue, AM (Invitrogen, C34853) for 15 minutes on ice. Finally, the stained cells were washed twice with FACS buffer and sorted on a BD SORP FACSAria II cell sorter using BD FACSDiva software and a 100-micron nozzle. Up to 250,000 viable immune cells (CD45⁺Calcein Blue⁺) were sorted into 1 ml of RPMI+10% FBS for Seq-Well (18). For Smart-Seq2 (58), individual cells were directly sorted into 10 μl of RLT (Qiagen)+1% BME in 96 well plates.

Single-cellRNA-Seq (scRNA-Seq) with Seq-Well

The Seq-Well platform was utilized as previously described (18) to capture the transcriptomes of single cells on barcoded mRNA capture beads. In brief, 10 μL of sorted CD45⁺Calcein Blue⁺ PBMCs were mixed 1:1 with the viability stain trypan blue and counted using a hemocytometer. The cells were resuspended in RPMI+10% FBS at a final concentration of ˜100,000 cells/mL, and 20,000-25,000 cells in 200 μL were added to each Seq-Well array preloaded with barcoded mRNA capture beads (ChemGenes). Two arrays were used for each sample to increase cell numbers. The arrays were then sealed with a polycarbonate membrane (pore size: 0.01 μm), cells were lysed, transcripts were hybridized to the beads, and the barcoded mRNA capture beads were recovered and pooled for reverse transcription using Maxima H-RT (Thermo Fisher EPO0753), and all subsequent steps. After an Exonuclease I treatment (NEB M0293L) to remove excess primers, whole transcriptome amplification (WTA) was carried out using KAPA HiFi PCR Mastermix (Kapa Biosystems KK2602) with 2,000 beads per 50 μL reaction volume. Libraries were then pooled in sets of eight (totaling 16,000 beads) and purified using Agencourt AMPure XP beads (Beckman Coulter, A63881) by a 0.6×SPRI followed by a 0.8×SPRI and quantified using Qubit hsDNA Assay (Thermo Fisher Q32854). Quality of the WTA product was assessed using the Agilent hsD5000 Screen Tape System (Agilent Genomics) with an expected peak >800 bp tailing off to beyond 3000 bp, and a small/non-existent primer peak, indicating a successful preparation. Libraries were then constructed using a Nextera XT DNA library preparation kit (Illumina FC-131-1096) on a total of 750 pg of pooled cDNA library from 16,000 recovered beads using index primers as previously described (18). Tagmented and amplified sequences were purified using a 0.8×SPRI ratio yielding library sizes with an average distribution of 500-750 bp in length as determined using an Agilent hsD1000 Screen Tape System (Agilent Genomics). Two Seq-Well arrays were sequenced per NextSeq500 sequencing run with an Illumina 75 Cycle NextSeq500/550 v2 kit (Illumina FC-404-2005) at a final concentration of 2.4 pM. The read structure was paired end with Read 1, starting from a custom read 1 primer, covering 20 bases inclusive of a 12-bp cell barcode and 8-bp unique molecular identifier (UMI), then an 8-bp index read, and finally Read 2 containing 50 bases of transcript sequence.

### Seq-Well Alignment, Cell Identification, Cell Type Separation

Read alignment, cell barcode discrimination and UMI/transcript collation were performed as in Ordovas-Montanes et al (8) using a hg19 reference. Initially, Applicants aligned the sequences from P1 to a combined HIV+hg19 genome using the consensus sequence of HIV clade C viruses from the HIV Sequence Database (www.hiv.lanl.gov/content/sequence/HIV/mainpage.html). After alignment, however, Applicants measured 0-2 cells with HIV transcript alignments per array; therefore, Applicants used the standard hgl9 reference for their analysis. UMI collapsed data was used as input into Seurat (102) (version 2.3.4) for cell and gene trimming, and downstream analysis. The following steps were performed on all of the arrays processed from a single individual, on an individual-by-individual basis. Any cell with fewer than 750 UMIs or greater than 6,000 UMIs (0-5 cells/array), and any gene expressed in fewer than 5 cells were discarded from downstream analysis. This cells-by-genes matrix was then used to create a Seurat object for each individual. Cells with >20% of UMIs mapping to mitochondrial genes were then removed (50-100 cells/array). These objects (one per individual) were then merged into one object for pre-processing and cell-type identification

The combined Seurat object was log-normalized with a size factor of 10,000, and scaled without centering. Additionally, linear regression was performed to remove unwanted variation due to cellular complexity (nUMI) and low-quality cells (percent.mito). Subsequently, 3,251 variable genes were identified using the “LogVMR” function, and the following cutoffs: x.low.cutoff=0.01, x.high.cutoff=10, y.cutoff=0.25. Principal Component Analysis (PCA) was performed over these genes, and the top 17 PCs were chosen for clustering and embedding based on the curve of variance described by each PC and the genes most contributing to each PC. Next, FindClusters (SNN graph+ modularity optimization) with resolution=0.5 was used to generate 13 clusters, and the Fourier transform tSNE implementation (103) with 2,000 iterations to embed the data into 2-dimensional space.

Cluster identity was assigned by finding differentially expressed genes using Seurat's implemented Wilcoxon rank sum test, and then comparing those cluster-specific genes to previously published datasets (18-21). The cluster exhibited no cluster-specific genes; the cells from this cluster were embedded centrally in the tSNE, and upon further investigation expressed both myeloid and lymphocyte markers. Therefore, these cells were removed as multiplets (when multiple cells enter the same well in the Seq-Well array). After multiplet removal, 65,842 cells were captured across all samples processed. The remaining 12 clusters included subsets of major circulating immune cells (see Table 2 for marker genes). These clusters were merged by parent cell type (T cell, cytotoxic T cell, B cell, plasmablast, DC, monocyte) for downstream analysis, as variation in the SNN graph parameters weakly affected cluster assignment to the subsets.

As NK cells share many markers transcriptionally with cytotoxic T cells (21), clustering in Applicants' data set did not separate these two cytotoxic cell types. NK cells were annotated based on expression of CD3 (CD3D, CD3E, CD3G), CD16 (FCRG3A), and KLRF1. CD56 (NCAM) was not highly expressed in Applicants' data, and therefore was not used to separate NK cells. Any cell with a cluster identity belonging to the cytotoxic T cell cluster that lacked CD3 expression or expressed CD16/KLRF1 was annotated as an NK cell. With this annotation, Applicants noted distinct transcriptional responses between NK cells and CTLs both as a function of time and gene membership (FIGS. 2C, 3C-3E).

For downstream analysis of temporal variation in expression, the dataset was separated by individual and cell type: CD4+ T cells, NK cells, CTLs, proliferating T cells, B cells, plasmablasts, mDCs, and monocytes.

### Cell Type Normalization

Once separated by cell type and individual, the single-cell transcriptomes were processed on a cell-type by cell-type basis across all time points. For each cell type, the presence of residual contaminant RNA or doublets was assayed by scoring every cell against a set of contaminant genes from other cell types built from Applicants' marker list used to discern cluster identity (see Table 8 for cell-type specific contaminant gene lists and cut-offs). Cells with high contamination scores (0-10% of cells) were subsequently removed from further analysis to avoid unwanted variation in the subsequent unsupervised module discovery. Following contamination filtering, the data underwent scaling and normalization, followed by variable gene discovery (˜400-1,000 genes, dependent on cell type and cell number). PCA was then applied on these limited set of genes, followed by projection to the rest of the genes in the dataset.

### Module Discovery

For the module analysis, Applicants subset the data on the top and bottom 50 genes, after projection, for the first 3-9 PCs (dependent on the variance described by each PC, and genes contributing to each PC) as input for WGCNA functions (27). Following the WGCNA tutorial (horvath.genetics.ucla.edu/html/CoexpressionNetwork/Rpackages/WGCNA/Tutorials/), an appropriate soft power threshold was chosen to calculate the adjacency matrix. When possible, a power was chosen as suggested by the authors of WGCNA (i.e., the first power with a scale free topology above 0.8); however, in instances where this power yielded few modules (fewer than 3), Applicants decreased the power. Next, an adjacency matrix was generated using the selected soft power, and it was transformed into a Topological Overlap Matrix (TOM). Subsequently, this TOM was hierarchically clustered and the cutreeDynamic function with method “tree” was used to generate modules of correlated genes (minimum module size=10). Similar modules were then merged using a dissimilarity threshold of 0.5 (i.e., a correlation of 0.5).

To test the significance of the correlation structure of a given module, a permutation test was implemented. Binning genes in the true module by average gene expression (# bins=10), genes with the same distribution of average expression from the total list of genes used for module discovery were randomly picked 10,000 times. For each of these random modules, a one-sided Mann-Whitney U test was performed to compare the distribution of dissimilarity values between the genes in the true module and the distribution of dissimilarity values between the genes in the random module. Correcting the resulting p-values for multiple hypothesis testing by Benjamini-Hochberg FDR correction, a module was considered significant if fewer than 500 tests (p<0.05) had FDR >0.05.

Since Applicants were interested in identifying modules of genes that changed in expression as a function of time, another permutation test was implemented to identify modules that significantly vary from pre-infection. First, every cell was scored for the genes within the module, using the AddModuleScore function in Seurat. As testing for differences in distribution may be sensitive to sample number, a sample size (s) was selected based on the number of cells present at any given time point within a cell type. The smallest s used was 10; this cutoff was chosen based on the least frequent cell types having ˜100 cells total across all time points within an individual. If a time-point had fewer than 10 cells, that point was not used in the testing. In the case of plasmablasts and mDCs in multiple individuals, more than three time points had fewer than 10 cells, and therefore no modules were considered significantly variant in time. To determine if module expression varied over time, 1,000 Mann-Whitney-U tests between the distribution of scores from s random cells at pre-infection and s random cells from each other time point were performed. For each time point, the p-values from the 1,000 tests were then averaged. After FDR correction, if q<0.05 for any time point, the module was considered to significantly vary in expression in time. The approach and tests have been written as functions in R and have been included here.

### Module Grouping and Gene Set Analysis

In order to more easily compare modules by temporal pattern within and between individuals, fuzzy c-means clustering was applied to all of the modules in a given individual using the Mfuzz package (60) (version 2.38.0). Applicants chose to use fuzzy c-means clustering to allow us to understand the extent of membership of a given module to its assigned cluster. For each individual, c was chosen to be 5-7 such that diverse temporal patterns were separated, minimizing the number of clusters containing fewer than 3 modules. These groupings of modules were then annotated by similar scoring patterns across patients, taking into consideration that infection time is not the same for every individual (FIG. 10).

Gene Set Analysis on modules was performed using Ingenuity Pathway Analysis (IPA, Qiagen Inc.). Only gene names were supplied for analysis, and submitted for Core Analysis with the Experimentally Observed confidence setting. In FIGS. 3A-3E, the pathways annotated were taken from either the Canonical Pathways or Diseases & Functions results. For the upstream driver analysis in FIGS. 3F-3G, upstream drivers were selected by the following criteria: significant (p<0.001) in at least two modules of any given cell type, with at least 5 genes in the gene set. As the gene sets annotated in IPA are quite large and share many genes, the edges in Applicants' network were restricted to only those upstream drivers who shared 3 or more genes. To achieve finer grain temporal resolution on putative inducers of immune response, the union of enriched genes for each upstream driver from modules within a given cell type was used to generate scores against the single-cell expression data. Only upstream driver scores that demonstrated temporal variability (as described above) were included. Applicants report the median scores at each time point for each upstream driver.

The gene set enrichment analysis in FIGS. 4A-4E was performed using parts of MSigDB v6.2 (104, 105) (software.broadinstitute.org/gsea/msigdb). Multiple hypothesis testing was corrected by the Benjamini-Hochberg FDR procedure. The specific collections of gene sets used are reported in the figure legends.

scRNA-seq of pDCs with SMART-Seq2 and Analysis

Reverse transcription, WTA, and library preparation of single pDCs in 96-well plates was performed as previously described (58). Samples were sequenced on an Illumina NextSeq 500/550 instrument with an Illumina 75 Cycle NextSeq500/550 v2 kit (Illumina FC-404-2005) using 30-bp paired-end reads. Given difficulties acquiring pDCs from pre-infection samples due to limited cell numbers, Applicants sequenced pDCs from the peak interferon response and the 1-year time points in each individual. Reads (5*10⁵-3*10⁶/cell) were aligned to the hg38 (GENCODE v21) transcriptome and genome using RSEM (106) and Tophat (107), respectively. After trimming low quality cells (cells with <25,000 mapped reads or <1,000 genes), the remaining cells had a median of 122,000 mapped reads and 2,866 genes. Pre-processing and differential expression analysis were conducted in Seurat (102) using the Wilcoxon rank sum test. To test for differences in IFN responsiveness, individual-specific IFN response gene lists were used to generate scores in the pDCs using the AddModuleScore function in Seurat. The gene list used to score in each individual was chosen by including any gene that appeared at least twice in the modules that belonged to MM3 for that individual (see FIG. 9D).

### Luminex Cytokine Measurements

Matching plasma cytokine levels were determined in duplicate using a multiplexed magnetic bead assay (Catalogue number: LHC6003M, Life Technologies) in accordance with the manufacturer's instructions. Briefly, a mixture of beads that were coated with anti-cytokine antibodies were prewashed and then incubated with the plasma samples. They were then co-incubated with a mixture of biotinylated detector antibodies followed by R-phycoerythrin (R-PE) conjugated streptavidin. A magnetic separator was used to wash the beads between incubations. Fluorescence intensity was determined on a Bio-Plex 200 system. Concentrations of the cytokines in the samples were then determined by interpolating on sigmoid 4-parameter logistic regression standard curves.

### Other Statistical Methods

To determine TRBV or TRAV overabundance, Applicants performed a χ² test with Yates continuity correction. This test was performed independently for TRBV and TRAV genes, taking the random sampling (scaled by transcript detection) to be:

$$\frac{1}{\# of detected TRAV or TRBV genes} * \frac{\# total alignments to TRAV or TRBV genes}{\# of detected TRAV or TRBV genes} with \# detected TRBV genes = 24 ;$$

$$\# detected TRAV genes = 35 ;$$

$$\# total alignments to TRBV genes = 379 ;$$

$$\# total alignments to TRAV genes = 525.$$

Tables

Lengthy table referenced here
US20220226464A1-20220721-T00001
Please refer to the end of the specification for access instructions.

Lengthy table referenced here
US20220226464A1-20220721-T00002
Please refer to the end of the specification for access instructions.

Lengthy table referenced here
US20220226464A1-20220721-T00003
Please refer to the end of the specification for access instructions.

Lengthy table referenced here
US20220226464A1-20220721-T00004
Please refer to the end of the specification for access instructions.

Lengthy table referenced here
US20220226464A1-20220721-T00005
Please refer to the end of the specification for access instructions.

Lengthy table referenced here
US20220226464A1-20220721-T00006
Please refer to the end of the specification for access instructions.

Lengthy table referenced here
US20220226464A1-20220721-T00007
Please refer to the end of the specification for access instructions.

Lengthy table referenced here
US20220226464A1-20220721-T00008
Please refer to the end of the specification for access instructions.

Lengthy table referenced here
US20220226464A1-20220721-T00009
Please refer to the end of the specification for access instructions.

Lengthy table referenced here
US20220226464A1-20220721-T00010
Please refer to the end of the specification for access instructions.

Lengthy table referenced here
US20220226464A1-20220721-T00011
Please refer to the end of the specification for access instructions.

Lengthy table referenced here
US20220226464A1-20220721-T00012
Please refer to the end of the specification for access instructions.

Lengthy table referenced here
US20220226464A1-20220721-T00013
Please refer to the end of the specification for access instructions.

Lengthy table referenced here
US20220226464A1-20220721-T00014
Please refer to the end of the specification for access instructions.

The results from Ingenuity Pathway Analysis for the Diseases & Functions on the modules in FIGS. 3A-3H. −log 10(p-value) is in Supplemental Table 9 of Kazer S W et al., Nature Medicine volume 26, pages 511-518(2020), which is incorporated by reference herein in its entirety.

TABLE 7A
Genes differentially expressed in CTLs at one week after peak
virema between P3/P4 (controllers) and P1/P2 (progressors)

| p_val | avg_logFC | pct.1 | pct.2 | p_val_adj | genes |
|---|---|---|---|---|---|
| 1.587E−24 | 0.41482792 | 1 | 0.991 | 2.6947E−20 | TPT1 |
| 3.285E−24 | −0.8658957 | 0.446 | 0.654 | 5.5779E−20 | RPS28 |
| 2.2101E−22 | 0.91312309 | 0.7 | 0.308 | 3.7527E−18 | SNHG5 |
| 1.5099E−21 | −0.9503124 | 0.502 | 0.686 | 2.5638E−17 | MTRNR2L12 |
| 1.6908E−20 | −0.8297842 | 0.431 | 0.679 | 2.8709E−16 | RPL9 |
| 8.9355E−19 | −0.4358677 | 1 | 1 | 1.5172E−14 | TMSB4X |
| 9.6026E−19 | 0.82793 | 0.685 | 0.377 | 1.6305E−14 | ZFP36 |
| 1.0571E−18 | −0.5693071 | 0.828 | 0.912 | 1.795E−14 | RPL39 |
| 1.1189E−18 | −1.180286 | 0.071 | 0.311 | 1.8999E−14 | STXBP5 |
| 1.9266E−18 | 0.86940708 | 0.64 | 0.283 | 3.2713E−14 | TNFAIP3 |
| 3.2464E−14 | −0.2690837 | 1 | 1 | 5.5123E−10 | B2M |
| 4.7478E−13 | 0.24302792 | 1 | 0.994 | 8.0618E−09 | HLA-B |
| 3.6041E−12 | 0.51455127 | 0.764 | 0.478 | 6.1198E−08 | TSC22D3 |
| 3.8735E−12 | 0.79600829 | 0.202 | 0.035 | 6.5772E−08 | RP11-302B13.5 |
| 4.567E−12 | −0.4450001 | 0.914 | 0.937 | 7.7548E−08 | RPL23A |
| 4.9497E−12 | −0.5684715 | 0.697 | 0.733 | 8.4046E−08 | MTRNR2L8 |
| 6.7444E−12 | −0.5239383 | 0.644 | 0.833 | 1.1452E−07 | PTMA |
| 7.019E−12 | 0.61676435 | 0.423 | 0.204 | 1.1918E−07 | GADD45B |
| 3.0682E−11 | 0.78357682 | 0.39 | 0.138 | 5.2098E−07 | FOSB |
| 3.2426E−11 | 0.61000774 | 0.509 | 0.236 | 5.506E−07 | IER2 |
| 4.2111E−11 | 0.69626651 | 0.479 | 0.258 | 7.1504E−07 | CLK1 |
| 5.0297E−11 | 0.43369812 | 0.929 | 0.814 | 8.5405E−07 | EIF1 |
| 6.7109E−11 | 0.54496465 | 0.835 | 0.648 | 1.1395E−06 | FOS |
| 2.3795E−10 | −0.3725871 | 0.921 | 0.947 | 4.0404E−06 | RPL26 |
| 2.6586E−10 | 0.85537747 | 0.187 | 0.038 | 4.5142E−06 | TNF |
| 5.8965E−10 | 0.65605273 | 0.202 | 0.035 | 1.0012E−05 | RPL7 |
| 1.0677E−09 | −0.2547093 | 0.712 | 0.645 | 1.813E−05 | MTRNR2L2 |
| 1.5101E−09 | −0.7071972 | 0.187 | 0.311 | 2.5642E−05 | CTSW |
| 1.7003E−09 | 0.52817101 | 0.521 | 0.286 | 2.8871E−05 | SRSF7 |
| 3.7003E−09 | −0.5715882 | 0.15 | 0.233 | 6.2831E−05 | MTRNR2L11 |
| 5.2713E−09 | −0.5056379 | 0.427 | 0.506 | 8.9507E−05 | RPL21 |
| 9.7155E−09 | −0.5830798 | 0.39 | 0.525 | 0.00016497 | PRF1 |
| 1.155E−08 | −0.1986827 | 0.356 | 0.311 | 0.00019611 | COX6A1 |
| 1.6163E−08 | −0.3827471 | 0.936 | 0.931 | 0.00027446 | RPL41 |
| 2.2157E−08 | 0.59711499 | 0.479 | 0.252 | 0.00037623 | PPP1R15A |
| 2.49E−08 | −0.2529402 | 0.206 | 0.192 | 0.00042281 | CNOT1 |
| 2.4948E−08 | 0.43344838 | 0.296 | 0.116 | 0.00042361 | SRSF2 |
| 4.655E−08 | 0.62794526 | 0.337 | 0.145 | 0.00079042 | YPEL5 |
| 4.7456E−08 | −0.5814974 | 0.12 | 0.201 | 0.0008058 | DRAP1 |
| 6.2587E−08 | 0.68366278 | 0.255 | 0.079 | 0.00106272 | NR4A2 |
| 6.7173E−08 | −0.2632658 | 0.24 | 0.242 | 0.00114059 | SET |
| 7.0879E−08 | 0.22291583 | 1 | 0.987 | 0.00120353 | RPL30 |
| 9.3728E−08 | 0.69540326 | 0.337 | 0.132 | 0.0015915 | RGS1 |
| 9.5467E−08 | −0.5510752 | 0.371 | 0.509 | 0.00162103 | RPS10 |
| 1.0057E−07 | 0.61682943 | 0.296 | 0.113 | 0.00170767 | PTGER4 |
| 1.1915E−07 | 0.40908473 | 0.738 | 0.525 | 0.00202316 | CXCR4 |
| 1.2953E−07 | 0.44411001 | 0.573 | 0.346 | 0.00219938 | HSPA8 |
| 1.565E−07 | 0.23187149 | 0.963 | 0.915 | 0.0026573 | RPS4X |
| 1.754E−07 | 0.48277602 | 0.375 | 0.201 | 0.0029783 | MAP1LC3B |
| 2.0982E−07 | 0.50030124 | 0.603 | 0.374 | 0.00356279 | CD69 |
| 2.1603E−07 | −0.6701546 | 0.206 | 0.352 | 0.00366823 | S100A8 |
| 2.1734E−07 | 0.16900176 | 0.996 | 0.991 | 0.0036905 | RPS12 |
| 2.5491E−07 | 0.3609168 | 0.169 | 0.063 | 0.00432831 | MARCH6 |
| 2.7968E−07 | 0.38526657 | 0.843 | 0.695 | 0.00474895 | UBC |
| 3.5933E−07 | 0.84471335 | 0.199 | 0.063 | 0.00610139 | XCL2 |
| 3.973E−07 | 0.33599704 | 0.547 | 0.368 | 0.00674616 | SRSF3 |
| 5.1518E−07 | 0.30503247 | 0.779 | 0.594 | 0.00874774 | ID2 |
| 5.164E−07 | 0.64606766 | 0.318 | 0.129 | 0.00876843 | PMAIP1 |
| 5.4451E−07 | −0.4957199 | 0.139 | 0.211 | 0.00924571 | GIMAP4 |
| 5.668E−07 | 0.24767576 | 0.139 | 0.057 | 0.00962429 | MT1E |
| 6.6725E−07 | −0.4636724 | 0.468 | 0.582 | 0.01132982 | RPL18A |
| 8.0971E−07 | 0.428291 | 0.674 | 0.478 | 0.01374891 | NFKBIA |
| 8.4714E−07 | 0.37734853 | 0.479 | 0.314 | 0.01438437 | HSPA5 |
| 8.7617E−07 | 0.16836722 | 0.397 | 0.283 | 0.01487741 | ENO1 |
| 1.0245E−06 | 0.35923405 | 0.476 | 0.28 | 0.01739673 | CALR |
| 1.0251E−06 | 0.15809066 | 0.442 | 0.324 | 0.0174066 | NAP1L1 |
| 1.0292E−06 | −0.1790277 | 0.79 | 0.708 | 0.01747599 | ACTB |
| 1.0415E−06 | −0.5808363 | 0.135 | 0.236 | 0.01768477 | C4orf3 |
| 1.0484E−06 | −0.1721316 | 0.288 | 0.258 | 0.01780189 | CLIC1 |
| 1.0679E−06 | 0.38311784 | 0.618 | 0.39 | 0.01813238 | GAS5 |
| 1.2235E−06 | 0.44437833 | 0.524 | 0.311 | 0.02077571 | EZR |
| 1.274E−06 | 0.58047813 | 0.139 | 0.022 | 0.021632 | METTL12 |
| 1.2753E−06 | 0.44901153 | 0.202 | 0.069 | 0.0216552 | ARHGAP4 |
| 1.4441E−06 | 0.41229481 | 0.367 | 0.186 | 0.02452102 | HIST1H1D |
| 1.5017E−06 | −0.4427653 | 0.566 | 0.597 | 0.02549962 | MT-CO1 |
| 1.6237E−06 | 0.15162583 | 0.184 | 0.094 | 0.02756987 | KAT6A |
| 1.6312E−06 | −0.1733064 | 0.974 | 0.972 | 0.02769826 | RPL27A |
| 1.8684E−06 | 0.37735689 | 0.18 | 0.06 | 0.03172466 | ENC1 |
| 1.9529E−06 | 0.38534561 | 0.161 | 0.05 | 0.03316038 | RMRP |
| 1.9549E−06 | −0.1470696 | 0.363 | 0.299 | 0.03319382 | ARPC5 |
| 2.0323E−06 | 0.26542813 | 0.206 | 0.11 | 0.03450881 | TANK |
| 2.0336E−06 | 0.10081721 | 0.18 | 0.107 | 0.03453029 | ANAPC5 |
| 2.1195E−06 | 0.26709551 | 0.959 | 0.877 | 0.03598892 | RPL3 |
| 2.2485E−06 | 0.46768684 | 0.326 | 0.148 | 0.03817901 | MAP3K8 |
| 2.6515E−06 | −0.2211429 | 0.985 | 0.994 | 0.04502265 | RPS27A |
| 2.7528E−06 | −0.2546377 | 0.914 | 0.871 | 0.04674268 | RPS27 |
| 3.2729E−06 | 0.15196714 | 0.127 | 0.063 | 0.0555746 | SSNA1 |
| 3.4847E−06 | 0.29264457 | 0.543 | 0.343 | 0.05917025 | AHNAK |
| 3.7557E−06 | 0.43446912 | 0.704 | 0.538 | 0.06377224 | KLF6 |
| 3.9094E−06 | 0.40907893 | 0.292 | 0.16 | 0.06638203 | CD47 |
| 3.9291E−06 | 0.17078202 | 0.996 | 0.991 | 0.06671549 | RPLP1 |
| 4.6258E−06 | 0.50443655 | 0.33 | 0.157 | 0.07854615 | DNTTIP2 |
| 5.1273E−06 | 0.21276274 | 0.303 | 0.167 | 0.08706215 | NOP58 |
| 5.1278E−06 | −0.1832204 | 0.262 | 0.239 | 0.0870707 | C9orf78 |
| 5.4854E−06 | −0.3151492 | 0.213 | 0.226 | 0.09314202 | ATP5I |
| 5.6227E−06 | −0.103654 | 0.206 | 0.157 | 0.09547362 | PTPN1 |
| 5.7104E−06 | −0.2787313 | 0.948 | 0.972 | 0.09696262 | HLA-C |
| 5.8946E−06 | −0.3076621 | 0.262 | 0.28 | 0.10009097 | PAPOLA |
| 6.6724E−06 | 0.39625423 | 0.315 | 0.154 | 0.11329767 | RSRC2 |
| 7.0762E−06 | −0.2916585 | 0.985 | 0.991 | 0.1201535 | RPS29 |
| 7.1185E−06 | 0.25312452 | 0.412 | 0.245 | 0.12087211 | TAPBP |
| 7.5109E−06 | −0.3048784 | 0.757 | 0.764 | 0.12753455 | GNLY |
| 8.0159E−06 | 0.29621106 | 0.161 | 0.069 | 0.13610933 | LGALS8 |
| 8.9672E−06 | −0.440189 | 0.386 | 0.478 | 0.15226372 | XAF1 |
| 9.2618E−06 | −0.5796992 | 0.127 | 0.239 | 0.15726568 | AC092580.4 |
| 9.3251E−06 | −0.127748 | 0.195 | 0.154 | 0.15833995 | PSMA4 |
| 1.0192E−05 | 0.31667611 | 0.341 | 0.189 | 0.1730566 | POLR1D |
| 1.0755E−05 | 0.38953266 | 0.217 | 0.088 | 0.1826181 | ACAP1 |
| 1.1177E−05 | 0.52563389 | 0.296 | 0.142 | 0.18977867 | SBDS |
| 1.1434E−05 | 0.26553183 | 0.573 | 0.403 | 0.19414829 | SF3B1 |
| 1.1607E−05 | −0.3348474 | 0.21 | 0.23 | 0.19708515 | OSBPL8 |
| 1.2102E−05 | −0.5218323 | 0.187 | 0.308 | 0.20549911 | NDUFA13 |
| 1.3243E−05 | 0.33817889 | 0.112 | 0.053 | 0.2248721 | ETNK1 |
| 1.3488E−05 | 0.41694769 | 0.315 | 0.198 | 0.22902587 | HNRNPH1 |
| 1.4492E−05 | 0.39179276 | 0.199 | 0.094 | 0.24607019 | ZNF394 |
| 1.4759E−05 | 0.43636818 | 0.127 | 0.041 | 0.25061034 | TUBB4B |
| 1.5786E−05 | −0.2458115 | 0.562 | 0.563 | 0.26805285 | HLA-F |
| 1.611E−05 | −0.1655615 | 0.419 | 0.415 | 0.27354734 | TCEB2 |
| 1.6889E−05 | 0.13254883 | 0.727 | 0.544 | 0.28677357 | JUNB |
| 1.713E−05 | −0.3868959 | 0.135 | 0.176 | 0.2908726 | NDUFB4 |
| 1.7804E−05 | 0.27117303 | 0.195 | 0.085 | 0.30231998 | ZFAND5 |
| 1.7878E−05 | −0.3433796 | 0.502 | 0.563 | 0.30356776 | ZFAS1 |
| 1.9025E−05 | 0.11661322 | 0.12 | 0.057 | 0.32303919 | FMNL1 |
| 2.0337E−05 | 0.40415943 | 0.165 | 0.053 | 0.34531389 | KLF9 |
| 2.2826E−05 | 0.33943624 | 0.281 | 0.142 | 0.38758472 | RAN |
| 2.3656E−05 | −0.4954853 | 0.101 | 0.17 | 0.40168372 | ATP5J2 |
| 2.5917E−05 | −0.3197518 | 0.165 | 0.189 | 0.4400714 | SNRPB2 |
| 2.6881E−05 | 0.28638066 | 0.307 | 0.173 | 0.45643729 | BRD2 |
| 2.716E−05 | −0.294964 | 0.202 | 0.214 | 0.46118204 | APOBEC3G |
| 2.9125E−05 | −0.5931879 | 0.225 | 0.318 | 0.49454602 | TRDC |
| 2.9991E−05 | −0.4239515 | 0.067 | 0.11 | 0.50925185 | RAC1 |
| 3.0896E−05 | 0.20784608 | 0.974 | 0.931 | 0.52460992 | RPS11 |
| 3.1711E−05 | −0.5495735 | 0.12 | 0.223 | 0.53845456 | MTRNR2L6 |
| 3.3217E−05 | 0.27946267 | 0.12 | 0.041 | 0.5640266 | RAB5A |
| 3.3913E−05 | 0.28820421 | 0.854 | 0.711 | 0.57583645 | RPL4 |
| 3.6797E−05 | 0.23528111 | 0.142 | 0.06 | 0.62480782 | STAT4 |
| 3.8632E−05 | 0.20573324 | 0.161 | 0.088 | 0.65596548 | USP48 |
| 4.0059E−05 | −0.2731036 | 0.24 | 0.248 | 0.68020832 | PSMB9 |
| 4.0376E−05 | 0.37231309 | 0.622 | 0.475 | 0.6855786 | RNA28S5 |
| 4.0796E−05 | 0.45275444 | 0.172 | 0.063 | 0.69272276 | SOD2 |
| 4.1041E−05 | 0.15353335 | 0.142 | 0.075 | 0.69686909 | FOSL2 |
| 4.1574E−05 | 0.12674786 | 0.288 | 0.204 | 0.70593165 | HNRNPU |
| 4.1799E−05 | 0.40255945 | 0.184 | 0.079 | 0.70974112 | SERTAD1 |
| 4.1923E−05 | −0.2177509 | 0.326 | 0.308 | 0.71185835 | C11orf58 |
| 4.4149E−05 | 0.34706398 | 0.105 | 0.031 | 0.74965792 | GLUL |
| 4.4949E−05 | −0.5461252 | 0.109 | 0.198 | 0.76324054 | TIPIN |
| 4.6109E−05 | 0.14857972 | 0.491 | 0.365 | 0.78292277 | RAC2 |
| 4.7298E−05 | −0.4999539 | 0.135 | 0.22 | 0.8031259 | NDUFB1 |
| 4.8008E−05 | 0.28995077 | 0.225 | 0.107 | 0.81517874 | TNFRSF14 |
| 4.9316E−05 | 0.13540073 | 0.139 | 0.072 | 0.83738508 | SNRPD3 |
| 4.9545E−05 | 0.23454108 | 0.101 | 0.038 | 0.84127459 | ADIPOR1 |
| 5.025E−05 | −0.1072795 | 0.217 | 0.192 | 0.85324153 | CD37 |
| 5.0678E−05 | −0.5895832 | 0.213 | 0.318 | 0.86050726 | TYROBP |
| 5.3704E−05 | 0.35552447 | 0.187 | 0.079 | 0.91189078 | PTPN2 |
| 5.532E−05 | 0.34901151 | 0.427 | 0.258 | 0.93933966 | PARP8 |
| 5.583E−05 | 0.11772358 | 0.124 | 0.06 | 0.94800162 | RAB6A |
| 5.6502E−05 | 0.25665898 | 0.648 | 0.465 | 0.95940446 | EEF1D |
| 5.8711E−05 | −0.5780681 | 0.243 | 0.393 | 0.99691124 | TRGC1 |
| 5.956E−05 | 0.31229108 | 0.412 | 0.248 | 1 | DNAJB1 |
| 6.1779E−05 | 0.22837521 | 0.352 | 0.239 | 1 | SLC38A1 |
| 6.2478E−05 | −0.1800032 | 0.131 | 0.119 | 1 | GLIPR2 |
| 6.5052E−05 | −0.2924796 | 0.599 | 0.607 | 1 | CST7 |
| 6.7058E−05 | 0.13832304 | 0.213 | 0.132 | 1 | RWDD1 |
| 6.8504E−05 | −0.2133255 | 0.288 | 0.318 | 1 | RAP1B |
| 6.9075E−05 | 0.29561089 | 0.206 | 0.104 | 1 | C9orf142 |
| 6.9816E−05 | 0.32348371 | 0.109 | 0.028 | 1 | ODC1 |
| 7.301E−05 | −0.447749 | 0.303 | 0.44 | 1 | S100A9 |
| 7.3482E−05 | 0.30903564 | 0.82 | 0.66 | 1 | JUN |
| 7.3558E−05 | 0.47569394 | 0.176 | 0.06 | 1 | NLRP1 |
| 7.3887E−05 | 0.3323375 | 0.101 | 0.022 | 1 | ELMO1 |
| 7.4926E−05 | −0.2314931 | 0.322 | 0.314 | 1 | TAGLN2 |
| 7.9987E−05 | 0.28876573 | 0.367 | 0.248 | 1 | DDX3X |
| 8.0128E−05 | 0.44265518 | 0.112 | 0.028 | 1 | TRABD |
| 8.0607E−05 | 0.39713095 | 0.288 | 0.148 | 1 | RPSAP58 |
| 8.1054E−05 | 0.19386757 | 0.12 | 0.05 | 1 | ERGIC3 |
| 8.2078E−05 | 0.30714921 | 0.64 | 0.462 | 1 | LAPTM5 |
| 8.6718E−05 | 0.19458458 | 0.888 | 0.758 | 1 | RPL28 |
| 8.7798E−05 | 0.19665385 | 0.116 | 0.057 | 1 | PSPC1 |
| 8.7974E−05 | 0.44951595 | 0.142 | 0.047 | 1 | RABGGTB |
| 9.0804E−05 | 0.15823731 | 0.157 | 0.088 | 1 | RAB14 |
| 9.3808E−05 | 0.26748324 | 0.135 | 0.053 | 1 | SASH3 |
| 9.442E−05 | 0.33362052 | 0.449 | 0.289 | 1 | MSN |
| 9.7599E−05 | −0.2697194 | 0.161 | 0.176 | 1 | GPX4 |
| 9.8453E−05 | −0.238385 | 0.419 | 0.437 | 1 | SRP14 |
| 9.9012E−05 | −0.2064185 | 0.745 | 0.726 | 1 | CD3D |
| 9.9835E−05 | −0.2740399 | 0.18 | 0.192 | 1 | BST2 |
| 0.00010261 | −0.1821572 | 0.184 | 0.198 | 1 | NDUFB9 |
| 0.00010461 | 0.4960359 | 0.169 | 0.066 | 1 | IFNGR1 |
| 0.0001057 | 0.27229135 | 0.109 | 0.041 | 1 | SEC61A1 |
| 0.00010609 | 0.18789848 | 0.116 | 0.047 | 1 | CSNK1D |
| 0.00010775 | 0.17689973 | 0.363 | 0.248 | 1 | SSR2 |
| 0.00010883 | 0.28246163 | 0.199 | 0.091 | 1 | SIGIRR |
| 0.00011334 | −0.2900449 | 0.712 | 0.689 | 1 | MTRNR2L1 |
| 0.00011623 | 0.24503785 | 0.225 | 0.119 | 1 | SH3GLB1 |
| 0.00011652 | −0.3061573 | 0.532 | 0.623 | 1 | IL32 |
| 0.00011667 | 0.10142818 | 0.993 | 0.959 | 1 | RPL13A |
| 0.00011773 | 0.33199021 | 0.498 | 0.321 | 1 | CCNL1 |
| 0.00012272 | −0.2788275 | 0.288 | 0.296 | 1 | SRRM1 |
| 0.0001272 | 0.52389978 | 0.116 | 0.025 | 1 | HLA-DQB1 |
| 0.00013706 | 0.15676817 | 0.315 | 0.208 | 1 | SETX |
| 0.0001393 | −0.4261033 | 0.161 | 0.242 | 1 | ROMO1 |
| 0.00014507 | 0.29962575 | 0.157 | 0.072 | 1 | EIF1B |
| 0.00014546 | 0.20159764 | 0.142 | 0.075 | 1 | STARD7 |
| 0.00014598 | 0.19515612 | 0.341 | 0.236 | 1 | ATM |
| 0.00014724 | 0.42529844 | 0.206 | 0.091 | 1 | AMD1 |
| 0.00015342 | 0.39336824 | 0.139 | 0.053 | 1 | RNF216 |
| 0.0001536 | 0.44781817 | 0.247 | 0.113 | 1 | TUBA4A |
| 0.00015886 | 0.31002368 | 0.12 | 0.041 | 1 | CDK17 |
| 0.00015887 | −0.2090797 | 0.243 | 0.22 | 1 | GCC2 |
| 0.00015937 | −0.324986 | 0.704 | 0.796 | 1 | PFN1 |
| 0.00016541 | 0.17026336 | 0.217 | 0.123 | 1 | SYTL1 |
| 0.00017784 | 0.27768102 | 0.135 | 0.053 | 1 | RP11-220I1.1 |
| 0.00017847 | 0.31041045 | 0.581 | 0.399 | 1 | CD74 |
| 0.00017961 | −0.294047 | 0.101 | 0.119 | 1 | NDFIP1 |
| 0.0001816 | −0.1159338 | 0.509 | 0.462 | 1 | COX6C |
| 0.00018324 | 0.25754176 | 0.33 | 0.214 | 1 | HLA-DRB1 |
| 0.00018327 | −0.3149587 | 0.105 | 0.138 | 1 | ATF7IP |
| 0.00018472 | 0.13183777 | 0.154 | 0.079 | 1 | SMARCC2 |
| 0.00018534 | 0.37060001 | 0.142 | 0.066 | 1 | KLF12 |
| 0.00018732 | −0.3193623 | 0.135 | 0.148 | 1 | GGNBP2 |
| 0.00018786 | 0.21045812 | 0.127 | 0.063 | 1 | ZDHHC20 |
| 0.0001924 | −0.4409466 | 0.067 | 0.119 | 1 | SPCS2 |
| 0.00019416 | 0.27121397 | 0.12 | 0.044 | 1 | DAZAP1 |
| 0.00019698 | −0.1174965 | 0.764 | 0.689 | 1 | MYL12A |
| 0.00020176 | 0.14330006 | 0.146 | 0.085 | 1 | RASGRP2 |
| 0.00021332 | 0.12981171 | 0.139 | 0.075 | 1 | PIN1 |
| 0.00021627 | −0.1003467 | 0.27 | 0.223 | 1 | SKAP1 |
| 0.00021706 | 0.34985142 | 0.105 | 0.035 | 1 | IL1B |
| 0.00021988 | 0.34266972 | 0.165 | 0.088 | 1 | MYO1G |
| 0.00022085 | 0.30029854 | 0.169 | 0.075 | 1 | HIST2H2AC |
| 0.00022501 | −0.2153987 | 0.464 | 0.465 | 1 | RPL22 |
| 0.00022535 | 0.33504861 | 0.124 | 0.038 | 1 | MKLN1-AS1 |
| 0.00022575 | 0.13632704 | 0.139 | 0.091 | 1 | EPRS |
| 0.00022733 | −0.4861189 | 0.094 | 0.17 | 1 | SMDT1 |
| 0.00022879 | 0.10349198 | 0.165 | 0.101 | 1 | SDF4 |
| 0.00023334 | −0.1946464 | 0.127 | 0.126 | 1 | LASP1 |
| 0.00023517 | 0.24013142 | 0.228 | 0.135 | 1 | MYO1F |
| 0.00023702 | −0.5325796 | 0.094 | 0.186 | 1 | NBEAL1 |
| 0.00023704 | −0.3824542 | 0.142 | 0.179 | 1 | S1PR5 |
| 0.00024008 | 0.2088531 | 0.509 | 0.355 | 1 | VIM |
| 0.00024048 | 0.27693782 | 0.172 | 0.079 | 1 | DPP7 |
| 0.00025187 | −0.2984896 | 0.232 | 0.252 | 1 | TPR |
| 0.00025559 | 0.42706836 | 0.36 | 0.204 | 1 | KLF2 |
| 0.0002583 | −0.2235911 | 0.139 | 0.148 | 1 | USP1 |
| 0.00026082 | 0.29601604 | 0.243 | 0.145 | 1 | AKNA |
| 0.00026731 | 0.14187107 | 0.112 | 0.053 | 1 | KCTD20 |
| 0.00026744 | −0.244554 | 0.109 | 0.11 | 1 | CCDC167 |
| 0.00026929 | −0.2042352 | 0.191 | 0.198 | 1 | TRAF3IP3 |
| 0.00028963 | −0.1226325 | 0.296 | 0.261 | 1 | ISG20 |
| 0.000295 | −0.2996459 | 0.21 | 0.211 | 1 | ISG15 |
| 0.00029777 | 0.62179332 | 0.247 | 0.116 | 1 | IFNG |
| 0.00030435 | −0.2463872 | 0.097 | 0.107 | 1 | SDHB |
| 0.00030626 | 0.2428343 | 0.255 | 0.145 | 1 | PGK1 |
| 0.00031308 | 0.16283007 | 0.805 | 0.657 | 1 | NACA |
| 0.00032344 | 0.10327536 | 0.296 | 0.198 | 1 | ARID4B |
| 0.00032883 | 0.43940393 | 0.18 | 0.069 | 1 | LPGAT1 |
| 0.00032928 | −0.3647871 | 0.228 | 0.296 | 1 | TMEM70 |
| 0.00033322 | 0.22068523 | 0.206 | 0.119 | 1 | FNBP4 |
| 0.00033406 | 0.34959331 | 0.36 | 0.217 | 1 | CYTIP |
| 0.00034437 | −0.2357886 | 0.393 | 0.418 | 1 | TMA7 |
| 0.00034881 | 0.11027499 | 0.236 | 0.179 | 1 | LCP2 |
| 0.00035019 | 0.10612122 | 0.176 | 0.107 | 1 | PRNP |
| 0.00035473 | −0.184846 | 0.273 | 0.274 | 1 | UQCRH |
| 0.00036513 | −0.1706192 | 0.18 | 0.164 | 1 | SHISA5 |
| 0.00036713 | −0.2428518 | 0.109 | 0.113 | 1 | C19orf10 |
| 0.00036818 | 0.21236699 | 0.135 | 0.063 | 1 | SPTAN1 |
| 0.00037177 | 0.41914564 | 0.12 | 0.031 | 1 | CSRNP1 |
| 0.00037521 | −0.3475444 | 0.105 | 0.142 | 1 | POLR2G |
| 0.00037933 | 0.41875638 | 0.154 | 0.053 | 1 | TMX1 |
| 0.00038036 | 0.19520427 | 0.36 | 0.242 | 1 | WIPF1 |
| 0.0003865 | −0.1264404 | 0.101 | 0.116 | 1 | IL16 |
| 0.00040274 | −0.1092642 | 0.15 | 0.129 | 1 | KMT2C |
| 0.00040433 | 0.30164907 | 0.165 | 0.072 | 1 | FBXW5 |
| 0.00040965 | −0.2394844 | 0.375 | 0.384 | 1 | MORF4L1 |
| 0.00041927 | −0.3098184 | 0.397 | 0.469 | 1 | TMBIM6 |
| 0.00042413 | 0.31410092 | 0.285 | 0.157 | 1 | TGFBR3 |
| 0.00045422 | 0.21170222 | 0.891 | 0.777 | 1 | H3F3B |
| 0.00046824 | 0.44616146 | 0.333 | 0.214 | 1 | CCL4 |
| 0.0004724 | −0.228003 | 0.255 | 0.261 | 1 | SPN |
| 0.00047346 | 0.20325746 | 0.199 | 0.107 | 1 | VCP |
| 0.00047474 | −0.2722721 | 0.086 | 0.107 | 1 | POLR2B |
| 0.0004813 | −0.2331243 | 0.427 | 0.437 | 1 | OST4 |
| 0.0004827 | 0.2109208 | 0.348 | 0.242 | 1 | MYH9 |
| 0.00048297 | 0.1819392 | 0.322 | 0.217 | 1 | LEPROTL1 |
| 0.00048397 | −0.343687 | 0.116 | 0.154 | 1 | ALOX5AP |
| 0.00049155 | −0.284353 | 0.461 | 0.487 | 1 | CDC42 |
| 0.00050061 | 0.13651837 | 0.247 | 0.16 | 1 | RASSF5 |
| 0.00050648 | −0.4306547 | 0.165 | 0.208 | 1 | MYBL1 |
| 0.00051046 | 0.35829592 | 0.172 | 0.104 | 1 | HERPUD2 |
| 0.00052075 | 0.24380265 | 0.21 | 0.129 | 1 | ATP5F1 |
| 0.00052201 | −0.1334492 | 0.176 | 0.151 | 1 | MTPN |
| 0.00052417 | 0.16426873 | 0.581 | 0.434 | 1 | RBM39 |
| 0.00053063 | 0.23091679 | 0.176 | 0.101 | 1 | SEC31B |
| 0.00053448 | −0.1724616 | 0.558 | 0.516 | 1 | HSP90AA1 |
| 0.00053598 | 0.16615021 | 0.352 | 0.236 | 1 | CYTH1 |
| 0.00053599 | 0.25760486 | 0.288 | 0.189 | 1 | TUBA1A |
| 0.00053674 | −0.3903487 | 0.285 | 0.374 | 1 | HMGB1 |
| 0.00054315 | 0.24242818 | 0.888 | 0.792 | 1 | RPLP0 |
| 0.00054385 | 0.24504766 | 0.142 | 0.079 | 1 | RPN1 |
| 0.00054539 | 0.19869406 | 0.273 | 0.192 | 1 | TMEM59 |
| 0.00055278 | 0.2720795 | 0.157 | 0.072 | 1 | SUMF2 |
| 0.0005571 | 0.12398947 | 0.161 | 0.101 | 1 | PPP2CA |
| 0.00055973 | 0.33643751 | 0.139 | 0.069 | 1 | DDX39A |
| 0.00056233 | 0.18012973 | 0.154 | 0.072 | 1 | TPP2 |
| 0.00056623 | 0.16130127 | 0.135 | 0.085 | 1 | C19orf66 |
| 0.00058076 | −0.5029455 | 0.225 | 0.336 | 1 | TRGC2 |
| 0.00058355 | −0.2619202 | 0.105 | 0.123 | 1 | MEAF6 |
| 0.00059032 | −0.1325226 | 0.303 | 0.289 | 1 | SERBP1 |
| 0.00059709 | 0.12463227 | 0.554 | 0.437 | 1 | CD48 |
| 0.00060211 | 0.31990368 | 0.734 | 0.601 | 1 | DUSP1 |
| 0.00060967 | 0.16766743 | 0.273 | 0.173 | 1 | CIB1 |
| 0.00061019 | 0.22976725 | 0.18 | 0.104 | 1 | EWSR1 |
| 0.0006123 | 0.34526741 | 0.217 | 0.104 | 1 | PTP4A1 |
| 0.00063027 | 0.17488186 | 0.502 | 0.358 | 1 | IL7R |
| 0.0006381 | 0.23669082 | 0.36 | 0.252 | 1 | ELF1 |
| 0.00065067 | 0.270999 | 0.15 | 0.079 | 1 | SP140 |
| 0.00065123 | −0.2653338 | 0.599 | 0.635 | 1 | RPL36AL |
| 0.00065397 | −0.108146 | 0.835 | 0.796 | 1 | RPL10 |
| 0.00067548 | 0.17676946 | 0.142 | 0.088 | 1 | C1orf56 |
| 0.00067599 | −0.4100967 | 0.086 | 0.129 | 1 | SUZ12 |
| 0.00067952 | 0.29790091 | 0.101 | 0.031 | 1 | POLR2A |
| 0.00068103 | 0.25263185 | 0.199 | 0.104 | 1 | TKT |
| 0.00069232 | −0.1261115 | 0.146 | 0.138 | 1 | ENSA |
| 0.0006931 | −0.2721285 | 0.3 | 0.324 | 1 | CD247 |
| 0.00069711 | 0.309899 | 0.24 | 0.132 | 1 | IDI1 |
| 0.00070269 | 0.15460197 | 0.592 | 0.443 | 1 | RHOA |
| 0.00070951 | 0.32658039 | 0.15 | 0.06 | 1 | CD5 |
| 0.0007172 | 0.34808252 | 0.146 | 0.063 | 1 | TMEM219 |
| 0.00073258 | 0.14169377 | 0.146 | 0.075 | 1 | PSMD7 |
| 0.00074574 | 0.29323793 | 0.18 | 0.085 | 1 | AC013394.2 |
| 0.00075141 | 0.29856864 | 0.39 | 0.261 | 1 | EIF4G2 |
| 0.0007624 | −0.4081996 | 0.21 | 0.27 | 1 | PYHIN1 |
| 0.00076971 | 0.19937943 | 0.371 | 0.277 | 1 | EIF3H |
| 0.00077327 | −0.2049474 | 0.172 | 0.154 | 1 | U2SURP |
| 0.00077694 | −0.3033117 | 0.468 | 0.465 | 1 | GZMB |
| 0.00079009 | 0.25248571 | 0.18 | 0.104 | 1 | C19orf43 |
| 0.00079358 | 0.27190669 | 0.127 | 0.063 | 1 | LPIN2 |
| 0.00080128 | 0.39396944 | 0.12 | 0.038 | 1 | SNHG12 |
| 0.00080364 | −0.1841777 | 0.135 | 0.132 | 1 | USP47 |
| 0.00081325 | −0.182436 | 0.217 | 0.211 | 1 | SEC62 |
| 0.00081439 | 0.11541999 | 0.135 | 0.072 | 1 | RBCK1 |
| 0.00081523 | 0.2200296 | 0.341 | 0.223 | 1 | PRMT2 |
| 0.00082572 | −0.3659695 | 0.318 | 0.425 | 1 | SP100 |
| 0.00082715 | 0.10054559 | 0.195 | 0.119 | 1 | GABARAPL2 |
| 0.00083631 | 0.15923249 | 0.161 | 0.088 | 1 | MDH2 |
| 0.00084501 | 0.37244346 | 0.127 | 0.047 | 1 | GIT2 |
| 0.00085066 | 0.21032709 | 0.36 | 0.236 | 1 | CANX |
| 0.00085125 | 0.25574678 | 0.12 | 0.057 | 1 | CCNG1 |
| 0.00085133 | 0.30072128 | 0.135 | 0.05 | 1 | PLIN2 |
| 0.00085338 | −0.1716869 | 0.345 | 0.349 | 1 | EID1 |
| 0.00086242 | −0.1739507 | 0.101 | 0.107 | 1 | PTPN11 |
| 0.00087224 | −0.1090841 | 0.101 | 0.085 | 1 | SLC44A2 |
| 0.00087287 | 0.31516745 | 0.169 | 0.085 | 1 | ITK |
| 0.00087702 | 0.31289012 | 0.154 | 0.072 | 1 | ANXA11 |
| 0.00088837 | 0.34389865 | 0.184 | 0.082 | 1 | NFKBIZ |
| 0.00089944 | 0.27605744 | 0.337 | 0.208 | 1 | DNAJA1 |
| 0.00089945 | 0.28797553 | 0.187 | 0.091 | 1 | CITED2 |
| 0.00091835 | −0.1671224 | 0.326 | 0.311 | 1 | MT-ATP6 |
| 0.00093529 | −0.1535752 | 0.292 | 0.283 | 1 | RARRES3 |
| 0.00094251 | 0.11682167 | 0.127 | 0.069 | 1 | ZNF800 |
| 0.00095497 | 0.14998079 | 0.21 | 0.126 | 1 | UGP2 |
| 0.00097514 | −0.1593848 | 0.131 | 0.129 | 1 | ATG12 |
| 0.00098273 | −0.427716 | 0.097 | 0.17 | 1 | RP11-835E18.5 |
| 0.00098634 | −0.3976663 | 0.067 | 0.119 | 1 | SH3BP5 |
| 0.00098734 | 0.14796848 | 0.296 | 0.201 | 1 | PPDPF |
| 0.00099416 | −0.2628057 | 0.27 | 0.302 | 1 | UBBP4 |
| 0.00100673 | 0.17716789 | 0.318 | 0.204 | 1 | APOL6 |
| 0.00102166 | 0.22490724 | 0.112 | 0.05 | 1 | ACTR1B |
| 0.00102564 | 0.14858689 | 0.217 | 0.135 | 1 | TAGAP |
| 0.00104628 | −0.1276945 | 0.348 | 0.314 | 1 | LYAR |
| 0.00105007 | 0.12006838 | 0.139 | 0.079 | 1 | CASP8 |
| 0.00105197 | 0.2929889 | 0.524 | 0.362 | 1 | EMB |
| 0.00108972 | −0.122709 | 0.131 | 0.107 | 1 | ARID4A |
| 0.00109474 | 0.13974926 | 0.165 | 0.094 | 1 | DOCK10 |
| 0.00109816 | −0.1242484 | 0.146 | 0.135 | 1 | PTPN4 |
| 0.00110104 | 0.46061029 | 0.176 | 0.085 | 1 | ZNF331 |
| 0.00111539 | 0.27610109 | 0.199 | 0.129 | 1 | PRKCB |
| 0.00112144 | 0.10742849 | 0.318 | 0.248 | 1 | EPSTI1 |
| 0.0011336 | −0.1707412 | 0.131 | 0.135 | 1 | BAZ1B |
| 0.00113673 | −0.1105449 | 0.18 | 0.151 | 1 | NEDD8 |
| 0.00114278 | −0.1187689 | 0.831 | 0.758 | 1 | NKG7 |
| 0.00115681 | −0.3110316 | 0.154 | 0.22 | 1 | NUB1 |
| 0.00116213 | −0.226483 | 0.169 | 0.182 | 1 | PPIG |
| 0.00116628 | −0.3544227 | 0.071 | 0.123 | 1 | EIF1AX |
| 0.00116672 | 0.16526866 | 0.228 | 0.192 | 1 | TNRC6B |
| 0.00118429 | 0.29749315 | 0.12 | 0.044 | 1 | SPTY2D1 |
| 0.00119989 | −0.2542094 | 0.157 | 0.189 | 1 | ANXA2 |
| 0.00121362 | 0.4280467 | 0.124 | 0.041 | 1 | MED15 |
| 0.00121369 | 0.20664043 | 0.146 | 0.085 | 1 | C16orf13 |
| 0.00125515 | 0.31796652 | 0.142 | 0.069 | 1 | EHD1 |
| 0.00127081 | 0.15299687 | 0.124 | 0.06 | 1 | ACADVL |
| 0.00127576 | −0.1108677 | 0.247 | 0.22 | 1 | GNG2 |
| 0.00129423 | 0.25184302 | 0.243 | 0.138 | 1 | MGEA5 |
| 0.00130427 | 0.17635699 | 0.36 | 0.252 | 1 | CAPN2 |
| 0.00132544 | 0.10085011 | 0.116 | 0.069 | 1 | RPAIN |
| 0.00133932 | 0.27966849 | 0.333 | 0.204 | 1 | ITGAL |
| 0.00136111 | −0.3206765 | 0.315 | 0.368 | 1 | ATPIF1 |
| 0.00136416 | −0.2399353 | 0.959 | 0.959 | 1 | CCL5 |
| 0.00138513 | 0.1611646 | 0.161 | 0.091 | 1 | FERMT3 |
| 0.00140123 | −0.2618393 | 0.127 | 0.142 | 1 | ZRANB2 |
| 0.0014937 | 0.17045095 | 0.292 | 0.192 | 1 | ZEB2 |
| 0.00151319 | −0.1789327 | 0.94 | 0.953 | 1 | RPL35 |
| 0.00151501 | 0.1481892 | 0.131 | 0.072 | 1 | KDM2A |
| 0.00156495 | −0.1681536 | 0.446 | 0.418 | 1 | ADD3 |
| 0.00156704 | 0.25710463 | 0.112 | 0.044 | 1 | HSH2D |
| 0.00156794 | 0.10051026 | 0.18 | 0.116 | 1 | CCT6A |
| 0.00158038 | −0.2103742 | 0.648 | 0.648 | 1 | COX7C |
| 0.0016147 | −0.1056872 | 0.678 | 0.619 | 1 | PTPRCAP |
| 0.00162789 | 0.26376108 | 0.131 | 0.06 | 1 | NFATC2 |
| 0.00164493 | 0.18175402 | 0.528 | 0.399 | 1 | HINT1 |
| 0.00165856 | 0.19857327 | 0.21 | 0.123 | 1 | SFPQ |
| 0.00166499 | 0.33167563 | 0.116 | 0.044 | 1 | SOS1 |
| 0.00167387 | −0.2616536 | 0.36 | 0.381 | 1 | PLAC8 |
| 0.0016936 | 0.25871392 | 0.109 | 0.041 | 1 | SETD5 |
| 0.00169696 | 0.10069849 | 0.157 | 0.097 | 1 | CNOT2 |
| 0.00169979 | 0.27478619 | 0.105 | 0.053 | 1 | C11orf21 |
| 0.00171368 | 0.15053586 | 0.285 | 0.198 | 1 | DDX6 |
| 0.00171559 | 0.17837989 | 0.127 | 0.063 | 1 | FKBP11 |
| 0.0017291 | −0.3188412 | 0.124 | 0.154 | 1 | ENY2 |
| 0.0017475 | −0.2980413 | 0.094 | 0.123 | 1 | FAIM3 |
| 0.00175444 | 0.2072194 | 0.124 | 0.066 | 1 | GSDMD |
| 0.00177192 | 0.16013058 | 0.109 | 0.06 | 1 | ZMYM5 |
| 0.00177296 | −0.1435274 | 0.146 | 0.135 | 1 | IK |
| 0.00178838 | 0.31330156 | 0.303 | 0.179 | 1 | GNAS |
| 0.0018513 | −0.1681528 | 0.12 | 0.116 | 1 | TMEM14B |
| 0.00188667 | 0.13365794 | 0.15 | 0.097 | 1 | PCGF5 |
| 0.00189285 | −0.2033593 | 0.217 | 0.245 | 1 | PNISR |
| 0.00189599 | 0.11369119 | 0.21 | 0.135 | 1 | TUBA1B |
| 0.00190263 | −0.3912669 | 0.064 | 0.104 | 1 | NSRP1 |
| 0.00191648 | 0.32964478 | 0.127 | 0.05 | 1 | RBM6 |
| 0.00192379 | −0.3041579 | 0.236 | 0.277 | 1 | ZNF90 |
| 0.00193019 | 0.31925901 | 0.124 | 0.044 | 1 | ARHGAP9 |
| 0.00194121 | 0.10581337 | 0.12 | 0.066 | 1 | KIAA1143 |
| 0.0019573 | 0.29139836 | 0.184 | 0.091 | 1 | SEPT1 |
| 0.00195838 | −0.4175362 | 0.142 | 0.226 | 1 | ATP5EP2 |
| 0.00199348 | 0.1353671 | 0.135 | 0.091 | 1 | CDKN2D |
| 0.00199924 | −0.2808527 | 0.139 | 0.148 | 1 | ZC3H13 |
| 0.00201276 | 0.25129981 | 0.18 | 0.101 | 1 | PPHLN1 |
| 0.00201574 | 0.38219289 | 0.105 | 0.038 | 1 | CPT1A |
| 0.00204367 | −0.1551449 | 0.184 | 0.17 | 1 | CALM3 |
| 0.00204639 | 0.12659935 | 0.472 | 0.355 | 1 | CORO1A |
| 0.00204649 | 0.1998955 | 0.112 | 0.05 | 1 | DOCK11 |
| 0.00206248 | 0.192218 | 0.367 | 0.255 | 1 | TLN1 |
| 0.00208338 | 0.38071583 | 0.124 | 0.057 | 1 | ATXN1 |
| 0.00211387 | −0.1051344 | 0.236 | 0.214 | 1 | C6orf62 |
| 0.00213287 | 0.13258353 | 0.307 | 0.226 | 1 | SLC25A3 |
| 0.00214958 | −0.3082222 | 0.082 | 0.101 | 1 | LSM6 |
| 0.00215825 | −0.2790766 | 0.139 | 0.17 | 1 | SHFM1 |
| 0.00216044 | 0.34472079 | 0.176 | 0.082 | 1 | CD6 |
| 0.00218135 | 0.34814305 | 0.21 | 0.104 | 1 | HELZ |
| 0.0022033 | −0.180734 | 0.109 | 0.107 | 1 | DCTN3 |
| 0.00220933 | 0.34311554 | 0.139 | 0.053 | 1 | CDK5RAP3 |
| 0.00222673 | 0.10662946 | 0.27 | 0.192 | 1 | ZAP70 |
| 0.00224488 | 0.19565579 | 0.356 | 0.255 | 1 | CDC42SE1 |
| 0.00227414 | −0.1982395 | 0.225 | 0.233 | 1 | TPI1 |
| 0.00230231 | 0.34754742 | 0.142 | 0.066 | 1 | RBM33 |
| 0.00230287 | 0.16443256 | 0.191 | 0.116 | 1 | CEP78 |
| 0.00236596 | −0.1120148 | 0.112 | 0.101 | 1 | EIF2A |
| 0.00237016 | −0.2060147 | 0.165 | 0.176 | 1 | PCNP |
| 0.00237337 | 0.1207051 | 0.101 | 0.053 | 1 | PRDX1 |
| 0.00237403 | 0.14067073 | 0.109 | 0.06 | 1 | KDM6A |
| 0.00237988 | −0.2185078 | 1 | 1 | 1 | MT-RNR2 |
| 0.00239196 | −0.3973232 | 0.199 | 0.261 | 1 | IFI6 |
| 0.00239839 | 0.15549092 | 0.24 | 0.154 | 1 | SEPT9 |
| 0.00241072 | −0.2227432 | 0.723 | 0.758 | 1 | RPS7 |
| 0.00243703 | 0.21831755 | 0.169 | 0.094 | 1 | UCP2 |
| 0.00248862 | 0.17758916 | 0.101 | 0.057 | 1 | UBE2F |
| 0.0024941 | 0.33919214 | 0.281 | 0.167 | 1 | LNPEP |
| 0.00252527 | 0.11028012 | 0.356 | 0.255 | 1 | N4BP2L2 |
| 0.00252831 | 0.17429262 | 0.876 | 0.792 | 1 | RPL5 |
| 0.00255159 | 0.34930918 | 0.206 | 0.123 | 1 | ARRDC3 |
| 0.00255556 | −0.30199 | 0.082 | 0.132 | 1 | RP11-349A22.5 |
| 0.00263141 | 0.20043507 | 0.161 | 0.091 | 1 | CSTB |
| 0.00263763 | 0.29155438 | 0.12 | 0.047 | 1 | OLA1 |
| 0.00264614 | −0.1611568 | 0.139 | 0.132 | 1 | AP2M1 |
| 0.00269058 | 0.196034 | 0.18 | 0.097 | 1 | ZCCHC6 |
| 0.00269339 | 0.12612044 | 0.255 | 0.179 | 1 | SNRPB |
| 0.00269404 | 0.34100305 | 0.101 | 0.035 | 1 | FBXO11 |
| 0.00277914 | −0.15585 | 0.12 | 0.107 | 1 | NOP10 |
| 0.00278412 | 0.2015025 | 0.112 | 0.05 | 1 | ESCO1 |
| 0.00280548 | 0.15854087 | 0.199 | 0.123 | 1 | TPM4 |
| 0.00280783 | −0.1451463 | 0.232 | 0.217 | 1 | HLA-G |
| 0.00281126 | −0.1277374 | 0.633 | 0.607 | 1 | LCP1 |
| 0.00285316 | 0.12329823 | 0.236 | 0.157 | 1 | SLC9A3R1 |
| 0.00285559 | −0.4910659 | 0.06 | 0.126 | 1 | IGKC |
| 0.00285776 | 0.13217726 | 0.427 | 0.314 | 1 | HNRNPK |
| 0.00287226 | −0.2077399 | 0.509 | 0.525 | 1 | RPL29 |
| 0.00293924 | 0.13130043 | 0.195 | 0.119 | 1 | TTC3 |
| 0.00294047 | −0.3174646 | 0.184 | 0.226 | 1 | ATP5J |
| 0.00294878 | −0.2852578 | 0.236 | 0.267 | 1 | RAD21 |
| 0.00295411 | 0.1116201 | 0.401 | 0.384 | 1 | MAN1A2 |
| 0.00298147 | 0.19667122 | 0.112 | 0.05 | 1 | CAPRIN1 |
| 0.00299006 | 0.11609558 | 0.296 | 0.248 | 1 | MOB1A |
| 0.00302059 | −0.2925763 | 0.24 | 0.286 | 1 | HLA-DPB1 |
| 0.00303093 | −0.1234649 | 0.135 | 0.126 | 1 | RASSF1 |
| 0.0030418 | 0.16659645 | 0.202 | 0.135 | 1 | POLR3GL |
| 0.00304313 | 0.30166932 | 0.288 | 0.173 | 1 | MCL1 |
| 0.00304359 | 0.21038604 | 0.101 | 0.063 | 1 | ATF6 |
| 0.0030716 | 0.23149914 | 0.154 | 0.082 | 1 | INPP5D |
| 0.00308717 | 0.13569005 | 0.12 | 0.088 | 1 | OSER1 |
| 0.00309325 | −0.47278 | 0.056 | 0.135 | 1 | ANKRD42 |
| 0.00309954 | 0.11958611 | 0.112 | 0.069 | 1 | ASCC3 |
| 0.00312089 | −0.2614072 | 0.536 | 0.572 | 1 | ETS1 |
| 0.00314256 | 0.12082572 | 0.251 | 0.192 | 1 | YWHAQ |
| 0.0031447 | −0.2603308 | 0.094 | 0.11 | 1 | RAB10 |
| 0.00319673 | −0.3170973 | 0.094 | 0.116 | 1 | SFT2D1 |
| 0.00321422 | 0.19191185 | 0.652 | 0.516 | 1 | SRSF5 |
| 0.00322497 | 0.2233421 | 0.831 | 0.73 | 1 | DDX5 |
| 0.0032481 | −0.2354304 | 0.288 | 0.343 | 1 | NUCKS1 |
| 0.00325111 | 0.29997464 | 0.112 | 0.047 | 1 | DDIT3 |
| 0.00325356 | 0.11894372 | 0.109 | 0.06 | 1 | IFNAR2 |
| 0.0032711 | 0.24095202 | 0.292 | 0.201 | 1 | HLA-DPA1 |
| 0.00328466 | −0.222814 | 0.075 | 0.107 | 1 | CEP290 |
| 0.00330529 | −0.2340829 | 0.285 | 0.308 | 1 | CTSS |
| 0.00335214 | 0.28483752 | 0.154 | 0.072 | 1 | EIF4A1 |
| 0.00335817 | 0.18416558 | 0.101 | 0.05 | 1 | RBM38 |
| 0.0034121 | 0.38307829 | 0.187 | 0.094 | 1 | JUND |
| 0.00341246 | 0.29302882 | 0.109 | 0.041 | 1 | BTG3 |
| 0.00341299 | 0.22271952 | 0.109 | 0.044 | 1 | USP36 |
| 0.00341471 | −0.3219956 | 0.27 | 0.311 | 1 | UQCRQ |
| 0.00341961 | −0.1073459 | 0.296 | 0.258 | 1 | DBI |
| 0.0034358 | −0.2374212 | 0.438 | 0.462 | 1 | HCST |
| 0.00344355 | 0.27898327 | 0.281 | 0.189 | 1 | ZC3HAV1 |
| 0.00344427 | 0.23456247 | 0.206 | 0.126 | 1 | ILF3 |
| 0.00345786 | 0.19486799 | 0.363 | 0.258 | 1 | EIF3E |
| 0.00348316 | 0.22436785 | 0.101 | 0.044 | 1 | ADCY7 |
| 0.00349702 | 0.16435339 | 0.112 | 0.075 | 1 | RTFDC1 |
| 0.00355507 | 0.12310314 | 0.124 | 0.069 | 1 | BIN1 |
| 0.00357536 | 0.11275935 | 0.112 | 0.072 | 1 | HDAC1 |
| 0.00360114 | 0.26448369 | 0.176 | 0.088 | 1 | NAA50 |
| 0.00371265 | 0.1423389 | 0.142 | 0.075 | 1 | ILF2 |
| 0.0037262 | −0.1931171 | 0.202 | 0.208 | 1 | LBH |
| 0.00376067 | 0.12088026 | 0.996 | 0.994 | 1 | RPL13 |
| 0.0038509 | −0.1048578 | 0.169 | 0.145 | 1 | POLR2L |
| 0.00388614 | −0.2148697 | 0.243 | 0.239 | 1 | GIMAP7 |
| 0.00389753 | 0.30633318 | 0.127 | 0.05 | 1 | CCT2 |
| 0.00390428 | −0.3151714 | 0.135 | 0.182 | 1 | PRDM1 |
| 0.00394274 | 0.1833097 | 0.165 | 0.097 | 1 | ASH1L |
| 0.00399396 | 0.12333113 | 0.21 | 0.135 | 1 | ARIH2 |
| 0.00404032 | −0.1152309 | 0.184 | 0.179 | 1 | LBR |
| 0.00408113 | −0.2159266 | 0.127 | 0.142 | 1 | PRKAR1A |
| 0.00416645 | −0.1731729 | 0.146 | 0.154 | 1 | GZMM |
| 0.00423268 | 0.16033905 | 0.547 | 0.453 | 1 | STK17B |
| 0.00425256 | −0.2205855 | 0.393 | 0.418 | 1 | COX7A2 |
| 0.00427343 | 0.18533994 | 0.846 | 0.736 | 1 | RPS5 |
| 0.00428444 | 0.29886343 | 0.12 | 0.047 | 1 | IFRD1 |
| 0.00436359 | −0.13518 | 0.139 | 0.142 | 1 | ATP6AP2 |
| 0.00436767 | 0.10239647 | 0.566 | 0.459 | 1 | ITM2B |
| 0.004371 | −0.130373 | 0.142 | 0.126 | 1 | NDUFA2 |
| 0.00437233 | −0.2142254 | 0.307 | 0.333 | 1 | NDUFB2 |
| 0.00438062 | 0.14053131 | 0.109 | 0.057 | 1 | ITPR2 |
| 0.00439149 | −0.1029148 | 0.172 | 0.145 | 1 | CEBPZ |
| 0.00440539 | 0.16031825 | 0.255 | 0.164 | 1 | TCF25 |
| 0.00445306 | −0.241466 | 0.157 | 0.182 | 1 | CD7 |
| 0.00448181 | 0.21824232 | 0.232 | 0.151 | 1 | CRBN |
| 0.00453095 | −0.1829161 | 0.131 | 0.148 | 1 | ARHGEF1 |
| 0.00453364 | 0.32718494 | 0.184 | 0.107 | 1 | CASP4 |
| 0.00456563 | 0.20339194 | 0.124 | 0.075 | 1 | DEF6 |
| 0.00461704 | 0.17658569 | 0.187 | 0.11 | 1 | IL2RB |
| 0.00464258 | 0.33130092 | 0.285 | 0.179 | 1 | MT-ND1 |
| 0.00465168 | 0.27580195 | 0.262 | 0.157 | 1 | ATF4 |
| 0.00468639 | 0.21744411 | 0.101 | 0.041 | 1 | ACLY |
| 0.00470495 | 0.16970858 | 0.285 | 0.208 | 1 | BTG2 |
| 0.00473837 | 0.17305004 | 0.846 | 0.73 | 1 | RPL10A |
| 0.00475123 | 0.16946204 | 0.307 | 0.226 | 1 | IRF1 |
| 0.00478242 | −0.389613 | 0.112 | 0.176 | 1 | F2R |
| 0.00483574 | −0.1987713 | 0.154 | 0.16 | 1 | KMT2A |
| 0.00484599 | 0.24851957 | 0.337 | 0.217 | 1 | TOB1 |
| 0.00484761 | −0.1932344 | 0.172 | 0.164 | 1 | FAM204A |
| 0.00490801 | 0.39394478 | 0.146 | 0.063 | 1 | VPS37B |
| 0.00494225 | 0.11751259 | 0.165 | 0.11 | 1 | RBM8A |
| 0.0049476 | 0.14768707 | 0.176 | 0.11 | 1 | RHOH |
| 0.00503899 | 0.16444259 | 0.184 | 0.11 | 1 | SATB1 |
| 0.00514303 | 0.13502335 | 0.18 | 0.138 | 1 | SLC38A2 |
| 0.00518212 | 0.24116347 | 0.258 | 0.164 | 1 | PHF3 |
| 0.00525694 | 0.32578577 | 0.135 | 0.063 | 1 | CCL3 |
| 0.00525895 | 0.19726392 | 0.176 | 0.116 | 1 | TIAL1 |
| 0.00527104 | −0.2237474 | 0.124 | 0.129 | 1 | VPS36 |
| 0.00528004 | 0.22693997 | 0.659 | 0.519 | 1 | EEF2 |
| 0.00528894 | 0.18604475 | 0.15 | 0.088 | 1 | RNF168 |
| 0.00530748 | 0.35187619 | 0.161 | 0.079 | 1 | UBR2 |
| 0.00532243 | 0.29479227 | 0.116 | 0.053 | 1 | DERL2 |
| 0.00532584 | 0.3481917 | 0.112 | 0.041 | 1 | CHIC2 |
| 0.00533353 | −0.1136336 | 0.15 | 0.129 | 1 | HSPE1 |
| 0.00535886 | −0.2007133 | 0.09 | 0.104 | 1 | PDIA4 |
| 0.00540389 | −0.3534598 | 0.116 | 0.157 | 1 | SNRPE |
| 0.00541163 | 0.24963462 | 0.288 | 0.179 | 1 | HCLS1 |
| 0.00541985 | 0.27846161 | 0.236 | 0.132 | 1 | COTL1 |
| 0.0054548 | 0.17968015 | 0.15 | 0.091 | 1 | IRF2BP2 |
| 0.00545584 | 0.31149637 | 0.127 | 0.053 | 1 | EP300 |
| 0.0054783 | −0.1914967 | 0.067 | 0.104 | 1 | MIEN1 |
| 0.00553251 | −0.1054646 | 0.154 | 0.142 | 1 | SYNCRIP |
| 0.00562141 | 0.32063072 | 0.101 | 0.035 | 1 | NMRAL1 |
| 0.00568632 | 0.27461365 | 0.109 | 0.047 | 1 | ATG3 |
| 0.00571282 | 0.13971969 | 0.135 | 0.085 | 1 | TCIRG1 |
| 0.00574222 | 0.15707944 | 0.322 | 0.226 | 1 | PAIP2 |
| 0.00574678 | −0.1589513 | 0.172 | 0.167 | 1 | CKLF |
| 0.00576758 | 0.12456634 | 0.187 | 0.129 | 1 | ATP5C1 |
| 0.00584695 | 0.35291315 | 0.12 | 0.05 | 1 | CD55 |
| 0.00600706 | −0.3398934 | 0.142 | 0.189 | 1 | TBCA |
| 0.00609436 | 0.20638627 | 0.112 | 0.075 | 1 | PSMD2 |
| 0.00610385 | −0.3505947 | 0.142 | 0.176 | 1 | KHDRBS1 |
| 0.0061086 | 0.13444662 | 0.109 | 0.066 | 1 | RAB37 |
| 0.00615767 | 0.32349087 | 0.105 | 0.038 | 1 | SUGP2 |
| 0.00626915 | 0.13516344 | 0.127 | 0.091 | 1 | CHD8 |
| 0.00629996 | 0.12387485 | 0.487 | 0.403 | 1 | ITGB2 |
| 0.00632537 | −0.352462 | 0.303 | 0.39 | 1 | CAST |
| 0.00636399 | 0.17987198 | 0.142 | 0.079 | 1 | MRPL41 |
| 0.00637447 | −0.1524482 | 0.273 | 0.28 | 1 | AES |
| 0.0063762 | 0.12526355 | 0.112 | 0.06 | 1 | TRANK1 |
| 0.00638862 | −0.2151255 | 0.124 | 0.135 | 1 | PDCD10 |
| 0.00660383 | 0.24091865 | 0.322 | 0.226 | 1 | MT2A |
| 0.00660564 | 0.31357912 | 0.337 | 0.211 | 1 | SELL |
| 0.00661513 | −0.1706882 | 0.082 | 0.104 | 1 | EIF2AK1 |
| 0.00668957 | 0.17184351 | 0.91 | 0.824 | 1 | RPS15 |
| 0.00676811 | 0.11855681 | 0.206 | 0.148 | 1 | BUD31 |
| 0.00678943 | −0.1835747 | 0.206 | 0.22 | 1 | SEC61B |
| 0.00684379 | 0.14692442 | 0.277 | 0.195 | 1 | CD97 |
| 0.00685369 | 0.23349177 | 0.101 | 0.044 | 1 | POLR2C |
| 0.00686703 | −0.1881243 | 0.472 | 0.475 | 1 | NDUFA4 |
| 0.00688173 | −0.3555951 | 0.079 | 0.119 | 1 | M6PR |
| 0.00689095 | −0.1341791 | 0.176 | 0.17 | 1 | ERP29 |
| 0.00690719 | 0.17341853 | 0.124 | 0.085 | 1 | TSPAN32 |
| 0.00695443 | −0.2066788 | 0.094 | 0.101 | 1 | CHST12 |
| 0.00696799 | 0.30678173 | 0.105 | 0.038 | 1 | RN7SK |
| 0.00702815 | 0.14974541 | 0.303 | 0.211 | 1 | TSPO |
| 0.00704374 | 0.11671699 | 0.116 | 0.072 | 1 | PUM2 |
| 0.00708867 | −0.1139524 | 0.127 | 0.113 | 1 | NDUFA12 |
| 0.00710466 | 0.16204079 | 0.116 | 0.075 | 1 | IARS2 |
| 0.00711239 | −0.1263192 | 0.12 | 0.119 | 1 | ARL14EP |
| 0.0071275 | −0.1267878 | 0.176 | 0.17 | 1 | PA2G4 |
| 0.00717094 | 0.12714095 | 0.154 | 0.097 | 1 | PRPF4B |
| 0.00719013 | −0.3256564 | 0.146 | 0.189 | 1 | ERH |
| 0.0072052 | 0.14766317 | 0.397 | 0.289 | 1 | CIRBP |
| 0.00721864 | 0.30529814 | 0.116 | 0.06 | 1 | PHACTR2 |
| 0.00722004 | 0.23673846 | 0.105 | 0.044 | 1 | ERO1L |
| 0.00722832 | 0.28751598 | 0.187 | 0.119 | 1 | STAG2 |
| 0.00728589 | 0.17238084 | 0.243 | 0.154 | 1 | ZC3H15 |
| 0.00729609 | 0.10852788 | 0.139 | 0.085 | 1 | RP11-51J9.5 |
| 0.00730688 | −0.1070876 | 0.124 | 0.113 | 1 | WDR82 |
| 0.0073919 | −0.1705208 | 0.292 | 0.299 | 1 | CD164 |
| 0.00741227 | 0.31447873 | 0.157 | 0.072 | 1 | WSB1 |
| 0.00746673 | −0.1045753 | 0.483 | 0.456 | 1 | MT-ND4 |
| 0.00750956 | 0.11431661 | 0.303 | 0.226 | 1 | HIST1H1E |
| 0.00757106 | 0.23571503 | 0.109 | 0.053 | 1 | SFT2D2 |
| 0.00759207 | 0.29264151 | 0.135 | 0.063 | 1 | HECA |
| 0.007612 | 0.30437561 | 0.112 | 0.047 | 1 | KLF10 |
| 0.00764877 | 0.14019047 | 0.105 | 0.053 | 1 | BAZ2A |
| 0.00767783 | −0.2184694 | 0.139 | 0.167 | 1 | SRP9 |
| 0.00770696 | 0.13264926 | 0.124 | 0.072 | 1 | CLIP1 |
| 0.00771018 | 0.1519907 | 0.105 | 0.053 | 1 | COPS5 |
| 0.00772929 | −0.4151932 | 0.109 | 0.192 | 1 | CARD16 |
| 0.00783667 | −0.2553352 | 0.315 | 0.343 | 1 | CALM2 |
| 0.0078479 | 0.174026 | 0.15 | 0.104 | 1 | TMEM30A |
| 0.00786146 | 0.17769577 | 0.352 | 0.255 | 1 | VPS13C |
| 0.00798552 | 0.15677402 | 0.176 | 0.119 | 1 | SNHG3 |
| 0.00802469 | 0.12251523 | 0.996 | 0.991 | 1 | RPL19 |
| 0.00812482 | 0.25713326 | 0.12 | 0.057 | 1 | IER5 |
| 0.00814687 | −0.2212103 | 0.378 | 0.437 | 1 | LYZ |
| 0.00815782 | −0.177339 | 0.345 | 0.352 | 1 | COX5B |
| 0.00820967 | −0.1051882 | 0.139 | 0.126 | 1 | NAA38 |
| 0.00833772 | 0.19891766 | 0.18 | 0.101 | 1 | SPON2 |
| 0.00842085 | −0.1712027 | 0.951 | 0.959 | 1 | RPS3 |
| 0.00848532 | 0.20337527 | 0.101 | 0.044 | 1 | ILKAP |
| 0.00860473 | −0.2403086 | 0.135 | 0.157 | 1 | SHOC2 |
| 0.00872618 | −0.1334345 | 0.131 | 0.129 | 1 | NONO |
| 0.00872698 | 0.11533848 | 0.124 | 0.101 | 1 | MIA3 |
| 0.00873202 | −0.1049448 | 0.213 | 0.208 | 1 | YME1L1 |
| 0.00873766 | 0.10461064 | 0.105 | 0.066 | 1 | HIAT1 |
| 0.00876922 | 0.18985437 | 0.109 | 0.072 | 1 | CD226 |
| 0.00890503 | 0.19530824 | 0.12 | 0.057 | 1 | SPTBN1 |
| 0.00903891 | 0.13755997 | 0.352 | 0.258 | 1 | CCND3 |
| 0.00904989 | 0.18415504 | 0.184 | 0.113 | 1 | PCM1 |
| 0.00907641 | 0.19527676 | 0.146 | 0.097 | 1 | DNAJB14 |
| 0.00910612 | −0.1847895 | 0.704 | 0.72 | 1 | ATP5E |
| 0.00913497 | 0.16220215 | 0.27 | 0.195 | 1 | SPOCK2 |
| 0.00920643 | −0.2847305 | 0.09 | 0.123 | 1 | ARL2BP |
| 0.00920744 | −0.1149178 | 0.161 | 0.173 | 1 | STOM |
| 0.00939379 | −0.2343749 | 0.213 | 0.242 | 1 | NDUFS5 |
| 0.00942904 | 0.11826872 | 0.247 | 0.195 | 1 | NEAT1 |
| 0.00945854 | −0.3353397 | 0.067 | 0.11 | 1 | COMMD1 |
| 0.00948514 | −0.1295515 | 0.21 | 0.208 | 1 | RAB7L1 |
| 0.00956223 | −0.2145597 | 0.161 | 0.182 | 1 | CHURC1 |
| 0.00966252 | −0.2750366 | 0.386 | 0.44 | 1 | GMFG |
| 0.00972629 | 0.10394233 | 0.101 | 0.06 | 1 | SPAG9 |
| 0.00975122 | −0.1531374 | 0.172 | 0.179 | 1 | NDUFB11 |
| 0.00980223 | 0.35577501 | 0.124 | 0.05 | 1 | CTSB |
| 0.00980946 | −0.1742264 | 0.127 | 0.138 | 1 | NDUFS7 |
| 0.00996717 | 0.19596831 | 0.131 | 0.066 | 1 | LSM5 |
| 0.00998146 | 0.19417123 | 0.719 | 0.619 | 1 | SH3BGRL3 |
| 0.00999249 | −0.2366137 | 0.09 | 0.123 | 1 | FLI1 |
| 0.01009086 | 0.31508725 | 0.255 | 0.151 | 1 | PDE4D |
| 0.01017667 | −0.1846657 | 0.206 | 0.252 | 1 | MIF |
| 0.01025018 | 0.1147385 | 0.101 | 0.057 | 1 | ARCN1 |
| 0.01025756 | 0.19721522 | 0.569 | 0.434 | 1 | EIF4A2 |
| 0.0103255 | 0.28707556 | 0.213 | 0.119 | 1 | PDIA6 |
| 0.01038292 | 0.17042479 | 0.146 | 0.113 | 1 | RNASET2 |
| 0.01040126 | 0.12030988 | 0.337 | 0.261 | 1 | COX7B |
| 0.01046674 | −0.4104099 | 0.052 | 0.126 | 1 | IGHG1 |
| 0.01048253 | −0.2298338 | 0.15 | 0.145 | 1 | LUC7L3 |
| 0.01050189 | 0.29141599 | 0.146 | 0.079 | 1 | OGT |
| 0.01050573 | 0.20208059 | 0.24 | 0.16 | 1 | SERP1 |
| 0.01058657 | 0.17150731 | 0.172 | 0.107 | 1 | PBXIP1 |
| 0.01059199 | −0.3539652 | 0.195 | 0.277 | 1 | MYEOV2 |
| 0.01067296 | 0.11796999 | 0.191 | 0.126 | 1 | SMAP2 |
| 0.01071943 | 0.23034091 | 0.281 | 0.182 | 1 | LDHA |
| 0.01072505 | 0.21358805 | 0.157 | 0.107 | 1 | SLC2A3 |
| 0.01080091 | −0.1001861 | 0.116 | 0.113 | 1 | SUPT16H |
| 0.01082908 | 0.13127226 | 0.408 | 0.318 | 1 | PCED1B-AS1 |
| 0.01083946 | −0.1099187 | 0.506 | 0.478 | 1 | YBX1 |
| 0.01084819 | −0.1776024 | 0.139 | 0.142 | 1 | SERPINB1 |
| 0.01085754 | 0.24187499 | 0.375 | 0.258 | 1 | SAT1 |
| 0.01103959 | −0.1728366 | 0.131 | 0.142 | 1 | LSM3 |
| 0.01119291 | −0.1927925 | 0.734 | 0.764 | 1 | RPL18 |
| 0.01127858 | 0.19624788 | 0.105 | 0.06 | 1 | NUDT21 |
| 0.01132757 | 0.20253337 | 0.539 | 0.406 | 1 | ANXA1 |
| 0.01139947 | −0.164365 | 0.172 | 0.167 | 1 | SELPLG |
| 0.01143107 | 0.21995239 | 0.135 | 0.072 | 1 | CEP85L |
| 0.01158319 | 0.1835399 | 0.869 | 0.777 | 1 | UBB |
| 0.01159885 | −0.3550166 | 0.049 | 0.101 | 1 | BCCIP |
| 0.01175225 | 0.22314182 | 0.142 | 0.094 | 1 | BLOC1S2 |
| 0.01176324 | 0.33075746 | 0.101 | 0.035 | 1 | CCNH |
| 0.01187289 | −0.2152263 | 0.109 | 0.138 | 1 | TCEA1 |
| 0.0120062 | 0.12637989 | 0.352 | 0.27 | 1 | KIAA1551 |
| 0.01221956 | −0.1260196 | 0.135 | 0.138 | 1 | NDUFB6 |
| 0.01222584 | 0.14255457 | 0.288 | 0.208 | 1 | EIF2S3 |
| 0.01232167 | 0.15129608 | 0.105 | 0.057 | 1 | TTC14 |
| 0.01232556 | −0.1270602 | 0.206 | 0.214 | 1 | DNAJC8 |
| 0.0123398 | −0.2164301 | 0.086 | 0.113 | 1 | MATK |
| 0.01236855 | −0.1745108 | 0.105 | 0.119 | 1 | SMC3 |
| 0.01237501 | 0.27071244 | 0.12 | 0.057 | 1 | ZKSCAN1 |
| 0.01240937 | 0.11544027 | 0.416 | 0.321 | 1 | PNRC1 |
| 0.01247026 | −0.2377544 | 0.12 | 0.148 | 1 | CTD-2090I13.1 |
| 0.01252647 | 0.14337619 | 0.191 | 0.129 | 1 | ZNF91 |
| 0.01259696 | −0.1378319 | 0.116 | 0.113 | 1 | ABLIM1 |
| 0.01271538 | 0.32294148 | 0.161 | 0.085 | 1 | PATL2 |
| 0.01274896 | −0.2289129 | 0.124 | 0.145 | 1 | FIS1 |
| 0.01275958 | 0.29748727 | 0.112 | 0.05 | 1 | GPI |
| 0.01278187 | −0.1691822 | 0.161 | 0.186 | 1 | REEP5 |
| 0.01283729 | −0.1776702 | 0.434 | 0.45 | 1 | CD3E |
| 0.01285275 | 0.15994534 | 0.165 | 0.107 | 1 | CCDC88C |
| 0.01305796 | 0.3647127 | 0.154 | 0.075 | 1 | SRSF6 |
| 0.01305867 | 0.10069468 | 0.195 | 0.132 | 1 | ZBTB38 |
| 0.01308432 | −0.134223 | 0.431 | 0.434 | 1 | CAP1 |
| 0.01331777 | −0.2061919 | 0.169 | 0.186 | 1 | C9orf16 |
| 0.01352942 | −0.1341538 | 0.172 | 0.173 | 1 | MRPL33 |
| 0.0135708 | −0.1494322 | 0.228 | 0.245 | 1 | ARPC4 |
| 0.01362381 | 0.26391801 | 0.165 | 0.085 | 1 | HEXIM1 |
| 0.01366818 | 0.2656065 | 0.243 | 0.145 | 1 | STK10 |
| 0.01407584 | −0.1115271 | 0.243 | 0.239 | 1 | NDUFA6 |
| 0.01409441 | 0.14599037 | 0.124 | 0.075 | 1 | MAPRE2 |
| 0.01410341 | −0.235405 | 0.124 | 0.138 | 1 | XRN2 |
| 0.01412681 | 0.20269881 | 0.236 | 0.17 | 1 | BUB3 |
| 0.01413162 | 0.16747982 | 0.236 | 0.157 | 1 | PPP1CB |
| 0.01414507 | 0.33007858 | 0.146 | 0.072 | 1 | TRAF5 |
| 0.01421926 | 0.17006067 | 0.24 | 0.176 | 1 | BHLHE40 |
| 0.01441861 | 0.15367271 | 0.127 | 0.088 | 1 | MPC1 |
| 0.01446749 | 0.25023674 | 0.206 | 0.132 | 1 | FAM46C |
| 0.01454288 | −0.14056 | 0.464 | 0.447 | 1 | KLRD1 |
| 0.01472604 | 0.16939428 | 0.116 | 0.072 | 1 | ANKRD36C |
| 0.01479736 | −0.2341909 | 0.112 | 0.123 | 1 | UPP1 |
| 0.01481569 | 0.16675971 | 0.146 | 0.085 | 1 | PSMA5 |
| 0.01499754 | 0.20960231 | 0.131 | 0.069 | 1 | ADD1 |
| 0.01500725 | 0.24871749 | 0.213 | 0.132 | 1 | PPP1R2 |
| 0.01510383 | −0.2298071 | 0.15 | 0.195 | 1 | YTHDC1 |
| 0.01514425 | 0.16870861 | 0.24 | 0.157 | 1 | UBE2D2 |
| 0.01522438 | 0.20431221 | 0.112 | 0.057 | 1 | MYO5A |
| 0.01568975 | 0.110112 | 0.135 | 0.091 | 1 | SLC25A36 |
| 0.01592009 | −0.2104465 | 0.18 | 0.186 | 1 | UBE2L3 |
| 0.01607052 | 0.27124628 | 0.12 | 0.06 | 1 | FAM172A |
| 0.01613058 | −0.1078506 | 0.142 | 0.129 | 1 | G3BP1 |
| 0.01618638 | 0.19984333 | 0.142 | 0.113 | 1 | IFI44 |
| 0.01619063 | 0.10872114 | 0.356 | 0.286 | 1 | FLNA |
| 0.01621387 | −0.1429125 | 0.131 | 0.129 | 1 | SEC63 |
| 0.0163125 | 0.16109653 | 0.172 | 0.107 | 1 | SMG1 |
| 0.01664337 | −0.3582702 | 0.097 | 0.142 | 1 | PSIP1 |
| 0.01692967 | 0.26031893 | 0.109 | 0.053 | 1 | PSTPIP1 |
| 0.0169362 | 0.15074338 | 0.135 | 0.079 | 1 | URI1 |
| 0.01696204 | 0.22829545 | 0.221 | 0.135 | 1 | ERAP2 |
| 0.01698954 | −0.1649619 | 0.116 | 0.129 | 1 | DHX36 |
| 0.01723224 | −0.2382833 | 0.625 | 0.704 | 1 | GZMA |
| 0.01725521 | 0.17720012 | 0.24 | 0.164 | 1 | BPTF |
| 0.01731207 | 0.2192127 | 0.139 | 0.072 | 1 | FAM214A |
| 0.0173183 | 0.14053163 | 0.165 | 0.101 | 1 | SEC11A |
| 0.0176021 | 0.47645837 | 0.157 | 0.097 | 1 | S100B |
| 0.01781494 | −0.2381506 | 0.131 | 0.173 | 1 | C1QBP |
| 0.01787403 | 0.29934085 | 0.172 | 0.091 | 1 | SPSB3 |
| 0.01813368 | 0.13007309 | 0.884 | 0.789 | 1 | PTPRC |
| 0.0181971 | 0.2194844 | 0.15 | 0.085 | 1 | TMC8 |
| 0.01822099 | −0.2536717 | 0.112 | 0.129 | 1 | FAM192A |
| 0.01831745 | −0.154142 | 0.12 | 0.164 | 1 | ATP6V0B |
| 0.01832859 | 0.20791415 | 0.187 | 0.116 | 1 | MARCH7 |
| 0.01834453 | −0.130334 | 0.292 | 0.296 | 1 | SNHG8 |
| 0.01839153 | 0.27404385 | 0.116 | 0.05 | 1 | PTPN6 |
| 0.01856074 | −0.177465 | 0.202 | 0.195 | 1 | EIF2AK2 |
| 0.01858287 | 0.17056812 | 0.191 | 0.123 | 1 | LMAN2 |
| 0.01866773 | 0.17133018 | 0.112 | 0.06 | 1 | TRAPPC1 |
| 0.01893233 | 0.23372852 | 0.109 | 0.047 | 1 | ZFAND2A |
| 0.01893308 | 0.35535096 | 0.247 | 0.148 | 1 | CYLD |
| 0.01898676 | 0.27767266 | 0.112 | 0.047 | 1 | NCOA3 |
| 0.01899161 | 0.19218931 | 0.217 | 0.151 | 1 | USP15 |
| 0.01903014 | −0.241527 | 0.09 | 0.116 | 1 | COX16 |
| 0.01921625 | 0.28780714 | 0.127 | 0.063 | 1 | PGLS |
| 0.01931917 | −0.1960535 | 0.176 | 0.201 | 1 | SEC61G |
| 0.01945106 | 0.2854051 | 0.213 | 0.123 | 1 | KPNB1 |
| 0.01948939 | −0.2839484 | 0.094 | 0.142 | 1 | SEPT2 |
| 0.0196517 | −0.1086114 | 0.142 | 0.142 | 1 | HMGN3 |
| 0.01982264 | −0.1090519 | 0.367 | 0.349 | 1 | CD96 |
| 0.01996913 | 0.11558386 | 0.105 | 0.057 | 1 | CD160 |
| 0.02010384 | −0.152368 | 0.109 | 0.104 | 1 | FYTTD1 |
| 0.02026598 | 0.12601245 | 0.112 | 0.075 | 1 | PICALM |
| 0.02043046 | 0.3587787 | 0.112 | 0.047 | 1 | HIPK1 |
| 0.02056261 | 0.14295805 | 0.176 | 0.126 | 1 | CD8B |
| 0.02070532 | 0.17509143 | 0.176 | 0.107 | 1 | CCT4 |
| 0.02071013 | 0.21407169 | 0.154 | 0.082 | 1 | CEP57 |
| 0.02074827 | 0.15305345 | 0.135 | 0.075 | 1 | RNF44 |
| 0.02076459 | −0.1699358 | 0.09 | 0.113 | 1 | MST4 |
| 0.02080143 | 0.10723955 | 0.382 | 0.324 | 1 | GUK1 |
| 0.02113939 | 0.26221086 | 0.255 | 0.164 | 1 | LYST |
| 0.02114044 | 0.1344105 | 0.112 | 0.075 | 1 | MOAP1 |
| 0.02119208 | −0.1340671 | 0.124 | 0.126 | 1 | ADAM10 |
| 0.02142917 | 0.18108463 | 0.116 | 0.075 | 1 | PIM1 |
| 0.02144909 | 0.14855373 | 0.109 | 0.072 | 1 | MMADHC |
| 0.02146595 | 0.19207685 | 0.109 | 0.072 | 1 | RASGRP1 |
| 0.02157024 | 0.18033864 | 0.184 | 0.113 | 1 | CCSER2 |
| 0.02164214 | 0.13272568 | 0.116 | 0.066 | 1 | ANXA4 |
| 0.02170657 | 0.1409826 | 0.195 | 0.132 | 1 | NSA2 |
| 0.02184956 | 0.27935818 | 0.157 | 0.082 | 1 | DNAJC3 |
| 0.02187479 | 0.12663578 | 0.187 | 0.123 | 1 | XRCC6 |
| 0.02188481 | −0.3170772 | 0.438 | 0.538 | 1 | MTRNR2L3 |
| 0.02217372 | −0.1980226 | 0.457 | 0.481 | 1 | ATP5L |
| 0.02226974 | −0.1259596 | 0.131 | 0.113 | 1 | C10orf118 |
| 0.02260862 | 0.29620182 | 0.131 | 0.063 | 1 | CTDSPL2 |
| 0.02261937 | −0.1982067 | 0.326 | 0.336 | 1 | HOPX |
| 0.02268954 | −0.1329949 | 0.105 | 0.107 | 1 | MRPS34 |
| 0.02274192 | 0.25442229 | 0.142 | 0.088 | 1 | GADD45GIP1 |
| 0.02276417 | 0.12418494 | 0.21 | 0.145 | 1 | UBXN1 |
| 0.02276538 | 0.32634129 | 0.131 | 0.063 | 1 | TOR1AIP1 |
| 0.02285163 | −0.1466781 | 0.978 | 0.978 | 1 | RPL37A |
| 0.02287114 | −0.1622257 | 0.105 | 0.101 | 1 | SRPK2 |
| 0.02299837 | 0.25743879 | 0.161 | 0.088 | 1 | XBP1 |
| 0.02332616 | −0.1069549 | 0.135 | 0.126 | 1 | GNPTAB |
| 0.02346846 | 0.19588818 | 0.131 | 0.072 | 1 | UQCRC2 |
| 0.02397025 | −0.1308686 | 0.09 | 0.101 | 1 | AIP |
| 0.02398221 | −0.1645646 | 0.195 | 0.214 | 1 | C19orf53 |
| 0.02413747 | −0.2089392 | 0.191 | 0.204 | 1 | PEBP1 |
| 0.02415883 | 0.11220165 | 0.12 | 0.082 | 1 | APLP2 |
| 0.02442588 | 0.27911827 | 0.154 | 0.079 | 1 | SNHG1 |
| 0.0244617 | −0.2105359 | 0.172 | 0.189 | 1 | GPR56 |
| 0.02469884 | 0.10907418 | 0.101 | 0.066 | 1 | ZNF655 |
| 0.02474579 | 0.25331447 | 0.146 | 0.079 | 1 | MDM2 |
| 0.02478937 | 0.10478743 | 0.101 | 0.06 | 1 | H2AFJ |
| 0.02481625 | −0.2649896 | 0.079 | 0.101 | 1 | SP140L |
| 0.02485871 | 0.23080676 | 0.116 | 0.053 | 1 | ZNF451 |
| 0.02486354 | 0.12656504 | 0.139 | 0.107 | 1 | PTEN |
| 0.02495014 | −0.2810131 | 0.071 | 0.101 | 1 | CD244 |
| 0.02508371 | 0.21599166 | 0.116 | 0.063 | 1 | ATL3 |
| 0.02545534 | 0.17185787 | 0.161 | 0.11 | 1 | SNHG15 |
| 0.02555794 | −0.3333603 | 0.12 | 0.186 | 1 | EMP3 |
| 0.02577588 | −0.3198698 | 0.071 | 0.116 | 1 | MRFAP1L1 |
| 0.02583251 | −0.1459822 | 0.288 | 0.28 | 1 | SYF2 |
| 0.02593117 | 0.10449863 | 0.146 | 0.094 | 1 | ST3GAL1 |
| 0.02597253 | −0.109392 | 0.281 | 0.267 | 1 | NIPBL |
| 0.02619701 | −0.1150588 | 0.446 | 0.44 | 1 | SEPT7 |
| 0.02629989 | −0.1829518 | 0.109 | 0.132 | 1 | LAMTOR1 |
| 0.0265787 | 0.11555402 | 0.352 | 0.277 | 1 | SKP1 |
| 0.02664545 | −0.1590606 | 0.18 | 0.182 | 1 | PRR13 |
| 0.02665316 | 0.13655857 | 0.127 | 0.091 | 1 | UFD1L |
| 0.02672317 | −0.3103506 | 0.105 | 0.148 | 1 | DNAJB6 |
| 0.02674372 | −0.1049518 | 0.154 | 0.138 | 1 | PHF11 |
| 0.02675357 | 0.12574282 | 0.251 | 0.182 | 1 | ZBTB1 |
| 0.02684095 | 0.27942429 | 0.247 | 0.157 | 1 | GBP2 |
| 0.0268632 | 0.18696816 | 0.142 | 0.104 | 1 | BIRC2 |
| 0.02692115 | 0.15728265 | 0.109 | 0.057 | 1 | SMAP1 |
| 0.02692491 | 0.21629904 | 0.277 | 0.192 | 1 | MT-ND3 |
| 0.02721594 | 0.25166229 | 0.124 | 0.06 | 1 | GATA3 |
| 0.02756657 | −0.1314733 | 0.273 | 0.28 | 1 | USMG5 |
| 0.02826558 | −0.3567539 | 0.112 | 0.167 | 1 | MPHOSPH8 |
| 0.02828264 | 0.10535797 | 0.101 | 0.072 | 1 | POLE3 |
| 0.02835018 | 0.24393414 | 0.221 | 0.145 | 1 | RNF149 |
| 0.02846222 | −0.1222682 | 0.165 | 0.16 | 1 | APBB1IP |
| 0.02857617 | −0.2030707 | 0.228 | 0.245 | 1 | LAMTOR4 |
| 0.02861659 | −0.1605965 | 0.187 | 0.192 | 1 | PRKCH |
| 0.02862901 | 0.24832894 | 0.112 | 0.057 | 1 | RCN2 |
| 0.02867854 | 0.38665126 | 0.131 | 0.06 | 1 | KRR1 |
| 0.02869751 | −0.2738267 | 0.15 | 0.173 | 1 | RBX1 |
| 0.02888426 | −0.1762261 | 0.528 | 0.519 | 1 | CD2 |
| 0.02908959 | −0.2001666 | 0.109 | 0.142 | 1 | REL |
| 0.02911743 | 0.15784744 | 0.449 | 0.362 | 1 | SLFN5 |
| 0.02936225 | 0.12721242 | 0.633 | 0.522 | 1 | ZFP36L2 |
| 0.02942689 | −0.1116038 | 0.139 | 0.129 | 1 | ARPC5L |
| 0.02965096 | 0.13051607 | 0.18 | 0.123 | 1 | MAT2B |
| 0.029899 | −0.2430517 | 0.105 | 0.129 | 1 | DDT |
| 0.03044567 | 0.13400008 | 0.581 | 0.475 | 1 | CLEC2B |
| 0.03051564 | 0.11421012 | 0.116 | 0.069 | 1 | TOP2B |
| 0.03060797 | −0.2291083 | 0.195 | 0.22 | 1 | GLIPR1 |
| 0.03065645 | 0.19815926 | 0.217 | 0.145 | 1 | DDX21 |
| 0.03067255 | −0.3310213 | 0.06 | 0.113 | 1 | MINOS1 |
| 0.0309003 | 0.12639336 | 0.539 | 0.428 | 1 | TMEM66 |
| 0.0309839 | 0.15068753 | 0.135 | 0.082 | 1 | FAM208A |
| 0.03106425 | 0.20490392 | 0.187 | 0.113 | 1 | HMHA1 |
| 0.03139183 | 0.10603013 | 0.157 | 0.107 | 1 | THOC2 |
| 0.03143642 | −0.2938032 | 0.09 | 0.129 | 1 | RSBN1L |
| 0.03152527 | 0.28505007 | 0.195 | 0.116 | 1 | EAPP |
| 0.03156533 | −0.1941195 | 0.498 | 0.497 | 1 | MT-CO3 |
| 0.03176952 | −0.1228172 | 0.213 | 0.223 | 1 | XRCC5 |
| 0.03184975 | 0.1451643 | 0.124 | 0.072 | 1 | GTF2A2 |
| 0.03191457 | 0.29235744 | 0.202 | 0.126 | 1 | ABI1 |
| 0.03205779 | 0.26896181 | 0.172 | 0.101 | 1 | DIP2A |
| 0.03208748 | 0.22802606 | 0.195 | 0.116 | 1 | PK3IP1 |
| 0.03219415 | −0.1236103 | 0.112 | 0.107 | 1 | ANP32A |
| 0.03235627 | 0.14424437 | 0.184 | 0.119 | 1 | GBP1 |
| 0.03252397 | 0.22693754 | 0.12 | 0.066 | 1 | ASAH1 |
| 0.032662 | 0.12920968 | 0.101 | 0.06 | 1 | RLF |
| 0.03272142 | 0.24185553 | 0.109 | 0.053 | 1 | FKBP5 |
| 0.03355279 | 0.15545114 | 0.131 | 0.085 | 1 | ZNF276 |
| 0.03395203 | 0.13620064 | 0.348 | 0.255 | 1 | ACTR3 |
| 0.03414141 | −0.1543487 | 0.288 | 0.318 | 1 | HIST1H4C |
| 0.03437417 | −0.2849897 | 0.199 | 0.245 | 1 | TMEM258 |
| 0.03456953 | −0.1697647 | 0.247 | 0.248 | 1 | PPP1CA |
| 0.03477884 | 0.24164623 | 0.101 | 0.047 | 1 | PER1 |
| 0.03509435 | 0.26455918 | 0.127 | 0.069 | 1 | NMRK1 |
| 0.03512654 | 0.14334928 | 0.813 | 0.714 | 1 | GNB2L1 |
| 0.03537469 | −0.1868049 | 0.124 | 0.126 | 1 | SNRPD1 |
| 0.03592666 | −0.1003382 | 0.135 | 0.129 | 1 | CASC4 |
| 0.03618219 | 0.10524001 | 0.154 | 0.11 | 1 | SSR1 |
| 0.03620559 | −0.176702 | 0.097 | 0.129 | 1 | SQSTM1 |
| 0.03639934 | 0.19374293 | 0.176 | 0.116 | 1 | EIF3I |
| 0.03655941 | 0.10123514 | 0.165 | 0.129 | 1 | CCDC59 |
| 0.03657452 | −0.2540174 | 0.082 | 0.113 | 1 | H3F3A |
| 0.03684462 | −0.2965373 | 0.049 | 0.107 | 1 | TSNAX |
| 0.03688935 | 0.15360336 | 0.131 | 0.091 | 1 | SNRNP200 |
| 0.03738003 | −0.3141879 | 0.075 | 0.107 | 1 | TIGIT |
| 0.0374826 | −0.2159174 | 0.094 | 0.104 | 1 | COMMD8 |
| 0.03763246 | 0.11787638 | 0.996 | 1 | 1 | TMSB10 |
| 0.03776794 | 0.12961281 | 0.112 | 0.072 | 1 | EXOC5 |
| 0.03819094 | −0.2382012 | 0.131 | 0.179 | 1 | SRP72 |
| 0.03837033 | −0.1908407 | 0.135 | 0.148 | 1 | RAB8A |
| 0.03873897 | −0.1203451 | 0.101 | 0.094 | 1 | SAMSN1 |
| 0.03886713 | 0.22509248 | 0.213 | 0.138 | 1 | IL10RA |
| 0.03893322 | 0.195476 | 0.195 | 0.129 | 1 | RNF7 |
| 0.03915106 | 0.11485614 | 0.176 | 0.116 | 1 | OFD1 |
| 0.03960269 | −0.151992 | 0.303 | 0.308 | 1 | CYBA |
| 0.03972726 | −0.1811349 | 0.266 | 0.302 | 1 | LY6E |
| 0.03973468 | 0.1545278 | 0.135 | 0.082 | 1 | SLMO2 |
| 0.03978269 | 0.17356204 | 0.109 | 0.057 | 1 | CYP20A1 |
| 0.0399881 | 0.12791071 | 0.221 | 0.167 | 1 | SH2D1A |
| 0.04001762 | 0.22788732 | 0.109 | 0.053 | 1 | TMEM173 |
| 0.04037424 | 0.28539227 | 0.112 | 0.05 | 1 | PPP6R3 |
| 0.04064227 | 0.15173983 | 0.124 | 0.072 | 1 | PRR5L |
| 0.04090516 | −0.1376606 | 0.955 | 0.947 | 1 | RPL37 |
| 0.04106443 | −0.2786877 | 0.071 | 0.107 | 1 | MTIF3 |
| 0.04159257 | −0.2654166 | 0.169 | 0.208 | 1 | CTSC |
| 0.04168538 | 0.15138331 | 0.105 | 0.069 | 1 | ZNF622 |
| 0.0418121 | −0.1669805 | 0.127 | 0.132 | 1 | VCL |
| 0.04195456 | −0.2120337 | 0.112 | 0.123 | 1 | UBTF |
| 0.04210862 | 0.20453042 | 0.101 | 0.05 | 1 | FCER1G |
| 0.04221145 | 0.16230423 | 0.154 | 0.094 | 1 | DR1 |
| 0.04233286 | 0.14995521 | 0.18 | 0.116 | 1 | DYNC1H1 |
| 0.04283175 | −0.295473 | 0.075 | 0.107 | 1 | TXN2 |
| 0.0428723 | 0.16211606 | 0.124 | 0.069 | 1 | TCP1 |
| 0.04298557 | −0.2054678 | 0.12 | 0.148 | 1 | CACYBP |
| 0.04298962 | −0.1558764 | 0.225 | 0.248 | 1 | PSMA6 |
| 0.04346931 | −0.127958 | 0.258 | 0.258 | 1 | FAM49B |
| 0.04381298 | 0.14089773 | 0.135 | 0.082 | 1 | TTC17 |
| 0.04385699 | −0.1265467 | 0.202 | 0.195 | 1 | TAF7 |
| 0.04394913 | 0.13943138 | 0.169 | 0.116 | 1 | CAMLG |
| 0.04407704 | 0.22248755 | 0.18 | 0.11 | 1 | TMC6 |
| 0.04428446 | 0.10404918 | 0.221 | 0.173 | 1 | GPR65 |
| 0.04438484 | 0.2585358 | 0.109 | 0.053 | 1 | CCNDBP1 |
| 0.0449084 | −0.204042 | 0.09 | 0.107 | 1 | PCMTD1 |
| 0.04498379 | −0.1495658 | 0.169 | 0.17 | 1 | NR3C1 |
| 0.04510644 | 0.14021742 | 0.195 | 0.135 | 1 | CEP350 |
| 0.0456219 | 0.11919563 | 0.33 | 0.274 | 1 | HMGB2 |
| 0.0462412 | −0.1867136 | 0.262 | 0.283 | 1 | NDUFB7 |
| 0.04657249 | 0.11967491 | 0.184 | 0.142 | 1 | FCRL6 |
| 0.04669812 | 0.17912215 | 0.603 | 0.487 | 1 | GAPDH |
| 0.04681836 | −0.3290153 | 0.199 | 0.245 | 1 | IFI27 |
| 0.04745549 | 0.18752175 | 0.161 | 0.097 | 1 | SYTL3 |
| 0.04765688 | −0.1274197 | 0.127 | 0.142 | 1 | ARHGDIA |
| 0.04767208 | 0.13459162 | 0.172 | 0.138 | 1 | CMPK1 |
| 0.04769225 | 0.28467022 | 0.131 | 0.066 | 1 | STRAP |
| 0.04793156 | −0.2175322 | 0.326 | 0.349 | 1 | SOD1 |
| 0.04938339 | 0.13337268 | 0.116 | 0.069 | 1 | MRPL34 |
| 0.04979874 | −0.1209656 | 0.105 | 0.101 | 1 | EVI2A |
| 0.04999353 | −0.1234545 | 0.348 | 0.355 | 1 | PPIB |
| 0.05019371 | −0.1160391 | 0.307 | 0.302 | 1 | SCAF11 |
| 0.05024873 | 0.15778938 | 0.124 | 0.079 | 1 | TSEN54 |
| 0.05041386 | 0.21118729 | 0.15 | 0.094 | 1 | RER1 |
| 0.05055869 | 0.31313426 | 0.101 | 0.044 | 1 | KB-1208A12.3 |
| 0.05101363 | 0.18011527 | 0.116 | 0.082 | 1 | PDS5A |
| 0.05130722 | −0.170565 | 0.315 | 0.33 | 1 | ATRX |
| 0.05171561 | −0.1242252 | 0.303 | 0.289 | 1 | DDX17 |
| 0.05174542 | −0.2261912 | 0.097 | 0.123 | 1 | SNRPG |
| 0.05186174 | 0.10745825 | 0.139 | 0.094 | 1 | TMED10 |
| 0.05224208 | −0.1041313 | 0.996 | 0.997 | 1 | RPS14 |
| 0.05250987 | 0.10565443 | 0.139 | 0.091 | 1 | DUT |
| 0.05332625 | −0.1680738 | 0.202 | 0.211 | 1 | PIP4K2A |
| 0.0535488 | 0.22977528 | 0.101 | 0.069 | 1 | USP9X |
| 0.05361754 | 0.10679164 | 0.169 | 0.129 | 1 | B4GALT1 |
| 0.05374332 | −0.137989 | 0.419 | 0.447 | 1 | HSP90B1 |
| 0.05382278 | 0.22410474 | 0.345 | 0.248 | 1 | SMCHD1 |
| 0.05409142 | 0.2368922 | 0.101 | 0.047 | 1 | SNRPA |
| 0.05430233 | −0.1751764 | 0.139 | 0.145 | 1 | RP11-94L15.2 |
| 0.05437606 | 0.12399915 | 0.536 | 0.443 | 1 | HSP90AB1 |
| 0.05446943 | −0.1228321 | 0.135 | 0.135 | 1 | AIMP1 |
| 0.05476149 | −0.1768802 | 0.925 | 0.956 | 1 | CD52 |
| 0.05481176 | −0.1460628 | 0.172 | 0.164 | 1 | LTB |
| 0.05489354 | 0.2006944 | 0.101 | 0.05 | 1 | MYLIP |
| 0.05511828 | 0.28604201 | 0.12 | 0.06 | 1 | PTTG1IP |
| 0.05544688 | −0.1481171 | 0.693 | 0.739 | 1 | SERF2 |
| 0.05568101 | −0.1880907 | 0.352 | 0.393 | 1 | EIF3K |
| 0.05587006 | −0.1540311 | 0.12 | 0.132 | 1 | TMED9 |
| 0.0561798 | −0.1324652 | 0.187 | 0.198 | 1 | SPCS1 |
| 0.05710015 | 0.16513917 | 0.712 | 0.61 | 1 | EEF1B2 |
| 0.0571266 | −0.1756724 | 0.157 | 0.176 | 1 | THRAP3 |
| 0.05715464 | 0.12365313 | 0.978 | 0.962 | 1 | RPS13 |
| 0.05715795 | −0.2851148 | 0.12 | 0.154 | 1 | IKZF2 |
| 0.05747623 | 0.24281624 | 0.187 | 0.126 | 1 | TMF1 |
| 0.05780024 | 0.29952413 | 0.199 | 0.119 | 1 | C1orf21 |
| 0.05810427 | −0.1538722 | 0.127 | 0.154 | 1 | FOXN3 |
| 0.05856638 | 0.31286635 | 0.157 | 0.088 | 1 | WTAP |
| 0.058707 | −0.2467395 | 0.124 | 0.148 | 1 | CASP1 |
| 0.05923654 | 0.10667569 | 0.472 | 0.396 | 1 | PDIA3 |
| 0.05935673 | 0.1451027 | 0.105 | 0.06 | 1 | ZNF644 |
| 0.05942971 | 0.14915066 | 0.12 | 0.075 | 1 | KRT10 |
| 0.05968779 | 0.16450481 | 0.116 | 0.079 | 1 | GNB2 |
| 0.05977147 | −0.2818508 | 0.094 | 0.123 | 1 | SSU72 |
| 0.05993169 | −0.1841462 | 0.12 | 0.138 | 1 | RGS10 |
| 0.06018195 | 0.17241962 | 0.105 | 0.06 | 1 | ANKLE2 |
| 0.06021821 | 0.12060891 | 0.139 | 0.091 | 1 | CD84 |
| 0.06050548 | −0.1735015 | 0.097 | 0.101 | 1 | BECN1 |
| 0.06064722 | −0.2762159 | 0.161 | 0.223 | 1 | TMEM50A |
| 0.06083502 | 0.18171066 | 0.273 | 0.192 | 1 | C6orf48 |
| 0.06131117 | 0.18608109 | 0.221 | 0.148 | 1 | C1orf63 |
| 0.06187432 | −0.112254 | 0.131 | 0.119 | 1 | RTF1 |
| 0.06293553 | −0.1152153 | 0.127 | 0.132 | 1 | ATRAID |
| 0.06347797 | −0.2921721 | 0.101 | 0.142 | 1 | OXNAD1 |
| 0.06355311 | −0.2533333 | 0.139 | 0.173 | 1 | LINC00861 |
| 0.0642225 | −0.2190929 | 0.116 | 0.132 | 1 | ZCRB1 |
| 0.06538805 | 0.12216377 | 0.124 | 0.082 | 1 | ETF1 |
| 0.06545405 | 0.12807283 | 0.101 | 0.06 | 1 | TBCC |
| 0.06590695 | −0.1248738 | 0.142 | 0.142 | 1 | ACIN1 |
| 0.06598898 | 0.16949121 | 0.127 | 0.088 | 1 | C1orf43 |
| 0.06652882 | −0.1919804 | 0.124 | 0.164 | 1 | MRPS21 |
| 0.06757002 | −0.2738359 | 0.202 | 0.245 | 1 | IFI44L |
| 0.06757938 | −0.2675854 | 0.067 | 0.101 | 1 | S100A11 |
| 0.06800965 | 0.16670343 | 0.202 | 0.138 | 1 | SIVA1 |
| 0.06913323 | 0.10835314 | 0.139 | 0.101 | 1 | LINC00657 |
| 0.06923118 | 0.101374 | 0.318 | 0.261 | 1 | LDHB |
| 0.06959096 | −0.2309716 | 0.075 | 0.101 | 1 | POLR2F |
| 0.06989282 | 0.10826841 | 0.101 | 0.063 | 1 | PRDX2 |
| 0.06996474 | −0.2365919 | 0.09 | 0.113 | 1 | DYNLL1 |
| 0.07016041 | 0.13855238 | 0.243 | 0.211 | 1 | RUNX3 |
| 0.07087591 | −0.1227234 | 0.109 | 0.107 | 1 | SRSF9 |
| 0.0721831 | 0.23856736 | 0.12 | 0.063 | 1 | LPXN |
| 0.07236468 | −0.1642377 | 0.157 | 0.167 | 1 | FBL |
| 0.07266388 | 0.18825562 | 0.139 | 0.091 | 1 | PFDN4 |
| 0.07349952 | 0.13206493 | 0.116 | 0.069 | 1 | MSL2 |
| 0.07397114 | 0.15920731 | 0.146 | 0.091 | 1 | TLK1 |
| 0.07420538 | 0.26436274 | 0.124 | 0.063 | 1 | PRKCQ |
| 0.07452411 | −0.225599 | 0.161 | 0.192 | 1 | CCDC69 |
| 0.07499329 | −0.1198542 | 0.105 | 0.104 | 1 | IRF7 |
| 0.07500366 | 0.15380429 | 0.221 | 0.157 | 1 | CSNK1G3 |
| 0.07571372 | −0.166536 | 0.139 | 0.157 | 1 | HSD17B11 |
| 0.07610153 | −0.201647 | 0.079 | 0.104 | 1 | BRK1 |
| 0.07682554 | 0.11673873 | 0.105 | 0.088 | 1 | ZBTB44 |
| 0.07846859 | −0.2308099 | 0.09 | 0.123 | 1 | EIF2S2 |
| 0.07852224 | 0.16409173 | 0.116 | 0.075 | 1 | ATP11B |
| 0.07957682 | 0.23624459 | 0.112 | 0.066 | 1 | APPL1 |
| 0.07961355 | −0.193815 | 0.105 | 0.126 | 1 | RBBP7 |
| 0.07971631 | 0.20792167 | 0.18 | 0.113 | 1 | HNRNPF |
| 0.08119416 | −0.1713139 | 0.127 | 0.142 | 1 | GBP4 |
| 0.08131257 | 0.2065409 | 0.157 | 0.104 | 1 | RFC1 |
| 0.08172205 | 0.19548718 | 0.124 | 0.072 | 1 | DECR1 |
| 0.08231158 | −0.130937 | 0.094 | 0.119 | 1 | COPS6 |
| 0.08358419 | 0.15243498 | 0.165 | 0.116 | 1 | DYNLT1 |
| 0.08508904 | 0.10959909 | 0.135 | 0.088 | 1 | EPB41L4A-AS1 |
| 0.0852773 | −0.1426397 | 0.127 | 0.138 | 1 | GNG5 |
| 0.08605449 | −0.181511 | 0.097 | 0.11 | 1 | TTC19 |
| 0.08614468 | 0.12025229 | 0.169 | 0.116 | 1 | JMJD1C |
| 0.08635473 | 0.12614731 | 0.161 | 0.126 | 1 | CNTRL |
| 0.08641492 | −0.1017497 | 0.101 | 0.107 | 1 | C4orf48 |
| 0.08665056 | −0.1065524 | 0.124 | 0.123 | 1 | BLOC1S1 |
| 0.087116 | 0.2296034 | 0.139 | 0.079 | 1 | ELOVL5 |
| 0.08713177 | 0.10740485 | 0.251 | 0.192 | 1 | HNRNPR |
| 0.08843863 | 0.10151848 | 0.101 | 0.063 | 1 | ATHL1 |
| 0.08970233 | −0.1169761 | 0.116 | 0.129 | 1 | CCND2 |
| 0.09006802 | −0.1500337 | 0.086 | 0.104 | 1 | LGALS3 |
| 0.09020294 | −0.1492946 | 0.127 | 0.135 | 1 | TMPO |
| 0.09114364 | 0.10919794 | 0.101 | 0.075 | 1 | IVNS1ABP |
| 0.09196347 | 0.12890317 | 0.191 | 0.138 | 1 | KLF3 |
| 0.09228476 | −0.1189558 | 0.232 | 0.239 | 1 | SAMD9 |
| 0.09251694 | −0.2508581 | 0.184 | 0.211 | 1 | BDP1 |
| 0.09499132 | 0.16190632 | 0.101 | 0.057 | 1 | UBA3 |
| 0.09532958 | 0.13301237 | 0.101 | 0.06 | 1 | SLBP |
| 0.09553974 | 0.14076113 | 0.116 | 0.069 | 1 | HBP1 |
| 0.09620228 | −0.1750504 | 0.101 | 0.104 | 1 | SPEN |
| 0.09669425 | −0.2420845 | 0.086 | 0.11 | 1 | ZMYND11 |
| 0.09700144 | −0.209121 | 0.101 | 0.132 | 1 | PPM1K |
| 0.09806129 | 0.21133303 | 0.12 | 0.066 | 1 | ETFB |
| 0.09832737 | 0.11071796 | 0.281 | 0.236 | 1 | SLC25A5 |
| 0.09920356 | −0.230031 | 0.082 | 0.101 | 1 | NDUFC1 |
| 0.09958244 | −0.204067 | 0.337 | 0.371 | 1 | KMT2E |
| 0.09976193 | 0.14966686 | 0.127 | 0.085 | 1 | PPP1CC |
| 0.10004386 | 0.2012085 | 0.112 | 0.063 | 1 | CTNNB1 |
| 0.10010214 | −0.1635736 | 0.184 | 0.214 | 1 | PCBP1 |
| 0.10068324 | 0.11718904 | 0.112 | 0.075 | 1 | NFE2L2 |
| 0.10247993 | −0.1003299 | 0.124 | 0.135 | 1 | BCAP31 |
| 0.10575624 | 0.20089991 | 0.172 | 0.11 | 1 | PHB2 |
| 0.10647207 | −0.2862175 | 0.18 | 0.242 | 1 | TERF2IP |
| 0.1070344 | −0.1021943 | 0.105 | 0.107 | 1 | SVIP |
| 0.10860783 | 0.22900149 | 0.154 | 0.094 | 1 | SMEK1 |
| 0.10875102 | 0.1210587 | 0.273 | 0.211 | 1 | RAP1A |
| 0.10948926 | −0.1442285 | 0.15 | 0.198 | 1 | ATP2B4 |
| 0.11004756 | −0.1413646 | 0.146 | 0.167 | 1 | PET100 |
| 0.11108437 | −0.125605 | 0.12 | 0.119 | 1 | CCDC107 |
| 0.11331356 | 0.15291147 | 0.18 | 0.142 | 1 | RSRC1 |
| 0.11368155 | 0.12836356 | 0.105 | 0.063 | 1 | KBTBD2 |
| 0.11465197 | 0.20489991 | 0.112 | 0.082 | 1 | AFG3L2 |
| 0.11521484 | −0.1365305 | 0.12 | 0.132 | 1 | RECQL |
| 0.11569556 | −0.1645122 | 0.127 | 0.129 | 1 | C5orf56 |
| 0.11718258 | −0.1849395 | 0.075 | 0.104 | 1 | UBE2J1 |
| 0.11729516 | 0.16620482 | 0.112 | 0.066 | 1 | AZIN1 |
| 0.11759088 | 0.16142086 | 0.12 | 0.075 | 1 | MDM4 |
| 0.11771988 | −0.1220428 | 0.109 | 0.113 | 1 | XPO1 |
| 0.11962627 | −0.1204517 | 0.865 | 0.849 | 1 | TXNIP |
| 0.12411702 | 0.20512957 | 0.12 | 0.069 | 1 | ARHGEF6 |
| 0.12544641 | 0.10700436 | 0.101 | 0.063 | 1 | VEZF1 |
| 0.12549399 | 0.11753832 | 0.116 | 0.085 | 1 | TTC39C |
| 0.12977207 | −0.1999177 | 0.154 | 0.201 | 1 | CYCS |
| 0.12985991 | 0.25618067 | 0.101 | 0.05 | 1 | CUL1 |
| 0.12992816 | −0.1713144 | 0.206 | 0.236 | 1 | DEK |
| 0.13001 | −0.2361509 | 0.24 | 0.289 | 1 | NACA2 |
| 0.13067919 | 0.29238821 | 0.202 | 0.138 | 1 | IL8 |
| 0.13105957 | 0.17477642 | 0.105 | 0.06 | 1 | CNOT4 |
| 0.13262465 | 0.11480664 | 0.116 | 0.082 | 1 | RNF114 |
| 0.13301797 | −0.235417 | 0.109 | 0.116 | 1 | TRGV9 |
| 0.13388617 | −0.1651033 | 0.266 | 0.286 | 1 | SAMD3 |
| 0.1394994 | −0.1893294 | 0.064 | 0.101 | 1 | C7orf73 |
| 0.1404209 | 0.22683897 | 0.124 | 0.075 | 1 | TRAT1 |
| 0.14050077 | 0.14832429 | 0.127 | 0.094 | 1 | SRRT |
| 0.14051167 | −0.1526593 | 0.217 | 0.233 | 1 | CD63 |
| 0.14059344 | 0.20083431 | 0.184 | 0.135 | 1 | FGFR1OP2 |
| 0.14060674 | −0.149526 | 0.105 | 0.138 | 1 | IFI27L2 |
| 0.14156621 | −0.1983486 | 0.071 | 0.101 | 1 | NOLC1 |
| 0.14216581 | 0.11119248 | 0.101 | 0.066 | 1 | MYSM1 |
| 0.14370506 | 0.18183962 | 0.172 | 0.116 | 1 | MAGOH |
| 0.14391719 | −0.2344474 | 0.097 | 0.132 | 1 | ARF6 |
| 0.14431285 | −0.1302194 | 0.112 | 0.119 | 1 | MDFIC |
| 0.14473556 | 0.12862868 | 0.15 | 0.101 | 1 | CSNK1A1 |
| 0.14529827 | −0.1284485 | 0.21 | 0.214 | 1 | BZW1 |
| 0.14631418 | 0.13516668 | 0.161 | 0.11 | 1 | BIRC6 |
| 0.14664416 | 0.29609597 | 0.105 | 0.057 | 1 | TAOK1 |
| 0.14790322 | 0.15037015 | 0.142 | 0.094 | 1 | SAFB2 |
| 0.14882215 | −0.1596301 | 0.169 | 0.179 | 1 | DDX18 |
| 0.15191612 | 0.11351174 | 0.116 | 0.079 | 1 | ORMDL3 |
| 0.15222754 | −0.1256366 | 0.225 | 0.233 | 1 | DSTN |
| 0.15245595 | −0.1453367 | 0.067 | 0.107 | 1 | DNAJB11 |
| 0.15316196 | −0.2165601 | 0.112 | 0.142 | 1 | COPE |
| 0.15471843 | 0.15000072 | 0.139 | 0.091 | 1 | ACBD5 |
| 0.15487094 | −0.1327055 | 0.195 | 0.217 | 1 | UXT |
| 0.15533562 | 0.11476435 | 0.221 | 0.164 | 1 | ARHGAP15 |
| 0.15561517 | −0.1308524 | 0.094 | 0.107 | 1 | VIMP |
| 0.15855509 | −0.1085665 | 0.978 | 0.978 | 1 | RPS23 |
| 0.16231824 | 0.2027275 | 0.116 | 0.066 | 1 | DYNLL2 |
| 0.16238749 | −0.2051503 | 0.124 | 0.151 | 1 | OCIAD1 |
| 0.16451608 | 0.16679467 | 0.142 | 0.113 | 1 | EIF5B |
| 0.16837507 | 0.1195688 | 0.607 | 0.525 | 1 | ACTG1 |
| 0.17191703 | 0.10157915 | 0.865 | 0.824 | 1 | SRGN |
| 0.17415112 | 0.11987961 | 0.3 | 0.252 | 1 | RORA |
| 0.17687915 | 0.11452903 | 0.124 | 0.082 | 1 | ITGB1BP1 |
| 0.17964634 | −0.1378494 | 0.101 | 0.107 | 1 | BOD1L1 |
| 0.18218081 | −0.221969 | 0.191 | 0.239 | 1 | IFITM3 |
| 0.18278682 | −0.1120869 | 0.24 | 0.236 | 1 | CELF2 |
| 0.19443083 | 0.19792485 | 0.105 | 0.06 | 1 | GTF2F1 |
| 0.19552566 | −0.2453047 | 0.169 | 0.208 | 1 | SAMD9L |
| 0.19759912 | −0.1519963 | 0.221 | 0.242 | 1 | PARK7 |
| 0.19903679 | 0.12411735 | 0.105 | 0.069 | 1 | GRPEL1 |
| 0.19920633 | −0.1677856 | 0.124 | 0.138 | 1 | VAMP5 |
| 0.20019332 | −0.1299533 | 0.236 | 0.264 | 1 | SELK |
| 0.20083107 | 0.11661754 | 0.131 | 0.094 | 1 | PPP1R18 |
| 0.20320362 | −0.1859238 | 0.127 | 0.151 | 1 | RBM17 |
| 0.2036916 | −0.1306148 | 0.105 | 0.126 | 1 | ATF7IP2 |
| 0.20490485 | −0.1182881 | 0.135 | 0.132 | 1 | SMARCE1 |
| 0.20555249 | 0.22376653 | 0.116 | 0.072 | 1 | NRBP1 |
| 0.20831252 | −0.1106296 | 0.146 | 0.16 | 1 | RNF19A |
| 0.20968309 | −0.1321374 | 0.996 | 1 | 1 | MALAT1 |
| 0.21137432 | −0.1163182 | 0.097 | 0.107 | 1 | CAMK4 |
| 0.21398292 | 0.14894375 | 0.127 | 0.082 | 1 | PSMD5-AS1 |
| 0.21590778 | −0.1335954 | 0.18 | 0.186 | 1 | TROVE2 |
| 0.21804605 | 0.10673442 | 0.157 | 0.116 | 1 | RHOC |
| 0.21957165 | 0.16222743 | 0.131 | 0.085 | 1 | PRKDC |
| 0.22426949 | 0.15569357 | 0.116 | 0.075 | 1 | SUPT5H |
| 0.22501044 | −0.1060397 | 0.277 | 0.286 | 1 | MBP |
| 0.22672752 | 0.12883999 | 0.101 | 0.063 | 1 | FIP1L1 |
| 0.22758381 | 0.13914882 | 0.191 | 0.145 | 1 | GOLGA4 |
| 0.23389146 | 0.16009813 | 0.131 | 0.091 | 1 | SPATA13 |
| 0.23921634 | −0.145432 | 0.199 | 0.22 | 1 | KIF2A |
| 0.23989578 | −0.1590793 | 0.109 | 0.126 | 1 | AC006129.2 |
| 0.24200954 | 0.15310408 | 0.139 | 0.094 | 1 | KARS |
| 0.24845793 | −0.1779248 | 0.142 | 0.167 | 1 | ISCU |
| 0.24857945 | 0.20199491 | 0.131 | 0.088 | 1 | TMEM170A |
| 0.24872731 | −0.1564901 | 0.21 | 0.23 | 1 | ATP5O |
| 0.25165481 | 0.10807903 | 0.127 | 0.091 | 1 | PHF20 |
| 0.25373086 | −0.1355551 | 0.124 | 0.129 | 1 | RALBP1 |
| 0.25517055 | 0.10297857 | 0.172 | 0.138 | 1 | RAB8B |
| 0.25861878 | −0.135361 | 0.124 | 0.126 | 1 | MX1 |
| 0.26001968 | −0.1764107 | 0.124 | 0.142 | 1 | SORL1 |
| 0.26046425 | 0.12561493 | 0.15 | 0.107 | 1 | OSTC |
| 0.2683719 | 0.22988996 | 0.101 | 0.063 | 1 | MESDC2 |
| 0.27076448 | 0.15196127 | 0.217 | 0.176 | 1 | KTN1 |
| 0.27421697 | 0.12285443 | 0.142 | 0.101 | 1 | AKIRIN1 |
| 0.27568629 | 0.10142611 | 0.135 | 0.119 | 1 | CCDC66 |
| 0.27639179 | −0.1090354 | 0.929 | 0.928 | 1 | RPL14 |
| 0.28155126 | 0.17193864 | 0.109 | 0.069 | 1 | ADSS |
| 0.28249284 | 0.17498541 | 0.135 | 0.088 | 1 | SAR1A |
| 0.28862842 | 0.12694553 | 0.124 | 0.085 | 1 | SNHG16 |
| 0.28956551 | −0.1265926 | 0.094 | 0.119 | 1 | SNRNP27 |
| 0.29215321 | 0.14807224 | 0.131 | 0.088 | 1 | PARP14 |
| 0.29757475 | 0.1741512 | 0.127 | 0.082 | 1 | C16orf54 |
| 0.29979404 | −0.1797331 | 0.116 | 0.129 | 1 | PAG1 |
| 0.30810752 | −0.1323932 | 0.228 | 0.258 | 1 | ATP5G3 |
| 0.31580373 | −0.113751 | 0.15 | 0.16 | 1 | ANAPC11 |
| 0.3196683 | −0.2214178 | 0.124 | 0.151 | 1 | PRSS23 |
| 0.32445128 | 0.12401005 | 0.109 | 0.079 | 1 | COQ10B |
| 0.3341337 | −0.1846736 | 0.109 | 0.138 | 1 | SNRPC |
| 0.33727939 | −0.1487209 | 0.12 | 0.135 | 1 | RBM26 |
| 0.34468986 | 0.12417171 | 0.105 | 0.069 | 1 | INPP4B |
| 0.35018689 | −0.1374198 | 0.213 | 0.226 | 1 | UQCR10 |
| 0.3576525 | −0.1175569 | 0.097 | 0.101 | 1 | COX17 |
| 0.35779439 | 0.1664594 | 0.12 | 0.079 | 1 | CCAR1 |
| 0.36270841 | −0.1377154 | 0.09 | 0.104 | 1 | TOX4 |
| 0.38078996 | −0.1150455 | 0.813 | 0.858 | 1 | S100A4 |
| 0.381727 | −0.1661245 | 0.086 | 0.101 | 1 | RP11-386114.4 |
| 0.39416953 | −0.10651 | 0.296 | 0.308 | 1 | PLEK |
| 0.39744675 | 0.12476058 | 0.154 | 0.113 | 1 | GPSM3 |
| 0.39787788 | 0.14682787 | 0.157 | 0.113 | 1 | H3F3C |
| 0.40176751 | −0.1044937 | 0.109 | 0.129 | 1 | MRP63 |
| 0.41180006 | −0.1274951 | 0.225 | 0.239 | 1 | MTRNR2L7 |
| 0.41732808 | 0.15666718 | 0.157 | 0.123 | 1 | RIOK3 |
| 0.41837471 | 0.19154726 | 0.112 | 0.075 | 1 | PRKAR2A |
| 0.4186041 | 0.14495302 | 0.221 | 0.17 | 1 | RBMX |
| 0.42164537 | 0.15217491 | 0.135 | 0.101 | 1 | MAF |
| 0.43031999 | 0.17859382 | 0.109 | 0.075 | 1 | DDX39B |
| 0.4403123 | −0.1424152 | 0.221 | 0.233 | 1 | BTN3A2 |
| 0.45886573 | −0.1433319 | 0.097 | 0.113 | 1 | CHMP5 |
| 0.4628816 | 0.11833004 | 0.109 | 0.075 | 1 | IDH2 |
| 0.4645011 | −0.1938808 | 0.071 | 0.101 | 1 | SBNO1 |
| 0.48568345 | −0.1001236 | 0.09 | 0.101 | 1 | ANKRD44 |
| 0.49050466 | 0.17114529 | 0.116 | 0.082 | 1 | VPS26A |
| 0.5276762 | −0.1415425 | 0.199 | 0.226 | 1 | UBXN4 |
| 0.55095142 | 0.13325111 | 0.116 | 0.085 | 1 | C1GALT1 |
| 0.56085484 | −0.1760559 | 0.079 | 0.104 | 1 | LINC00152 |
| 0.58769832 | 0.10497066 | 0.116 | 0.091 | 1 | AL592183.1 |
| 0.58915237 | −0.1325543 | 0.086 | 0.101 | 1 | ZNF22 |
| 0.59667357 | −0.1156846 | 0.116 | 0.129 | 1 | MTRNR2L13 |
| 0.61430359 | 0.1204126 | 0.109 | 0.082 | 1 | DAP3 |
| 0.61578958 | −0.1587071 | 0.127 | 0.151 | 1 | U2AF1 |
| 0.63282103 | 0.13530956 | 0.187 | 0.154 | 1 | FBXW7 |
| 0.63777047 | −0.1085296 | 0.303 | 0.302 | 1 | CD8A |
| 0.64841424 | 0.10668194 | 0.206 | 0.167 | 1 | NCOR1 |
| 0.65004484 | 0.11142668 | 0.142 | 0.113 | 1 | PSMA3 |
| 0.71047085 | −0.1202729 | 0.094 | 0.113 | 1 | SRSF4 |
| 0.71096655 | −0.1645657 | 0.105 | 0.129 | 1 | BRWD1 |
| 0.74652116 | −0.1627393 | 0.097 | 0.113 | 1 | UBE2Q2 |
| 0.74953413 | −0.1394617 | 0.082 | 0.101 | 1 | AMICA1 |

TABLE 7B
Genes differentially expressed between CTLs and Proliferating T Cells

| p_val | avg_logFC | pct.1 | pct.2 | p_val_adj | genes |
|---|---|---|---|---|---|
| 0 | −1.2155049 | 0.542 | 0.918 | 1E−300 | ACTG1 |
| 0 | −1.101727 | 0.546 | 0.912 | 1E−300 | GAPDH |
| 0 | −1.0592375 | 0.773 | 0.974 | 1E−300 | ACTB |
| 0 | 1.16743629 | 1 | 0.966 | 1E−300 | MALAT1 |
| 1.243E−289 | −1.7443685 | 0.045 | 0.526 | 1.666E−285 | STMN1 |
| 1.348E−274 | −1.5534563 | 0.006 | 0.419 | 1.806E−270 | KIAA0101 |
| 1.536E−254 | 1.03364238 | 0.861 | 0.596 | 2.057E−250 | TXNIP |
| 2.103E−250 | −1.3154383 | 0.165 | 0.65 | 2.817E−246 | COTL1 |
| 1.269E−248 | −1.3460759 | 0.338 | 0.734 | 1.7E−244 | HMGB2 |
| 3.292E−235 | −1.5144128 | 0.007 | 0.373 | 4.411E−231 | MKI67 |
| 1.259E−226 | −0.7445706 | 0.772 | 0.96 | 1.687E−222 | PFN1 |
| 1.072E−179 | −1.1130449 | 0.003 | 0.286 | 1.437E−175 | TYMS |
| 1.957E−174 | −1.3572404 | 0.168 | 0.522 | 2.622E−170 | TUBA1B |
| 4.238E−173 | −1.4003043 | 0.357 | 0.63 | 5.678E−169 | HIST1H4C |
| 1.109E−170 | −0.7949428 | 0.389 | 0.799 | 1.486E−166 | CORO1A |
| 3.528E−167 | −1.2990951 | 0.01 | 0.296 | 4.726E−163 | CENPF |
| 3.454E−160 | −1.2523855 | 0.072 | 0.413 | 4.627E−156 | TUBB |
| 3.804E−160 | −1.1813836 | 0.042 | 0.369 | 5.096E−156 | PCNA |
| 1.921E−157 | 1.38282932 | 0.48 | 0.162 | 2.573E−153 | IL7R |
| 1.627E−153 | −1.557078 | 0.03 | 0.317 | 2.18E−149 | HIST1H1B |
| 6.594E−153 | −1.1062818 | 0.17 | 0.532 | 8.833E−149 | H2AFZ |
| 3.013E−151 | −1.199822 | 0.003 | 0.244 | 4.036E−147 | TOP2A |
| 9.305E−150 | −0.6864528 | 0.576 | 0.874 | 1.246E−145 | CFL1 |
| 1.357E−146 | −1.0231072 | 0.004 | 0.243 | 1.818E−142 | RRM2 |
| 1.126E−145 | −1.1439653 | 0.128 | 0.482 | 1.508E−141 | DUT |
| 1.178E−143 | −0.7504915 | 0.551 | 0.82 | 1.578E−139 | HNRNPA2B1 |
| 9.204E−141 | −0.9312293 | 0.179 | 0.549 | 1.233E−136 | HMGN2 |
| 1.099E−138 | −0.9608521 | 0.014 | 0.268 | 1.473E−134 | MCM4 |
| 9.428E−138 | −0.8337514 | 0.242 | 0.622 | 1.263E−133 | SLC25A5 |
| 4.389E−133 | −0.9079165 | 0.001 | 0.212 | 5.88E−129 | BIRC5 |
| 5.837E−130 | −0.808922 | 0.104 | 0.426 | 7.819E−126 | IDH2 |
| 9.849E−130 | −1.0231209 | 0.102 | 0.428 | 1.319E−125 | SMC4 |
| 6.181E−128 | −0.680727 | 0.291 | 0.664 | 8.28E−124 | ACTR3 |
| 1.717E−127 | 0.82978484 | 0.614 | 0.398 | 2.3E−123 | TSC22D3 |
| 2.66E−126 | −0.5648535 | 0.682 | 0.912 | 3.563E−122 | ARHGDIB |
| 1.708E−125 | 0.58451961 | 0.814 | 0.73 | 2.287E−121 | DDX5 |
| 3.653E−125 | 0.78593828 | 0.688 | 0.529 | 4.894E−121 | JUNB |
| 1.069E−123 | −0.8005187 | 0.182 | 0.532 | 1.432E−119 | SH2D1A |
| 3.096E−123 | −1.2965907 | 0.018 | 0.246 | 4.147E−119 | HIST1H2AJ |
| 6.202E−121 | −0.9324348 | 0.278 | 0.61 | 8.308E−117 | HIST1H1D |
| 3.197E−120 | −0.7855736 | 0.124 | 0.448 | 4.282E−116 | HNRNPF |
| 6.416E−120 | −0.765248 | 0.326 | 0.67 | 8.594E−116 | ENO1 |
| 7.982E−118 | −0.7482524 | 0.213 | 0.56 | 1.069E−113 | CBX3 |
| 1.441E−116 | −0.6735872 | 0.231 | 0.577 | 1.931E−112 | PPP1CA |
| 3.511E−115 | −0.7721684 | 0.178 | 0.513 | 4.704E−111 | PSMA4 |
| 1.224E−114 | −0.7132413 | 0.168 | 0.498 | 1.64E−110 | ATP5C1 |
| 2.919E−113 | −0.6268982 | 0.426 | 0.761 | 3.91E−109 | ARPC1B |
| 3.712E−113 | −0.6923048 | 0.268 | 0.622 | 4.973E−109 | COX8A |
| 4.005E−113 | −0.6339097 | 0.268 | 0.616 | 5.365E−109 | PSMB8 |
| 5.914E−113 | −0.6927694 | 0.344 | 0.694 | 7.922E−109 | ARPC5 |
| 1.808E−112 | −0.675256 | 0.304 | 0.652 | 2.422E−108 | LDHB |
| 2.141E−110 | −0.9087718 | 0.142 | 0.452 | 2.869E−106 | TMPO |
| 7.107E−110 | 0.52502525 | 0.963 | 0.916 | 9.52E−106 | RPS27 |
| 2.637E−109 | −0.7768782 | 0.004 | 0.194 | 3.532E−105 | TK1 |
| 6.527E−109 | −0.7571602 | 0.104 | 0.399 | 8.744E−105 | YWHAE |
| 6.724E−108 | −0.714291 | 0.174 | 0.496 | 9.007E−104 | COX5A |
| 8.335E−106 | −0.62925 | 0.309 | 0.65 | 1.117E−101 | PSME2 |
| 2.262E−105 | −0.8887783 | 0.011 | 0.21 | 3.03E−101 | NUSAP1 |
| 3.259E−105 | −0.7768973 | 0.026 | 0.242 | 4.366E−101 | MCM3 |
| 6.281E−105 | −0.7278599 | 0.128 | 0.426 | 8.414E−101 | CACYBP |
| 7.052E−105 | −0.7126788 | 0.148 | 0.457 | 9.447E−101 | SHFM1 |
| 5.51E−104 | −0.6677572 | 0.248 | 0.582 | 7.381E−100 | ATP5G3 |
| 9.273E−104 | −0.5000926 | 0.749 | 0.922 | 1.242E−99 | MYL6 |
| 1.888E−103 | 0.63647806 | 0.664 | 0.548 | 2.53E−99 | RPL9 |
| 2.807E−102 | 0.72109812 | 0.57 | 0.428 | 3.7597E−98 | ZFP36L2 |
| 4.56E−102 | −0.6738237 | 0.083 | 0.345 | 6.109E−98 | MIR4435-1HG |
| 5.847E−102 | −0.7775932 | 0.12 | 0.414 | 7.833E−98 | NASP |
| 6.285E−102 | −0.6598485 | 0.264 | 0.592 | 8.419E−98 | ANP32B |
| 1.041E−101 | −0.6206674 | 0.395 | 0.716 | 1.3948E−97 | SUB1 |
| 3.562E−101 | −0.7424586 | 0.22 | 0.536 | 4.7713E−97 | DEK |
| 7.718E−101 | −0.7852373 | 0 | 0.158 | 1.0338E−96 | FCGR3A |
| 8.838E−101 | −0.7710081 | 0.02 | 0.227 | 1.1839E−96 | MCM5 |
| 2.088E−100 | −0.7951722 | 0.004 | 0.179 | 2.7966E−96 | TPX2 |
| 5.908E−100 | −0.6896825 | 0.009 | 0.192 | 7.914E−96 | CDCA7 |
| 1.591E−99 | −0.7748497 | 0.07 | 0.33 | 2.1313E−95 | RPA3 |
| 3.2599E−98 | −0.6989108 | 0.058 | 0.303 | 4.3669E−94 | HMGA1 |
| 3.5622E−98 | −0.7705557 | 0.04 | 0.27 | 4.772E−94 | EZH2 |
| 5.966E−98 | −0.7707007 | 0.035 | 0.259 | 7.9921E−94 | MCM7 |
| 9.4485E−98 | −0.6199111 | 0.152 | 0.442 | 1.2657E−93 | ATP5F1 |
| 1.4855E−97 | −0.7426415 | 0.008 | 0.189 | 1.99E−93 | MLF1IP |
| 5.8711E−97 | −0.6237097 | 0.211 | 0.522 | 7.865E−93 | CAPZA1 |
| 8.3476E−97 | −0.6955692 | 0.132 | 0.419 | 1.1182E−92 | GBP1 |
| 1.7264E−96 | −0.587003 | 0.29 | 0.612 | 2.3127E−92 | IFI16 |
| 2.3837E−96 | −0.6685547 | 0.07 | 0.314 | 3.1932E−92 | TXNDC17 |
| 3.3124E−96 | −0.6297285 | 0.196 | 0.5 | 4.4373E−92 | CALM3 |
| 3.4382E−96 | −0.6433823 | 0.12 | 0.398 | 4.6058E−92 | H2AFV |
| 1.3155E−95 | −0.6110226 | 0.319 | 0.642 | 1.7623E−91 | COX7B |
| 1.4413E−95 | −0.6066578 | 0.225 | 0.538 | 1.9307E−91 | ATP5A1 |
| 2.4067E−95 | 0.71025811 | 0.649 | 0.486 | 3.2241E−91 | NFKBIA |
| 2.641E−95 | −0.6669937 | 0.065 | 0.306 | 3.5378E−91 | MT1E |
| 8.1156E−95 | −0.5759597 | 0.189 | 0.487 | 1.0872E−90 | PSMB3 |
| 9.1863E−95 | −0.6024502 | 0.13 | 0.405 | 1.2306E−90 | PPM1G |
| 3.917E−94 | −0.7796779 | 0.228 | 0.515 | 5.2473E−90 | HIST1H1C |
| 5.5775E−94 | −0.7022 | 0.044 | 0.268 | 7.4717E−90 | CD38 |
| 5.0096E−93 | −0.6492474 | 0.116 | 0.385 | 6.7108E−89 | CCDC167 |
| 7.1145E−93 | −0.6407166 | 0.352 | 0.66 | 9.5306E−89 | HMGB1 |
| 7.7316E−93 | −0.5843505 | 0.24 | 0.549 | 1.0357E−88 | PSMA6 |
| 1.5531E−91 | 0.60822342 | 0.663 | 0.546 | 2.0806E−87 | DUSP1 |
| 3.606E−91 | −0.6298033 | 0.106 | 0.364 | 4.8306E−87 | PSMA5 |
| 5.4176E−91 | −0.6873579 | 0.22 | 0.51 | 7.2574E−87 | RAN |
| 2.7967E−90 | −0.6689231 | 0.144 | 0.424 | 3.7465E−86 | SNRPG |
| 6.2854E−90 | −0.6486636 | 0.038 | 0.244 | 8.4199E−86 | PRDX3 |
| 1.9802E−89 | −0.7419939 | 0.156 | 0.434 | 2.6527E−85 | HSPD1 |
| 1.2242E−88 | −0.5010872 | 0.331 | 0.636 | 1.64E−84 | COX6B1 |
| 2.0567E−88 | 1.07843751 | 0.398 | 0.2 | 2.7551E−84 | KLRB1 |
| 3.069E−88 | −0.6028412 | 0.043 | 0.252 | 4.1112E−84 | SKA2 |
| 4.5693E−88 | −0.6152573 | 0.086 | 0.33 | 6.1211E−84 | NDUFB3 |
| 2.7995E−87 | −0.5005021 | 0.3 | 0.599 | 3.7502E−83 | CAPZB |
| 3.663E−87 | −0.5458039 | 0.144 | 0.403 | 4.9069E−83 | ATP5J2 |
| 1.3556E−86 | 0.60383648 | 0.644 | 0.537 | 1.8159E−82 | KLF6 |
| 6.6966E−86 | 0.73344553 | 0.503 | 0.338 | 8.9708E−82 | PIK3R1 |
| 2.9283E−85 | −0.613021 | 0.15 | 0.421 | 3.9228E−81 | HNRNPD |
| 5.3619E−85 | −0.5229692 | 0.191 | 0.466 | 7.1829E−81 | ATP5B |
| 6.6272E−85 | −0.7095957 | 0.026 | 0.215 | 8.8778E−81 | SMC2 |
| 1.5912E−84 | −0.6535606 | 0.029 | 0.218 | 2.1315E−80 | PTTG1 |
| 1.7792E−84 | −0.6009758 | 0.304 | 0.595 | 2.3834E−80 | NUCKS1 |
| 3.001E−84 | −0.5622579 | 0.097 | 0.334 | 4.0201E−80 | DECR1 |
| 3.2103E−84 | −0.5217112 | 0.128 | 0.378 | 4.3006E−80 | SNRPE |
| 8.555E−84 | −0.5296298 | 0.354 | 0.66 | 1.146E−79 | CHCHD2 |
| 1.3033E−83 | −0.5324115 | 0.059 | 0.264 | 1.7459E−79 | HN1 |
| 1.9788E−83 | −0.6197065 | 0.088 | 0.325 | 2.6508E−79 | CCT5 |
| 2.1392E−83 | −0.618152 | 0.003 | 0.144 | 2.8656E−79 | CCNA2 |
| 3.7163E−83 | −0.5509658 | 0.066 | 0.28 | 4.9783E−79 | CLTA |
| 4.0413E−83 | −0.5262491 | 0.134 | 0.382 | 5.4138E−79 | RBX1 |
| 6.6491E−83 | −0.6266495 | 0.177 | 0.441 | 8.9071E−79 | ANXA2 |
| 2.3368E−82 | −0.5188237 | 0.11 | 0.342 | 3.1303E−78 | LINC00152 |
| 8.7853E−82 | −0.5238835 | 0.187 | 0.46 | 1.1769E−77 | NDUFB1 |
| 1.0436E−81 | −0.6409753 | 0.003 | 0.146 | 1.398E−77 | CLSPN |
| 1.5319E−81 | 0.79782047 | 0.41 | 0.242 | 2.0522E−77 | MAN1A2 |
| 2.0474E−81 | −0.626121 | 0.04 | 0.236 | 2.7427E−77 | LMNB1 |
| 2.5503E−81 | 0.50912498 | 0.628 | 0.55 | 3.4164E−77 | RPL21 |
| 1.2117E−80 | −0.6014802 | 0.004 | 0.144 | 1.6232E−76 | KIF11 |
| 1.2757E−80 | −0.520083 | 0.363 | 0.664 | 1.7089E−76 | COX6A1 |
| 1.9778E−79 | −0.5039514 | 0.284 | 0.574 | 2.6495E−75 | UQCRQ |
| 2.773E−79 | −0.5298718 | 0.369 | 0.662 | 3.7148E−75 | PPIA |
| 3.6415E−79 | 0.63890244 | 0.741 | 0.52 | 4.8782E−75 | GNLY |
| 1.5239E−78 | −0.6205689 | 0.06 | 0.268 | 2.0415E−74 | CBX5 |
| 1.6859E−78 | −0.5742631 | 0.204 | 0.48 | 2.2584E−74 | TXN |
| 3.1353E−78 | −0.5633287 | 0.152 | 0.41 | 4.2001E−74 | ERH |
| 3.136E−78 | −0.5073439 | 0.266 | 0.55 | 4.201E−74 | DBI |
| 3.1885E−78 | 0.58912814 | 0.739 | 0.665 | 4.2713E−74 | JUN |
| 4.6636E−78 | −0.6820755 | 0.02 | 0.188 | 6.2474E−74 | HELLS |
| 1.1285E−77 | 0.59447028 | 0.527 | 0.4 | 1.5117E−73 | BTG1 |
| 4.5994E−77 | −0.5782616 | 0.154 | 0.41 | 6.1614E−73 | SNRPD1 |
| 1.0355E−76 | −0.7713767 | 0.054 | 0.26 | 1.3871E−72 | HIST1H2AM |
| 1.1296E−76 | −0.5237172 | 0.232 | 0.505 | 1.5132E−72 | XRCC5 |
| 4.2774E−76 | −0.5572783 | 0.223 | 0.498 | 5.73E−72 | SEPT6 |
| 5.0386E−76 | −0.5615733 | 0.03 | 0.206 | 6.7497E−72 | RRM1 |
| 5.7028E−76 | −0.5571753 | 0.096 | 0.319 | 7.6394E−72 | PSMB2 |
| 6.1692E−76 | −0.5955222 | 0.176 | 0.439 | 8.2643E−72 | ANP32E |
| 8.891E−76 | −0.6209042 | 0 | 0.121 | 1.191E−71 | ASPM |
| 1.2E−75 | −0.6013482 | 0.029 | 0.202 | 1.6075E−71 | MCM6 |
| 1.254E−75 | −0.5041307 | 0.126 | 0.356 | 1.6798E−71 | CCT8 |
| 1.896E−75 | −0.5749433 | 0.168 | 0.424 | 2.5399E−71 | CNN2 |
| 5.353E−75 | −0.5114392 | 0.145 | 0.388 | 7.1709E−71 | LSM3 |
| 6.0766E−75 | −0.5880642 | 0.001 | 0.124 | 8.1403E−71 | FAM111B |
| 6.9268E−74 | −0.6034791 | 0.016 | 0.171 | 9.2791E−70 | PAICS |
| 1.2733E−73 | −0.5450921 | 0.126 | 0.36 | 1.7058E−69 | NDUFB6 |
| 1.1255E−72 | −0.5005029 | 0.104 | 0.322 | 1.5077E−68 | NMI |
| 3.6213E−72 | 0.58160804 | 0.642 | 0.514 | 4.8511E−68 | ID2 |
| 3.8263E−72 | −0.6813674 | 0.071 | 0.284 | 5.1257E−68 | CD27 |
| 1.2245E−71 | −0.5143117 | 0.12 | 0.344 | 1.6403E−67 | DYNLL1 |
| 2.1091E−71 | −0.509305 | 0.046 | 0.222 | 2.8254E−67 | MRPL13 |
| 4.3586E−71 | −0.6177665 | 0.014 | 0.166 | 5.8388E−67 | CHI3L2 |
| 4.5415E−71 | −0.5402733 | 0.03 | 0.197 | 6.0837E−67 | NUDT1 |
| 3.8084E−70 | −0.5782633 | 0.014 | 0.156 | 5.1017E−66 | FANCI |
| 2.0118E−69 | −0.5448211 | 0.119 | 0.343 | 2.695E−65 | H2AFY |
| 1.3887E−68 | 0.56428233 | 0.668 | 0.576 | 1.8603E−64 | SYNE2 |
| 3.3883E−68 | −0.5660157 | 0.146 | 0.379 | 4.5389E−64 | USP1 |
| 5.0753E−68 | −0.500794 | 0.42 | 0.682 | 6.7989E−64 | VIM |
| 1.3147E−67 | −0.6029523 | 0.098 | 0.317 | 1.7612E−63 | LIMSI |
| 1.3415E−67 | −0.5261274 | 0.036 | 0.202 | 1.7971E−63 | CARHSP1 |
| 1.6201E−67 | −0.5598194 | 0.034 | 0.2 | 2.1703E−63 | CKS2 |
| 3.2873E−67 | −0.5007731 | 0.008 | 0.138 | 4.4037E−63 | CENPW |
| 8.7239E−67 | −0.5376488 | 0.196 | 0.439 | 1.1687E−62 | ANXA5 |
| 3.623E−66 | −0.505496 | 0.14 | 0.361 | 4.8533E−62 | UCP2 |
| 3.9373E−66 | 0.66881995 | 0.41 | 0.272 | 5.2744E−62 | ANKRD12 |
| 1.113E−65 | −0.558509 | 0.09 | 0.295 | 1.491E−61 | DENND2D |
| 1.3928E−65 | −0.5126764 | 0.079 | 0.268 | 1.8658E−61 | SLBP |
| 4.0853E−65 | −0.5995948 | 0.272 | 0.516 | 5.4726E−61 | HIST1H1E |
| 7.6514E−65 | −0.6225926 | 0.008 | 0.134 | 1.025E−60 | CENPE |
| 2.4224E−64 | −0.5252996 | 0.23 | 0.481 | 3.2451E−60 | SNRPB |
| 9.7969E−64 | −0.5116166 | 0.148 | 0.372 | 1.3124E−59 | TPM4 |
| 1.2427E−63 | 0.54013645 | 0.593 | 0.48 | 1.6647E−59 | CXCR4 |
| 2.6024E−62 | −0.5315922 | 0.062 | 0.239 | 3.4862E−58 | DNAJC9 |
| 3.8246E−62 | 0.60807869 | 0.676 | 0.59 | 5.1234E−58 | MTRNR2L12 |
| 3.2734E−61 | −0.5038619 | 0.002 | 0.11 | 4.3851E−57 | HIST1H3B |
| 4.685E−61 | −0.5045527 | 0 | 0.102 | 6.276E−57 | DLGAP5 |
| 7.3305E−61 | 0.54938276 | 0.549 | 0.435 | 9.8199E−57 | ZFP36 |
| 1.222E−59 | 0.52944992 | 0.746 | 0.686 | 1.6369E−55 | MTRNR2L8 |
| 5.383E−59 | −0.5012348 | 0.036 | 0.186 | 7.2111E−55 | PRIM1 |
| 2.6112E−56 | 0.76828309 | 0.18 | 0.044 | 3.4979E−52 | MYBL1 |
| 9.8279E−55 | 0.58610007 | 0.374 | 0.266 | 1.3166E−50 | XIST |
| 1.5625E−54 | −0.520214 | 0.084 | 0.262 | 2.0931E−50 | TIGIT |
| 1.1669E−53 | 0.57947185 | 0.398 | 0.296 | 1.5631E−49 | SYNE1 |
| 1.8223E−53 | 0.5331925 | 0.37 | 0.276 | 2.4411E−49 | KMT2E |
| 1.5249E−52 | −0.8322223 | 0.104 | 0.288 | 2.0428E−48 | IFI27 |
| 1.0755E−51 | −0.5308362 | 0.038 | 0.182 | 1.4408E−47 | ATAD2 |
| 4.1156E−51 | 0.50110424 | 0.754 | 0.702 | 5.5132E−47 | MTRNR2L1 |
| 2.1293E−48 | −0.5279877 | 0.018 | 0.135 | 2.8524E−44 | FBXO5 |
| 1.0619E−47 | 0.69348905 | 0.212 | 0.086 | 1.4225E−43 | KLRG1 |
| 6.6409E−47 | 0.67620135 | 0.302 | 0.158 | 8.8962E−43 | CTSW |
| 8.5055E−46 | 0.55561423 | 0.284 | 0.198 | 1.1394E−41 | TOB1 |
| 1.5528E−45 | 0.59644706 | 0.276 | 0.188 | 2.0802E−41 | TRGC1 |
| 1.3519E−39 | 0.57876999 | 0.316 | 0.208 | 1.811E−35 | TYROBP |
| 3.9061E−38 | 0.57521697 | 0.209 | 0.107 | 5.2326E−34 | DDIT4 |
| 2.1991E−36 | 0.56353316 | 0.216 | 0.126 | 2.9459E−32 | FBXW7 |
| 2.7322E−36 | 0.53627895 | 0.223 | 0.132 | 3.66E−32 | CSNK1G3 |
| 6.6693E−36 | 0.75848367 | 0.232 | 0.104 | 8.9341E−32 | TRDC |
| 6.1901E−35 | 0.50858444 | 0.27 | 0.175 | 8.2923E−31 | SBDS |
| 3.8475E−29 | 0.53686963 | 0.237 | 0.136 | 5.154E−25 | LTB |
| 1.963E−27 | 0.5345181 | 0.164 | 0.078 | 2.6296E−23 | ZNF331 |

TABLE 7C
Genes differentially expressed between the
sub-clusters found in Proliferating T Cells

| p_val | avg_logFC | pct.1 | pct.2 | p_val_adj | cluster | gene |
|---|---|---|---|---|---|---|
| 7.684E−236 | 1.16039329 | 0.996 | 0.769 | 1.305E−231 | 0 | CCL5 |
| 1.3421E−89 | 0.69714647 | 0.905 | 0.65 | 2.2789E−85 | 0 | NKG7 |
| 8.2514E−61 | 0.65970632 | 0.703 | 0.404 | 1.4011E−56 | 0 | CD8A |
| 2.2693E−57 | 0.77927698 | 0.627 | 0.365 | 3.8532E−53 | 0 | DUSP2 |
| 2.4899E−53 | 0.7723361 | 0.608 | 0.366 | 4.2279E−49 | 0 | TNFAIP3 |
| 1.0846E−52 | 0.78444483 | 0.59 | 0.327 | 1.8417E−48 | 0 | FGFBP2 |
| 5.4312E−39 | 0.61509701 | 0.623 | 0.409 | 9.2222E−35 | 0 | GZMH |
| 8.6968E−39 | 0.50681862 | 0.787 | 0.642 | 1.4767E−34 | 0 | CLEC2B |
| 3.2122E−35 | 0.5123612 | 0.629 | 0.421 | 5.4543E−31 | 0 | CST7 |
| 1.0164E−34 | 0.53117746 | 0.748 | 0.603 | 1.7259E−30 | 0 | JUN |
| 9.6111E−32 | 0.60265623 | 0.441 | 0.25 | 1.632E−27 | 0 | CD8B |
| 1.4955E−31 | 0.64626673 | 0.511 | 0.315 | 2.5394E−27 | 0 | RGS1 |
| 1.7946E−28 | 0.55794341 | 0.54 | 0.361 | 3.0472E−24 | 0 | ZFP36 |
| 2.7364E−24 | 0.55529402 | 0.348 | 0.187 | 4.6464E−20 | 0 | TGFBR3 |
| 2.0026E−22 | 0.50383725 | 0.473 | 0.319 | 3.4005E−18 | 0 | DNAJB1 |
| 2.2413E−20 | 0.5634587 | 0.395 | 0.261 | 3.8058E−16 | 0 | PDCD4 |
| 1.1396E−15 | 0.72890883 | 0.385 | 0.272 | 1.935E−11 | 0 | CMC1 |
| 2.7246E−15 | 0.50430128 | 0.351 | 0.223 | 4.6264E−11 | 0 | IFNG |
| 1.41E−198 | 1.27749391 | 0.869 | 0.333 | 2.394E−194 | 1 | TUBA1B |
| 6.586E−155 | 1.64909984 | 0.861 | 0.508 | 1.118E−150 | 1 | HIST1H4C |
| 7.208E−147 | 0.8996297 | 0.939 | 0.615 | 1.224E−142 | 1 | HMGB2 |
| 7.992E−135 | 1.15930072 | 0.519 | 0.092 | 1.357E−130 | 1 | RRM2 |
| 2.216E−133 | 1.51991603 | 0.516 | 0.099 | 3.762E−129 | 1 | HIST1H2AJ |
| 4.232E−132 | 0.98898929 | 0.78 | 0.32 | 7.187E−128 | 1 | DUT |
| 6.531E−130 | 1.0015806 | 0.798 | 0.389 | 1.109E−125 | 1 | H2AFZ |
| 2.494E−123 | 1.08545715 | 0.684 | 0.261 | 4.234E−119 | 1 | TUBB |
| 1.246E−120 | 0.86289023 | 0.818 | 0.364 | 2.116E−116 | 1 | STMN1 |
| 1.448E−112 | 0.99105402 | 0.644 | 0.216 | 2.459E−108 | 1 | PCNA |
| 1.725E−110 | 1.47963654 | 0.56 | 0.174 | 2.929E−106 | 1 | HIST1H1B |
| 5.168E−107 | 0.95037185 | 0.715 | 0.304 | 8.775E−103 | 1 | TMPO |
| 2.998E−106 | 0.95753233 | 0.541 | 0.14 | 5.091E−102 | 1 | TYMS |
| 1.202E−104 | 0.62422885 | 0.95 | 0.75 | 2.042E−100 | 1 | HNRNPA2B1 |
| 7.7913E−98 | 0.92485495 | 0.625 | 0.214 | 1.323E−93 | 1 | MKI67 |
| 4.6631E−97 | 0.94103006 | 0.664 | 0.285 | 7.9179E−93 | 1 | KIAA0101 |
| 1.5738E−94 | 1.1586774 | 0.474 | 0.118 | 2.6724E−90 | 1 | TOP2A |
| 1.0533E−92 | 0.92905135 | 0.324 | 0.037 | 1.7886E−88 | 1 | CCNA2 |
| 1.2589E−89 | 0.54341549 | 0.962 | 0.851 | 2.1375E−85 | 1 | PTMA |
| 3.0748E−86 | 0.52561476 | 0.973 | 0.872 | 5.221E−82 | 1 | GAPDH |
| 3.956E−83 | 0.69673417 | 0.836 | 0.564 | 6.7172E−79 | 1 | HMGB1 |
| 7.5198E−82 | 0.78499988 | 0.496 | 0.143 | 1.2769E−77 | 1 | MCM4 |
| 8.2272E−79 | 0.75206276 | 0.481 | 0.139 | 1.397E−74 | 1 | MCM7 |
| 1.3141E−76 | 0.83502772 | 0.654 | 0.301 | 2.2314E−72 | 1 | SMC4 |
| 4.9055E−76 | 0.73371915 | 0.742 | 0.411 | 8.3295E−72 | 1 | DEK |
| 1.8657E−75 | 0.7532881 | 0.728 | 0.395 | 3.1679E−71 | 1 | RAN |
| 1.673E−74 | 0.8079511 | 0.442 | 0.124 | 2.8407E−70 | 1 | LMNB1 |
| 7.414E−73 | 0.84904238 | 0.412 | 0.11 | 1.2589E−68 | 1 | SMC2 |
| 1.0015E−72 | 0.8112238 | 0.259 | 0.029 | 1.7005E−68 | 1 | HIST1H3B |
| 8.7361E−71 | 0.84355027 | 0.394 | 0.101 | 1.4834E−66 | 1 | NUSAP1 |
| 2.9253E−70 | 0.74286146 | 0.383 | 0.094 | 4.9672E−66 | 1 | MLF1IP |
| 1.2558E−68 | 0.80959098 | 0.634 | 0.322 | 2.1323E−64 | 1 | HSPD1 |
| 3.3661E−68 | 0.69804456 | 0.379 | 0.093 | 5.7157E−64 | 1 | TK1 |
| 3.4936E−67 | 0.74848975 | 0.587 | 0.257 | 5.9321E−63 | 1 | USP1 |
| 2.401E−66 | 0.77926738 | 0.386 | 0.106 | 4.0769E−62 | 1 | RRM1 |
| 5.6418E−66 | 0.56783733 | 0.891 | 0.71 | 9.5797E−62 | 1 | HSP90AA1 |
| 2.0417E−65 | 0.77057385 | 0.299 | 0.056 | 3.4668E−61 | 1 | KIF11 |
| 2.1863E−65 | 0.68198443 | 0.648 | 0.319 | 3.7124E−61 | 1 | ANP32E |
| 1.5468E−63 | 0.69766208 | 0.617 | 0.294 | 2.6265E−59 | 1 | NASP |
| 4.7011E−61 | 0.58797496 | 0.812 | 0.589 | 7.9824E−57 | 1 | ENO1 |
| 8.9825E−61 | 0.77504726 | 0.304 | 0.065 | 1.5252E−56 | 1 | CLSPN |
| 1.0511E−60 | 0.70768519 | 0.29 | 0.055 | 1.7848E−56 | 1 | FBXO5 |
| 1.0605E−59 | 0.77196602 | 0.245 | 0.038 | 1.8007E−55 | 1 | HIST1H2AH |
| 1.2081E−59 | 0.7658141 | 0.34 | 0.086 | 2.0514E−55 | 1 | TPX2 |
| 1.241E−59 | 0.72808976 | 0.381 | 0.111 | 2.1072E−55 | 1 | BIRC5 |
| 6.8225E−58 | 0.67873725 | 0.389 | 0.119 | 1.1585E−53 | 1 | RANBP1 |
| 1.756E−57 | 0.75650726 | 0.229 | 0.033 | 2.9817E−53 | 1 | DLGAP5 |
| 1.4988E−56 | 0.71663532 | 0.35 | 0.096 | 2.545E−52 | 1 | ATAD2 |
| 7.2346E−56 | 0.63954787 | 0.223 | 0.031 | 1.2284E−51 | 1 | DTL |
| 1.1217E−55 | 0.6863784 | 0.642 | 0.366 | 1.9046E−51 | 1 | LDHA |
| 2.6736E−55 | 0.72128062 | 0.328 | 0.088 | 4.5398E−51 | 1 | PAICS |
| 8.0012E−55 | 0.63617036 | 0.263 | 0.05 | 1.3586E−50 | 1 | FAM111B |
| 1.3579E−54 | 0.6348237 | 0.256 | 0.049 | 2.3057E−50 | 1 | FEN1 |
| 9.7834E−54 | 0.70599339 | 0.24 | 0.042 | 1.6612E−49 | 1 | HIST1H2AL |
| 2.7308E−53 | 0.65420567 | 0.41 | 0.143 | 4.6368E−49 | 1 | DNAJC9 |
| 1.5754E−52 | 0.7073908 | 0.407 | 0.144 | 2.675E−48 | 1 | MCM3 |
| 5.4124E−52 | 0.6502625 | 0.28 | 0.064 | 9.1902E−48 | 1 | CENPW |
| 4.8984E−51 | 0.64636755 | 0.445 | 0.173 | 8.3175E−47 | 1 | CBX5 |
| 6.2195E−51 | 0.55310844 | 0.769 | 0.498 | 1.0561E−46 | 1 | NUCKS1 |
| 1.6351E−50 | 0.64123294 | 0.207 | 0.03 | 2.7764E−46 | 1 | HMMR |
| 9.4698E−49 | 0.62445247 | 0.596 | 0.318 | 1.608E−44 | 1 | SNRPG |
| 2.8827E−48 | 0.64300604 | 0.385 | 0.137 | 4.8949E−44 | 1 | MCM5 |
| 3.4325E−48 | 0.8209126 | 0.418 | 0.168 | 5.8283E−44 | 1 | HIST1H2AM |
| 1.1424E−47 | 0.64733788 | 0.476 | 0.215 | 1.9397E−43 | 1 | HMGA1 |
| 1.256E−47 | 0.62143671 | 0.342 | 0.108 | 2.1327E−43 | 1 | CDCA7 |
| 4.5273E−47 | 0.65608809 | 0.279 | 0.072 | 7.6874E−43 | 1 | HIST1H4E |
| 2.5671E−46 | 0.61345564 | 0.309 | 0.091 | 4.3589E−42 | 1 | HNRNPAB |
| 5.1915E−46 | 0.64559272 | 0.735 | 0.542 | 8.8151E−42 | 1 | HSP90AB1 |
| 6.6052E−46 | 0.58220799 | 0.352 | 0.118 | 1.1216E−41 | 1 | MCM6 |
| 1.1496E−45 | 0.61564288 | 0.493 | 0.226 | 1.952E−41 | 1 | CCT5 |
| 2.0727E−45 | 0.57365519 | 0.605 | 0.336 | 3.5194E−41 | 1 | TPI1 |
| 3.3483E−45 | 0.70550841 | 0.663 | 0.424 | 5.6855E−41 | 1 | HIST1H1E |
| 4.463E−45 | 0.5575602 | 0.203 | 0.035 | 7.5782E−41 | 1 | CDK1 |
| 2.393E−44 | 0.61324622 | 0.292 | 0.084 | 4.0633E−40 | 1 | GMNN |
| 3.9735E−44 | 0.51178799 | 0.749 | 0.496 | 6.7469E−40 | 1 | ANP32B |
| 4.776E−44 | 0.64889284 | 0.37 | 0.136 | 8.1096E−40 | 1 | PTTG1 |
| 5.2497E−43 | 0.56215852 | 0.54 | 0.274 | 8.9141E−39 | 1 | SIVA1 |
| 5.6656E−43 | 0.69100077 | 0.457 | 0.2 | 9.6201E−39 | 1 | CENPF |
| 9.4292E−43 | 0.6081652 | 0.344 | 0.12 | 1.6011E−38 | 1 | CKS2 |
| 3.481E−40 | 0.53456104 | 0.636 | 0.389 | 5.9107E−36 | 1 | TXN |
| 5.5587E−40 | 0.51307538 | 0.471 | 0.213 | 9.4387E−36 | 1 | DDX39A |
| 8.6716E−40 | 0.53986134 | 0.425 | 0.181 | 1.4724E−35 | 1 | SLBP |
| 1.6242E−39 | 0.51304521 | 0.25 | 0.067 | 2.7579E−35 | 1 | MAD2L1 |
| 2.0911E−39 | 0.56243996 | 0.234 | 0.06 | 3.5507E−35 | 1 | MCM2 |
| 6.3634E−39 | 0.54445587 | 0.439 | 0.195 | 1.0805E−34 | 1 | PRKDC |
| 9.7898E−39 | 0.50160518 | 0.704 | 0.489 | 1.6623E−34 | 1 | CBX3 |
| 3.7162E−38 | 0.65207077 | 0.249 | 0.07 | 6.31E−34 | 1 | CENPE |
| 6.1323E−38 | 0.55910745 | 0.207 | 0.047 | 1.0413E−33 | 1 | HIST1H2BH |
| 7.1555E−38 | 0.51269874 | 0.201 | 0.044 | 1.215E−33 | 1 | ALYREF |
| 8.712E−38 | 0.50532679 | 0.205 | 0.046 | 1.4793E−33 | 1 | CDKN3 |
| 9.6732E−38 | 0.60141257 | 0.315 | 0.112 | 1.6425E−33 | 1 | HELLS |
| 6.2269E−37 | 0.54968213 | 0.279 | 0.09 | 1.0573E−32 | 1 | FANCI |
| 8.0064E−37 | 0.52950195 | 0.597 | 0.36 | 1.3595E−32 | 1 | HNRNPF |
| 8.886E−37 | 0.51790556 | 0.314 | 0.112 | 1.5088E−32 | 1 | PRIM1 |
| 1.74E−36 | 0.51649488 | 0.553 | 0.311 | 2.9546E−32 | 1 | YWHAE |
| 1.8001E−36 | 0.58292045 | 0.222 | 0.057 | 3.0565E−32 | 1 | ASPM |
| 2.1921E−36 | 0.51269263 | 0.496 | 0.247 | 3.7223E−32 | 1 | SMC3 |
| 3.0369E−36 | 0.60485773 | 0.471 | 0.242 | 5.1567E−32 | 1 | HSPE1 |
| 7.0279E−36 | 0.51760693 | 0.27 | 0.086 | 1.1933E−31 | 1 | HAT1 |
| 1.6055E−35 | 0.57943913 | 0.213 | 0.054 | 2.7261E−31 | 1 | HIST1H2AG |
| 1.7162E−35 | 0.51190422 | 0.459 | 0.22 | 2.9142E−31 | 1 | TXNDC17 |
| 1.6081E−34 | 0.55102002 | 0.398 | 0.182 | 2.7306E−30 | 1 | PRDX1 |
| 3.5712E−34 | 0.52892999 | 0.385 | 0.168 | 6.0639E−30 | 1 | SMC1A |
| 5.3489E−34 | 0.57308122 | 0.229 | 0.066 | 9.0824E−30 | 1 | HIST1H2AE |
| 1.2004E−33 | 0.51300908 | 0.324 | 0.126 | 2.0383E−29 | 1 | EIF4A3 |
| 6.3862E−33 | 0.51914475 | 0.423 | 0.207 | 1.0844E−28 | 1 | TCP1 |
| 8.5173E−33 | 0.51079305 | 0.566 | 0.342 | 1.4462E−28 | 1 | HNRNPD |
| 1.4677E−32 | 0.51321012 | 0.286 | 0.104 | 2.4922E−28 | 1 | NME1 |
| 1.7593E−31 | 0.69830913 | 0.693 | 0.561 | 2.9873E−27 | 1 | HIST1H1D |
| 1.9736E−31 | 0.51276373 | 0.536 | 0.311 | 3.3512E−27 | 1 | C1QBP |
| 6.3092E−31 | 0.50444561 | 0.327 | 0.137 | 1.0713E−26 | 1 | SRSF1 |
| 2.6521E−29 | 0.51839893 | 0.493 | 0.282 | 4.5032E−25 | 1 | CCT6A |
| 3.0406E−26 | 0.59048481 | 0.619 | 0.453 | 5.1629E−22 | 1 | HIST1H1C |
| 3.4512E−24 | 0.51311538 | 0.376 | 0.197 | 5.8602E−20 | 1 | HIST2H2AC |
| 5.9037E−81 | 0.58147085 | 1 | 0.997 | 1.0025E−76 | 2 | TMSB4X |
| 6.1064E−68 | 0.7711348 | 0.273 | 0.031 | 1.0369E−63 | 2 | RCAN3 |
| 5.6718E−65 | 1.05303089 | 0.487 | 0.129 | 9.6307E−61 | 2 | GPR183 |
| 2.1363E−63 | 0.82469475 | 0.245 | 0.026 | 3.6274E−59 | 2 | CCR7 |
| 3.4506E−62 | 1.10727111 | 0.412 | 0.094 | 5.8591E−58 | 2 | LTB |
| 3.0942E−58 | 0.75025423 | 0.228 | 0.025 | 5.2539E−54 | 2 | RP5-1028K7.2 |
| 2.6655E−56 | 0.77468831 | 0.253 | 0.033 | 4.5261E−52 | 2 | SESN3 |
| 1.5911E−53 | 0.86450383 | 0.326 | 0.065 | 2.7017E−49 | 2 | LEF1 |
| 1.9407E−49 | 0.91757279 | 0.44 | 0.131 | 3.2953E−45 | 2 | TCF7 |
| 7.5457E−45 | 0.60731041 | 0.209 | 0.029 | 1.2813E−40 | 2 | AQP3 |
| 1.034E−42 | 0.81664569 | 0.685 | 0.363 | 1.7557E−38 | 2 | ISG20 |
| 2.0766E−40 | 0.7759959 | 0.563 | 0.237 | 3.5261E−36 | 2 | CD27 |
| 1.1037E−39 | 0.91929094 | 0.359 | 0.107 | 1.8742E−35 | 2 | LGALS3 |
| 5.4626E−35 | 0.78363586 | 0.443 | 0.169 | 9.2754E−31 | 2 | FAIM3 |
| 1.0323E−33 | 0.77181555 | 0.588 | 0.297 | 1.7528E−29 | 2 | NOSIP |
| 1.6043E−31 | 0.66910309 | 0.568 | 0.272 | 2.7241E−27 | 2 | LIMS1 |
| 8.5365E−30 | 0.70625731 | 0.437 | 0.178 | 1.4495E−25 | 2 | FOXP1 |
| 8.7333E−30 | 0.59437255 | 0.362 | 0.126 | 1.4829E−25 | 2 | PBXIP1 |
| 1.9176E−29 | 0.56901434 | 0.284 | 0.084 | 3.256E−25 | 2 | CD28 |
| 2.7201E−29 | 0.55524084 | 0.359 | 0.123 | 4.6187E−25 | 2 | SPOCK2 |
| 2.5173E−28 | 0.5443913 | 0.237 | 0.061 | 4.2744E−24 | 2 | CTLA4 |
| 6.4243E−26 | 0.530619 | 0.343 | 0.124 | 1.0909E−21 | 2 | PIK3IP1 |
| 5.66E−25 | 0.52581362 | 0.287 | 0.095 | 9.6107E−21 | 2 | DGKA |
| 1.5954E−24 | 0.88350757 | 0.476 | 0.246 | 2.7091E−20 | 2 | GZMK |
| 4.7192E−24 | 0.58036506 | 0.638 | 0.381 | 8.0132E−20 | 2 | CNN2 |
| 1.0516E−21 | 0.57055497 | 0.471 | 0.23 | 1.7856E−17 | 2 | SPTBN1 |
| 1.6146E−20 | 0.52300819 | 0.889 | 0.776 | 2.7416E−16 | 2 | S100A6 |
| 3.6754E−20 | 0.519896 | 0.643 | 0.412 | 6.2408E−16 | 2 | FAM65B |
| 7.9889E−20 | 0.57085352 | 0.446 | 0.227 | 1.3565E−15 | 2 | CLDND1 |
| 1.4847E−19 | 0.50261134 | 0.245 | 0.086 | 2.521E−15 | 2 | ITGB2-AS1 |
| 6.0109E−18 | 0.54177341 | 0.337 | 0.152 | 1.0207E−13 | 2 | IL16 |
| 1.3404E−17 | 0.52201019 | 0.312 | 0.135 | 2.276E−13 | 2 | IL7R |
| 4.8742E−17 | 0.50562607 | 0.669 | 0.472 | 8.2765E−13 | 2 | SEPT6 |
| 1.141E−154 | 1.56476783 | 0.567 | 0.051 | 1.937E−150 | 3 | TRDC |
| 2.187E−139 | 1.32322926 | 0.396 | 0.02 | 3.713E−135 | 3 | KLRF1 |
| 5.76E−119 | 1.76884003 | 0.967 | 0.468 | 9.78E−115 | 3 | GNLY |
| 2.5E−85 | 1.46759311 | 0.856 | 0.36 | 4.245E−81 | 3 | GZMB |
| 1.7385E−81 | 1.25757359 | 0.544 | 0.114 | 2.952E−77 | 3 | CTSW |
| 3.1512E−66 | 1.19587667 | 0.589 | 0.175 | 5.3507E−62 | 3 | TYROBP |
| 4.2156E−65 | 1.18145487 | 0.422 | 0.083 | 7.1581E−61 | 3 | FCER1G |
| 3.9036E−57 | 1.1791068 | 0.315 | 0.049 | 6.6284E−53 | 3 | XCL2 |
| 9.3385E−53 | 0.83531528 | 0.293 | 0.044 | 1.5857E−48 | 3 | KLRC1 |
| 1.6712E−51 | 1.08193362 | 0.474 | 0.131 | 2.8377E−47 | 3 | FCGR3A |
| 8.8365E−46 | 0.9788669 | 0.526 | 0.176 | 1.5004E−41 | 3 | HOPX |
| 2.9007E−41 | 0.95327828 | 0.748 | 0.425 | 4.9254E−37 | 3 | IFITM2 |
| 5.7953E−41 | 0.86644663 | 0.311 | 0.067 | 9.8403E−37 | 3 | GNPTAB |
| 2.8875E−38 | 0.77800424 | 0.837 | 0.488 | 4.9029E−34 | 3 | STMN1 |
| 7.9301E−38 | 0.87982444 | 0.719 | 0.403 | 1.3465E−33 | 3 | PRF1 |
| 6.5609E−32 | 0.72163016 | 0.215 | 0.041 | 1.114E−27 | 3 | TXK |
| 4.2977E−31 | 0.63769583 | 0.256 | 0.057 | 7.2976E−27 | 3 | RHOC |
| 3.3009E−30 | 0.97282007 | 0.474 | 0.198 | 5.6049E−26 | 3 | IFITM3 |
| 5.4238E−29 | 0.78756163 | 0.463 | 0.181 | 9.2096E−25 | 3 | KLRB1 |
| 4.0903E−27 | 0.69424284 | 0.685 | 0.376 | 6.9454E−23 | 3 | KLRD1 |
| 2.4166E−26 | 0.64153657 | 0.733 | 0.453 | 4.1034E−22 | 3 | DUT |
| 5.601E−26 | 0.82362291 | 0.456 | 0.204 | 9.5105E−22 | 3 | CD63 |
| 7.096E−26 | 0.74519476 | 0.796 | 0.578 | 1.2049E−21 | 3 | TXNIP |
| 7.3891E−26 | 0.80678277 | 0.337 | 0.113 | 1.2547E−21 | 3 | IFI44L |
| 1.8849E−25 | 0.79248785 | 0.67 | 0.428 | 3.2005E−21 | 3 | GSTP1 |
| 1.5275E−24 | 0.69533361 | 0.47 | 0.211 | 2.5938E−20 | 3 | EFHD2 |
| 3.0872E−24 | 0.5980065 | 0.27 | 0.078 | 5.242E−20 | 3 | S1PR5 |
| 9.4043E−24 | 0.6151869 | 0.63 | 0.336 | 1.5968E−19 | 3 | PCNA |
| 1.2881E−23 | 0.71673494 | 0.319 | 0.109 | 2.1873E−19 | 3 | APLP2 |
| 5.6754E−23 | 0.82611396 | 0.404 | 0.167 | 9.6369E−19 | 3 | SPON2 |
| 1.007E−22 | 0.67975162 | 0.378 | 0.152 | 1.7099E−18 | 3 | IL2RB |
| 1.1584E−22 | 0.5506336 | 0.985 | 0.964 | 1.967E−18 | 3 | MALAT1 |
| 3.1315E−22 | 0.60715181 | 0.811 | 0.605 | 5.3173E−18 | 3 | IFITM1 |
| 2.9225E−21 | 0.55463958 | 0.233 | 0.066 | 4.9624E−17 | 3 | TTC38 |
| 5.9631E−21 | 0.67731319 | 0.689 | 0.463 | 1.0125E−16 | 3 | NFKBIA |
| 2.235E−20 | 0.51734054 | 0.644 | 0.379 | 3.7951E−16 | 3 | PLAC8 |
| 1.8462E−18 | 0.59143337 | 0.23 | 0.072 | 3.1348E−14 | 3 | SORL1 |
| 2.7263E−18 | 0.67699031 | 0.326 | 0.135 | 4.6293E−14 | 3 | CD7 |
| 4.1348E−18 | 0.57672938 | 0.496 | 0.257 | 7.021E−14 | 3 | TYMS |
| 3.4538E−17 | 0.66771878 | 0.315 | 0.13 | 5.8646E−13 | 3 | MAP3K8 |
| 5.0037E−17 | 0.59143661 | 0.533 | 0.316 | 8.4964E−13 | 3 | CD247 |
| 5.1208E−16 | 0.72183132 | 0.359 | 0.175 | 8.6951E−12 | 3 | TK1 |
| 2.5332E−15 | 0.50738692 | 0.27 | 0.104 | 4.3013E−11 | 3 | GPR56 |
| 4.9788E−15 | 0.61812765 | 0.363 | 0.176 | 8.4539E−11 | 3 | MLF1IP |
| 5.6165E−15 | 0.57898844 | 0.444 | 0.243 | 9.5369E−11 | 3 | XIST |
| 1.1119E−14 | 0.56419801 | 0.489 | 0.286 | 1.888E−10 | 3 | NUCB2 |
| 1.1299E−13 | 0.52897935 | 0.267 | 0.112 | 1.9186E−09 | 3 | BHLHE40 |
| 1.2167E−13 | 0.5861332 | 0.348 | 0.172 | 2.066E−09 | 3 | TRGC1 |
| 3.1265E−13 | 0.522249 | 0.43 | 0.24 | 5.3088E−09 | 3 | MCM7 |
| 8.7005E−13 | 0.5309669 | 0.285 | 0.131 | 1.4774E−08 | 3 | MGST3 |
| 1.4695E−12 | 0.50107465 | 0.237 | 0.097 | 2.4953E−08 | 3 | DDIT4 |
| 2.8996E−12 | 0.54843155 | 0.585 | 0.39 | 4.9235E−08 | 3 | TUBB |
| 1.5718E−11 | 0.50275626 | 0.326 | 0.17 | 2.6689E−07 | 3 | ATAD2 |
| 1.9364E−11 | 0.5076629 | 0.43 | 0.255 | 3.288E−07 | 3 | EIF2AK2 |
| 1.9997E−11 | 0.72413173 | 0.374 | 0.21 | 3.3955E−07 | 3 | CCL4 |
| 2.3983E−11 | 0.56578837 | 0.226 | 0.099 | 4.0723E−07 | 3 | SVIP |
| 1.077E−10 | 0.51596788 | 0.348 | 0.187 | 1.8287E−06 | 3 | NUSAP1 |
| 1.5718E−10 | 0.65628563 | 0.237 | 0.112 | 2.6689E−06 | 3 | FAM111B |
| 2.6602E−10 | 0.55132555 | 0.281 | 0.142 | 4.517E−06 | 3 | FANCI |
| 1.4023E−09 | 0.54259684 | 0.211 | 0.097 | 2.3812E−05 | 3 | BRCA2 |

TABLE 7D
Genes used for scoring against CD8, gdT and NK cells
from Gutierrez-Arcelus et al., Nat Commun, 2019.

| Cell type | Genes |
|---|---|
| CD8 | CD8B TRAC CCR7 AIF1 CD8A RGS10 LDHB LEF1 LINC02446 IL7R RCAN3 COTL1 |
| | CD27 LTB C6orf48 MYC |
| gdT | TRDC TRGC1 TRGC2 IL32 CD3D KLRG1 LINC01871 DUSP2 |
| NK | FCER1G KLRF1 TYROBP GZMB SPON2 GNLY PRF1 NKG7 FGFBP2 CLIC3 IGFBP7 |
| | KLRD1 AKR1C3 SRGN MYOM2 KLRB1 FCGR3A PLAC8 CD7 GZMA DDIT4 CST7 ID2 |
| | CD247 HOPX JAK1 CHST2 SH2D1B CTSW IL2RB IFITM2 HSH2D TXK PRSS23 CCL4 |
| | RAP1B RHOC ALOX5AP TTC38 PTPN12 GPR65 APMAP MYO1F C1orf162 CTSD CD160 |
| | EFHD2 CEBPD IFITM1 CD63 GPATCH8 CCND3 ABHD17A MBP TCF25 CX3CR1 |
| | GNPTAB IFITM3 STARD3NL |

TABLE 8
Gene lists used to assay RNA contamination in each cell type

| Cell Types | Genes |
|---|---|
| B cells | LYZ CD14 TRBC2 TRDC CD3E CD3G CD3D CCL5 MNDA VCAN FCGR3A NKG7 |
| Plasmablast | LYZ CD14 TRBC2 TRDC CD3E CD3G CD3D CCL5 |
| CD4+ cells | LYZ CD14 IGJ IGHM IGHG1 IGHG2 IGHG3 IGHG4 IGHD IGHA1 FCGR3A VCAN |
| NK cells | LYZ CD14 IGJ IGHM IGHG1 IGHG2 IGHG3 IGHG4 IGHD IGHA1 CD3E CD3D CD3G |
| | TRAC |
| Monocytes | CD3E CD3G CD3D IGJ IGHM IGHG1 IGHG2 IGHG3 IGHG4 IGHD IGHA1 TRBC2 |
| | TRDC |
| CD1C+ DC | CD3E CD3G CD3D IGJ IGHM IGHG1 IGHG2 IGHG3 IGHG4 IGHD IGHA1 TRBC2 |
| | TRDC |
| CTL | LYZ CD14 IGJ IGHM IGHG1 IGHG2 IGHG3 IGHG4 IGHD IGHA1 |
| Prolif T | LYZ CD14 IGJ IGHM IGHG1 IGHG2 IGHG3 IGHG4 IGHD IGHA1 |

### Example 2—Integrated Single-Cell Analysis of Multicellular Immune Dynamics During Hyperacute HIV-1 Infection

Cellular immunity is critical for controlling intracellular pathogens, but individual cellular dynamics and cell-cell cooperativity in evolving human immune responses remain poorly understood. Single-cell RNA-sequencing (scRNA-seq) represents a powerful tool for dissecting complex multicellular behaviors in health and disease and nominating testable therapeutic targets. Its application to longitudinal samples could afford an opportunity to uncover cellular factors associated with the evolution of disease progression without potentially confounding inter-individual variability. This example shows an experimental and computational methodology that used scRNA-seq to characterize dynamic cellular programs and their molecular drivers and its application to HIV infection. By performing scRNA-seq on peripheral blood mononuclear cells from four untreated individuals before and longitudinally during acute infection⁵, Applicants were powered within each to discover gene response modules that vary by time and cell subset. Beyond previously unappreciated individual- and cell-type-specific interferon-stimulated gene upregulation, Applicants described temporally aligned gene expression responses obscured in bulk analyses, including those involved in proinflammatory T cell differentiation, prolonged monocyte major histocompatibility complex II upregulation and persistent natural killer (NK) cell cytolytic killing. Applicants further identified response features arising in the first weeks of infection, for example proliferating natural killer cells, which potentially may associate with future viral control. Overall, the approach provides a unified framework for characterizing multiple dynamic cellular responses and their coordination.

Despite advances in pre-exposure prophylaxis, there were a million new cases of HIV infection in 2018 (ref 6), highlighting the need for effective HIV vaccines. A better understanding of key immune responses during the earliest stages of infection, especially Fiebig stage I and II, before and at peak viral load, respectively, could help identify prophylactic and therapeutic targets⁷. Using historical samples, collected before standard-of-care included treatment during acute infection, from the Females Rising through Education, Support and Health (FRESH) study⁵, Applicants assayed evolving immune responses during hyperacute (1-2 weeks post-detection) and acute (3 weeks to 6 months) HIV infection.

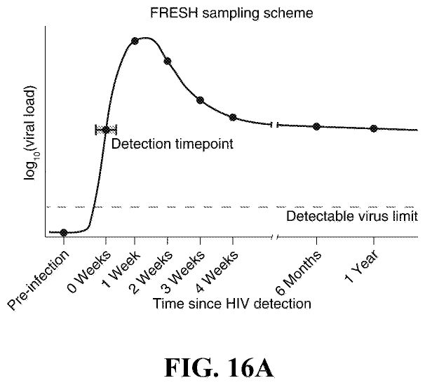

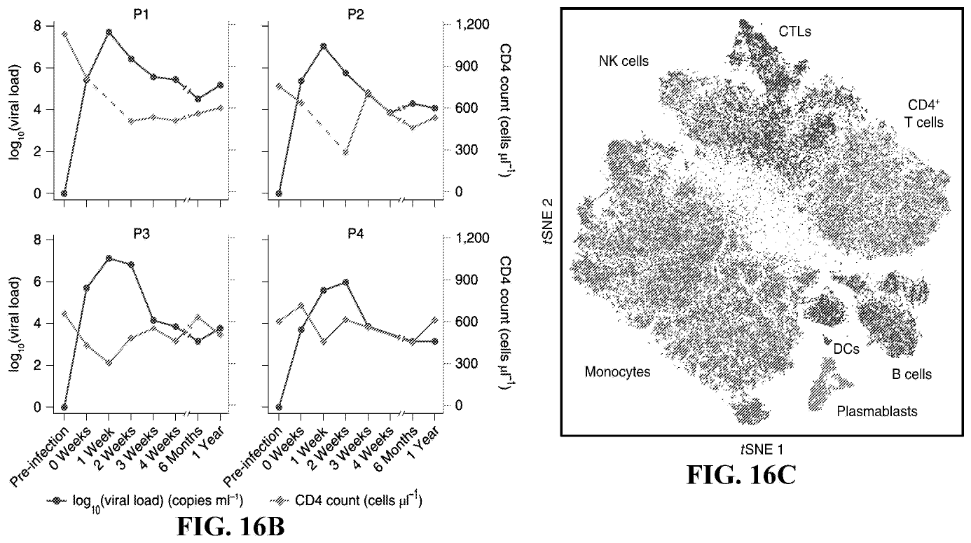

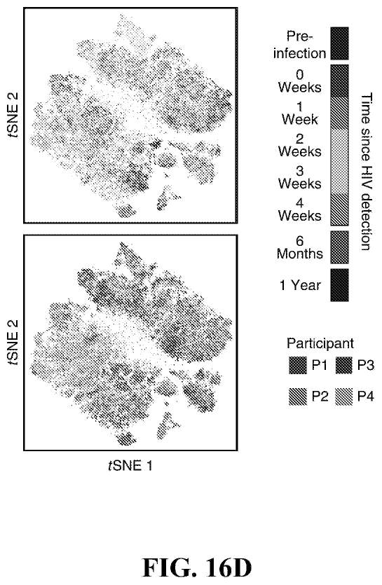

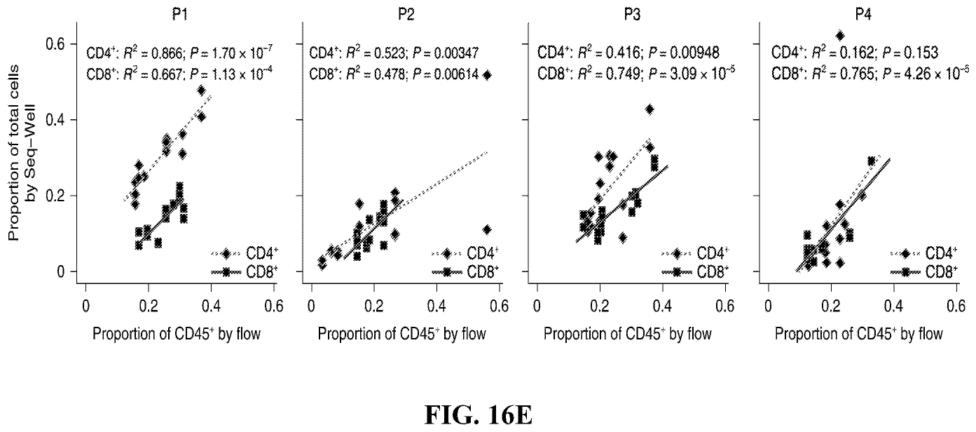

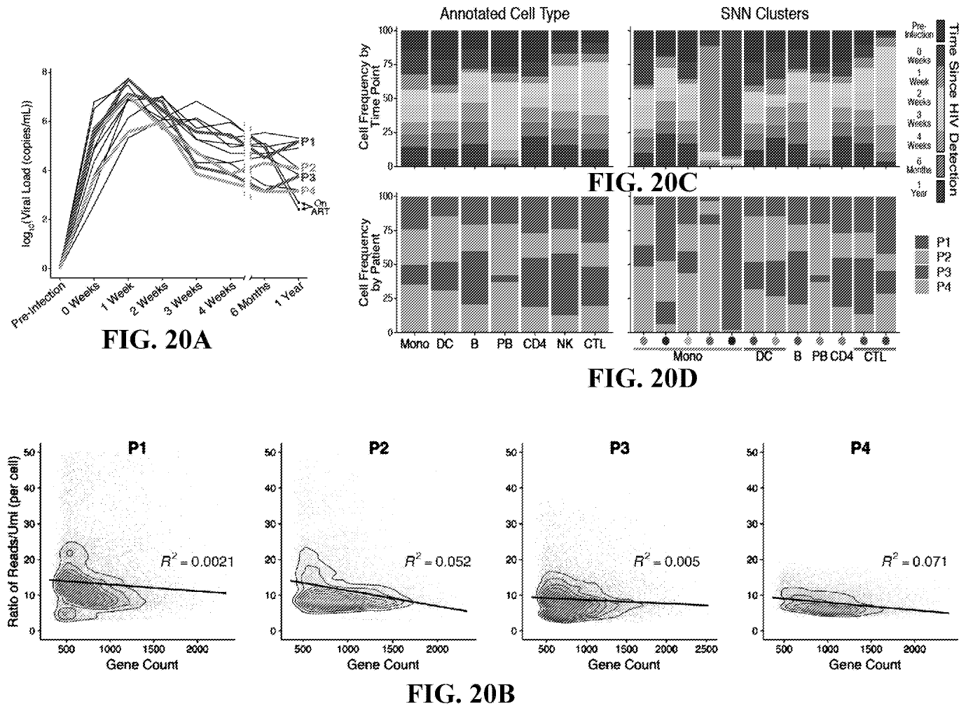

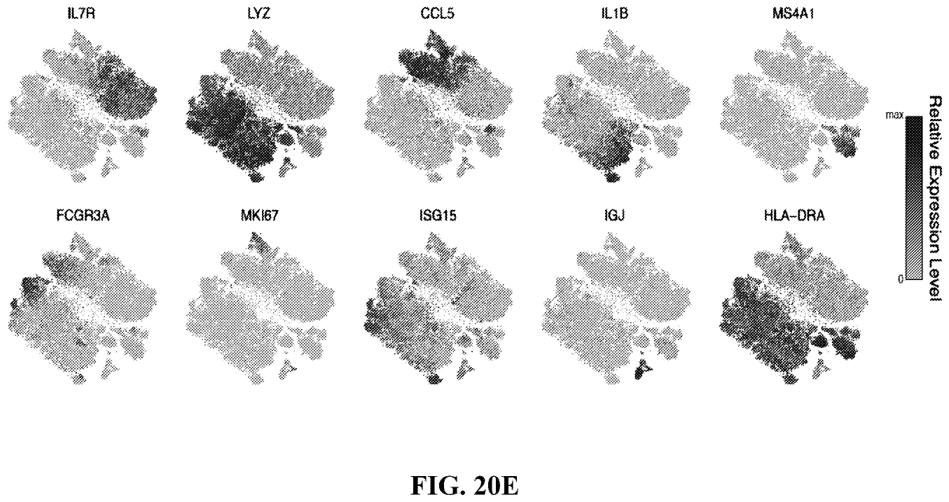

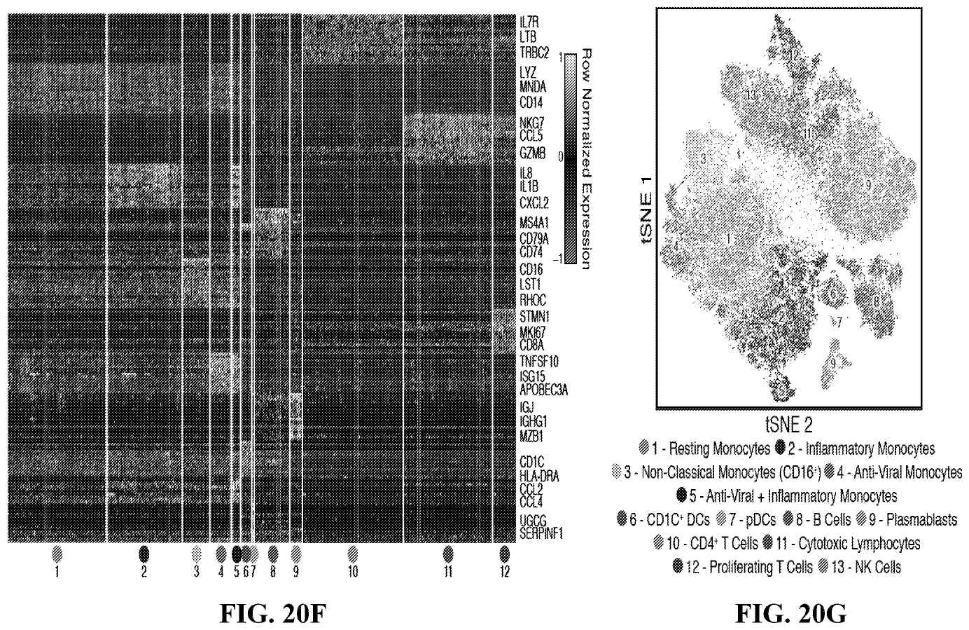

Applicants performed Seq-Well-based massively parallel scRNA-seq on peripheral blood mononuclear cells (PBMCs) from four FRESH participants who became infected with HIV during study. Applicants analyzed multiple timepoints from pre-infection through 1 year following viral detection (FIG. 16A) over which all four demonstrated a rapid rise in plasma viremia and a drop in CD4+ T cell counts⁸ (FIGS. 16B and 20A). Altogether, Applicants captured 59,162 cells after performing quality controls, with an average of 1,976 cells per participant per timepoint (FIG. 20B).

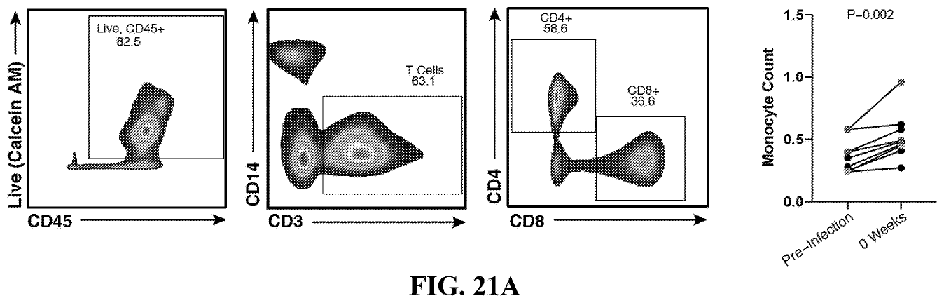

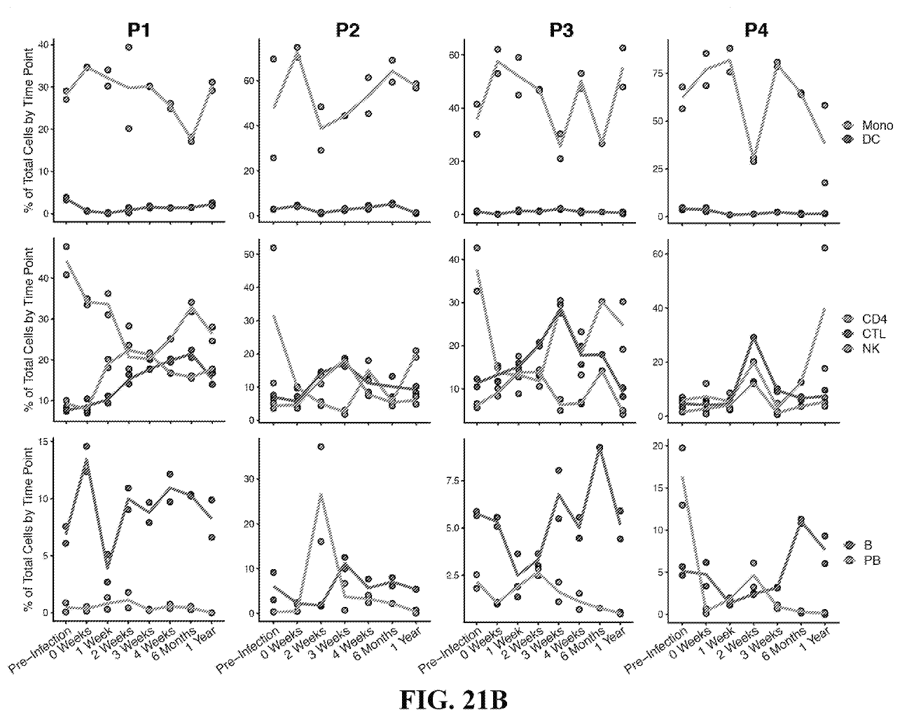

To assign cellular identity, Applicants analyzed the combined data from all participants and timepoints (Methods). These analyses yielded few participant-specific features, suggesting that disease biology, rather than technical artifact, is the main driver of variation (FIG. 16D, FIGS. 20C-20D). Applicants annotated clusters by comparing differentially expressed genes defining each to known lineage markers and previously published datasets (FIGS. 20E-20F). These clusters recapitulate several well-established PBMC subsets (FIG. 16C), revealed phenotypic subgroupings of both monocytes (antiviral, inflammatory and nonclassical) and cytotoxic T cells (CTLs) (CD8+CTL, proliferating; FIG. 20G) and highlighted subset frequency dynamics such as natural killer (NK) cell expansion after 2-3 weeks. Flow cytometry measurements of CD45+CD3+CD4+ and CD45+CD3+CD8+ frequencies over the course of infection correlated with those measured by Seq-Well (FIGS. 21A, 21B and FIG. 16E). Whole blood monocyte counts, meanwhile, confirmed monocyte expansion following infection (FIG. 21C).

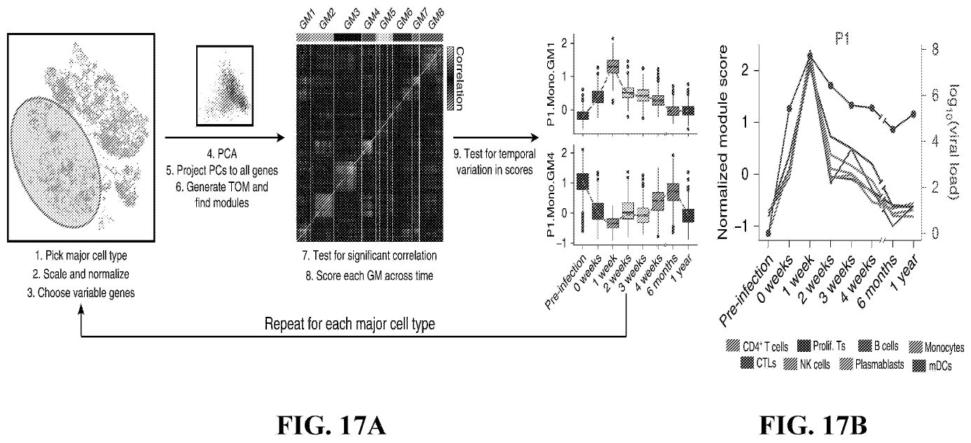

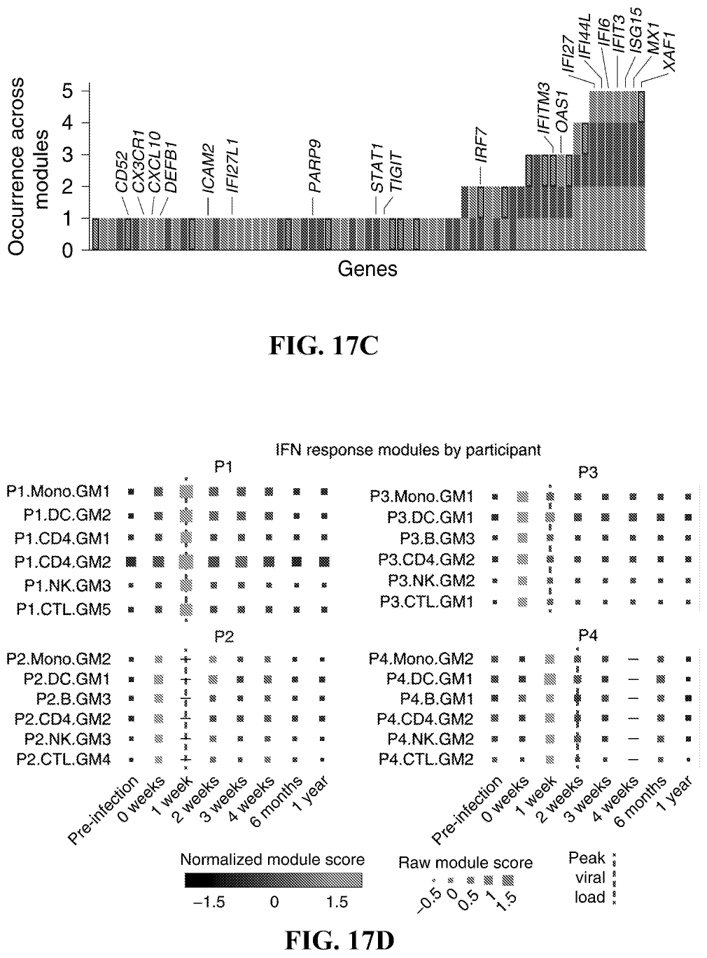

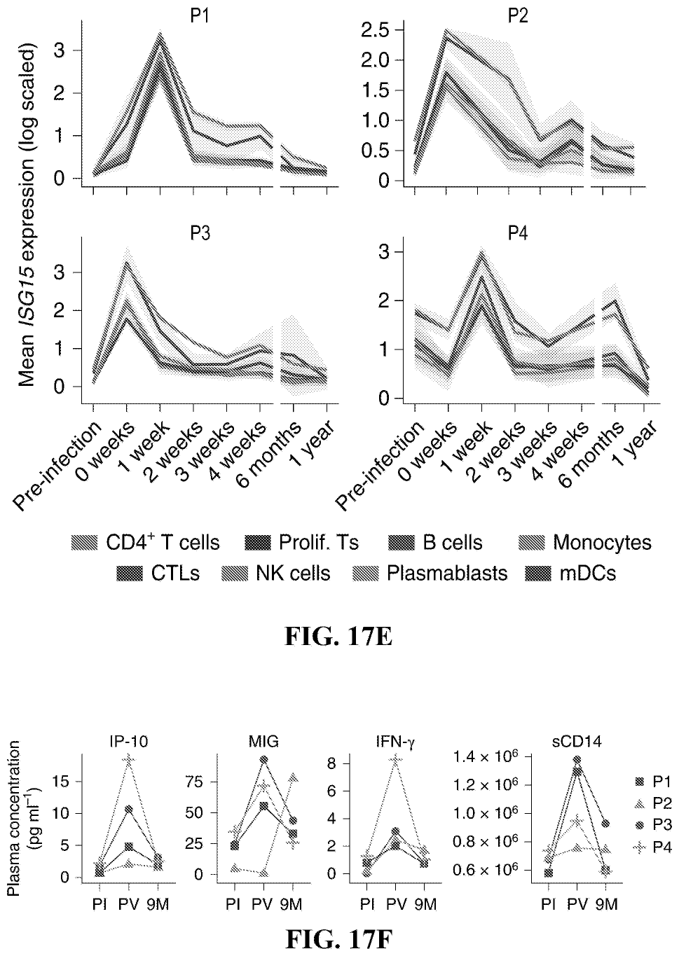

Having mapped cell type frequency dynamics during acute HIV-1 infection, Applicants next examined how different cellular phenotypes shifted over time. Previous applications of scRNA-seq to evolving cellular responses have either emphasized pseudotemporal ordering in development⁹ to delineate well-ordered progressions through cell fate¹⁰ or identified transcriptional differences¹¹ associated with disease treatment¹². As the dataset included multiple, noncontiguous timepoints and complex nonlinear dynamics spaced over days to weeks, it needed distinct treatment. Therefore, Applicants developed a framework to examine how each cell type varied in phenotype over the course of infection by adapting weighted gene correlation network analysis (WGCNA) to discover, in an unbiased manner at single-cell resolution, gene modules (GMs) whose expression varied significantly over time (FIG. 17A). Given the small number of participants and heterogeneity in disease response, Applicants opted to characterize each participant and cell type independently to: 1) identify cellular responses associated with plasma viremia; 2) group modules within individuals over time; and 3) nominate molecular drivers and potential cell-cell signaling.

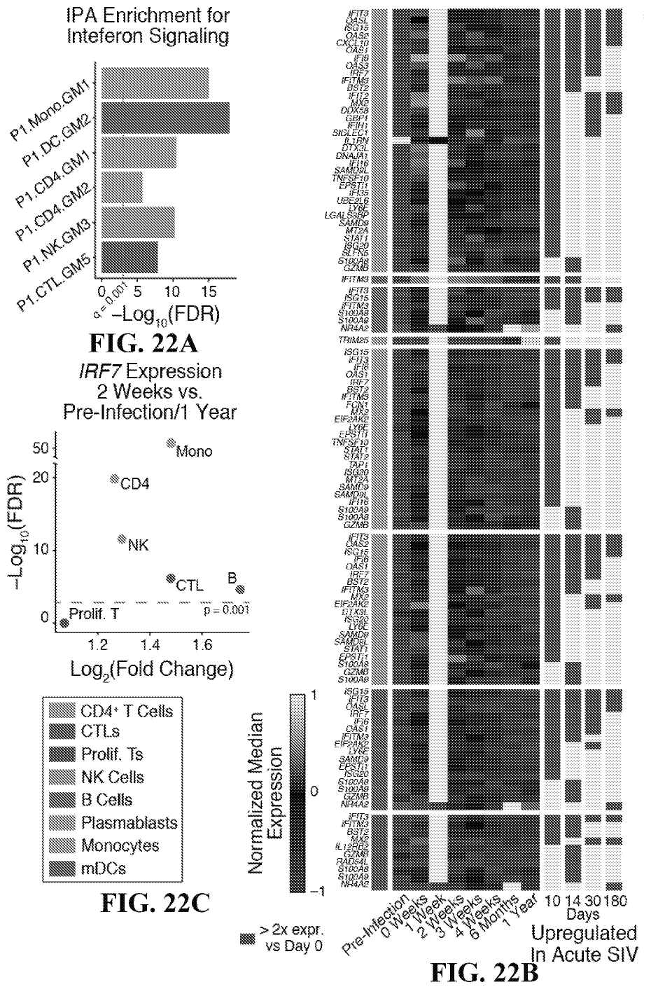

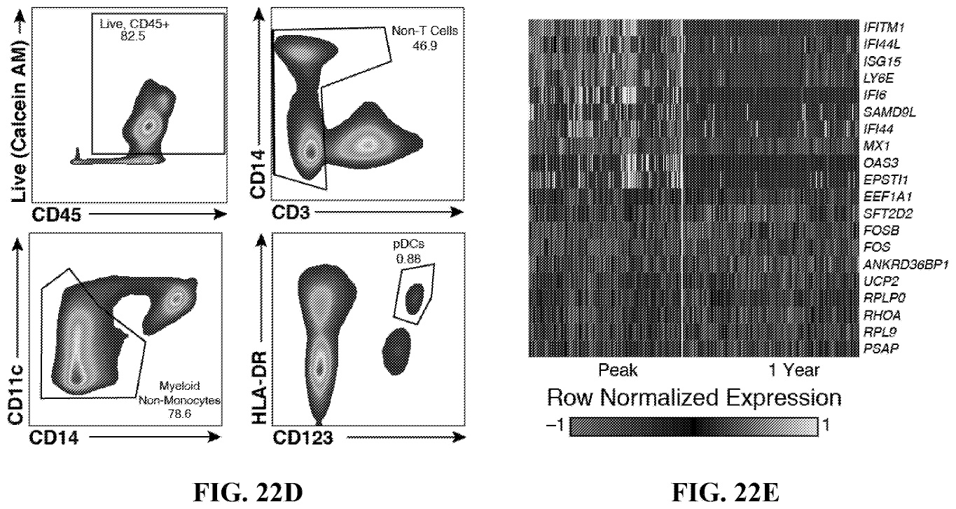

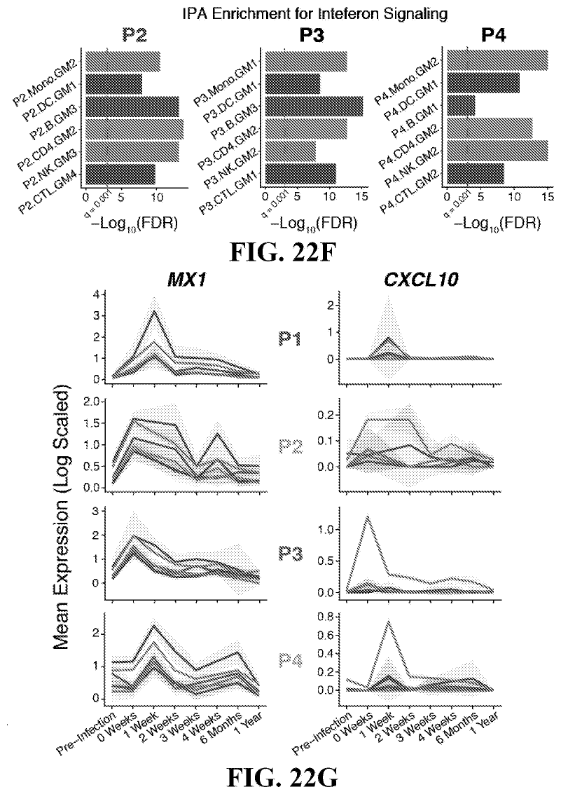

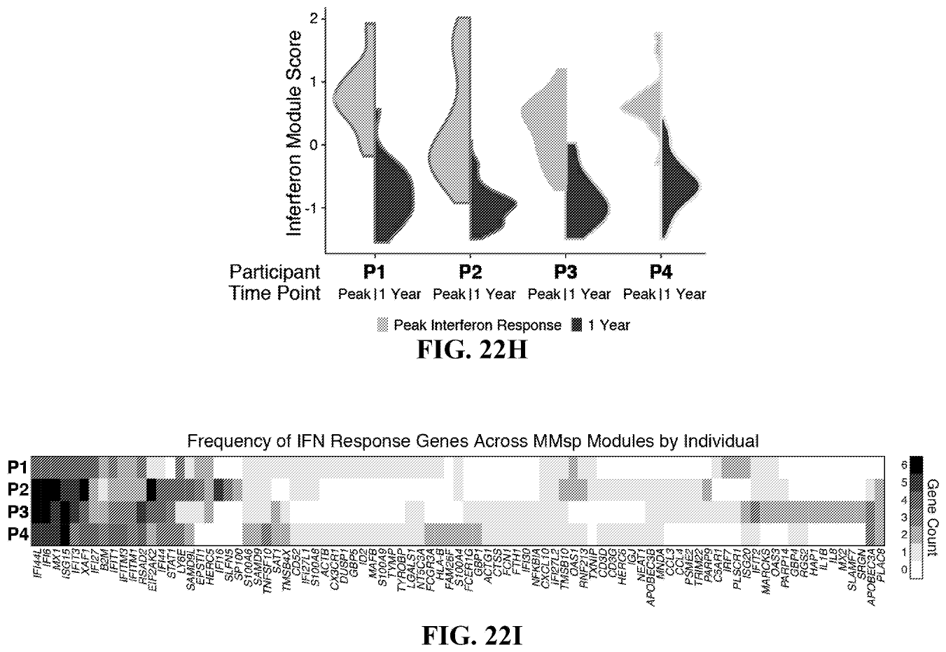

Within each individual, the discovered GMs demonstrated common transient patterns over the course of infection, indicating the utility of the approach in uncovering responses with shared dynamics across multiple cell types. Looking at GMs associated with changes in plasma viral load in participant 1 (P1), Applicants identified a set of six spanning multiple cell types all sharing their highest relative module score at peak viremia (FIG. 17B). Despite being generated in distinct cell types, each GM included IFI27, IFI44L, IFI6, IFIT3, ISG15 and XAF1 (FIG. 17B and FIG. 22A), in addition to other interferon (IFN)-stimulated genes (ISGs)¹³. Collectively, these expression patterns revealed cell-type-specific genes and functions correlated with a core ISG signature in P1, including monocyte antiviral activity (CXCL10, DEFB1)^{14,15} dendritic cell (DC) activation (PARP9, STAT1)^{16,17}, naive CD4+ T cell differentiation (CD52, TIGIT)^{18,19} and NK cell trafficking (CX3CR1, ICAM2)²⁰. Moreover, in P1, monocytes and DCs uniquely expressed genes (CXCL10, LGALS3BP) measured in bulk responses in acute simian immunodeficiency virus (SIV) infection in rhesus macaques²¹, which may shed light on the cellular sources of these antiviral molecules (FIG. 22B).

Applicants characterized the expression of their upstream regulator IRF7 to infer which cell type(s) may be responsible for their production22. In P1, six of eight cell types studied demonstrated higher expression of IRF7 at peak viremia compared to pre-infection and 1-year timepoints (FIG. 22C). Applicants also assayed plasmacytoid DCs (pDCs), which produced IFN-α and IFN-β in response to HIV23, at peak viremia and 1-year post-infection (FIGS. 22D and 22E) but did not find IFN-I gene expression or a significant change in IRF7 expression (two-sided Wilcoxon rank-sum test, false discovery rate (FDR) corrected q<1). The three other participants studied (P2-P4) each had pDC responses and sets of ISG GMs similar to P1 at, or the week before, peak viremia, which Applicants corroborated at the individual gene level (FIGS. 17D, 17E and FIGS. 22F-22H). Comparing GMs across individuals, Applicants noted common ISGs (present in three or more cell types) that were shared in two or more participants (ISG15, IFIT3, XAF1) as well as some specific to a single participant (APOBEC3A, IFI27, STAT1; FIG. 22I). To independently confirm the presence of IFNs and downstream cytokines, Applicants measured IFN-γ, MIG (CXCL9) and IP-10 (CXCL10; previously associated with disease progression and infection outcome²⁴; FIG. 17F). All participants demonstrated higher levels of IFN-γ and IP-10 at peak viremia and three demonstrated elevated MIG. Applicants also observed increased soluble CD14, known to be associated with monocyte activation²⁵.

Given concerted and cell-type-specific IFN responses during hyperacute HIV infection, Applicants next explored whether other modules exhibited shared expression dynamics. Applicants applied fuzzy c-means clustering to the median module scores at each timepoint across all cell types on a participant-by-participant basis, generating clusters of modules which Applicants refer to as meta-modules (MMs) (Methods). MMs represented gene programming across distinct cell types with coordinated temporal dynamics—here, synchronized responses to infection—enabling Applicants to link cellularly discrete but contemporaneous behaviors to both common and unique propagators.

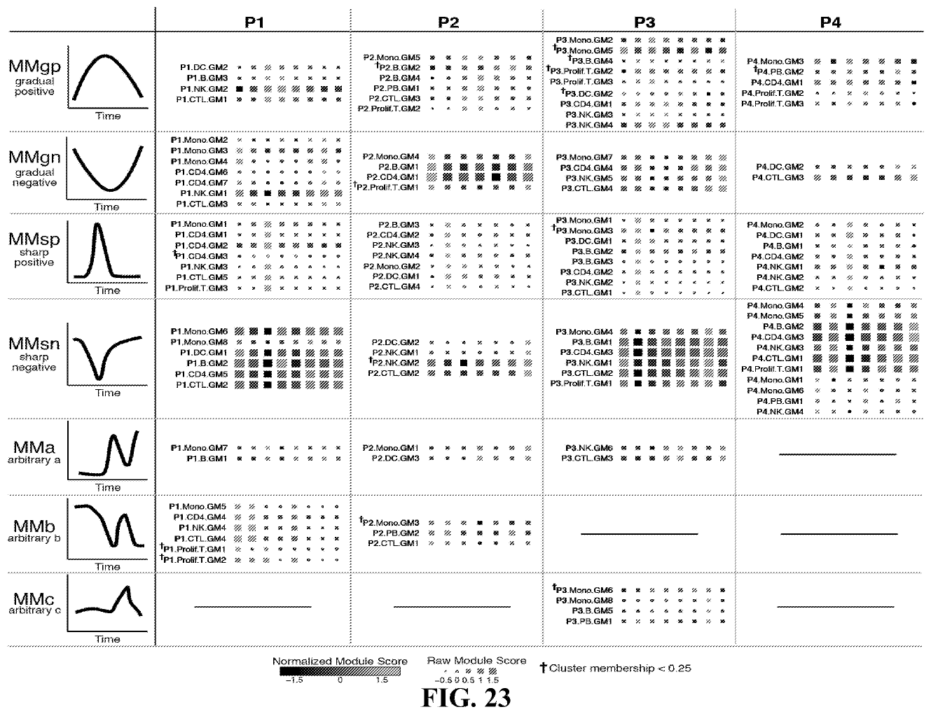

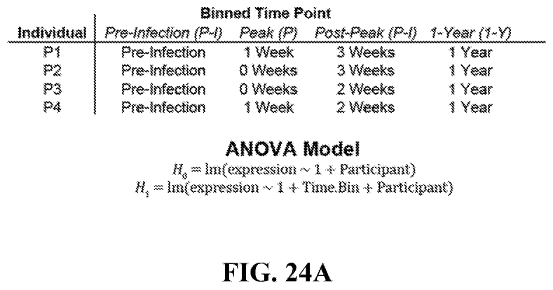

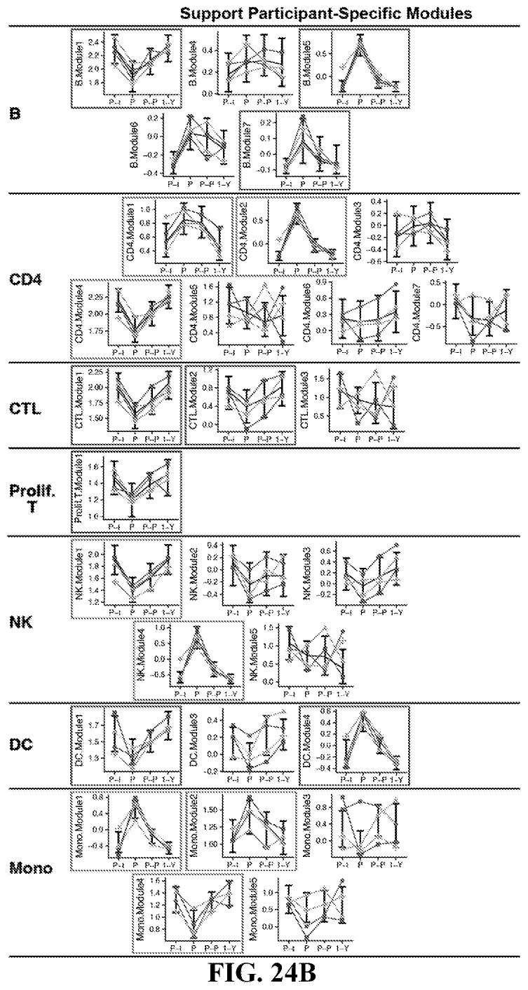

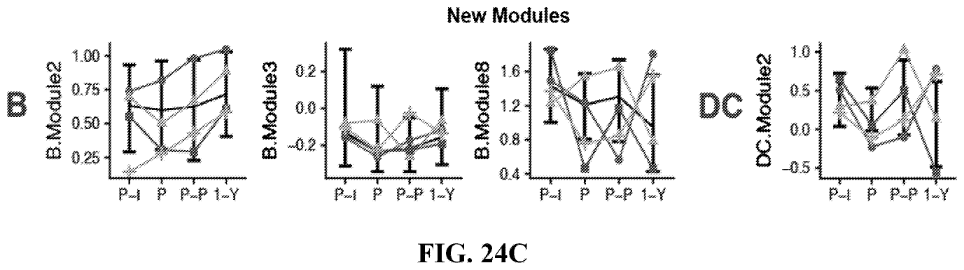

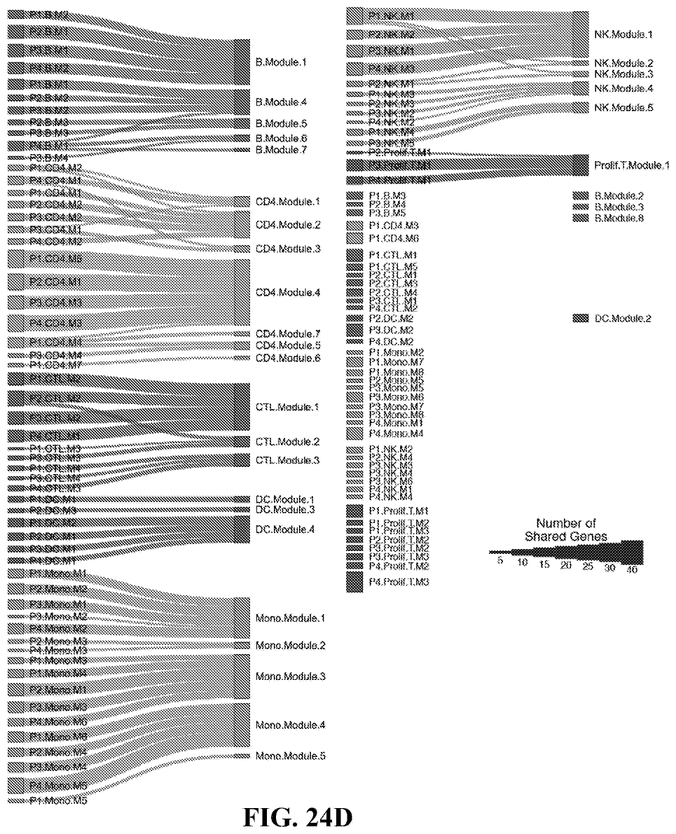

Applicants next identified MMs from every participant and grouped them by their expression dynamics (FIG. 23). Applicants labeled four of these on the basis of their transient peak expression score patterns: sharp positive (MMsp), sharp negative (MMsn), gradual positive (MMgp) and gradual negative (MMgn); three additional MMs, labeled a-c, demonstrated more complex patterns. Besides MMsp, which contained the majority of the ISG modules, only MMsn, enriched for ribosomal protein-coding genes previously shown to indicate cellular quiescence²⁶, spanned five or more cell types. In parallel, Applicants attempted to discover conserved modules across individuals, using cells from all four participants binning timepoints by viral load (FIG. 24A). All but four of these cross-participant modules recapitulated those found in the participant-specific approach (FIGS. 24B-24D). However, this pan-participant analysis did not reveal any GMs with consistent expression trends (in at least three out of four participants) besides the already identified ISG (MMsp) and ribosomal protein (MMsn) modules and failed to discover several participant-specific modules within MMgp.

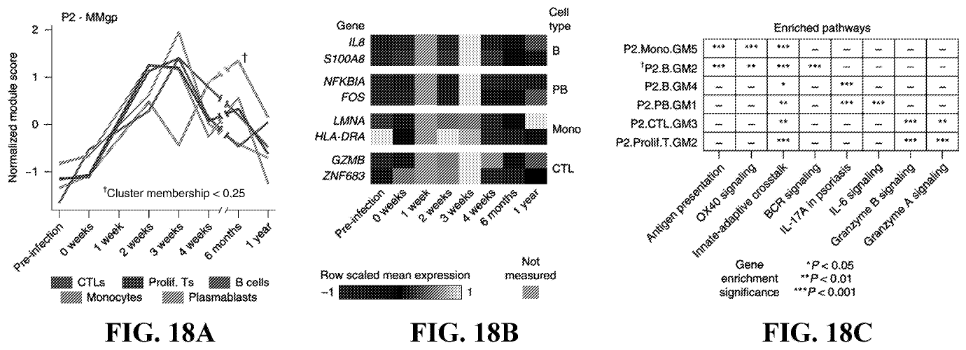

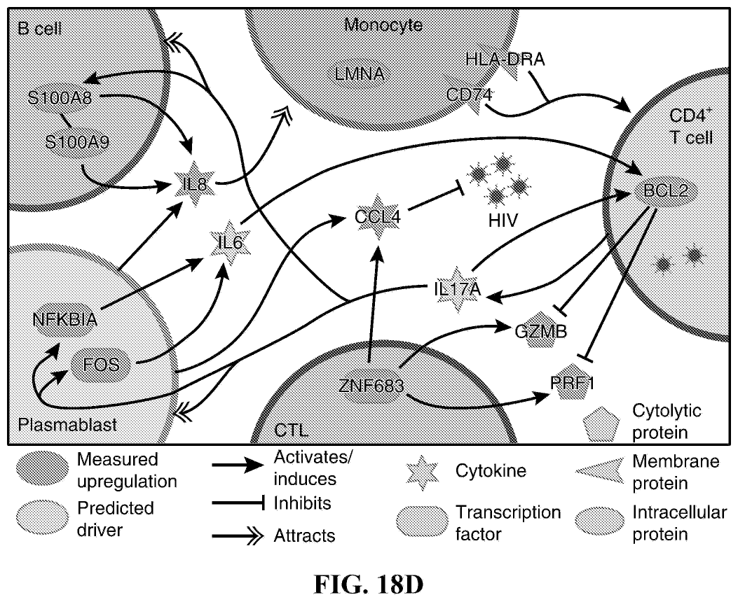

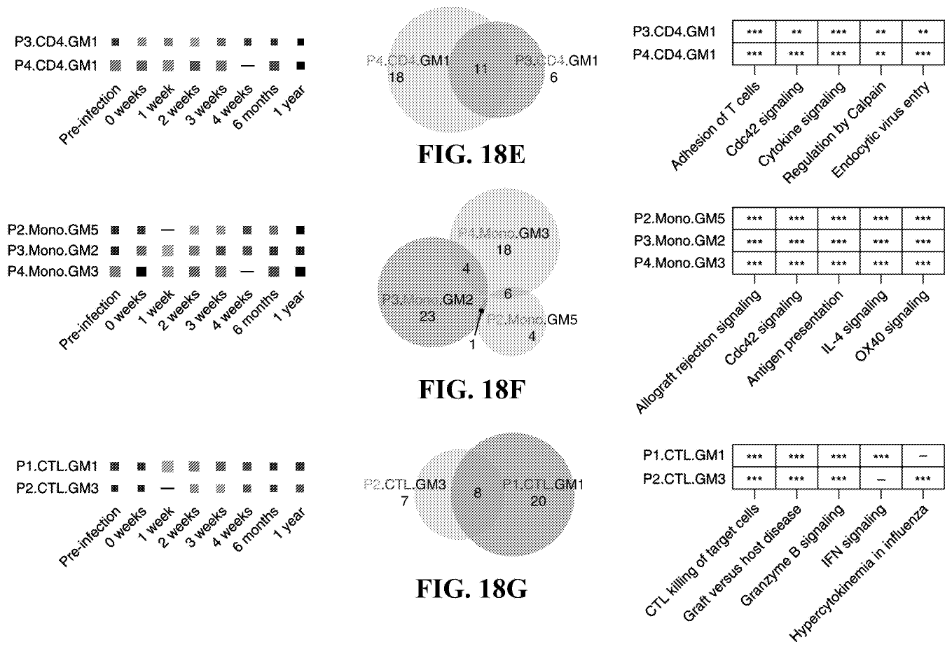

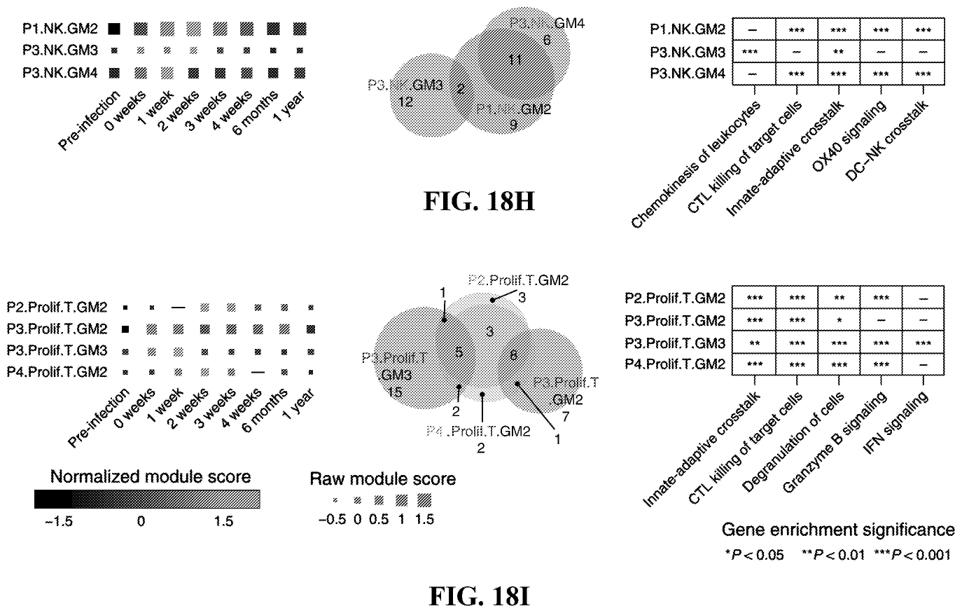

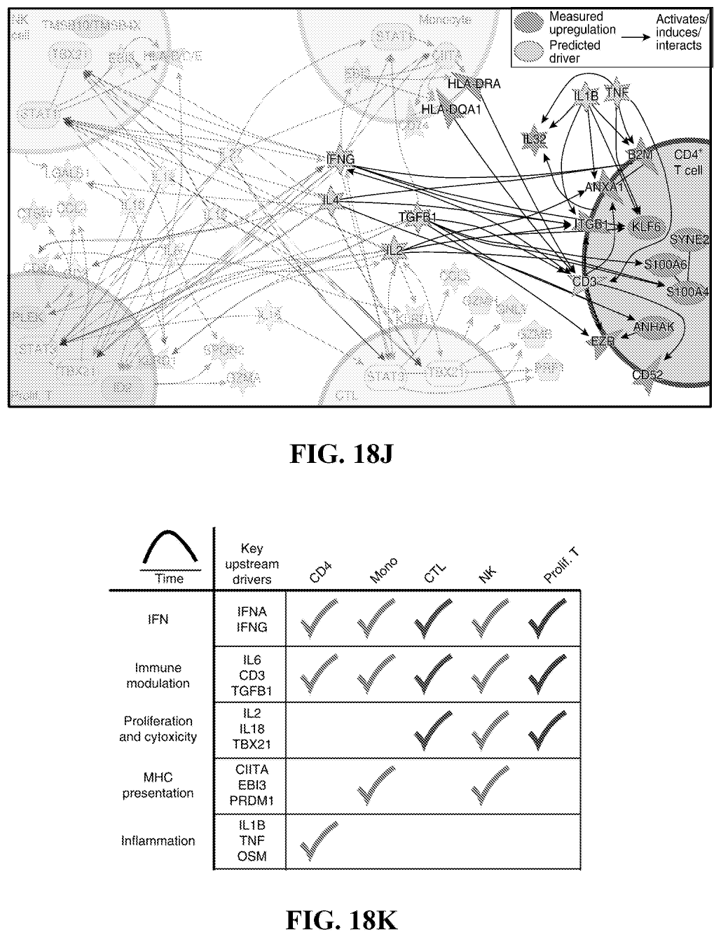

Notably, MMgp had responses sustained throughout acute infection, but implicated different cell types in each participant. For example, in P2, MMgp consisted of monocyte, B cell, plasmablast, CTL and proliferating T cell GMs (FIG. 18A). Unlike MMsp (ISGs), these GMs spanned several distinct gene expression pro-grams, such as antigen presentation (monocytes and B cells), interleukin (IL)-6 and IL-8 production (plasmablasts) and granzyme B production (CTLs; FIGS. 18B and 18C 7). As these overlap in time, they may represent cell subsets responding to common stimuli and/or one another. Looking for known relationships between genes within and across cell types, Applicants generated a network model describing potential axes of cell-cell signaling, both direct (via receptor-ligand) and indirect (signaling via chemokines and cytokines), in P2 (FIG. 18D and Methods). Expression of IL-8 and IL-6 in B cells and plasmablasts²⁷ may attract monocytes presenting antigen to prime CD4+ T cells, potentially leading to IL-17 production²⁸ and BCL2 upregulation, known to restrict CTL-mediated killing of infected cells²⁹. Together, this suggests the IL-6-IL-8-IL-17 signaling axis as a potential target for HIV treatment.

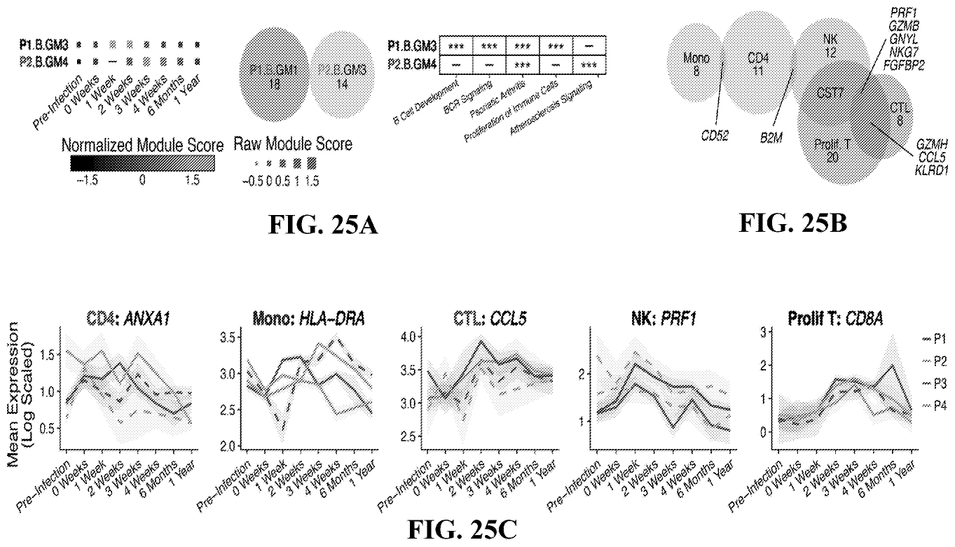

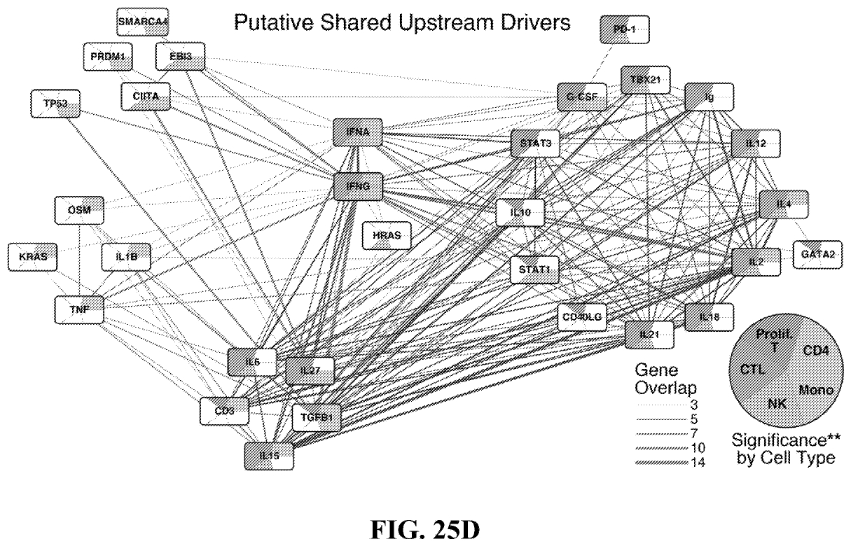

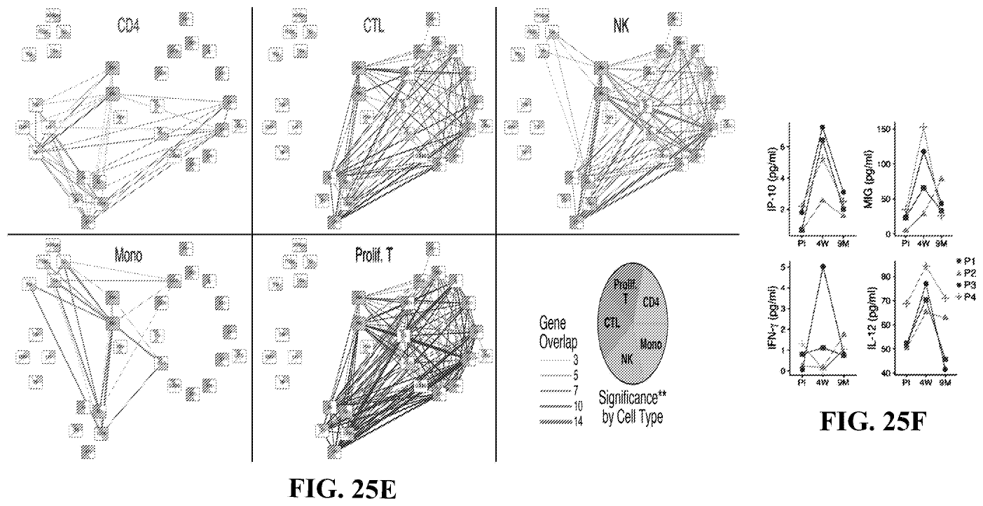

Given the diverse, participant-specific GMs in MMgp (FIG. 23), Applicants next looked whether any acute infection responses were present in multiple participants. In CD4+ T cells, mono-cytes, NK cells, CTLs and proliferating T cells, Applicants found GMs in MMgp that shared genes in two or more participants (FIGS. 18E-18I). While DCs and B cells also expressed multiple GMs within MMgp, some did not share any genes across participants (FIG. 25A) or had low membership scores and were thus excluded (membership <0.25, labeled with t in FIG. 23; Methods).

Applicants next qualitatively compared GM functional annotations within MMgp for each cell type across participants (FIGS. 18E-18I). Despite variable temporal dynamics and unique gene memberships, Applicants observed significant enrichment for ≥15 of the same underlying pathways and functions in at least two participants (P<0.01), suggesting the existence of common features across individuals despite heterogeneity in infection response. For example: 1) CD4+ T cells (P3+P4) expressed genes associated with nonclassical viral entry by endocytosis³⁰ and adhesion, suggesting migration and viral dissemination throughout the body; 2) monocytes (P2+P3+P4) expressed genes associated with antigen presentation, potentially indicating generalized IFN responses or the potential to promote active T helper and CTL responses31; and 3) NK cells (P1+P3), CTLs (P1+P2) and proliferating T cells (P2+P3+P4) upregulated genes associated with killing of target cells by perforin and granzyme release, highlighting the joint role of innate and adaptive lymphocytes in combating viremia^{32,33} (see FIG. 25B for shared genes). Gene expression data corroborate these GM expression trends (FIG. 25C).

Applicants found that there may be a common set of immune drivers coordinating these gene responses during infection. To identify potential inducers of the GMs in MMgp, Applicants generated a list of predicted upstream drivers for each. Using hits that were significant for two or more GMs, Applicants constructed a network detailing putative upstream signaling (FIGS. 25D and 25E), highlighting potential roles for: 1) IFN-α and IFN-γ across all five cell types; 2) IL-15, IL-12 and IL-21 in CTLs, NK and proliferating T cells; and 3) IL-10 and tumor necrosis factor (TNF) restricted to CD4+ T cells. Parallel Luminex measurements confirmed increased IP-10, MIG and IL-12, but not IFN-γ, in plasma at 4 weeks, near when MMgp peaked in each individual (FIG. 25F).

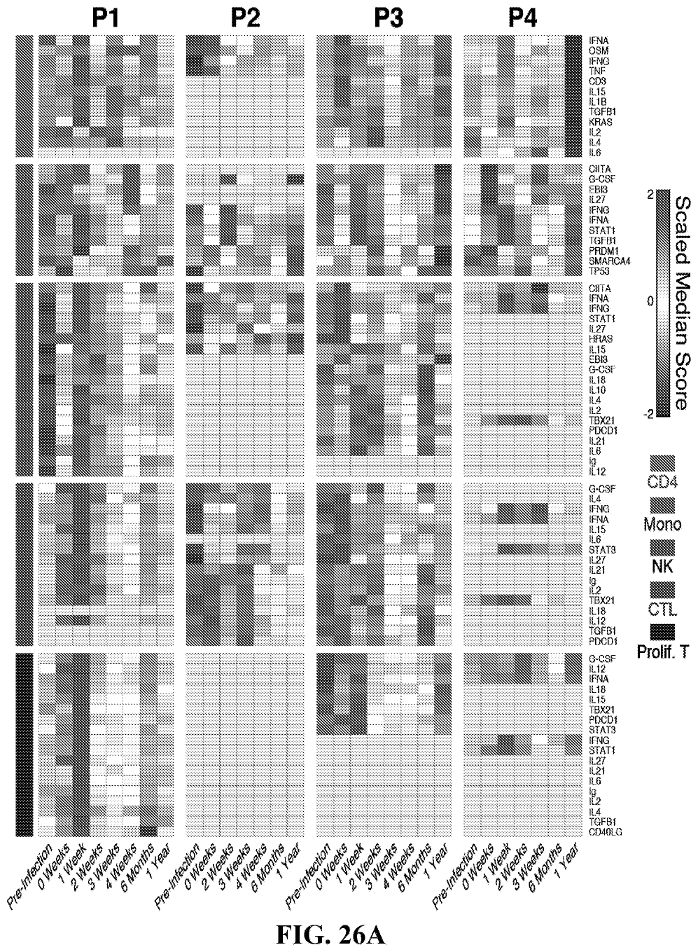

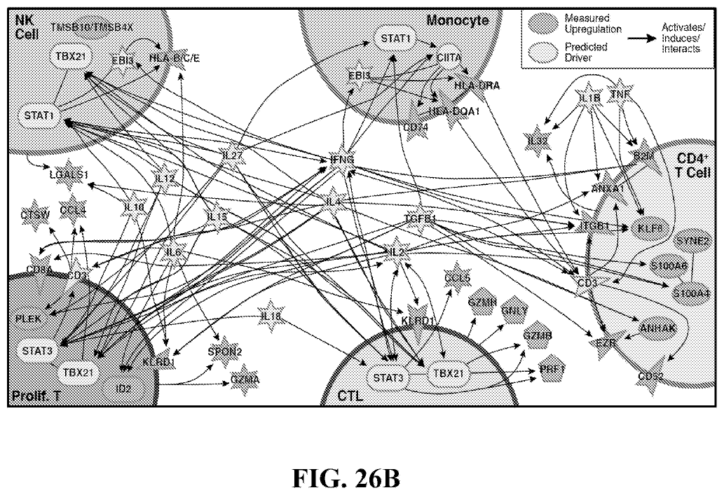

Re-scoring cell types against enriched genes for each driver revealed variable kinetics in the onset, intensity and length of immune responses across different cell types (FIG. 26A). Applicants noted the following gene-programming upregulation trends in all participants: 1) CD4+ T cells activity from before peak viremia throughout acute infection; 2) CTL and proliferating T cell programs are induced during hyperacute infection; and 3) NK cell and monocyte activity persists throughout the first month of infection, highlighting a persistent role for innate immunity throughout acute infection. Based on cell type, gene and functional enrichments, Applicants summarize the shared (≥2 participants) immune responses with sustained gene expression over the course of the first month of HIV infection, their potential drivers and putative cell-cell signaling, emphasizing CD4+ T cells, which was the only cell type expressing genes downstream of proinflammatory cytokines (FIGS. 18J, 18K and FIG. 26B). Thus, the module discovery approach readily revealed immune responses and potential interactions among several cell types during acute HIV infection.

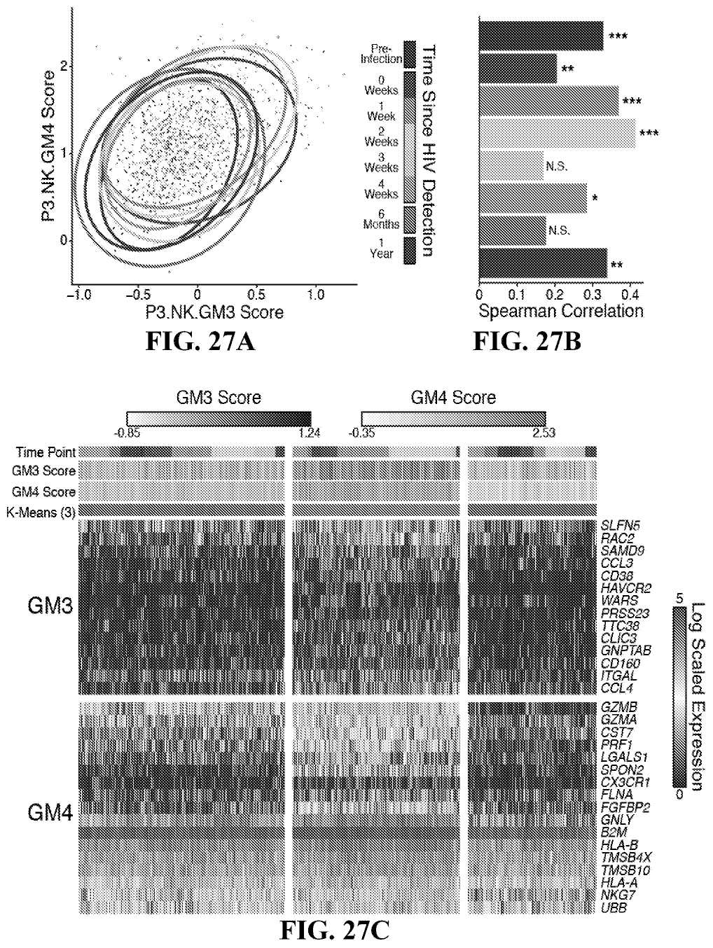

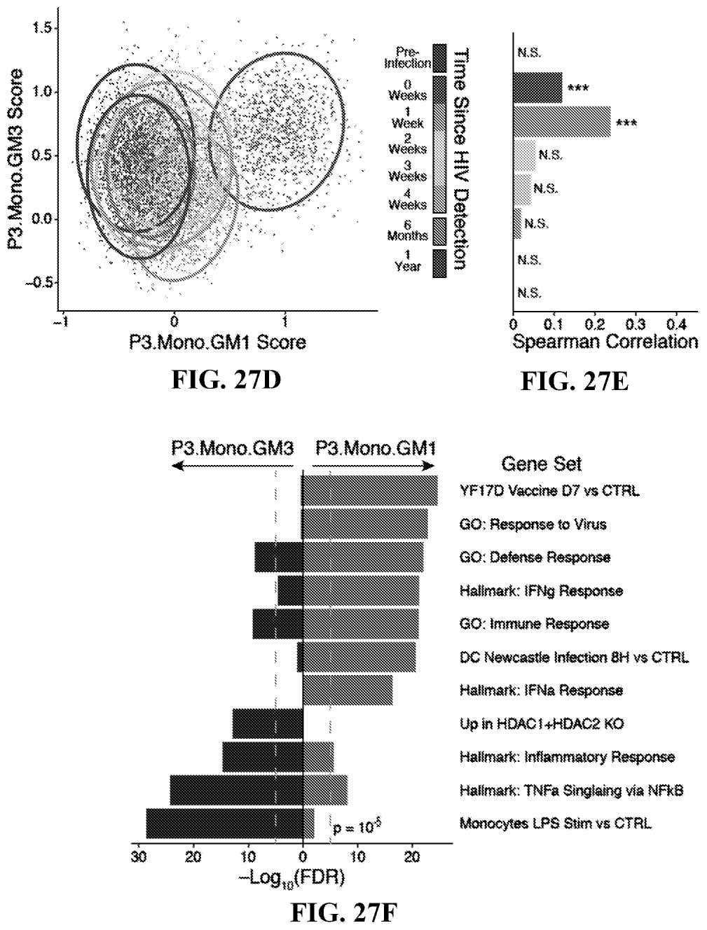

In the analyses, Applicants observed GMs that demonstrated similar temporal response patterns within the same cell type but distinct pathway enrichments, implying orthogonal biological functionality: for example, the NK cytokine signaling GM3 module (CCL3, CCL4) and the cytotoxic GM4 module (PRF1, GZMB) in P3 (FIG. 18H). To understand how these GMs might be linked, Applicants looked across single cells for module coexpression (obscured in bulk approaches). Surprisingly, the strength of the correlation between expression of these modules across single NK cells changed with time, decreasing later in infection (FIG. 27A, 27B). K-means clustering separated cells by variable expression of GM3 and GM4 (FIG. 27C). Variation in the correlation of GM3 and GM4 may reflect NK cell plasticity with dual cytotoxic and signaling programming near peak viremia.

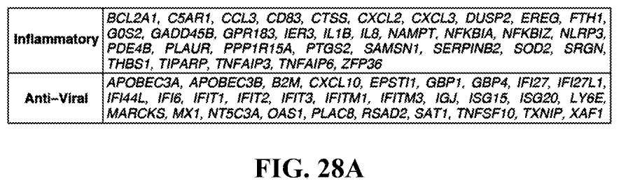

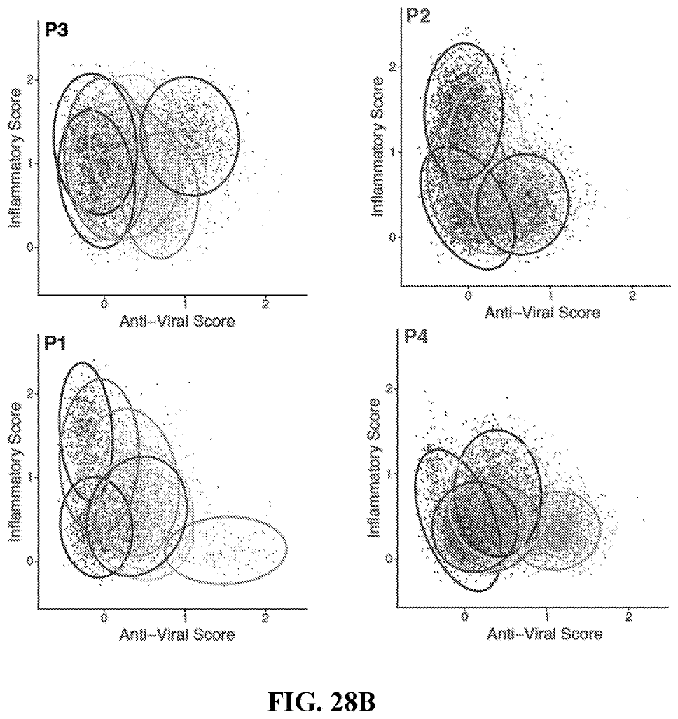

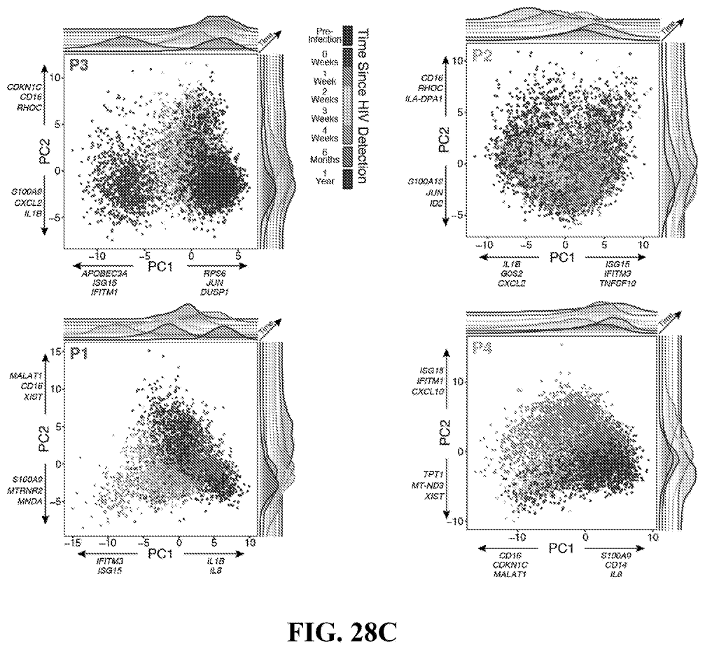

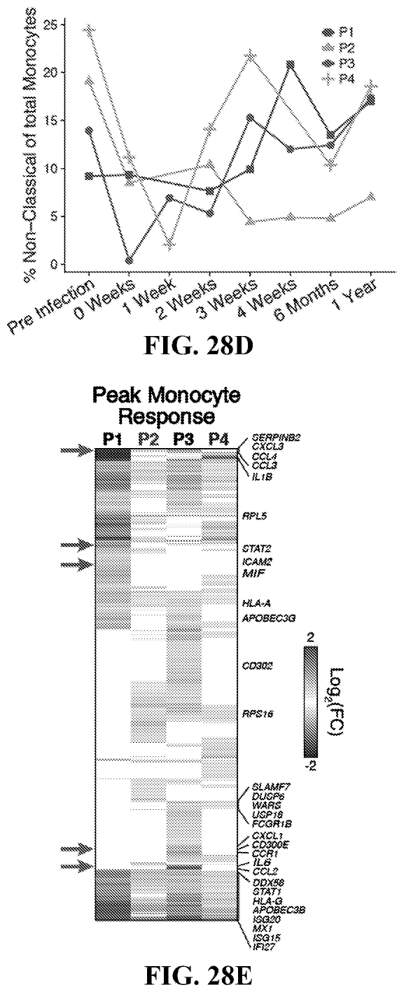

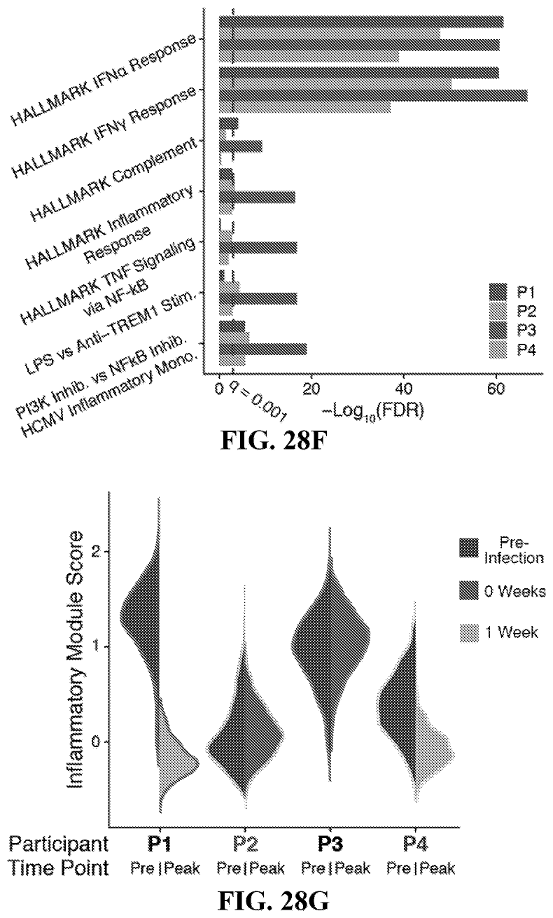

Examining MMsp (ISG GMs), Applicants also observed that P3 exhibited temporally similar modules in monocytes (GM1 and GM3); however, these did not variably correlate over time. Instead, they were highly coexpressed, but only at HIV-detection (FIGS. 27D, 27E). Gene-set analysis demonstrated that monocyte GM1 consisted of antiviral response genes, while GM3 was enriched for genes associated with inflammation (FIG. 27F). Thus, monocytes in P3 at the time of HIV detection are simultaneously expressing both antiviral and inflammatory gene programs, a previously unappreciated phenotype. While both gene programs strongly contributed to the major axes of monocyte variation in all individuals, Applicants were unable to identify polyfunctional monocytes in the other participants (FIGS. 28A-28C). Meanwhile, non-classical monocytes displayed disparate temporal dynamics across participants (FIGS. 20D and 28D). Comparing differentially expressed genes at peak response timepoints (1-2 weeks) further highlighted other participant-specific differences: monocytes in all participants produced antiviral factors (FIGS. 28E, 28F), but only P2 and P3 were enriched for inflammatory responses and only P3 for TNF signaling via NF-κB (q<0.001). Chronic inflammation has been associated with susceptibility to infection34 and the data show variable inflammatory gene expression before infection with subsequent mixed expression changes in hyperacute infection across participants (FIG. 28G).

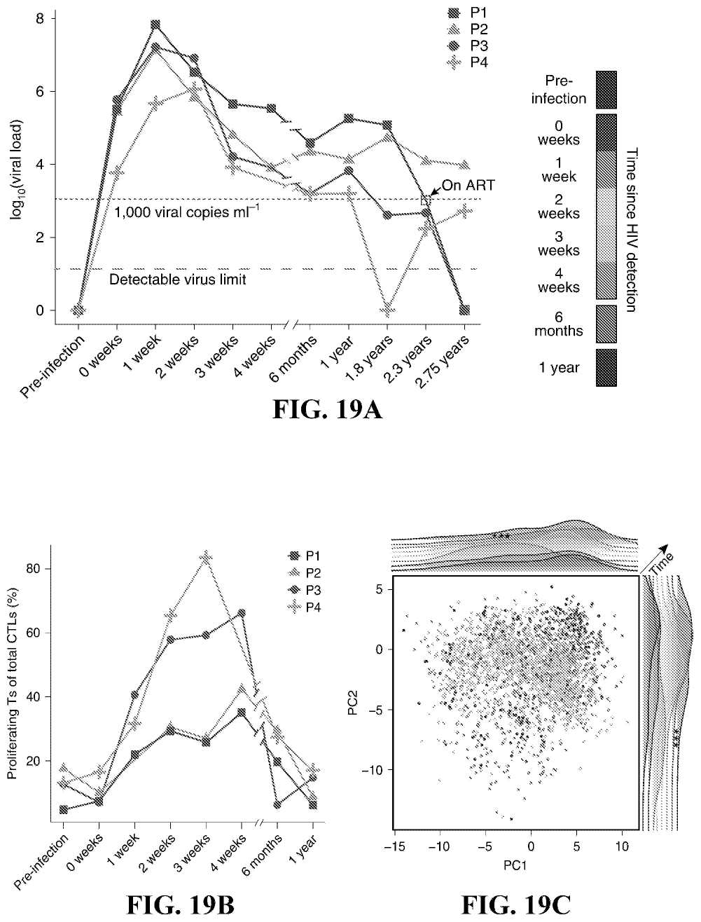

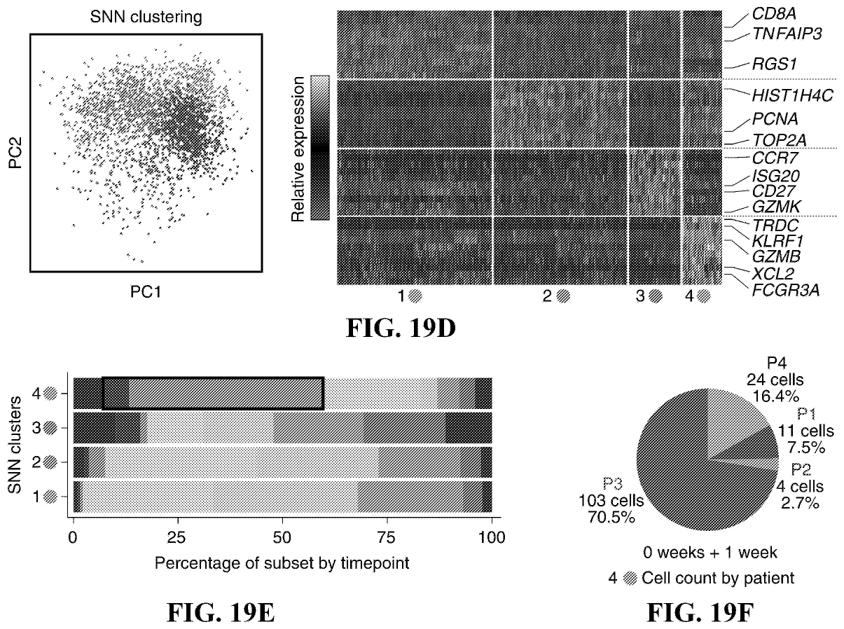

Natural control of HIV is associated with diverse cellular phenotypes in CTLs35 and DCs4. Applicants looked to see whether the presence of polyfunctional monocytes in P3 might link to disease progression in chronic infection. Applicants observed that both P3 and P4 maintained low levels of viremia (<1,000 viral copies ml-1) at 2.75 years after infection in the absence of antiretroviral therapy (ART) (FIG. 19A). HIV infected persons who naturally maintain low levels of viremia in chronic infection (HIV controllers) demonstrated enhanced immune responses systemically^{4,36}.

As CD8+ T cells contribute to controlling chronic HIV infection^{35,36}, Applicants also analyzed CTLs from all participants, noting increasing levels of PRF1 and GZMB during acute infection (FIG. 18G). Further unsupervised and directed approaches did not demonstrate significant differences in CTL responses across participants (FIGS. 29A, 29B). In FRESH, Applicants demonstrated that the majority of proliferating CTLs in acute infection are HIV specific³⁷. Therefore, Applicants looked for differences in proliferating T cell responses by participant. On average, proliferating T cells expressed similar levels of cytotoxic genes as non-proliferating CTLs. Differential expression analysis highlighted genes associated with cell cycle and memory for proliferating and nonproliferating CTLs, respectively (FIGS. 29C, 29D). T cell receptor (TCR) pulldown and enrichment (TCR-β CDR3) revealed few expanded clones (FIGS. 29E, 29F); this, however, may be affected by sample size (CDR3s were detected in 982 proliferating T cells). Relative to P1 and P2, both controllers (P3 and P4) displayed higher frequencies of proliferating cytotoxic cells within the first month of infection compared to pre-infection (FIG. 19B).

Applicants next used unsupervised analyses to examine differences in proliferating T cell responses over time among participants (FIG. 19C and FIG. 29G). Clustering over all proliferating T cells, Applicants identified four subsets of cells with distinct gene programs (FIG. 19D): traditional CD8+ T cells, hyperproliferative CD8+ T cells, naive CD4+ T cells and a subset of cells that were CD8A− but TRDC+ and FCGR3A+ (CD16). Using signatures from a single-cell study of cytotoxic cells, Applicants determined that the FCGR3A+ cells were NK cells (FIG. 29H). Looking at the distribution of cells within each of these clusters, the NK cluster contained the highest proportion of proliferating cells at HIV detection and 1 week thereafter (FIGS. 19E, 19F). The majority of these were from P3 and P4. Thus, the data show that the two participants who maintain viral loads <1,000 viral copies ml⁻¹ at 2.75 years after infection without ART exhibit a subset of proliferative, cytotoxic NK cells during the earliest stages of acute infection before the majority of HIV-specific CD8+ T cells arise.

Here, Applicants present and applied a novel scRNA-seq-based framework to a unique longitudinal study of human infection in order to characterize conserved immune response dynamics, as well as cell subsets and gene programs with potential therapeutic and preventative applications. By analyzing hundreds of cells per timepoint and cell type, Applicants were powered to identify significant changes in abundant cellular phenotypes over time in each participant. Applicants discovered interrelated temporal GM expression patterns in distinct cell types and nominated mechanisms by which multiple components of the immune system may respond collectively-sometimes with different gene programs—to HIV infection. By identifying upstream drivers that may induce the MMs, Applicants found when and how various cytokines, chemokines and transcription factors might orchestrate immune responses during infection. Together, this work affords a unique reference dataset for studying the earliest moments of HIV infection after detection and suggests potentially new roles for monocytes, NK cells and CD4+ T cells in acute infection.

The single-cell approach also enabled identification cellular sub-sets present during hyperacute HIV infection in two individuals (P3 and P4) who maintained low viremia in chronic infection. In addition to polyfunctional monocytes identified in P3, Applicants found a subset of cytotoxic, proliferating NK cells in P3 and P4. In other infection settings38,39, NK cells have demonstrated antigenic memory, suggesting that these cells could be responding to some previously encountered antigen. These proliferating NK cells may function alongside CTLs early in infection, mitigating CTL antigenic load and subsequent exhaustion⁴⁰. However, there were ethical and practical difficulties associated with collecting additional samples from untreated HIV infected persons.

### REFERENCES

1. Regev, A. et al. The human cell atlas. eLife 6, e27041 (2017).
2. Gomes, T., Teichmann, S. A. & Talavera-López, C. Immunology driven by large-scale single-cell sequencing. Trends Immunol. 40, 1011-1021 (2019).
3. Shalek, A. K. & Benson, M. Single-cell analyses to tailor treatments. Sci. Transl. Med. 9, eaan4730 (2017).
4. Martin-Gayo, E. et al. A reproducibility-based computational framework identifies an inducible, enhanced antiviral state in dendritic cells from HIV-1 elite controllers. Genome Biol. 19, 10 (2018).
5. Ndung'u, T., Dong, K. L., Kwon, D. S. & Walker, B. D. A FRESH approach: combining basic science and social good. Sci. Immunol. 3, eaau2798 (2018).
6. Joint United Nations Programme on HIV/AIDS. UNAIDS Data 2019. (UNAIDS, 2019).
7. Robb, M. & Ananworanich, J. Lessons from acute HIV infection. Curr. Opin. HIV AIDS 11, 555-560 (2016).
8. Fiebig, E. et al. Dynamics of HIV viremia and antibody seroconversion in plasma donors: implications for diagnosis and staging of primary HIV infection. AIDS 17, 1871-1879 (2003).
9. Pijuan-Sala, B., Guibentif, C. & Göttgens, B. Single-cell transcriptional profiling: a window into embryonic cell-type specification. Nat. Rev. Mol. Cell Biol. 19, 399 (2018).
10. Saelens, W., Cannoodt, R., Todorov, H. & Saeys, Y. A comparison of single-cell trajectory inference methods. Nat. Biotechnol. 37, 547-554 (2019).
11. Wang, T., Li, B., Nelson, C. E. & Nabavi, S. Comparative analysis of differential gene expression analysis tools for single-cell RNA-sequencing data. BMC Bioinformatics 20, 40 (2019).
12. Tirosh, I. & Suva, M. L. Deciphering human tumor biology by single-cell expression profiling. Annu. Rev. Cancer Biol. 3, 151-166 (2019).
13. Schoggins, J. W. & Rice, C. M. Interferon-stimulated genes and their antiviral effector functions. Curr. Opin. Virol. 1, 519-525 (2011).
14. Corleis, B. et al. Early type I Interferon response induces upregulation of human (3-defensin 1 during acute HIV-1 infection. PLoS ONE 12, e0173161 (2017).
15. Vargas-Inchaustegui, D. A. et al. CXCL10 production by human monocytes in response to Leishmania braziliensis infection. Infect. Immun. 78, 301-308 (2010).
16. Luban, J. Innate immune sensing of HIV-1 by dendritic cells. Cell Host Microbe 12, 408-418 (2012).
17. Ng, D. & Gommerman, J. L. The regulation of immune responses by DC derived type I IFN. Front. Immunol. 4, 94 (2013).
18. Kurtulus, S. et al. TIGIT predominantly regulates the immune response via regulatory T cells. J. Clin. Invest. 125, 4053-4062 (2015).
19. Samten, B. CD52 as both a marker and an effector molecule of T cells with regulatory action: identification of novel regulatory T cells. Cell Mol. Immunol. 10, 456-458 (2013).
20. Lugli, E., Marcenaro, E. & Mavilio, D. NK cell subset redistribution during the course of viral infections. Front. Immunol. 5, 390 (2014).
21. Bosinger, S. E. et al. Global genomic analysis reveals rapid control of a robust innate response in SIV-infected sooty mangabeys. J. Clin. Invest. 119, 3556-3572 (2009).
22. Bosinger, S. E. et al. Intact type I interferon production and IRF7 function in sooty mangabeys. PLoS Pathogens 9, e1003597 (2013).
23. O'Brien, M., Manches, O. & Bhardwaj, N. Plasmacytoid dendritic cells in HIV infection. Adv. Exp. Med. Biol. 762, 71-107 (2013).
24. Jiao, Y. et al. Plasma IP-10 is associated with rapid disease progression in early HIV-1 infection. Viral Immunol. 25, 333-337 (2012).
25. Shive, C. L., Jiang, W., Anthony, D. D. & Lederman, M. M. Soluble CD14 is a nonspecific marker of monocyte activation. AIDS 29, 1263-1265 (2015).
26. Athanasiadis, E. I. et al. Single-cell RNA-sequencing uncovers transcriptional states and fate decisions in haematopoiesis. Nat. Commun. 8, 2045 (2017).
27. Matsusaka, T. et al. Transcription factors NF-IL6 and NF-xB synergistically activate transcription of the inflammatory cytokines, interleukin 6 and interleukin 8. Proc. Natl Acad. Sci. USA 90, 10193-10197 (1993).
28. Yue, F. Y. et al. Virus-specific interleukin-17-producing CD4+ T cells are detectable in early human immunodeficiency virus type 1 infection. J. Virol. 82, 6767-6771 (2008).
29. Hou, W., Jin, Y.-H., Kang, H. S. & Kim, B. S. Interleukin-6 (IL-6) and IL-17 synergistically promote viral persistence by inhibiting cellular apoptosis and cytotoxic T cell function. J. Virol. 88, 8479-8489 (2014).
30. Sloan, R. D. et al. Productive entry of HIV-1 during cell-to-cell transmission via dynamin-dependent endocytosis. J. Virol. 87, 8110-8123 (2013).
31. Jakubzick, C. V., Randolph, G. J. & Henson, P. M. Monocyte differentiation and antigen-presenting functions. Nat. Rev. Immunol. 17, 349-362 (2017).
32. Gulzar, N. & Copeland, K. F. T. CD8+ T-cells: function and response to HIV infection. Curr. HIV Res. 2, 23-37 (2004).
33. Scully, E. & Alter, G. NK cells in HIV disease. Curr. HIV AIDS Rep. 13, 85-94 (2016).
34. Kaspersen, K. A. et al. Low-grade inflammation is associated with susceptibility to infection in healthy men: results from the Danish blood donor study (DBDS). PLoS ONE 11, e0164220 (2016).
35. Ranasinghe, S. et al. Antiviral CD8+ T cells restricted by human leukocyte antigen class II exist during natural HIV infection and exhibit clonal expansion. Immunity 45, 917-930 (2016).
36. Walker, B. D. & Yu, X. G. Unravelling the mechanisms of durable control of HIV-1. Nat. Rev. Immunol. 13, 487-498 (2013).
37. Ndhlovu, Z. M. et al. Magnitude and kinetics of CD8+ T cell activation during hyperacute HIV infection impact viral set point. Immunity 43, 591-604 (2015).
38. Reeves, R. K. et al. Antigen-specific NK cell memory in rhesus macaques. Nat. Immunol. 16, 927-932 (2015).
39. Cerwenka, A. & Lanier, L. L. Natural killer cell memory in infection, inflammation and cancer. Nat. Rev. Immunol. 16, 112-123 (2016).
40. Hoffmann, M. et al. Exhaustion of activated CD8 T cells predicts disease progression in primary HIV-1 infection. PLoS Pathogens 12, e1005661 (2016).

Methods

Study participants. All participants in this study were enrolled in the FRESH cohort5,41. This prospective study recruited women who were HIV negative, aged 18-24 years and were tested for HIV-1 RNA in plasma twice weekly for 1 year. Each time the women came to the study center, they participated in peer-support groups and received a stipend. In addition to semi-weekly virus testing by PCR with reverse transcription, whole blood was collected four times (including during enrollment) throughout the year from participants. If a plasma test came back positive, the participant was asked to come back to the clinic that day to collect a blood sample. Samples were then collected weekly through the first 6 weeks of infection and regularly afterward as long as the participant continues to return to the study center. In the arm of the study described herein, participants were initiated on ART when their CD4 count fell below 350 cells μl⁻¹, per standard treatment guidelines at the time of enrollment. A second arm of the study was initiated in 2014 and is currently still in place; in that arm, participants who tested positive for viral RNA were initiated on ART when they were called back into the study center for their first post-infection sample collection. To the best of Applicants knowledge, all participants in this study had not yet started ART for the timepoints processed here. FRESH was performed in accordance with protocols approved by the Institutional Review Board at Partners (Massachusetts General Hospital), MIT and the Biomedical Research Ethics Committee of the University of KwaZulu-Natal. All FRESH participants consented for genetic and genomic data collection and analysis.

Cell preparation, flow cytometry and cell sorting. The Life Sciences Reporting Summary contained information on the sample preparation, antibodies, gating strategy and sort strategy used in this study.

Single-cell RNA-seq with Seq-Well. The Seq-Well platform was utilized as previously described⁴² to capture the transcriptomes of single cells on barcoded mRNA capture beads. In brief, 10 μl of sorted CD45+Calcein Blue+ PBMCs were mixed at 1:1 dilution with Trypan blue and counted using a hemocytometer.

The cells were resuspended in RPMI+10% FBS at a final concentration of ˜100,000 live cells ml⁻¹ and 20,000-25,000 cells in 200 μl were added to each Seq-Well array preloaded with barcoded mRNA capture beads (ChemGenes).

Two arrays were used for each sample to increase cell numbers. The arrays were then sealed with a polycarbonate membrane (pore size of 0.01 μm), cells were lysed, transcripts were hybridized to the beads and the barcoded mRNA capture beads were recovered and pooled for reverse transcription using Maxima H-RT (Thermo Fisher EPO0753) and all subsequent steps. After an Exonuclease I treatment (NEB M0293L) to remove excess primers, whole transcriptome amplification (WTA) was carried out using KAPA HiFi PCR Mastermix (Kapa Biosystems KK2602) with 2,000 beads per 50 μl of reaction volume. Libraries were then pooled in sets of eight (totaling 16,000 beads) and purified using Agencourt AMPure XP beads (Beckman Coulter, A63881) by a 0.6×volume wash followed by a 0.8× volume wash and quantified using Qubit hsDNA Assay (Thermo Fisher Q32854). Quality of the WTA product was assessed using the Agilent hsD5000 Screen Tape System (Agilent Genomics) with an expected peak >800 bp tailing off to beyond 3,000 bp, and a small or non-existent primer peak, indicating a successful preparation. Libraries were then constructed using a Nextera XT DNA library preparation kit (Illumina FC-131-1096) on a total of 750 pg of pooled cDNA library from 16,000 recovered beads using index primers as previously described43. Tagmented and amplified sequences were purified using a 0.8× volume AMPure XP bead wash yielding library sizes with an average distribution of 500-750 bp in length as determined using an Agilent hsD1000 Screen Tape System (Agilent Genomics). Two Seq-Well arrays were sequenced per NextSeq500 sequencing run with an Illumina 75 Cycle NextSeq500/550 v2 kit (Illumina FC-404-2005) at a final concentration of 2.4 pM. The read structure was paired end with Read 1, starting from a custom read 1 primer, covering 20 bases inclusive of a 12-bp cell barcode and 8-bp unique molecular identifier (UMI), then an 8-bp index read and finally Read 2 containing 50 bases of transcript sequence.

Seq-Well alignment, cell identification and cell type separation. Read alignment, cell barcode discrimination and UMI per transcript collation were performed as by Ordovas-Montanes et al.⁴³ using a hgl9 reference. Initially, Applicants aligned the sequences from P1 to a combined HIV+hg19 genome using the consensus sequence of HIV clade C viruses from the HIV Sequence Database (www.hiv.lanl.gov/content/sequence/HIV/mainpage.html). After alignment, however, Applicants measured 0-2 cells with HIV transcript alignments per array; therefore, Applicants used the standard hgl9 reference for the analysis. UMI-collapsed data were used as input into Seurat44 (v.2.3.4) for cell and gene trimming and downstream analysis. The following steps were performed on all of the arrays processed from a single participant, on a participant-by-participant basis. Any cell with <750 UMIs or >6,000 UMIs (0-5 cells per array) and any gene expressed in fewer than five cells were discarded from downstream analysis. This cells-by-genes matrix was then used to create a Seurat object for each participant. Cells with >20% of UMIs mapping to mitochondrial genes were then removed (50-100 cells per array). These objects (one per participant) were then merged into one object for pre-processing and cell type identification.

The combined Seurat object was log-normalized with a size factor of 10,000 and scaled without centering. Additionally, linear regression was performed to remove unwanted variation due to cellular complexity (nUMI) and low-quality cells (percent.mito). Subsequently, 3,251 variable genes were identified using the ‘LogVMR’ function and the following cutoffs: x.low.cutoff=0.01, x.high.cutoff=10 and y.cutoff=0.25. PCA was performed over these genes and the top 17 PCs were chosen for clustering and embedding on the basis of the curve of variance described by each PC and the genes most contributing to each PC. Next, FindClusters (SNN graph+ modularity optimization) with a resolution of 0.5 was used to generate 13 clusters and the Fourier transform t-distributed stochastic neighbor embedding (tSNE) implementation⁴⁵ with 2,000 iterations to embed the data into two-dimensional space.

Cluster identity was assigned by finding differentially expressed genes using Seurat's implemented Wilcoxon rank-sum test and then comparing those cluster-specific genes to previously published datasets⁴⁶⁻⁴⁸. One cluster exhibited no cluster-specific genes; the cells from this cluster were embedded centrally in the tSNE, and on further investigation expressed both myeloid and lymphocyte markers. Therefore, these cells were removed as multiplets (when multiple cells enter the same well in the Seq-Well array). After multiplet removal, 59,162 cells were captured across all samples processed. The remaining 12 clusters included subsets of major circulating immune cells. These clusters were merged by parent cell type (T cell, cytotoxic T cell, B cell, plasmablast, DC and monocyte) for downstream analysis, as variation in the SNN graph parameters weakly affected cluster assignment to the subsets.

As NK cells share many markers transcriptionally with cytotoxic T cells⁴⁶, clustering in the dataset did not separate these two cytotoxic cell types. NK cells were annotated based on lacking expression of CD3 (CD3D, CD3E, CD3G) and nonzero expression of CD16 (FCRG3A) and KLRF1. CD56 (NCAM) was not highly expressed in the data and therefore was not used to separate NK cells. Any cell with a cluster identity belonging to the cytotoxic T cell cluster that lacked CD3 expression or expressed CD16/KLRF1 was annotated as an NK cell. With this annotation, Applicants noted distinct transcriptional responses between NK cells and CTLs both as a function of time and gene membership (FIG. 17C and FIGS. 18G, 18H).

For downstream analysis of temporal variation in expression, the dataset was separated by participant and cell type: CD4+ T cells, NK cells, CTLs, proliferating T cells, B cells, plasmablasts, mDCs and monocytes. The expression matrix and associated metadata can be accessed online through the Single Cell Portal hosted by Broad Institute of MIT and Harvard (see Data Availability; singlecell.broadinstitute.org/single_cell/study/SCP256).

Cell type normalization. Once separated by cell type and participant, the single-cell transcriptomes were processed on a cell-type-by-cell-type basis across all timepoints. For each cell type, the presence of residual contaminant RNA or doublets was assayed by scoring every cell against a set of contaminant genes from other cell types built from the marker list used to discern cluster identity. Cells with high contamination scores (0-10% of cells) were subsequently removed from further analysis to avoid unwanted variation in the subsequent unsupervised module discovery. Following contamination filtering, data underwent scaling and normalization, followed by variable gene discovery (˜400-1,000 genes, dependent on cell type and cell number). PCA was then applied on the limited set of genes, followed by projection to the rest of the genes in the dataset.

Module discovery. For the module analysis, Applicants subset the data on the top and bottom 50 genes, after projection, for the first 3-9 PCs (dependent on the variance described by each PC and genes contributing to each PC) as input for WGCNA49,50 functions. Following the WGCNA tutorial (horvath.genetics.ucla.edu/html/CoexpressionNetwork/Rpackages/WGCNA/Tutorials/), an appropriate soft-power threshold was chosen to calculate the adjacency matrix. As scRNA-seq data was impacted by transcript dropout (failed capture events), adjacency matrices with high power further inflate the impact of this technical limitation and yield few correlated modules. Therefore, when possible, a power was chosen as suggested by the authors of WGCNA (that is, the first power with a scale-free topology >0.8); however, in instances where this power yielded few modules (fewer than three), Applicants decreased the power. As a rule, smaller soft powers lead to fewer large-sized modules, whereas larger soft powers lead to more small-sized modules. In the analysis, there was frequently a distinct tipping point where, as Applicants increased soft power, modules would fail to be identified by WGCNA (due to low connectivity). Applicants ran the analyses with several soft powers to find an appropriate balance to generate a maximal number of modules without losing GM membership. Next, an adjacency matrix was generated using the selected soft power and it was transformed into a TOM. Subsequently, this TOM was hierarchically clustered and the cutreeDynamic function with method ‘tree’ was used to generate modules of correlated genes (minimum module size of ten). Similar modules were then merged using a dissimilarity threshold of 0.5 (that is, a correlation of 0.5); WGCNA typically suggests dissimilarity thresholds of 0.8-0.95, but Applicants sought to avoid any spurious cluster separation potentially associated with the chosen soft power.

To test the significance of the correlation structure of a given module, a permutation test was implemented. Binning genes in the true module by average gene expression (number of bins was ten), genes with the same distribution of average expression from the total list of genes used for module discovery were randomly picked 10,000 times. For each of these random modules, a one-sided Mann-Whitney U-test was performed to compare the distribution of dissimilarity values between the genes in the true module and the distribution of dissimilarity values between the genes in the random module. Correcting the resulting P values for multiple hypothesis testing by Benjamini-Hochberg FDR correction, a module was considered significant if <500 tests (P<0.05) had FDR>0.05. Applicants note that if Applicants chose a smaller soft power for TOM generation, which in turn resulted in larger modules with fewer excluded genes, fewer modules passed this permutation test, likely due to noisier genes that maintained weak correlations with all other genes in the analysis.

Since Applicants were interested in identifying modules of genes that changed in expression as a function of time, another permutation test was implemented to identify modules that significantly vary from pre-infection. First, every cell was scored for the genes within the module, using the AddModuleScore function in Seurat. This function calculated an average module score by calculating the mean expression of the genes within the module corrected for expression of other genes with similar means across the dataset. Thus, this score functions as an expression estimate of the genes within a module in any given single cell. As testing for differences in distribution is sensitive to sample number, a sample size (s) was selected based on the number of cells present at any given timepoint within a cell type. The smallest s used was ten; this cutoff was chosen based on the least frequent cell types having ˜100 cells total across all timepoints within a participant. If a timepoint had fewer than ten cells, that point was not used in the testing. In the case of plasmablasts and mDCs in multiple participants, more than three timepoints had fewer than ten cells and therefore no modules were considered significantly variant in time. To determine whether module expression varied over time, 1,000 two-sided Mann-Whitney U-tests between the distribution of scores from s random cells at pre-infection and s random cells from each other timepoint were performed. For each timepoint, the P values from the 1,000 tests were averaged. After FDR correction, if q<0.05 for any timepoint, the module was considered to significantly vary in expression in time. The approach and tests have been written as functions in R and have been included as Supplementary Software.

Applicants were powered to identify temporally similar modules active in distinct subsets of cells both within and across time, and Applicants used each cell of a specific type as a well-controlled, independent biological replicate to identify, from a single sample, essential response features and their putative upstream drivers. Compared to a directed approach, this discovery-based identification of temporally variant modules enabled unbiased selection of coordinated genes and pathways and immediately reveals differences in response dynamics among cell types, states and participants.

For the cross-participant module discovery analysis (FIGS. 24A-24D), Applicants applied the WGCNA framework to all cells of a given cell type across all four participants at all timepoints sampled. Here, the number of genes input into the framework varied between ˜350-850 genes by choosing the top and bottom 100 genes from the most significant PCs, determined by finding the asymptote in the PC elbow plot (ranked s.d. of each PC). These modules were then tested for significant correlation against random sets of genes using the same permutation test outlined above. To test for temporal variability in module expression across all four participants, Applicants binned timepoints into pre-infection, peak, post-peak and 1-year and implemented an analysis of variance (ANOVA) across binned timepoints accounting for participant heterogeneity (see FIG. 24A).

Specifically, Applicants fitted a linear regression to the data across binned timepoints using two models: (1) null hypothesis ˜1+participant; and (2) alternative hypothesis ˜1+participant+time.bin. Applicants then calculated the F statistic for the ANOVA between these two models. Peak and post-peak timepoints were chosen based on the score maxima for the modules discovered in each participant in MMsp and MMgp (see FIGS. 19A-19F).

Module grouping and gene-set analysis. To more easily compare modules by temporal pattern within and between participants, fuzzy c-means clustering was applied to all of the modules in a given participant using the Mfuzz package51 (v.2.38.0). Applicants chose to use fuzzy c-means clustering to allow us to understand the extent of membership of a given module to its assigned cluster. For each participant, c was chosen to be 5-7, such that diverse temporal patterns were separated, minimizing the number of clusters containing fewer than three modules. These groupings of modules were then annotated by similar scoring patterns across participants, taking into consideration that infection time is not the same for every participant (FIGS. 19A-19F). Applicants named four of these MMs on the basis of the transient module score dynamics of each: MMsp, MMsn, MMgp and MMgn. The remaining three MMs were named a-c given their more complex score dynamics.

Gene-set analysis on modules was performed using IPA (Qiagen) given its better performance with low gene numbers; the modules were sized between 10-66 genes. Only gene names were supplied for analysis and submitted for core analysis with the experimentally observed confidence setting. In FIGS. 18D-18I, the pathways annotated were taken from either the canonical pathways or diseases and functions results. For the upstream driver analysis in FIGS. 25D, 25E, upstream drivers were selected by the following criteria: significant (P<0.001) in at least two modules of any given cell type, with at least five genes in the gene set. As the gene sets annotated in IPA are quite large and share many genes, the edges in the network were restricted to only those upstream drivers that shared three or more genes. To achieve finer grain temporal resolution on putative inducers of immune response, the union of enriched genes for each upstream driver from modules within a given cell type was used to generate scores against the single-cell expression data. Only upstream driver scores that demonstrated temporal variability (as described above) were included. Applicants report the median scores at each timepoint for each upstream driver.

Applicants chose to use parts of MSigDB v.6.2 (software.broadinstitute.org/gsea/msigdb) for the gene-set enrichment analysis in FIGS. 27A-27F and 28A-28G, given higher gene numbers (>100), allowing for more conservative P values. Multiple-hypothesis testing was corrected by the Benjamini-Hochberg FDR procedure. The specific collections of gene sets used are reported in the figure legends.

Cell-cell signaling network curation. The cell-cell signaling networks in FIGS. 18D, 18J and FIG. 26B were generated using connections annotated in IPA52. Molecules of interest were chosen from genes in the modules belonging to MMgp in P2 (FIG. 18D) or shared among at least two participants (FIG. 18J, FIG. 26B), respectively. Applicants also included select upstream drivers found to be significant by IPA given enrichment of downstream genes within the modules. Edges were drawn between all nodes (genes or predicted upstream drivers) with the ‘Connect’ tool in ‘My Pathways’ using both ‘Direct’ and ‘Indirect’ interactions. Subsequently, edges were manually trimmed by looking at the provided support for the connections and discarding any connections not supported by demonstrations of expression or activation in the literature. For FIG. 18J, any predicted upstream driver-gene edge that connected to a cell for which that upstream driver was not significantly enriched was also trimmed (for example, only edges between IL-10 and nodes for genes in CD4+ T cells were kept). Contextualization of these cell-cell signaling networks may be further explored online: shaleklab.com/resource/immune-dynamics-of-acute-hiv-infection.

Comparison to microarray data generated in acute SIV infection. To contextualize the single-cell results against previously published datasets in SIV infection, Applicants compared genes differentially expressed at peak viremia (compared to pre-infection) in all eight cell types studied in P1 to genes upregulated in rhesus macaques 0-180 d after SIV infection21. Any genes found to be differentially expressed (FDR-corrected q<0.05) in the data were depicted alongside any gene that demonstrated log 2(fold change) upregulation at any timepoint in the Bosinger et al. microarray experiments²¹ (FIG. 22B). Applicants note that compared to many of the ISGs upregulated in acute SIV infection, where upregulation persists for >2 weeks, most ISGs in P1 were only differentially expressed at the 1-week timepoint, indicating potential differences in the evolution of immune responses between humans and macaques.

scRNA-seq of pDCs with SMART-seq2 and analysis. Reverse transcription, WTA and library preparation of single pDCs in 96-well plates was performed as previously described⁵³. Samples were sequenced on an Illumina NextSeq500/550 instrument with an Illumina 75 Cycle NextSeq500/550 v2 kit (Illumina FC-404-2005) using 30-bp paired-end reads. Given difficulties acquiring pDCs from pre-infection samples due to limited cell numbers, Applicants sequenced pDCs from the peak IFN response and the 1-year timepoints in each participant. Reads (5×105 to 3×106 per cell) were aligned to the hg38 (GENCODE v.21) transcriptome and genome using RSEM54 and Tophatss, respectively. After trimming low-quality cells (cells with <25,000 mapped reads or <1,000 genes), the remaining cells had a median of 122,000 mapped reads and 2,866 genes. Pre-processing and differential expression analysis were conducted in Seurat44 using the Wilcoxon rank-sum test. To test for differences in IFN responsiveness, participant-specific IFN response gene lists were used to generate scores in the pDCs using the AddModuleScore function in Seurat. The gene list used to score in each participant was chosen by including any gene that appeared at least twice in the modules that belonged to MM3 for that participant (see FIG. 22I).

Luminex and ELISA cytokine measurements. Matching plasma cytokine levels were determined in duplicate using a multiplexed magnetic bead assay (catalog no. LHC6003M, Life Technologies) in accordance with the manufacturer's instructions. Briefly, a mixture of beads that were coated with anticytokine antibodies were prewashed and then incubated with the plasma samples. They were then co-incubated with a mixture of biotinylated detector antibodies followed by R-phycoerythrin (R-PE)-conjugated streptavidin. A magnetic separator was used to wash the beads between incubations. Fluorescence intensity was determined on a Bio-Plex 200 system. Concentrations of the cytokines in the samples were determined by interpolating on sigmoid four-parameter logistic regression standard curves.

Matching plasma soluble CD14 levels were measured using human CD14 DuoSet ELISA Kit (catalog no. DY383, R&D Systems) in accordance with the manufacturer's instructions. Briefly, a 96-well microplate was coated with anti-CD14 capture antibody overnight. The plates were blocked with reagent diluent for 1 h and then incubated with recombinant standards or plasma samples (diluted 1:600 in reagent diluent) for 1.5 h. They were further incubated with detection antibody for 1.5 h, followed by streptavidin-HRP for 20 min. The substrate was added for 20 min for color development. The reaction was stopped by adding stop solution. Optical density (OD) of each well was determined at 450 nm (corresponding ODs at 530 nm were subtracted for wavelength correction). The concentrations of soluble CD14 in the samples were determined by interpolating on a sigmoid four-parameter logistic regression standard curve. All incubations were done at room temperature.

T cell receptor CDR3 pulldown and analysis. To directly sequence the CDR3s from proliferating T cells assayed by Seq-Well, Applicants applied a recently published TCR pulldown method56 to WTA products from the 2-week, 3-week and 4-week timepoint samples from all four participants. Briefly, biotinylated capture probes from the TRBC region were annealed to melted WTA cDNA. Magnetic streptavidin beads were then used to pull down cDNA enriched for TRBC; this cDNA was subsequently amplified using KAPA HiFi Mastermix (Kapa Biosystems) and purified using 0.75× SeraPure beads to select for 0.8-1-kb sized DNA fragments. To select for sequences with full CDR3 regions, a pool of V-region primers was used to further amplify sequences of interest. Step-out PCR was used to add sequencing handles and the resulting libraries were sequenced on a NextSeq 550 using a 150-cycle NextSeq kit with 148 cycles for Read 1 (CDR3) and 20 cycles for Index 1 (BC+UMI). Sequences of the primers used are available in Tu et al.56. CDR3 consensus sequences were aligned and determined as outlined previously. Across the entire dataset, Applicants detected ˜50% of TCR-β chain CDR3s.

Determining the cellular identity of early proliferating cytotoxic cells. To determine whether TRDC+FCGR3A+ cells were γδT or NK cells, Applicants scored them, as well as nonproliferating CTLs and NK cells, against gene signatures described in that study (FIG. 29G). Based on score similarity to NK cells, and the relative downregulation of CD3 compared to the other proliferating T cell subsets (FDR-corrected Wilcoxon rank-sum test; CD3D: log(FC)=−0.895, q=2.7×10−42; CD3G: log(FC)=−0.923, q=8.9×10−37), Applicants determined cluster 4 (lilac) to be proliferating NK cells.

Statistics. WGCNA module significance was tested using a permutation test (n=10,000) on dissimilarity values and compared to the distribution of true values using a one-sided Mann-Whitney U-test. After FDR correction, modules that failed <500 tests (P<0.05) were considered significant. Participant-specific modules were tested for temporal variation in score using a two-sided Mann-Whitney U-test with 1,000 subsamplings. Modules discovered across all participants were tested for temporal variation with binned samples using an ANOVA with two models: (1) null ˜1+ participant; and (2) alternative ˜1+participant+time.bin (P<0.05). Gene-set analysis was performed on GMs using IPA (Qiagen). For the differential expression analysis in monocytes, Applicants performed hypergeometric tests using gene lists from MSigDB v.6.2 and corrected for multiple-hypothesis testing using the FDR procedure. Differential expression analysis between groups of single cells was performed using either a two-sided Wilcoxon rank-sum test or the ‘bimod’ test as implemented in Seurat.

### REFERENCES

Dong, K. L. et al. Detection and treatment of Fiebig stage I HIV-1 infection in young at-risk women in South Africa: a prospective cohort study. Lancet HIV 5, e35-e44 (2018).
Gierahn, T. M. et al. Seq-Well: portable, low-cost RNA sequencing of single cells at high throughput. Nat. Methods 14, 395-398 (2017).
Ordovas-Montanes, J. et al. Allergic inflammatory memory in human respiratory epithelial progenitor cells. Nature 560, 649 (2018).
Butler, A., Hoffman, P., Smibert, P., Papalexi, E. & Satija, R. Integrating single-cell transcriptomic data across different conditions, technologies and species. Nat. Biotechnol. 36, 411-420 (2018).
Linderman, G. C., Rachh, M., Hoskins, J. G., Steinerberger, S. & Kluger, Y. Fast interpolation-based t-SNE for improved visualization of single-cell RNA-seq data. Nat. Methods 16, 243 (2019).
Gutierrez-Arcelus, M. et al. Lymphocyte innateness defined by transcriptional states reflects a balance between proliferation and effector functions. Nat. Commun. 10, 687 (2019).
Villani, A. C. et al. Single-cell RNA-seq reveals new types of human blood dendritic cells, monocytes, and progenitors. Science 356, eaah4573 (2017).
Kang, H. M. et al. Multiplexed droplet single-cell RNA-sequencing using natural genetic variation. Nat. Biotechnol. 36, 89-94 (2018).
Zhang, B. & Horvath, S. A general framework for weighted gene co-expression network analysis. Stat. Appl. Genet. Mol. Biol. 4, 17 (2005).
Langfelder, P. & Horvath, S. WGCNA: an R package for weighted correlation network analysis. BMC Bioinformatics 9, 559 (2008).
Kumar, L. & E. Futschik, M. Mfuzz: a software package for soft clustering of microarray data. Bioinformation 2, 5-7 (2007).
Kramer, A., Green, J., Pollard, J. & Tugendreich, S. Causal analysis approaches in ingenuity pathway analysis. Bioinformatics 30, 523-530 (2014).
Trombetta, J. J. et al. Preparation of single-cell RNA-seq libraries for next generation sequencing. Curr. Protoc. Mol. Biol. 107, 1-17 (2014).
Li, B. & Dewey, C. N. RSEM: accurate transcript quantification from RNA-seq data with or without a reference genome. BMC Bioinformatics 12, 323 (2011).
Trapnell, C., Pachter, L. & Salzberg, S. L. TopHat: discovering splice junctions with RNA-seq. Bioinformatics 25, 1105-1111 (2009).
Tu, A. A. et al. TCR sequencing paired with massively parallel 3′ RNA-seq reveals clonotypic T cell signatures. Nat. Immunol. 20, 1692-1699 (2019).

Addition data related to the results presented in this example includes Supplemental Tables 1-11 in Kazer S W et al., Nature Medicine volume 26, pages 511-518 (2020), which is incorporated by reference herein in its entirety.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

LENGTHY TABLES
The patent application contains a lengthy table section. A copy of the table is available in electronic form from the USPTO web site (https://seqdata.uspto.gov/?pageRequest=docDetail&DocID=US20220226464A1). An electronic copy of the table will also be available from the USPTO upon request and payment of the fee set forth in 37 CFR 1.19(b)(3).

What is claimed is:

1. A method of treating or preventing a viral infection, the method comprising administrating an effective amount of a modulating agent that induces proliferation of γδ T cells and/or Natural killer (NK) cells to a subject in need thereof.

2. The method of claim 1, wherein the viral infection is a chronic viral infection.

3. The method of claim 2, wherein the viral infection is a human immunodeficiency virus (HIV) infection.

4. A method of treating or preventing a viral infection, the method comprising administrating an effective amount of a vaccine composition to a subject in need thereof, the vaccine composition comprising one or more modulating agents that induces proliferation of γδ T cells and/or NK cells.

5. The method of claim 4, wherein the one or more modulating agent modulates one or more biomarkers GAPDH, STMN1, KIAA0101, MKI67, MALAT1, TXNIP, IL7R, and KLRB1.

6. The method of claim 4, wherein the one or more modulating agents increases KLRB1 expression in the γδ T cells and/or NK cells.

7. A method of modulating an immune response to reduce baseline inflammation comprising administrating an effective amount of one or more modulating agents that increases expression or activity of APOBEC3A, IFITM1, IFITM3, or a combination thereof in one or more immune cells.

8. The method of claim 7, wherein the one or more immune cells comprise monocytes, CD4+ T cells, cytotoxic T lymphocytes (CTLs), proliferating T cells, NK cells, B cells, plasmablasts, and myeloid dendritic cells.

9. The method of claim 7, wherein the immune response is to a viral infection.

10. The method of claim 9, wherein the viral infection is an HIV infection.

11. A method of modulating an immune response comprising administering an effective amount of:
one or more modulating agents that increases activity or expression of PRF1 and/or GZMB in proliferating CTLs; and/or
one or more modulating agents that increases activity or expression of CCL3 and/or CCL4 in NK cells.

12. The method of claim 11, wherein the immune response is to a viral infection.

13. The method of claim 12, wherein the viral infection is an HIV infection.

14. A method of modulating an immune response, comprising administering one or more modulating agents that induces formation of polyfunctional monocytes.

15. The method of claim 14, wherein the polyfunctional monocytes express one or more anti-viral and inflammatory expression modules.

16. The method of claim 15, wherein the one or more anti-viral and inflammatory expression modules comprise RIG-1, STAT1, HLA-G, APOBEC3B, ISG20, MX1, ISG15, IFI27, or a combination thereof.

17. The method of claim 15, wherein the one or more anti-viral and inflammatory expression modules comprise SLAMF7, DUSP6, WARS, USP18, or a combination thereof.

18. The method of claim 15, wherein the one or more anti-viral and inflammatory expression modules comprise RIG-1, APOBEC3B, MX1, or a combination thereof.

19. A method of treating or preventing a viral infection, the method comprising administrating an effective amount of a modulating agent that modulates expression and/or activity of IL-6, IL-8, IL-17, or a combination thereof to a subject in need thereof.

20. A method of treating or preventing a viral infection, the method comprising administrating an effective amount of:
one or more modulating agents that modulate IFN-α, IFN-γ, or a combination thereof in proliferating T cells, CD4+ T cells, CTLs, monocytes, and NK cells;
one or more modulating agents that modulate IL-15, IL-12, IL-21, or a combination thereof in CTLs, NK cells, and proliferating T cells;
one or more modulating agents that modulate IL-1β, TNF, or a combination thereof in CD4+ T cells; or
any combination thereof.

21. A method of detecting stage of viral infection, comprising:
detecting an expression level of IFI44IL, IFI6, IFIT3, ISG15, XAF1, APOBEC3A, IF27, STAT1 or a combination thereof wherein the expression level relative to a suitable control indicates a hyper-acute or acute stage of viral infection.

22. The method of claim 19, further comprising detection of CXCL10, DEFB1, IFI27L1, or a combination thereof.

23. The method of claim 19, further comprising detection of PARP9, STAT1, or a combination thereof.

24. The method of claim 21, further comprising detection of CD52, TIGIT, TRAC, or a combination thereof.

25. The method of claim 21, further comprising detection of CX3CR1, ICAM2, or a combination thereof.

26. The method of claim 21, further comprising detection of CXCL10, LGALS3BP, or a combination thereof.

27. The method of claim 21, further comprising detection of SLAMF7, DUSP6, WARS, USP18, or a combination thereof.

28. The method of claim 21, further comprising administering one or more modulating agents to modulate expression and/or activity of the detected one or more biomarkers.

29. A method for treating a subject with an infection, the method comprising:
a. detecting expression or activity of one or more biomarkers in one or more types of immune cells; and
b. administering one or more modulating agents to modulate expression and/or activity of the detected one or more biomarkers.

30. A method of screening for one or more agents capable of modulating an immune response, the method comprising:
a. contacting one or more immune cells with one or more candidate modulating agents;
b. detecting expression and/or activity of one or more biomarkers in response to the one or more candidate modulating agents; and
c. selecting modulating agents that cause change in expression and/or activity of one or more biomarkers compared to expression and/or activity of the one or more biomarkers before (a).

31. The method of claim 30, wherein the immune response is to a viral infection.

32. The method of claim 30, wherein the one or more immune cells comprise CD4+ T cells, cytotoxic T lymphocytes (CTLs), proliferating T cells, NK cells, B cells, plasmablasts, myeloid dendritic cells, or a combination thereof.

33. A method of modulating immune response to an infection in a subject, the method comprising contacting CD4+ T cells, monocytes, cytotoxic lymphocytes (CTLs), natural killer (NK) cells, and/or proliferating T cells in the subject with one or more modulating agents, wherein the one or more modulating agents modulate biomarkers in one or more of the following pathways and cell populations:
a. adhesion of T cells, Cdc42 signaling, cytokine signaling, regulation by calpain, endocytic virus entry, or a combination thereof, in CD+4 T cells;
b. allograft rejection signaling, Cdc42 signaling, antigen presentation, IL-4 signaling, OX40 signaling, or a combination thereof, in monocytes;
c. CTL killing or target cells, graft-vs-host disease signaling, Granzyme B signaling, interferon signaling, hypercytokinemia in flu, or a combination thereof, in CTLs;
d. chemokinesis of leukocytes, CTL killing of target cells, innate-adaptive crosstalk, OX40 signaling, dendric cell (DC)-NK crosstalk, or a combination thereof, in NK cells; or
e. innate-adaptive crosstalk, CTL killing of target cells, degranulation of cells, granzyme B signaling, and interferon signaling, or a combination thereof, in proliferating T cells.

34. The method of claim 33, wherein the one or more modulating agents further modulate biomarkers in one or more pathways in Table 6A.

35. The method of claim 33, wherein the one or more modulating agents modulate:
a. biomarkers in cluster 1 of Table 2 in CD4+ T cells;
b. biomarkers in cluster 2 of Table 2 in resting monocytes;
c. biomarkers in cluster 3 of Table 2 in cytotoxic lymphocytes;
d. biomarkers in cluster 4 of Table 2 in inflammatory monocytes;
e. biomarkers in cluster 5 of Table 2 in B cells;
f. biomarkers in cluster 6 of Table 2 in non-classical monocytes;
g. biomarkers in cluster 7 of Table 2 in proliferating T cells;
h. biomarkers in cluster 8 of Table 2 in anti-viral monocytes;
i. biomarkers in cluster 9 of Table 2 in plasmablasts;
j. biomarkers in cluster 10 of Table 2 in CD1C+ dendric cells (DCs);
k. biomarkers in cluster 11 of Table 2 in both anti-viral monocytes and inflammatory monocytes;
l. biomarkers in cluster 12 of Table 2 in CD1C+ plasmacytoid dendric cells (pDCs); or
m. any combination thereof.

36. The method of claim 33, wherein the one or more modulating agents modulate:
a. biomarkers in one or more of the following modules in Tables 3A-3D: P1.B.M1, P1.B.M2, P1.B.M3, P2.B.M1, P2.B.M2, P2.B.M3, P2.B.M4, P3.B.M1, P3.B.M2, P3.B.M3, P3.B.M4, P3.B.M5, P4.B.M1, P4.B.M2, in B cells;
b. biomarkers in one or more of the following modules in Tables 3A-3D: P1.CD4.M1, P1.CD4.M2, P1.CD4.M3, P1.CD4.M4, P1.CD4.M5, P1.CD4.M6, P1.CD4.M7, P2.CD4.M1, P2.CD4.M2, P3.CD4.M1, P3.CD4.M2, P3.CD4.M3, P3.CD4.M4, P4.CD4.M1, P4.CD4.M2, P4.CD4.M3, in CD4+ T cells;
c. biomarkers in one or more of the following modules in Tables 3A-3D: P1. P1.Prolif.T.M1, P1.Prolif.T.M2, P1.Prolif.T.M3, P2.Prolif.T.M1, P2.Prolif.T.M2, P3.Prolif.T.M1, P3.Prolif.T.M2, P3.Prolif.T.M3, P4.Prolif.T.M1, P4.Prolif.T.M2, P4.Prolif.T.M3, in proliferating T cells;
d. biomarkers in one or more of the following modules in Tables 3A-3D: P1.DC.M1, P1.DC.M2, P2.DC.M1, P2.DC.M2, P2.DC.M3, P3.DC.M1, P3.DC.M2, P4.DC.M1, P4.DC.M2, in dendric cells;
e. biomarkers in one or more of the following modules in Tables 3A-3D: P1.Mono.M1, P1.Mono.M2, P1.Mono.M3, P1.Mono.M4, P1.Mono.M5, P1.Mono.M6, P1.Mono.M7, P1.Mono.M8, P2.Mono.M1, P2.Mono.M2, P2.Mono.M3, P2.Mono.M4, P2.Mono.M5, P3.Mono.M1, P3.Mono.M2, P3.Mono.M3, P3.Mono.M4, P3.Mono.M5, P3.Mono.M6, P3.Mono.M7, P3.Mono.M8, P4.Mono.M1, P4.Mono.M2, P4.Mono.M3, P4.Mono.M4, P4.Mono.M5, P4.Mono.M6, in monocytes;
f. biomarkers in one or more of the following modules in Tables 3A-3D: P1.NK.M1, P1.NK.M2, P1.NK.M3, P1.NK.M4, P2.NK.M1, P2.NK.M2, P2.NK.M3, P2.NK.M4, P3.NK.M1, P3.NK.M2, P3.NK.M3, P3.NK.M4, P3.NK.M5, P3.NK.M6, P4.NK.M1, P4.NK.M2, P4.NK.M3, P4.NK.M4, in NK cells; or
g. biomarkers in one or more of the following modules in Tables 3A-3D: P2.PB.M1, P2.PB.M2, P3.PB.M1, P4.PB.M1, P4.PB.M2, in plasmablasts.

37. The method of claim 33, wherein the one or more modulating agents modulate IFI27, IFI44L, IFI6, IFIT3, ISG15, XAF1, or a combination thereof.

38. The method of claim 33, wherein the one or more modulating agents modulate CXCL10, DEFB1, IFI27L1, or a combination thereof, in monocytes.

39. The method of claim 33, wherein the one or more modulating agents modulate PARP9, STAT1, or a combination thereof, in dendric cells.

40. The method of claim 33, wherein the one or more modulating agents modulate CD52, TIGIT, TRAC, or a combination thereof, in CD4+ T cells.

41. The method of claim 33, wherein the one or more modulating agents modulate CX3CR1, ICAM2, or a combination thereof, in NK cells.

42. The method of claim 33, wherein the one or more modulating agents modulate B2M, S100A4, KLF6, ANXA1, ITGB1, SYNE2, EZR, S100A6, AHNAK, CD52, IL32, or a combination thereof, in CD4+ T cells.

43. The method of claim 33, wherein the one or more modulating agents modulate HLA-DQB1, HLA-DPB1, HLA-DPA1, CD74, HLA-DRA, HLA-DQA1, HLA-DRB1, CD52, or a combination thereof, in monocytes.

44. The method of claim 33, wherein the one or more modulating agents modulate GZMB, GZMH, GNLY, FGFBP2, NKG7, PRF1, KLRD1, CCL5, or a combination thereof, in CTLs.

45. The method of claim 33, wherein the one or more modulating agents modulate GNPTAB, PRSS23, GZMB, GNLY, B2M, FGFBP2, NKG7, PRF1, LGALS1, TMSB4X, TMSB10, CST7, or a combination thereof in NK cells.

46. The method of claim 33, wherein the one or more modulating agents modulate GPR56, CST7, GZMA, KLRD1, FGFBP2, GZMH, NKG7, CCL5, CCL4, CTSW, HOPX, PRF1, GZMBGNLY, PLEK, ID2, CD8A, UBB, SPON2, FCGR3A, or a combination thereof, in proliferating T cells.

47. The method of claim 33, wherein the one or more modulating agents modulate PRF1, GZMB, GNYL, NKG7, FGFBP2, or a combination thereof, in CTLs, NK cells, and proliferating T cells.

48. The method of claim 33, wherein the one or more modulating agents modulate CD52 in CD4+ T cells and monocytes.

49. The method of claim 33, wherein the one or more modulating agents modulate B2M in CD4+ T cells and NK cells.

50. The method of claim 33, wherein the one or more modulating agents modulate GZMH, CCL5, KLRD1, or a combination thereof, in CTLs and proliferating T cells.

51. The method of claim 33, wherein the one or more modulating agents modulate CST7 in NK cells and proliferating T cells.

52. The method of claim 33, wherein the one or more modulating agents modulate PRF1, GZMB, GNYL, NKG7, FGFBP2, or a combination thereof, in NK cells, proliferating T cells, and CTL.

53. The method of claim 33, further comprising contacting monocytes with the one or more modulating agents modulate genes in the following pathways: IFNα response, IFNγ response, complement, inflammatory response, TNF signaling via NF-κB, LPS stimulation, anti-TREM1 stimulation, PI3K inhibition, NFκB inhibition of HCMV inflammatory monocytes, or a combination thereof.

54. The method of claim 33, further comprising contacting monocytes with the one or more modulating agents that modulate SERPINB2, CXCL3, CCL4, CCL3, IL1B, RPL5, STAT2, ICAM2, MIF, HLA-A, APOBEC3G, CD302, RPS16, SLAMF7, DUSP6, WARS, USP18, FCGR1B, CXCL1, CD300E, CCR1, IL6, CCL2, RIG-1, STAT1, HLA-G, APOBEC3B, ISG20, MX1, ISG15, IF127, or a combination thereof.

55. The method of claim 33, further comprising modulating:
a. biomarkers in clusters 0 in Table 7C in CD8+ T cells,
b. biomarkers in clusters 1 in Table 7C in hyper-proliferative CD8+ T cells,
c. biomarkers in clusters 2 in Table 7C in naïve CD4+ T cells,
d. biomarkers in clusters 30 in Table 7C in CD8−/TRDC+/FCGR3A+ T cells, or
e. a combination thereof.

56. The method of claim of 55, wherein the one or more modulating agents modulate CD8A, TNFAIP3, RGS1, HIST1H4C, PCNA, TOP2A, CCR7, ISG20, CD27, GZMK, TRDC, KLRF1, GZMB, XCL2, FCGR3A, or a combination thereof.

57. The method of claim of 55, wherein the one or more modulating agents modulate one or more biomarkers in Table 7C.

58. The method of claim 33, wherein the one or more modulating agents modulate IL7R, LTB, TRBC2, LYZ, MNDA, CD14, NKG7, CCL5, GZMB, IL8, IL1B, CXCL2, MS4A1, CD79A, CD74, CD16, LST1, RHOC, STMN1, MKI67, CD8A, TNFSF10, ISG15, APOBEC3A, IGJ, IGHG1, MZB1, CD1C, HLA-DRA, CCL2, CCL4, UGCG, SERPINF1, or a combination thereof.

59. The method of claim 33, further comprising contacting resting monocytes, inflammatory monocytes, CD16+ monocytes, anti-viral monocytes, anti-viral/inflammatory monocytes, CD1C+ dendric cells, plasmacytoid dendric cells, B cells, plasmablasts, or a combination thereof.

60. The method of claim 33, further comprising contacting plasmacytoid dendric cells with the one or more modulating agents that modulate IFITM1, IFI44L, ISG15, LY6E, IFI6, SAMD9L, IFI44, MX1, OAS3, EPSTI1, EEF1A1, SFT2D2, FOSB, FOS, ANKRD36BP1, UCP2, RPLP0, RHOA, RPL9, PSAP, or a combination thereof.

61. The method of claim 33, further comprising contacting B cells with the one or more modulating agents that modulate biomarkers in one or more of following pathways: B cell development, BCR signaling, psoriatic arthritis, proliferation of immune cells, or atherosclerosis signaling.

62. The method of claim 33, further comprising contacting inflammatory monocytes with the one or more modulating agents that modulate BCL2A1, C5AR1, CCL3, CO83, CTSS, CXCL2, CXCL3, DUSP2, EREG, FTH1, G0S2, GADD45B, GPR183, IER3, IL 1 B, IL8, NAM PT, NFKBIA, NFKBIZ, NLRP3, PDE4B, PLAUR, PPP1R15A, PTGS2, SAMSN1, SERPINB2, SOD2, SRGN, THBS1, TIPARP, TNFAIP3, TNFAIP6, ZFP36, or a combination thereof.

63. The method of claim 33, further comprising contacting anti-viral monocytes with the one or more modulating agents that modulate APOBEC3A, APOBEC3B, B2M, CXCL10, EPSTl1, GBP1, GBP4, IFl27, IFl27L 1, IFl44L, IFl6, IFIT1, IFIT2, IFIT3, IFITM1, IFITM3, IGJ, ISG15, ISG20, L Y6E, MARCKS, MX1, NT5C3A, OAS1, PLAC8, RSAD2, SAT1, TNFSF10, TXNIP, XAF1, or a combination thereof.

64. The method of claim 33, further comprising contacting CTLs with the one or more modulating agents that modulate one or more biomarkers in Table 7A.

65. The method of claim 33, further comprising contacting CTLs and/or proliferating T cells with the one or more modulating agents that modulate one or more biomarkers in Table 7B.

66. The method of claim 33, further comprising contacting proliferating T cells with the one or more modulating agents that modulate one or more TRBV28, TRAV4, TRBV20-1, or a combination thereof.

67. The method of claim 33, wherein the one or more modulating agents further modulate CIITA, EBI3, G-CSF, HRAS, IL6, IFNA, IL10, Ig, IL12, IL4, IL2, TBX21, IFNG, IL21, IL27, STAT1, IL15, PDCD1, IL18, or a combination thereof.

68. The method of claim 33, wherein the one or more modulating agents further modulate IFNG, TGFB1, STAT1, IFNA, PRDM1, SMARCA4, TP53, CIITA, G-CSF, EBI3, IL27, or a combination thereof.

69. The method of claim 33, wherein the one or more modulating agents further modulate IL2, IFNA, IFNG, TNF, KRAS, CD3, IL15, IL4, IL1B, TGFB1, OSM, or a combination thereof.

70. The method of claim 33, wherein the modulating agents further modulate IL4, G-CSF, IL2, IL27, IFNA, IFNG, IL6, STAT3, IL12, Ig, IL15, IL21, TBX21, or a combination thereof.

71. The method of claim 33, wherein the modulating agents further modulate G-CSF, IL12, IFNA, IL18, CD40LG, IL4, Ig, IL15, IL2, IFNG, STAT1, IL27, PDCD1, IL21, IL6, TBX21, STAT3, TGFB1, or a combination thereof.

72. The method of claim 33, further comprising contacting CD4+ T cells with the one or more modulating agents that modulate IFNA, OSM, IFNG, TNF, CD3, IL15, IL1B, TGFB1, KRAS, IL2, IL4, IL6, or a combination thereof.

73. The method of claim 33, further comprising contacting monocytes with the one or more modulating agents that modulate CIITA, G-CSF, EBI3, IL27, IFNG, IFNA, STAT1, TGFB1, PRDM1, SMARCA4, TP53, or a combination thereof.

74. The method of claim 33, further comprising contacting NK cells with the one or more modulating agents that modulate CIITA, IFNA, IFNG, STAT1, IL27, HRAS, IL15, EBI3, G-CSF, IL18, IL10, IL4, IL2, TBX21, PDCD1, IL21, IL6, Ig, IL12, or a combination thereof.

75. The method of claim 33, further comprising contacting CTLs with the one or more modulating agents that modulate G-CSF, IL4, IFNG, IFNA, IL15, IL6, STAT3, IL27, IL21, Ig, IL2, TBX21, IL18, IL12, TGFB1, PDCD1, or a combination thereof.

76. The method of claim 33, further comprising contacting proliferating T cells with the one or more modulating agents that modulate G-CSF, IL12, IFNA, IL18, IL15, TBX21, PDCD1, STAT3, IFNG, STAT1, IL27, IL21, IL6, Ig, IL2, IL4, TGFB1, CD40LG or a combination thereof.

77. The method of claim 33, wherein the one or more modulating agents modulate one or more biomarkers in Table 6B.

78. The method of claim 33, wherein the one or more modulating agents are administered 1 week, 2 weeks, 3 weeks, 4 weeks, 6 months, or 1 year after the infection.

79. The method of claim 33, wherein the subject does not have the infection.

80. The method of claim 33, wherein the infection is a virus infection.

81. The method of claim 33, wherein the infection is an HIV infection.

82. The method of claim 33, wherein the infection is an acute infection or hyper-acute infection.

83. The method of claim 33, wherein the infection is a chronic infection.

84. The method of claim 33, wherein the subject has viremia.

85. The method of any of the proceeding claims, wherein the one or more modulating agents comprises a Cas protein and one or more guide molecules comprising guide sequences capable of forming a complex with the Cas protein and directing binding of the complex to one or more target polynucleotides, thereby modulating one or more genes comprising the one or more target polynucleotides.

86. The method of claim 85, wherein the Cas protein is a class 2, Type II Cas protein.

87. The method of claim 86, wherein the class 2, Type II Cas protein is Cas9 or a variant thereof.

88. The method of claim 85, wherein the Cas protein is a class 2, Type V Cas protein.

89. The method of claim 88, wherein the class 2, Type V protein is Cas12a, Cas12b, Cas12c, or Cas12d.

90. The method of claim 85, wherein the Cas protein is a class 2, Type VI Cas protein.

91. The method of claim 90, wherein the class 2, Type VI Cas protein is Cas13a, Cas13b, Cas13c, or Cas13d.

92. The method of any one of the proceeding claims, wherein the one or more modulating agents modulate RNA molecules encoded by one or more target genes.
